(19) United States
(12) Patent Application Publication
(10) Pub. No.: US 2020/0064357 A1
(43) Pub. Date: Feb. 27, 2020

(54) METHODS FOR DETERMINING AND MONITORING GASTROINTESTINAL INFLAMMATION

(71) Applicant: Protagonist Therapeutics, Inc., Newark, CA (US)

(72) Inventors: Xiaoli CHENG, Mountain View, CA (US); Roopa TARANATH, Newark, CA (US)

(21) Appl. No.: 16/348,293

(22) PCT Filed: Nov. 9, 2017
(86) PCT No.: PCT/US2017/060944

§ 371 Date: May 8, 2019

Related U.S. Application Data
(60) Provisional application No. 62/419,683, filed on Nov. 9, 2016.
(60) Provisional application No. 62/430,088, filed on Dec. 5, 2016.
(60) Provisional application No. 62/502,452, filed on May 5, 2017.

(51) Int. Cl. G01N 33/68

(52) U.S. Cl. CPC G01N 33/6869; G01N 2800/52; G01N 2800/06

(57) ABSTRACT

The present invention provides methods for determining levels of biomarkers of inflammation in samples from human patients, e.g., patients undergoing treatment with a peptide inhibitor of interleukin-23 receptor. Such methods are useful, for example, in monitoring the therapeutic effect of treatment of an inflammatory disease or disorder with a peptide inhibitor of interleukin-23 receptor.

### CROSS-REFERENCE TO RELATED APPLICATIONS

This application claims priority to U.S. Provisional Application No. 62/419,683, filed on Nov. 9, 2016; U.S. Provisional Application No. 62/430,088, filed on Dec. 5, 2016; and U.S. Provisional Application No. 62/502,452, filed on May 5, 2017; all of which are incorporated by reference herein in their entireties.

### TECHNICAL FIELD

The present invention relates generally to methods for determining an amount of a biomarker of inflammation in a biological sample, e.g., a biological sample obtained from a subject treated for an inflammatory disease or disorder with a peptide inhibitor of an interleukin-23 receptor. Such methods are useful, for example, in monitoring inflammation and the effectiveness of such treatment.

### BACKGROUND

The interleukin-23 (IL-23) cytokine has been implicated as playing a crucial role in the pathogenesis of autoimmune inflammation and related diseases and disorders, such as multiple sclerosis, asthma, rheumatoid arthritis, psoriasis, and inflammatory bowel diseases (IBDs), e.g., ulcerative colitis and Crohn's disease. Studies in acute and chronic mouse models of IBD revealed a primary role of IL-23R and downstream effector cytokines in disease pathogenesis. IL-23R is expressed on various adaptive and innate immune cells including Th17 cells, γδ T cells, natural killer (NK) cells, dendritic cells, macrophages, and innate lymphoid cells, which are found abundantly in the intestine. At the intestine mucosal surface, the gene expression and protein levels of IL-23R are found to be elevated in IBD patients. It is believed that IL-23 mediates this effect by promoting the development of a pathogenic CD4⁺ T cell population that produces IL-6, IL-17, and tumor necrosis factor (TNF).

IL-23 is a heterodimer composed of a unique p19 subunit and the p40 subunit of IL-12, which is a cytokine involved in the development of interferon-γ (IFN-γ)-producing T helper 1 (T_{H}1) cells. Although IL-23 and IL-12 both contain the p40 subunit, they have different phenotypic properties. For example, animals deficient in IL-12 are susceptible to inflammatory autoimmune diseases, whereas IL-23 deficient animals are resistant, presumably due to a reduced number of CD4⁺ T cells producing IL-6, IL-17, and TNF in the CNS of IL-23-deficient animals. IL-23 binds to IL-23R, which is a heterodimeric receptor composed of IL-12Rβ1 and IL-23R subunits. Binding of IL-23 to IL-23R activates the Jak-stat signaling molecules, Jak2, Tyk2, and Stat1, Stat 3, Stat 4, and Stat 5, although Stat4 activation is substantially weaker and different DNA-binding Stat complexes form in response to IL-23 as compared with IL-12. IL-23R associates constitutively with Jak2 and in a ligand-dependent manner with Stat3. In contrast to IL-12, which acts mainly on naive CD4(+) T cells, IL-23 preferentially acts on memory CD4(+) T cells.

Efforts have been made to identify therapeutic moieties that inhibit the IL-23 pathway, for use in treating IL-23-related diseases and disorders. A number of antibodies that bind to IL-23 or IL-23R have been identified, including ustekinumab, a humanized antibody that binds IL-23, which has been approved for the treatment of psoriasis. Clinical trials in Crohn's Disease or psoriasis with ustekinumab and briakinumab (which target the common p40 subunit) and tildrakizumab, guselkumab, MEDI2070, and BI-655066 (which target the unique p19 subunit of IL-23) highlight the potential of IL-23 signaling blockade in treatment of human inflammatory diseases. More recently, polypeptide inhibitors that bind to IL-23R and inhibit the binding of IL-23 to IL-23R have been identified (see, e.g., US Patent Application Publication No. US2013/0029907).

However, there remains a need in the art for methods of monitoring the effectiveness of treatment with therapeutic moieties that inhibit the IL-23 pathway, including peptide inhibitors that bind to IL-23R, e.g., to optimize dosing regimens for patients.

### BRIEF SUMMARY OF THE INVENTION

The present disclosure provides methods for determining the level of one or more biomarker in a biological sample, and particular applications of such methods in determining the presence of inflammatory disease in a subject and treating a subject.

In one embodiment, the disclosure provides a method of determining a level of one or more biomarker in a biological sample, comprising: contacting the sample with a reagent that binds the biomarker; and detecting the presence or absence of, or an amount of, the reagent bound to the biomarker, and thereby determining the level of the biomarker in the biological sample. In particular embodiments, the biological sample is feces or a liquid matrix, wherein the liquid matrix is optionally serum, plasma, blood, or urine. In certain embodiments, the one or more biomarker is selected from the group consisting of: LCN2/lipocalin, MPO, IL-1β, IL-6, IL-17A, IL-17F, IL-22, and pSTAT3. In some embodiments, the biological sample was obtained from a subject to whom a peptide inhibitor of an interleukin-23 receptor had been administered. In certain embodiments, said detecting is performed using an immunoassay, optionally selected from the group consisting of cloned enzyme donor immunoassay (CEDIA), turbidity assay, and competitive ELISA.

In another embodiments, the disclosure provides a method for determining the efficacy of treatment of a subject having an inflammatory disease or disorder with a peptide inhibitor of interleukin-23 receptor (IL-23R), comprising: contacting a biological sample obtained from the subject after treatment with the IL-23R inhibitor with a reagent that binds the biomarker; and detecting the presence or absence of, or an amount of, the reagent bound to the biomarker, and thereby determining the level of the biomarker in the biological sample, wherein if the level is reduced as compared to a pre-determined cut-off value or the level of the biomarker before treatment with the IL-23R inhibitor, it indicates that the treatment is efficacious, and wherein if the level is the same or increased as compared to a pre-determined cut-off value or the level of the biomarker before treatment with the IL-23R inhibitor, it indicates that the treatment is not efficacious. In particular embodiment, the biological sample is feces or a liquid matrix, wherein the liquid matrix is optionally serum, plasma, blood, or urine. In certain embodiments, the one or more biomarker is selected from the group consisting of: LCN2/lipocalin, MPO, IL-1β, IL-6, IL-17A, IL-17F, IL-22, and pSTAT3.

In further related embodiments, the disclosure provides a method of treating an inflammatory disease or disorder in a subject in need thereof, comprising: (i) providing to the subject an effective amount of a peptide inhibitor of an interleukin-23 receptor; (ii) waiting for a first period of time; and (iii) determining the level of a biomarker in a biological sample obtained from the subject according to the methods disclosed herein. In certain embodiments, the method further comprises: providing an additional amount of the peptide inhibitor, or an amount of the peptide inhibitor greater than the effective amount of step (i), to the subject after step (iii), if the determined level of the biomarker is equal to or above a cut-off value; or not providing an additional amount of the peptide inhibitor, or providing an additional amount of the peptide inhibitor less than the effective amount of step (i) to the subject after step (iii), if the determined level of the biomarker is below a cut-off value.

In another embodiment, the disclosure provides a method for determining or monitoring the efficacy of treatment of a subject having an inflammatory disease or disorder, optionally gastrointestinal inflammation, with a peptide inhibitor of interleukin-23 receptor (IL-23R), comprising: contacting a biological sample obtained from the subject during or after treatment with the IL-23R inhibitor with one or more reagent that binds one or more biomarker of inflammation, wherein one or more of the biomarkers are selected from the group consisting of: myeloperoxidase (MPO), interleukin-1β (IL-1β), interleukin-6 (IL-6), interleukin-22 (IL-22), interleukin-17A (IL-17A), interleukin-17F (IL-17F), lipocalin 2 (LCN2), matrix metallopeptidase 9 (MMP9), S100 calcium-binding protein A8 (S100A8), microRNA-223-3p (miR223-3p), and phosphorylated signal transducer and activator of transcription 3 (pSTAT3) proteins, polynucleotides encoding any of the proteins, and polynucleotides comprising a region complementary to microRNA-223-3p or any of the polynucleotides that encode any of the proteins; and detecting the presence or absence of, or determining an amount of, the reagent bound to the biomarker, and thereby determining the level of the one or more biomarker in the biological sample, wherein if the level is reduced as compared to a pre-determined cut-off value or the level of the biomarker before treatment with the IL-23R inhibitor, it indicates that the treatment is efficacious, and wherein if the level is the same or increased as compared to a pre-determined cut-off value or the level of the biomarker before treatment with the IL-23R inhibitor, it indicates that the treatment is not efficacious.

Another related embodiment includes a method for determining or monitoring the efficacy of treatment of a subject having an inflammatory disease or disorder, optionally gastrointestinal inflammation, with a peptide inhibitor of interleukin-23 receptor (IL-23R), comprising: contacting a biological sample obtained from the subject during or after treatment with the IL-23R inhibitor with one or more reagent that binds one or more biomarker of inflammation, wherein one or more of the biomarkers is a Claudin 8 (CLDN8) biomarker, optionally a protein, a polynucleotide that encodes the CLDN8 protein, or a polynucleotide comprising a region complementary to the polynucleotide that encodes the CLDN8 protein; and detecting the presence or absence of, or determining an amount of, the reagent bound to the CLDN8 biomarker, and thereby determining the level of the CLDN8 biomarker in the biological sample, wherein if the level is increased as compared to a pre-determined cut-off value or the level of the CLDN8 biomarker before treatment with the IL-23R inhibitor, it indicates that the treatment is efficacious, and wherein if the level is the same or reduced as compared to a pre-determined cut-off value or the level of the CLDN8 biomarker before treatment with the IL-23R inhibitor, it indicates that the treatment is not efficacious.

In particular embodiments of any of the methods disclosed herein, the biological sample is a gastrointestinal tissue sample, optionally a colon tissue sample, and one or more of the biomarker is selected from the group consisting of: LCN2, MPO, IL-1β, IL-6, IL-17A, IL-17F, IL-22, pSTAT3, S100A8, CLDN8, and miR-223-3p. In certain embodiments, the biological sample is feces, and one or more biomarker is selected from the group consisting of: LCN2, MPO, and MMP9. In certain embodiments, the biological sample is a liquid matrix, wherein the liquid matrix is optionally serum, plasma, blood, or urine. In certain embodiments, the liquid matrix is serum, and one of the biological markers is LCN2, MPO, and MMP9, optionally LCN2.

In particular embodiments of any of the methods disclosed herein, the biomarker is a polypeptide. In certain embodiments, detecting is performed using an immunoassay, optionally selected from the group consisting of cloned enzyme donor immunoassay (CEDIA), turbidity assay, and competitive ELISA.

In particular embodiments of any of the methods disclosed herein, the biomarker is a polynucleotide. In certain embodiments, the detecting is performed by polymerase chain reaction (PCR), optionally quantitative reverse transcription-PCR (RT-PCR), or RNA sequencing.

Particular embodiments of any of the methods disclosed herein further comprise determining the levels of the one or more biomarkers before treatment.

In a further embodiment, the disclosure provides a method of determining a level of one or more biomarker of intestinal inflammation in a serum sample, wherein one or more of the biomarkers is lipocalin 2 (LCN2), comprising: contacting the serum sample with a reagent that binds LCN2; and detecting the presence or absence of, or determining an amount of, the reagent bound to the one or more biomarker, and thereby determining the level of the one or more biomarker in the biological sample.

In another embodiment, the present disclosure provides a method of determining the presence of an inflammatory disease or disorder, optionally intestinal inflammation, in a subject, comprising: contacting a serum sample obtained from the subject with a reagent that binds lipocalin 2 (LCN2); and detecting the presence or absence of, or determining an amount of, the reagent bound to the LCN2, and thereby determining the level of LCN2 in the serum sample, wherein if the level of LCN2 in the serum sample is greater than 294 ng/mL, intestinal inflammation is determined to be present in the subject with a sensitivity of 89% and a specificity of 95%. In certain embodiments, the subject was previously treated with a peptide inhibitor of an interleukin-23 receptor. In certain embodiments, said detecting is performed using an immunoassay, optionally selected from the group consisting of cloned enzyme donor immunoassay (CEDIA), turbidity assay, and competitive ELISA.

In a further related embodiments, the disclosure provides a method of determining a level of one or more biomarker of intestinal inflammation in a feces sample, wherein one or more of the biomarkers is lipocalin 2 (LCN2), myeloperoxidase (MPO), or matrix metallopeptidase 9 (MMP9), comprising: extracting proteins from the feces to produce extracted fecal proteins; contacting the extracted fecal proteins with one or more reagent that binds the one or more biomarkers; and detecting the presence or absence of, or determining an amount of, the reagent bound to the one or more biomarker, and thereby determining the level of the one or more biomarker in the feces sample.

Another embodiment disclosed herein is a method of determining the presence of an inflammatory disease or disorder, optionally intestinal inflammation, in a subject, comprising: extracting proteins from a feces sample obtained from the subject to produce extracted fecal proteins; contacting the extracted fecal proteins with one or more reagent that binds to one or more biomarkers of inflammation, wherein one of the biomarkers is lipocalin 2 (LCN2), myeloperoxidase (MPO), or matrix metallopeptidase 9 (MMP9); and detecting the presence or absence of, or determining an amount of, the reagent bound to the one or more biomarkers, and thereby determining the level of the one or more biomarkers in the feces sample, wherein if the level of the one or more biomarkers is greater than a predetermined cut-off value or significantly greater than an average value obtained using feces obtained from healthy donors, intestinal inflammation is determined to be present in the subject. In certain embodiments, the subject was previously treated with a peptide inhibitor of an interleukin-23 receptor. In particular embodiments, detecting is performed using an immunoassay, optionally selected from the group consisting of cloned enzyme donor immunoassay (CEDIA), turbidity assay, and competitive ELISA.

The disclosure further provides a method of treating an inflammatory disease or disorder in a subject in need thereof, comprising: (i) providing to the subject an effective amount of a peptide inhibitor of an interleukin-23 receptor; (ii) waiting for a first period of time; (iii) after the first period of time, contacting a biological sample obtained from the subject with one or more reagent that binds one or more biomarker of inflammation, wherein one or more of the biomarkers are selected from the group consisting of: myeloperoxidase (MPO), interleukin-1β (IL-1β), interleukin-6 (IL-6), interleukin-22 (IL-22), interleukin-17A (IL-17A), interleukin-17F (IL-17F), lipocalin 2 (LCN2), matrix metallopeptidase 9 (MMP9), S100 calcium-binding protein A8 (S100A8), microRNA-223-3p (miR223-3p), Claudin 8 (CLDN8), and phosphorylated signal transducer and activator of transcription 3 (pSTAT3) proteins, polynucleotides encoding any of the proteins, and polynucleotides comprising a region complementary to microRNA-223-3p or any of the polynucleotides that encode any of the proteins; and (iv) detecting the presence or absence of, or determining an amount of, the reagent bound to the biomarker, and thereby determining the level of the one or more biomarker in the biological sample. In particular embodiments, the method further comprises: providing an additional amount of the peptide inhibitor, or an amount of the peptide inhibitor greater than the effective amount of step (i), to the subject after step (iv), if the determined level of the one or more biomarker is equal to or above a pre-determined cut-off value if the biomarker is MPO, IL-1β, IL-6, IL-22, IL-17A, IL-17F, LCN2, MMP9, S100A8, miR223-3p, or pSTAT3, or equal to or below a pre-determined cut-off value if the biomarker is CLDN8; or not providing an additional amount of the peptide inhibitor, or providing an additional amount of the peptide inhibitor less than the effective amount of step (i) to the subject after step (iv), if the determined level of the biomarker is below a pre-determined cut-off value if the biomarker is MPO, IL-1β, IL-6, IL-22, IL-17A, IL-17F, LCN2, MMP9, S100A8, miR223-3p, or pSTAT3, or above a pre-determined cut-off value if the biomarker is CLDN8.

In certain embodiments of any of the methods disclosed herein related to an inflammatory disease or disorder, the inflammatory disease or disorder is an Inflammatory Bowel Disease (IBD), ulcerative colitis, Crohn's disease, Celiac disease (nontropical Sprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-1, chronic granulomatous disease, glycogen storage disease type 1b, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, or graft versus host disease.

In particular embodiments of any of the methods disclosed herein related to a peptide inhibitor, the peptide inhibitor has a structure or sequence of Formula (Xa), (I), (II), or (III), or a pharmaceutically acceptable salt thereof, or is disclosed in any of Tables 2-6. In certain embodiments, the peptide inhibitor or pharmaceutically acceptable salt thereof comprises or consists of an amino acid sequence or structure as follows:

[Palm]-[isoGlu]-[PEG4]-[Pen]-NTWQ-[Pen]-[Phe[4-(2-aminoethoxy)]-[2-Nal]-[Aib]-[Lys(Ac)]-NNNH₂;

Ac-[Pen]-NTWQ-[Pen]-[Phe[4-(2-aminoethoxy)]-[2-Nal]-[Aib]-[Lys(PEG4-isoGlu-Palm)]-NN-NH₂;

Ac-[Pen]-QTWQ-[Pen]-Phe(4-CONH₂)-[2-Nal]-[α-MeLys(Ac)]-[Lys(Ac)]-NN-NH₂;

[Octanyl]-[IsoGlu]-[PEG4]-[Pen]-NTWQ-[Pen]-[Phe[4-(2-aminoethoxy)]-[2-Nal]-[Aib]-[Lys(Ac)]-NN-NH₂;

[Octanyl]-[PEG4]-[Pen]-NTWQ-[Pen]-[Phe[4-(2-aminoethoxy)]-[2-Nal]-[Aib]-[Lys(Ac)]-NN-NH₂;

[Palm]-[PEG4]-[Pen]-NTWQ-[Pen]-[Phe[4-(2-aminoethoxy)]-[2-Nal]-[Aib]-[Lys(Ac)]-NN-NH₂;

Ac-[Pen]-NTWQ-[Pen]-[Phe[4-(2-aminoethoxy)]-[2-Nal]-[Aib]-[Lys(PEG4-Octanyl)]-NN-NH₂;

Ac-[Pen]-NTWQ-[Pen]-[Phe[4-(2-aminoethoxy)]-[2-Nal]-[Aib]-[Lys(PEG4-Palm)]-NN-NH₂;

Ac-[Pen]-NTWQ-[Pen]-[Phe[4-(2-aminoethoxy)-(PEG4-Palm)]-[2-Nal]-[Aib]-[Lys(Ac)]NN-NH₂;

Ac-[Pen]-NTWQ-[Pen]-[Phe[4-(2-aminoethoxy)-(PEG4-Lauryl)]-[2-Nal]-[Aib]-[Lys(Ac)]-NN-NH₂;

Ac-[Pen]-QTWQ-[Pen]-Phe(4-CONH₂)-[2-Nal]-[α-MeLys(PEG4-Palm)-[Lys(Ac)]-NN-NH₂;

Ac-[Pen]-QTWQ-[Pen]-Phe(4-CONH₂)-[2-Nal]-[α-MeLys(PEG4-Lauryl)]-[Lys(Ac)]-NN-NH₂;

Ac-[Pen]-NTWQ-[Pen]-[Phe[4-(2-aminoethoxy)-(PEG4-IsoGlu-Palm)]-[2-Nal]-[Aib]-[Lys(Ac)]-NN-NH₂;

Ac-[Pen]-NTWQ-[Pen]-[Phe[4-(2-aminoethoxy)-(PEG4-IsoGLu-Lauryl)]-[2-Nal]-[Aib]-[Lys(Ac)]-NN-NH₂;

Ac-[Pen]-QTWQ-[Pen]-Phe(4-CONH₂)-[2-Nal]-[α-MeLys(PEG4-IsoGlu-Palm)]-[Lys(Ac)]-NN-NH₂;

Ac-[Pen]-QTWQ-[Pen]-Phe(4-CONH₂)-[2-Nal]-[α-MeLys(PEG4-IsoGlu-Lauryl)]-[Lys(Ac)]-NN-NH₂;

Ac-[Pen]-QTWQ-[Pen]-Phe(4-CONH₂)-[2-Nal]-[α-MeLys(IVA)]-[Lys(Ac)]-NN-NH₂;

Ac-[Pen]-QTWQ-[Pen]-Phe(4-CONH₂)-[2-Nal]-[α-MeLys(Biotin)]-[Lys(Ac)]-NN-NH₂;

Ac-[Pen]-QTWQ-[Pen]-Phe(4-CONH₂)-[2-Nal]-[α-MeLys(Octanyl)]-[Lys(Ac)]-NN-NH₂;

Ac-[Pen]-[Lys(IVA)]-TWQ-[Pen]-[Phe[4-(2-aminoethoxy)]-[2-Nal]-[Aib]-[Lys(Ac)]-NN-NH₂;

Ac-[Pen]-NTWQ-[Pen]-[Phe[4-(2-aminoethoxy)]-[2-Nal]-[Aib]-[Lys(Ac)]-[Lys(IVA)]-N-NH₂;

Ac-[Pen]-[Lys(Biotin)]-TWQ-[Pen]-[Phe[4-(2-aminoethoxy)]-[2-Nal]-[Aib]-[Lys(Ac)]-NN-NH₂;

Ac-[Pen]-NTWQ-[Pen]-[Phe[4-(2-aminoethoxy)]-[2-Nal]-[Aib]-[Lys(Ac)]-[Lys(Biotin)]-N-NH₂;

Ac-[Pen]-[Lys(Octanyl)]-TWQ-[Pen]-[Phe[4-(2-aminoethoxy)]-[2-Nal]-[Aib]-[Lys(Ac)]-NN-NH₂;

Ac-[Pen]-NTWQ-[Pen]-[Phe[4-(2-aminoethoxy)]-[2-Nal]-[Aib]-[Lys(Ac)]-[Lys(octanyl)]-N-NH₂;

Ac-[Pen]-[Lys(Palm)]-TWQ-[Pen]-[Phe[4-(2-aminoethoxy)]-[2-Nal]-[Aib]-[Lys(Ac)]-NN-NH₂;

Ac-[Pen]-NTWQ-[Pen]-[Phe[4-(2-aminoethoxy)]-[2-Nal]-[Aib]-[Lys(Ac)]-Lys(Palm)]-N-NH₂;

Ac-[Pen]-[Lys(PEG8)]-TWQ-[Pen]-[Phe[4-(2-aminoethoxy)]-[2-Nal]-[Aib]-[Lys(Ac)]-NN-NH₂;

Ac-[Pen]-NTWQ-[Pen]-[Phe[4-(2-aminoethoxy)]-[2-Nal]-[Aib]-[Lys(Ac)]-[Lys(PEG8)]-N-NH₂;

Ac-[Pen]-K(Peg11-Palm)TWQ-[Pen]-[Phe[4-(2-aminoethoxy)]-[2-Nal]-[Aib]-[Lys(Ac)]-NN-NH₂;

Ac-[Pen]-NTWQ-[Pen]-[Phe[4-(2-aminoethoxy)]-[2-Nal]-[Aib]-[Lys(Ac)]-[Lys(Peg11-palm)]-N-NH₂;

Ac-[Pen]-[Cit]-TW-[Cit]-[Pen]-[Phe[4-(2-aminoethoxy)]-[2-Nal]-[Aib]-[Lys(Ac)]-NN-NH₂;

Ac-[Pen]-[Lys(Ac)]-TW-[Cit]-[Pen]-[Phe[4-(2-aminoethoxy)]-[2-Nal]-[Aib]-[Lys(Ac)]-NN-NH₂;

Ac-[Pen]-NT-[Phe(3,4-OCH3)2]-Q-[Pen]-[Phe[4-(2-aminoethoxy)]-[2-Nal]-[Aib]-[Lys(Ac)]-NN-NH₂;

Ac-[Pen]-NT-[Phe(2,4-CH3)2]-Q-[Pen]-[Phe[4-(2-aminoethoxy)]-[2-Nal]-[Aib]-[Lys(Ac)]-NN-NH₂;

Ac-[Pen]-NT-[Phe(3-CH3)]-Q-[Pen]-[Phe[4-(2-aminoethoxy)]-[2-Nal]-[Aib]-[Lys(Ac)]-NN-NH₂;

Ac-[Pen]-NT-[Phe(4-CH3)]-Q-[Pen]-[Phe[4-(2-aminoethoxy)]-[2-Nal]-[Aib]-[Lys(Ac)]-NN-NH₂;

Ac[(D)Arg]-[Pen]-NTWQ-[Pen]-[Phe[4-(2-aminoethoxy)]-[2-Nal]-[Aib]-[Lys(Ac)]-N-[βAla]-NH₂;

Ac-[(D)Tyr]-[Pen]-NTWQ-[Pen]-[Phe[4-(2-aminoethoxy)]-[2-Nal]-[Aib]-[Lys(Ac)]-N-[βAla]-NH₂;

Ac-[Pen]-NTWQ-[Pen]-[Phe[4-(2-aminoethoxy)]-[2-Nal]-[Aib]-[Lys(Ac)]-QN-NH₂;

Ac-[Pen]-NTWQ-[Pen]-[Phe[4-(2-aminoethoxy)]-[2-Nal]-[Aib]-[Lys(Ac)]-[Lys(Ac)]-N-NH₂;

Ac-[Pen]-NTWQ-[Pen]-[Phe[4-(2-aminoethoxy)]-[2-Nal]-[Aib]-[Lys(Ac)]-N-[Lys(Ac)]-NH₂;

Ac-[Pen]-NTWQ-[Pen]-[Phe[4-(2-aminoethoxy)]-[2-Nal]-[Aib]-[Lys(Ac)]-QQ-NH₂;

Ac-[Pen]-NTWQ-[Pen]-[Phe[4-(2-aminoethoxy)]-[2-Nal]-[Aib]-[Lys(Ac)]-Q-[βAla]-NH₂;

Ac-[Pen]-NTWQ-[Pen]-[Phe[4-(2-aminoethoxy)]-[2-Nal]-[Aib]-[Lys(Ac)]-N-[Cit]-NH₂;

Ac-[Pen]-NTWQ-[Pen]-[Phe[4-(2-aminoethoxy)]-[2-Nal]-[Aib]-[Lys(Ac)]-[Cit]-NH₂;

Ac-[Pen]-NTWQ-[Pen]-[Phe[4-(2-aminoethoxy)]-[2-Nal]-[Aib]-[Lys(Ac)]-[Cit]-Q-NH₂;

Ac-[Pen]-NTWQ-[Pen]-[Phe[4-(2-aminoethoxy)]-[2-Nal]-[Aib]-[Lys(Ac)]-[Cit]-[Lys(Ac)]-NH₂;

Ac-[Pen]-NTWQ-[Pen]-[Phe[4-(2-aminoethoxy)]-[2-Nal]-[Aib]-[Lys(Ac)]-[Lys(Ac)]-[Cit]-NH₂;

Ac-[Pen]-NTWQ-[Pen]-[Phe[4-(2-aminoethoxy)]-[2-Nal]-[Aib]-QN-[βAla]-NH₂;

Ac-[Pen]-NTWQ-[Pen]-[Phe[4-(2-aminoethoxy)]-[2-Nal]-[Aib]-E-[Cit]-Q-NH₂;

Ac-[Pen]-NTWQ-[Pen]-[Phe[4-(2-aminoethoxy)]-[2-Nal]-[Aib]-[Cit]-N-[Cit]-NH₂;

Ac-[Pen]-NTWQ-[Pen]-[Phe[4-(2-aminoethoxy)]-[2-Nal]-[Aib]-[Cit]-Q-[Cit]-NH₂;

Ac-[Pen]-[Cit]-TWQ-[Pen]-[Phe[4-(2-aminoethoxy)]-[2-Nal]-[Aib]-[Lys(Ac)]-NN-NH₂;

Ac-[Pen]-NTWQ-[Pen]-[Phe[4-(2-aminoethoxy)]-[2-Nal]-[Aib]-[Lys(Ac)]-NN-NH₂;

Ac-[Pen]-NTWQ-[Pen]-[Phe[4-(2-aminoethoxy)]-[2-Nal]-[Aib]-QNN-NH₂;

Ac-[Pen]-NTWQ-[Pen]-[Phe[4-(2-aminoethoxy)]-[2-Nal]-[Aib]-ENQ-NH₂;

Ac-[Pen]-GPWQ-[Pen]-[Phe[4-(2-aminoethoxy)]-[2-Nal]-[Aib]-[Lys(Ac)]-NN-NH₂;

Ac-[Pen]-PGWQ-[Pen]-[Phe[4-(2-aminoethoxy)]-[2-Nal]-[Aib]-[Lys(Ac)]-NN-NH₂;

Ac-[Pen]-NTWN-[Pen]-[Phe[4-(2-aminoethoxy)]-[2-Nal]-[Aib]-[Lys(Ac)]-NN-NH₂;

Ac-[Pen]-NSWQ-[Pen]-[Phe[4-(2-aminoethoxy)]-[2-Nal]-[Aib]-[Lys(Ac)]-NN-NH₂;

Ac-[Pen]-N-[Aib]-WQ-[Pen]-[Phe[4-(2-aminoethoxy)]-[2-Nal]-[Aib]-[Lys(Ac)]-NN-NH₂;

Ac-[Pen]-NTW-[Aib]-[Pen]-[Phe[4-(2-aminoethoxy)]-[2-Nal]-[Aib]-[Lys(Ac)]N-[Aib]-NH₂;

Ac-[Pen]-QTW-[Lys(Ac)]-[Pen]-[Phe[4-(2-aminoethoxy)]-[2-Nal]-[Aib]-[Lys(Ac)]-NN-NH₂;

Ac-[Pen]-[Lys(Ac)]-TWQ-[Pen]-[Phe[4-(2-aminoethoxy)]-[2-Nal]-[Aib]-[Lys(Ac)]NNNH₂;

Ac-[Pen]-QVWQ-[Pen]-[Phe[4-(2-aminoethoxy)]-[2-Nal]-[Aib]-[Lys(Ac)]-NN-NH₂;

Ac-[Pen]-NT-[2-Nal]-Q-[Pen]-[Phe[4-(2-aminoethoxy)]-[2-Nal]-[Aib]-[Lys(Ac)]-NN-NH₂;

Ac-[Pen]-NT-[1-Nal]-Q-[Pen]-[Phe[4-(2-aminoethoxy)]-[2-Nal]-[Aib]-[Lys(Ac)]-NN-NH₂;

Ac-[Pen]-NTWQ-[Pen]-[Phe[4-(2-aminoethoxy)]-[2-Nal]-[α-MeLeu]-[Lys(Ac)]-NN-NH₂;

Ac-[Pen]-NTWQ-[Pen]-[Phe[4-(2-aminoethoxy)]-[2-Nal]-[α-MeLys]-[Lys(Ac)]-NN-NH₂;

Ac-[Pen]-NTWQ-[Pen]-[Phe[4-(2-aminoethoxy)]-[2-Nal]-[4-amino-4-carboxy-tetrahydropyran]-[Lys(Ac)]-NN-NH₂;

Ac-[Pen]-NTWQ-[Pen]-[Phe[4-(2-aminoethoxy)]-[2-Nal]-[α-MeLeu]-[Lys(Ac)]-N-[βAla]-NH₂;

Ac-[Pen]-NTWQ-[Pen]-[Phe[4-(2-aminoethoxy)]-[2-Nal]-[α-MeLys]-[Lys(Ac)]-N-[βAla]-NH₂;

Ac-[Pen]-NTWQ-[Pen]-[Phe[4-(2-aminoethoxy)]-[2-Nal]-[4-amino-4-carboxy-tetrahydropyran]-[Lys(Ac)]-N-[βAla]-NH₂;

Ac-[Pen]-NTWQ-[Pen]-[Phe[4-(2-aminoethoxy)]-[2-Nal]-[Aib]-[Lys(Ac)]-LN-NH₂;

Ac-[Pen]-NTWQ-[Pen]-[Phe[4-(2-aminoethoxy)]-[2-Nal]-[Aib]-[Lys(Ac)]-GN-NH₂;

Ac-[Pen]-NTWQ-[Pen]-[Phe[4-(2-aminoethoxy)]-[2-Nal]-[Aib]-[Lys(Ac)]-SN-NH₂;

Ac-[Pen]-NTWQ-[Pen]-[Phe[4-(2-aminoethoxy)]-[2-Nal]-[Aib]-[Lys(Ac)]-[Aib]-N-NH₂;

Ac-[Pen]-NTWQ-[Pen]-[Phe[4-(2-aminoethoxy)]-[2-Nal]-[Aib]-[Lys(Ac)]-FN-NH₂;

Ac-[Pen]-NTW-[Cit]-[Pen]-[Phe[4-(2-aminoethoxy)]-[2-Nal]-[Aib]-[Lys(Ac)]-NN-NH₂;

Ac-[Pen]-NTWQ-[Pen]-[Phe[4-(2-aminoethoxy)]-[2-Nal]-[Aib]-[Lys(Ac)]-[Tic]-[βAla]-NH₂;

Ac-[Pen]-NTWQ-[Pen]-[Phe[4-(2-aminoethoxy)]-[2-Nal]-[Aib]-[Lys(Ac)]-[nLeu]-[βAla]-NH₂;

Ac-[Pen]-NTWQ-[Pen]-[Phe[4-(2-aminoethoxy)]-[2-Nal]-[Aib]-[Lys(Ac)]-G-[βAla]-NH₂;

Ac-[Pen]-NTWQ-[Pen]-[Phe[4-(2-aminoethoxy)]-[2-Nal]-[Aib]-[Lys(Ac)]-R-[βAla]-NH₂;

Ac-[Pen]-NTWQ-[Pen]-[Phe[4-(2-aminoethoxy)]-[2-Nal]-[Aib]-[Lys(Ac)]-W-[βAla]-NH₂;

Ac-[Pen]-NTWQ-[Pen]-[Phe[4-(2-aminoethoxy)]-[2-Nal]-[Aib]-[Lys(Ac)]-S-[βAla]-NH₂;

Ac-[Pen]-NTWQ-[Pen]-[Phe[4-(2-aminoethoxy)]-[2-Nal]-[Aib]-[Lys(Ac)]-L-[βAla]-NH₂;

Ac-[Pen]-NTWQ-[Pen]-[Phe[4-(2-aminoethoxy)]-[2-Nal]-[Aib]-[Lys(Ac)]-[AIB]-[βAla]-NH₂;

Ac-[Pen]-NTWQ-[Pen]-[Phe[4-(2-aminoethoxy)]-[2-Nal]-[Aib]-[Lys(Ac)]-[N-MeAla]-[βAla]-NH₂;

Ac-[Pen]-NTWQ-[Pen]-[Phe[4-(2-aminoethoxy)]-[2-Nal]-[Aib]-[Lys(Ac)]-[2-Nap]-[βAla]-NH₂;

Ac-[Pen]-NTWQ-[Pen]-[Phe[4-(2-aminoethoxy)]-[2-Nal]-[Aib]-[Lys(Ac)]-F-[βAla]-NH₂;

Ac-[(D)Arg]-[Pen]-NTWQ-[Pen]-[Phe[4-(2-aminoethoxy)]-[2-Nal]-[4-amino-4-carboxy-tetrahydropyran]-[Lys(Ac)]NN-NH₂;

Biotin-[PEG4]-cyclo[[Abu]-QTWQC]-[Phe[4-(2-aminoethoxy)]-[2-Nal]-[4-amino-4-carboxy-tetrahydropyran]-ENN-NH₂;

Ac-cyclo[[Abu]-QTWQC]-[Phe[4-(2-aminoethoxy)]-[2-Nal]-[4-amino-4-carboxy-tetrahydropyran]-[Lys(Ac)]-NN-NH₂;

Ac-[(D)Arg]-cyclo[[Abu]-QTWQC]-[Phe[4-(2-aminoethoxy)]-[2-Nal]-[4-amino-4-carboxy-tetrahydropyran]-[Lys(Ac)]-NN-NH₂;

Ac-[(D)Arg]-cyclo[[Abu]-QTWQC]-[Phe[4-(2-aminoethoxy)]-[2-Nal]-[4-amino-4-carboxy-tetrahydropyran]-ENN-NH₂;

Ac-E-[(D)Arg]-cyclo[[Abu]-QTWQC]-[Phe[4-(2-aminoethoxy)]-[2-Nal]-[4-amino-4-carboxy-tetrahydropyran]-ENN-NH₂;

Ac-[(D)Asp]-[(D)Arg]-cyclo[[Abu]-QTWQC]-[Phe[4-(2-aminoethoxy)]-[2-Nal]-[4-amino-4-carboxy-tetrahydropyran]-ENN-NH₂;

Ac-R-[(D)Arg]-cyclo[[Abu]-QTWQC]-[Phe[4-(2-aminoethoxy)]-[2-Nal]-[4-amino-4-carboxy-tetrahydropyran]-ENN-NH₂;

inoethoxy)]-[2-Nal]-[4-amino-4-carboxy-tetrahydropyran]-ENN-NH₂;

Ac-F-[(D)Arg]-cyclo[[Abu]-QTWQC]-[Phe[4-(2-aminoethoxy)]-[2-Nal]-[4-amino-4-carboxy-tetrahydropyran]-ENN-NH₂;

Ac-[(D)Phe]-[(D)Arg]-cyclo[[Abu]-QTWQC]-[Phe[4-(2-aminoethoxy)]-[2-Nal]-[4-amino-4-carboxy-tetrahydropyran]-ENN-NH₂;

Ac-[2-Nal]-[(D)Arg]-cyclo[[Abu]-QTWQC]-[Phe[4-(2-aminoethoxy)]-[2-Nal]-[4-amino-4-carboxy-tetrahydropyran]-ENN-NH₂;

Ac-T-[(D)Arg]-cyclo[[Abu]-QTWQC]-[Phe[4-(2-aminoethoxy)]-[2-Nal]-[4-amino-4-carboxy-tetrahydropyran]-ENN-NH₂;

Ac-L-[(D)Arg]-cyclo[[Abu]-QTWQC]-[Phe[4-(2-aminoethoxy)]-[2-Nal]-[4-amino-4-carboxy-tetrahydropyran]-ENN-NH₂;

Ac-[(D)Gln]-[(D)Arg]-cyclo[[Abu]-QTWQC]-[Phe[4-(2-aminoethoxy)]-[2-Nal]-[4-amino-4-carboxy-tetrahydropyran]-ENN-NH₂;

Ac-[(D)Asn]-[(D)Arg]-cyclo[[Abu]-QTWQC]-[Phe[4-(2-aminoethoxy)]-[2-Nal]-[4-amino-4-carboxy-tetrahydropyran]-ENN-NH₂;

Ac-cyclo[[Abu]-QTWQC]-[Phe[4-(2-aminoethoxy)-(PEG4-Alexa488)]-[2-Nal]-[4-amino-4-carboxy-tetrahydropyran]-ENN-NH₂;

[Alexa488]-[PEG4]-cyclo[[Abu]-QTWQC]-[Phe[4-(2-aminoethoxy)]-[2-Nal]-[4-amino-4-carboxy-tetrahydropyran]-ENN-NH₂;

[Alexa647]-[PEG4]-cyclo[[Abu]-QTWQC]-[Phe[4-(2-aminoethoxy)]-[2-Nal]-[4-amino-4-carboxy-tetrahydropyran]-ENN-NH₂;

[Alexa-647]-[PEG4]-[(D)Arg]-cyclo[[Abu]-QTWQC]-[Phe[4-(2-aminoethoxy)]-[2-Nal]-[4-amino-4-carboxy-tetrahydropyran]-[Lys(Ac)]-NN-NH₂;

[Alexa647]-[PEG2]-[(D)Arg]-cyclo[[Abu]-QTWQC]-[Phe[4-(2-aminoethoxy)]-[2-Nal]-[4-amino-4-carboxy-tetrahydropyran]-[Lys(Ac)]-NN-NH₂; and

[Alexa488]-[PEG4]-[(D)Arg]-cyclo[[Abu]-QTWQC]-[Phe[4-(2-aminoethoxy)]-[2-Nal]-[4-amino-4-carboxy-tetrahydropyran]-[Lys(Ac)]-NN-NH₂,

wherein the peptide inhibitor is cyclized via a disulfide bond between the two Pen residues or by a thioether bond between the Abu and the Cys or Pen residue, and wherein the peptide inhibitor inhibits the binding of an interleukin-23 (IL-23) to an IL-23 receptor.

### BRIEF DESCRIPTION OF THE DRAWINGS

FIGS. 1A-1E show levels of disease and IL-23 directed biomarkers measured in colons from rats in the sham (not TNBS-exposed) experimental group, or TNBS-exposed experimental groups that received treatment with vehicle or Peptide 993 (65 mg/kg/day: 10 mg/kg TID, 0.3 mg/ml in drinking water). Data is shown for MPO (FIG. 15A), IL-6 (FIG. 15B), IL-1 beta (FIG. 15C), IL-22 (FIG. 15D), and IL-17A (FIG. 15E). For all experiments, statistical comparisons between groups were performed with a 1-Way ANOVA followed by a post hoc test: * p<0.05; ** p<0.01; *** p<0.001; **** p<0.0001; ns, not significant.

FIGS. 2A-2B show levels of disease and IL-23 directed biomarkers measured in colons from rats in the sham (not TNBS-exposed) experimental group, or TNBS-exposed experimental groups that received treatment with vehicle or Peptide 980 (37 mg/kg/day: 10 mg/kg BID, 0.2 mg/ml in drinking water). Data is shown for MPO (FIG. 16A) and IL-22 (FIG. 16B). For all experiments, statistical comparisons between groups were performed with a 1-Way ANOVA followed by a post hoc test: * p<0.05; ** p<0.01; *** p<0.001; **** p<0.0001; ns, not significant.

FIGS. 3A-3B show levels of LCN2 protein detected in serum (FIG. 3A) or feces (FIG. 3B) collected from acute colitis rats treated with vehicle alone or Peptide 993. For all experiments, statistical comparisons between groups were performed with a 1-Way ANOVA followed by a post hoc test: * p<0.05; ** p<0.01; *** p<0.001; **** p<0.0001; ns, not significant.

FIG. 4 shows levels of MPO protein detected in feces collected from acute colitis rats treated with vehicle alone or Peptide 993. For all experiments, statistical comparisons between groups were performed with a 1-Way ANOVA followed by a post hoc test: * p<0.05; ** p<0.01; *** p<0.001; **** p<0.0001; ns, not significant.

FIGS. 5A-5C show levels of various markers detected in feces collected from acute colitis rats treated with vehicle alone or treated with Peptide 993 at 31 mg/kg/day. FIG. 5A shows LCN2 protein expression levels; FIG. 5B shows MPO protein expression levels; and FIG. 5C shows MMP-9 protein expression levels. For all experiments, statistical comparisons between groups were performed with a 1-Way ANOVA followed by a post hoc test: * p<0.05; ** p<0.01; *** p<0.001; **** p<0.0001; ns, not significant.

FIGS. 6A-6C show levels of various markers detected in the distal colon of acute colitis rats treated with vehicle alone or treated with antibody (mAb) or Peptide 993 at 31 mg/kg/day. FIG. 6A shows LCN2 mRNA expression levels; FIG. 6B shows S100A8 mRNA expression levels; and FIG. 6C shows CLDN8 mRNA expression levels. For all experiments, statistical comparisons between groups were performed with a 1-Way ANOVA followed by a post hoc test: * p<0.05; ** p<0.01; *** p<0.001; **** p<0.0001; ns, not significant.

FIG. 7 shows relative expression of the miR223-3p miRNA detected in distal colon of acute colitis rats treated with vehicle alone or treated with Peptide 993. For all experiments, statistical comparisons between groups were performed with a 1-Way ANOVA followed by a post hoc test: * p<0.05; ** p<0.01; *** p<0.001; **** p<0.0001; ns, not significant.

FIG. 8 shows levels of the miR223-3p miRNA detected in serum of acute colitis rats treated with vehicle alone or treated with Peptide 993 at 31 mg/kg/day. For all experiments, statistical comparisons between groups were performed with a 1-Way ANOVA followed by a post hoc test: * p<0.05; ** p<0.01; *** p<0.001; **** p<0.0001; ns, not significant.

FIGS. 9A-9C show histopathological characterization of acute colitis animals treated with peptide inhibitors. Weight and length were from entire colon (FIG. 9A); colonic score was evaluated as sum of adhesion (0-2), stricture (0-3), ulcer (0-5), and colon wall thickness (0-2) (FIG. 9B); and histology (FIG. 9C) was evaluated by a pathologist as sum of mucosal/submucosal inflammation (0-5), transmural inflammation (0-5), erosion (0-5), and gland loss (0-5) (FIG. 5C). Values shown as mean±SD. Statistical significance was assessed by One-way ANOVA with post-hoc Dunnett's vs. Vehicle control: **p≤0.01; ***p≤0.001; ****p≤0.0001; ns, not significant.

FIGS. 10A-10E show levels of biomarkers in acute colitis animals, including Myeloperoxidase (MPO) (FIG. 10A), IL-17A (FIG. 10B), and IL-22 (FIG. 10C) detected from sampled tissue and quantified by enzyme-linked immunosorbent assay (ELISA); and percentage of phosphorylated Signal Transducer and Activator of Transcription 3 (Stat3) normalized to the area of the distal colon and quantified by immuno-histochemistry (IHC) (FIG. 10D). FIG. 10E shows representative images from each group for IHC analysis of pStat3 expression. Values shown as mean±SD. Statistical significance was assessed by One-way ANOVA with post-hoc Dunnett's vs. Vehicle control: *p≤0.05; **p≤0.01; ***p≤0.001; ****p≤0.0001; ns, not significant.

FIG. 11A shows levels of LCN2 protein detected in serum and colonic score for sham-treated animals (triangles); animals treated with Peptide 993 (circles), and animals treated with vehicle only (Xs). FIG. 11B provides an ROC curve demonstrating the correlation between disease state and LCN2 expression in serum following treatment with Peptide 993.

FIGS. 12A-12D show levels of IL-17A, IL17F, and IL-22 gene expression normalized to that of HPRT1 (FIGS. 12A-12C) and IL22 protein expression (FIG. 12D) in distal colon of 5% DSS acute colitis rats. Relative expression values are shown as geometric mean±95% confidence interval. Protein concentration is shown as mean±SD. Statistical significance was assessed by One-way ANOVA with post-hoc Dunnett's vs. Vehicle control: *p≤0.05; **p≤0.01; ***p≤0.001; ns, not significant.

FIGS. 13A-13B show levels of CLDN8 gene expression in distal colon of TNBS acute colitis rats (FIG. 8A) and the correlation of CLDN8 expression to TNBS colonic score (0-12) (FIG. 8B), respectively, following treatment with sham, vehicle, or Peptide 993. Relative expression values are shown as geometric mean±95% confidence interval. Statistical significance was assessed by One-way ANOVA with post-hoc Dunnett's vs. Vehicle control: *p≤0.05; ****p≤0.0001.

### DETAILED DESCRIPTION

The present disclosure provides assays useful for measuring inflammation, and for determining the effectiveness of therapeutic moieties, e.g., therapeutic moieties that inhibit the IL-23 pathway, in reducing inflammation, including inflammation in the gastrointestinal system associated with immune disorders such as inflammatory bowel diseases.

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

As used herein, the following terms have the meanings ascribed to them unless specified otherwise.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

The singular forms “a,” “an,” and “the” include the plurals unless the context clearly dictates otherwise.

The term “including” is used to mean “including but not limited to.” “Including” and “including but not limited to” are used interchangeably.

The terms “patient,” “subject,” and “individual” may be used interchangeably and refer to either a human or a non-human animal. These terms include mammals such as humans, primates, livestock animals (e.g., bovines, porcines), companion animals (e.g., canines, felines) and rodents (e.g., mice and rats).

The term “peptide,” as used herein, refers broadly to a sequence of two or more amino acids joined together by peptide bonds. It should be understood that this term does not connote a specific length of a polymer of amino acids, nor is it intended to imply or distinguish whether the polypeptide is produced using recombinant techniques, chemical or enzymatic synthesis, or is naturally occurring.

The recitations “sequence identity”, “percent identity”, “percent homology”, or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity.

Calculations of sequence similarity or sequence identity between sequences (the terms are used interchangeably herein) can be performed as follows. To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes). In certain embodiments, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position.

The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two amino acid sequences is determined using the Needleman and Wunsch, (1970, J. Mol. Biol. 48: 444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using an NWSgapdna.CMP matrix and a gap weight of 40, 50, 60, 70, or 80 and a length weight of 1, 2, 3, 4, 5, or 6. Another exemplary set of parameters includes a Blossum 62 scoring matrix with a gap penalty of 12, a gap extend penalty of 4, and a frameshift gap penalty of 5. The percent identity between two amino acid or nucleotide sequences can also be determined using the algorithm of E. Meyers and W. Miller (1989, Cabios, 4: 11-17) which has been incorporated into the ALIGN program (version 2.0), using a PAM120 weight residue table, a gap length penalty of 12 and a gap penalty of 4.

The peptide sequences described herein can be used as a “query sequence” to perform a search against public databases to, for example, identify other family members or related sequences. Such searches can be performed using the NBLAST and XBLAST programs (version 2.0) of Altschul, et al., (1990, J. Mol. Biol, 215: 403-10). BLAST nucleotide searches can be performed with the NBLAST program, score=100, wordlength=12 to obtain nucleotide sequences homologous to nucleic acid molecules of the invention. BLAST protein searches can be performed with the XBLAST program, score=50, wordlength=3 to obtain amino acid sequences homologous to protein molecules of the invention. To obtain gapped alignments for comparison purposes, Gapped BLAST can be utilized as described in Altschul et al. (Nucleic Acids Res. 25:3389-3402, 1997). When utilizing BLAST and Gapped BLAST programs, the default parameters of the respective programs (e.g., XBLAST and NBLAST) can be used.

The term “conservative substitution” as used herein denotes that one or more amino acids are replaced by another, biologically similar residue. Examples include substitution of amino acid residues with similar characteristics, e.g., small amino acids, acidic amino acids, polar amino acids, basic amino acids, hydrophobic amino acids and aromatic amino acids. See, for example, the table below. In some embodiments of the invention, one or more Met residues are substituted with norleucine (Nle) which is a bioisostere for Met, but which, as opposed to Met, is not readily oxidized. Another example of a conservative substitution with a residue normally not found in endogenous, mammalian peptides and proteins is the conservative substitution of Arg or Lys with, for example, ornithine, canavanine, aminoethylcysteine or another basic amino acid. In some embodiments, one or more cysteines of a peptide analogue of the invention may be substituted with another residue, such as a serine. For further information concerning phenotypically silent substitutions in peptides and proteins, see, for example, Bowie et. al. Science 247, 1306-1310, 1990. In the scheme below, conservative substitutions of amino acids are grouped by physicochemical properties. I: neutral, hydrophilic, II: acids and amides, III: basic, IV: hydrophobic, V: aromatic, bulky amino acids.

| I | II | III | IV | V |
|---|---|---|---|---|
| A | N | H | M | F |
| S | D | R | L | Y |
| T | E | K | I | W |
| P | Q | | V | |
| G | | | C | |

In the scheme below, conservative substitutions of amino acids are grouped by physicochemical properties. VI: neutral or hydrophobic, VII: acidic, VIII: basic, IX: polar, X: aromatic.

| VI | VII | VIII | IX | X |
|---|---|---|---|---|
| A | E | H | M | F |
| L | D | R | S | Y |
| I | | K | T | W |
| P | | | C | |
| G | | | N | |
| V | | | Q | |

The term “amino acid” or “any amino acid” as used here refers to any and all amino acids, including naturally occurring amino acids (e.g., a-amino acids), unnatural amino acids, modified amino acids, and non-natural amino acids. It includes both D- and L-amino acids. Natural amino acids include those found in nature, such as, e.g., the 23 amino acids that combine into peptide chains to form the building-blocks of a vast array of proteins. These are primarily L stereoisomers, although a few D-amino acids occur in bacterial envelopes and some antibiotics. The 20 “standard,” natural amino acids are listed in the above tables. The “non-standard,” natural amino acids are pyrolysine (found in methanogenic organisms and other eukaryotes), selenocysteine (present in many noneukaryotes as well as most eukaryotes), and N-formylmethionine (encoded by the start codon AUG in bacteria, mitochondria and chloroplasts). “Unnatural” or “non-natural” amino acids are non-proteinogenic amino acids (i.e., those not naturally encoded or found in the genetic code) that either occur naturally or are chemically synthesized. Over 140 unnatural amino acids are known and thousands of more combinations are possible. Examples of “unnatural” amino acids include β-amino acids (β³ and β²), homo-amino acids, proline and pyruvic acid derivatives, 3-substituted alanine derivatives, glycine derivatives, ring-substituted phenylalanine and tyrosine derivatives, linear core amino acids, diamino acids, D-amino acids, alpha-methyl amino acids and N-methyl amino acids. Unnatural or non-natural amino acids also include modified amino acids. “Modified” amino acids include amino acids (e.g., natural amino acids) that have been chemically modified to include a group, groups, or chemical moiety not naturally present on the amino acid. According to certain embodiments, a peptide inhibitor comprises an intramolecular bond between two amino acid residues present in the peptide inhibitor. It is understood that the amino acid residues that form the bond will be altered somewhat when bonded to each other as compared to when not bonded to each other. Reference to a particular amino acid is meant to encompass that amino acid in both its unbonded and bonded state. For example, the amino acid residue homoSerine (hSer) or homoSerine(Cl) in its unbonded form may take the form of 2-aminobutyric acid (Abu) when participating in an intramolecular bond according to the present invention. The present invention includes both peptide inhibitors containing cross-links between X4 and X9, as well as the peptide inhibitors that do not contain cross-links between X4 and X9, e.g., before cross-link formation. As such, the names hSer and Abu are intended to indicate the same amino acids and are used interchangeably.

For the most part, the names of naturally occurring and non-naturally occurring aminoacyl residues used herein follow the naming conventions suggested by the IUPAC Commission on the Nomenclature of Organic Chemistry and the IUPAC-IUB Commission on Biochemical Nomenclature as set out in “Nomenclature of α-Amino Acids (Recommendations, 1974)” Biochemistry, 14(2), (1975). To the extent that the names and abbreviations of amino acids and aminoacyl residues employed in this specification and appended claims differ from those suggestions, they will be made clear to the reader. Some abbreviations useful in describing the invention are defined below in the following Table 1.

TABLE 1
Abbreviations of Non-Natural Amino Acids and Chemical Moieties (for
amino acid derivatives, all L unless stated)

| Abbreviation | Definition |
|---|---|
| Ac- | Acetyl |
| Hy | Hydrogen (Free N-terminal) |
| Dap | L-Diaminopropionic acid |
| Dab | L-Diaminobutyric acid |
| Orn | L-Ornathine |
| Pen | L-Penicillamine |
| Sarc | Sarcosine |
| Cit | L-Citrulline |
| Cav | L-Cavanine |
| Phe-(4-Guanidino) | 4-Guanidine-L-Phenylalanine |
| N-MeArg | N-Methyl-L-Arginine |
| N-MeTrp | N-Methyl-L-Tryptophan |
| N-MeGln | N-Methyl-L-Glutamine |
| N-MeAla | N-Methyl-L-Alanine |
| N-MeLys | N-Methyl-Lysine |
| N-MeAsn | N-Methyl-L-Asparagine |
| 6-ChloroTrp | 6-Chloro-L-Tryptophan |
| 5-HydroxyTrp | 5-Hydroxy-L-Tryptophan |
| 1,2,3,4-tetrahydro- | L-1,2,3,4-tetrahydro-norharman |
| norharman | |
| 2-Nal | L-2-Napthylalanine |
| (also referred to | |
| as 2-Nap) | |
| 1-Nal | L-1-Napthylalanine |
| (also referred to | |
| as 1-Nap) | |
| Phe(4-OMe) | 4-Methoxy-L-phenylalanine |
| Abu | 2-Aminobutyric acid |
| Bip | L-4,4′-Biphenylalanine |
| βAla | beta-Alanine |
| βhTyr | beta homo-L-Tyrosine |
| βhTrp | beta homo-L-Trptophan |
| βhAla | beta homo-L-Alanine |
| βhLeu, | beta homo-L-Leucine |
| βhVal | beta homo-L-Valine |
| Aib | 2-aminoisobutyric acid |
| Azt | L-azetidine-2-carboxylic acid |
| Tic | (3S)-1,2,3,4-Tetrahydroisoquinoline- |
| | 7-hydroxy-3-carboxylic Acid |
| Phe(4-OMe) | 4-methoxy-L-phenylalanine |
| N-Me-Lys | N-Methyl-L-Lysine |
| N-Me-Lys(Ac) | N-ϵ-Acetyl-D-lysine |
| CONH₂ | Carboxamide |
| COOH | Acid |
| 3-Pal | L-3-Pyridylalanine |
| Phe(4-F) | 4-Fluoro-L-Phenylalanine |
| DMT | 2,6-DimethylTyrosine |
| Phe(4-OMe) | 4-Methoxyphenylalanine |
| hLeu | L-homoLeucine |
| hArg | L-homoArginine |
| α-MeLys | alpha-methyl-L-Lysine |
| α-MeOrn | alpha-methyl-L-Ornathine |
| α-MeLeu | alpha-methyl-L-Leucine |
| α-MeTrp | alpha-methyl-L-Tryptophan |
| α-MePhe | alpha-methyl-L-Phenylalanine |
| α-MeTyr | alpha-methyl-L-Tyrosine |
| α-DiethylGly | α-DiethylGlycine |
| Lys(Ac) | N-ϵ-acetyl-L-Lysine |
| DTT | Dithiothreotol |
| Nle | L-Norleucine |
| βhTrp | L-β-homoTrypophan |
| βhPhe | L-β-homophenylalanine |
| βhPro | L-β-homoproline |
| Phe(4-CF₃) | 4-Trifluoromethyl-L-Phenylalanine |
| β-Glu | L-β-Glutamic acid |
| βhGlu | L-β-homoglutamic acid |
| 2-2-Indane | 2-Aminoindane-2-carboxylic acid |
| 1-1-Indane | 1-Aminoindane-1-carboxylic acid |
| hCha | L-homocyclohexylalanine |
| Cyclobutyl | L-cyclobutylalanine |
| βhPhe | L-β-homo-phenylalanine |
| Gla | Gama-Carboxy-L-Glutamic acid |
| Cpa | Cyclopentyl-L-alanine |
| Cha | Cyclohexyl-L-alanine |
| Octgly | L-Octylglycine |
| t-butyl-Ala | 3-(tert-butyl)-L-Ala-OH |
| t-butyl-Gly | tert-butyl-glycine |
| AEP | 3-(2-aminoethoxy)propanoic acid |
| AEA | (2-aminoethoxy)acetic acid |
| Phe(4-Phenoxy)] | 4-Phenoxy-L-phenylalanine |
| Phe(4-OBzl) | O-Benzyl-L-tyrosine |
| Phe(4-CONH₂) | 4-Carbamoyl-L-phenylalanine |
| Phe(4-CO₂H) | 4-Carboxy-L-phenylalanine |
| Phe(3,4-Cl₂) | 3,4 dichloro-L-phenylalanine |
| Tyr(3-t-Bu) | 3-t-butyl-L-tyrosine |
| Phe(t-Bu) | t-butyl-L-phenylalanine |
| Phe[4-(2- aminoethoxy)] | 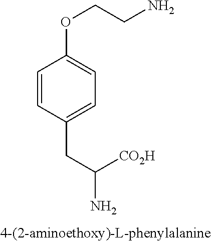 [SMILES: NCCOc1ccc(CC(N)C(=O)O)cc1] |
| Phe(4-CN) | 4-cyano-L-phenylalanine |
| Phe(4-Br) | 4-bromo-L-phenylalanine |
| Phe(4-NH₂) | 4-amino-L-phenylalanine |
| Phe(4-Me) | 4-methyl-L-phenylalanine |
| 4-Pyridylalanine | 4-L-Pyridylalanine |
| 4-amino-4- carboxy-piperidine | 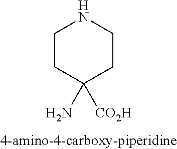 |
| hPhe(3,4-dimethoxy) | 3,4-dimethoxy-L-homophenylalanine |
| Phe(2,4-Me₂) | 2,4-dimethyl-L-phenylalanine |
| Phe(3,5-F₂) | 3,5-difluoro-L-phenylalanine |
| Phe(penta-F) | pentafluoro-L-phenylalanine |
| 2,5,7-tert butyl Trp | 2,5,7-Tris-tert-butyl-L-tryptophan |
| Tic | 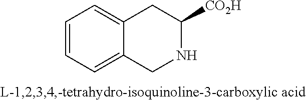 [SMILES: O=C(O)[C@@H]1Cc2ccccc2CN1] |
| Phe(4-OAllyl) | O-Allyl-L-Tyrosine |
| Phe(4-N₃) | 4-azidophenylalanine |
| Achc | 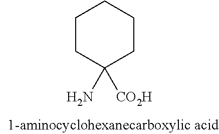 |
| Acvc | 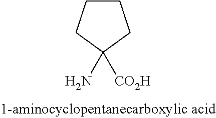 |
| Acbc | 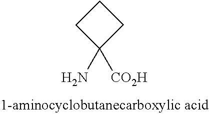 |
| Acpc | 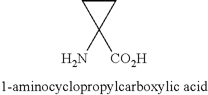 |
| 4-amino-4-carboxy- tetrahydropyran (also referred as THP) | 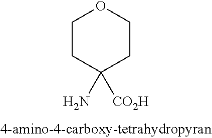 |

Throughout the present specification, unless naturally occurring amino acids are referred to by their full name (e.g. alanine, arginine, etc.), they are designated by their conventional three-letter or single-letter abbreviations (e.g. Ala or A for alanine, Arg or R for arginine, etc.). Unless otherwise indicated, three-letter and single-letter abbreviations of amino acids refer to the L-isomeric form of the amino acid in question. The term “L-amino acid,” as used herein, refers to the “L” isomeric form of a peptide, and conversely the term “D-amino acid” refers to the “D” isomeric form of a peptide (e.g., Dasp, (D)Asp or D-Asp; Dphe, (D)Phe or D-Phe). Amino acid residues in the D isomeric form can be substituted for any L-amino acid residue, as long as the desired function is retained by the peptide. D-amino acids may be indicated as customary in lower case when referred to using single-letter abbreviations.

In the case of less common or non-naturally occurring amino acids, unless they are referred to by their full name (e.g. sarcosine, ornithine, etc.), frequently employed three- or four-character codes are employed for residues thereof, including, Sar or Sarc (sarcosine, i.e. N-methylglycine), Aib (α-aminoisobutyric acid), Dab (2,4-diaminobutanoic acid), Dapa (2,3-diaminopropanoic acid), γ-Glu (γ-glutamic acid), Gaba (γ-aminobutanoic acid), β-Pro (pyrrolidine-3-carboxylic acid), and 8Ado (8-amino-3,6-dioxaoctanoic acid), Abu (2-amino butyric acid), βhPro (β-homoproline), βhPhe (β-homophenylalanine) and Bip (β,β diphenylalanine), and Ida (Iminodiacetic acid).

As is clear to the skilled artisan, the peptide sequences disclosed herein are shown proceeding from left to right, with the left end of the sequence being the N-terminus of the peptide and the right end of the sequence being the C-terminus of the peptide. Among sequences disclosed herein are sequences incorporating a “Hy-” moiety at the amino terminus (N-terminus) of the sequence, and either an “—OH” moiety or an “—NH₂” moiety at the carboxy terminus (C-terminus) of the sequence. In such cases, and unless otherwise indicated, a “Hy-” moiety at the N-terminus of the sequence in question indicates a hydrogen atom, corresponding to the presence of a free primary or secondary amino group at the N-terminus, while an “—OH” or an “—NH₂” moiety at the C-terminus of the sequence indicates a hydroxy group or an amino group, corresponding to the presence of an amido (CONH₂) group at the C-terminus, respectively. In each sequence of the invention, a C-terminal “—OH” moiety may be substituted for a C-terminal “—NH₂” moiety, and vice-versa.

The term “NH₂,” as used herein, can refer to a free amino group present at the amino terminus of a polypeptide. The term “OH,” as used herein, can refer to a free carboxy group present at the carboxy terminus of a peptide. Further, the term “Ac,” as used herein, refers to Acetyl protection through acylation of the C- or N-terminus of a polypeptide. In certain peptides shown herein, the NH₂ locates at the C-terminus of the peptide indicates an amino group.

The term “carboxy,” as used herein, refers to —CO₂H.

The term “cyclized,” as used herein, refers to a reaction in which one part of a polypeptide molecule becomes linked to another part of the polypeptide molecule to form a closed ring, such as by forming a disulfide bridge or other similar bond.

The term “subunit,” as used herein, refers to one of a pair of polypeptide monomers that are joined to form a dimer peptide composition.

The term “linker moiety,” as used herein, refers broadly to a chemical structure that is capable of linking or joining together two peptide monomer subunits to form a dimer.

The term “pharmaceutically acceptable salt,” as used herein, represents salts or zwitterionic forms of the peptides or compounds of the present invention which are water or oil-soluble or dispersible, which are suitable for treatment of diseases without undue toxicity, irritation, and allergic response; which are commensurate with a reasonable benefit/risk ratio, and which are effective for their intended use. The salts can be prepared during the final isolation and purification of the compounds or separately by reacting an amino group with a suitable acid. Representative acid addition salts include acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, formate, fumarate, hydrochloride, hydrobromide, hydroiodide, 2-hydroxyethanesulfonate (isethionate), lactate, maleate, mesitylenesulfonate, methanesulfonate, naphthylenesulfonate, nicotinate, 2-naphthalenesulfonate, oxalate, pamoate, pectinate, persulfate, 3-phenylproprionate, picrate, pivalate, propionate, succinate, tartrate, trichloroacetate, trifluoroacetate, phosphate, glutamate, bicarbonate, para-toluenesulfonate, and undecanoate. Also, amino groups in the compounds of the present invention can be quatemized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. A pharmaceutically acceptable salt may suitably be a salt chosen, e.g., among acid addition salts and basic salts. Examples of acid addition salts include chloride salts, citrate salts and acetate salts. Examples of basic salts include salts where the cation is selected among alkali metal cations, such as sodium or potassium ions, alkaline earth metal cations, such as calcium or magnesium ions, as well as substituted ammonium ions, such as ions of the type N(R1)(R2)(R3)(R4)+, where R1, R2, R3 and R4 independently will typically designate hydrogen, optionally substituted C1-6-alkyl or optionally substituted C2-6-alkenyl. Examples of relevant C1-6-alkyl groups include methyl, ethyl, 1-propyl and 2-propyl groups. Examples of C2-6-alkenyl groups of possible relevance include ethenyl, 1-propenyl and 2-propenyl. Other examples of pharmaceutically acceptable salts are described in “Remington's Pharmaceutical Sciences”, 17th edition, Alfonso R. Gennaro (Ed.), Mark Publishing Company, Easton, Pa., USA, 1985 (and more recent editions thereof), in the “Encyclopaedia of Pharmaceutical Technology”, 3rd edition, James Swarbrick (Ed.), Informa Healthcare USA (Inc.), NY, USA, 2007, and in J. Pharm. Sci. 66: 2 (1977). Also, for a review on suitable salts, see Handbook of Pharmaceutical Salts: Properties, Selection, and Use by Stahl and Wermuth (Wiley-VCH, 2002). Other suitable base salts are formed from bases which form non-toxic salts. Representative examples include the aluminum, arginine, benzathine, calcium, choline, diethylamine, diolamine, glycine, lysine, magnesium, meglumine, olamine, potassium, sodium, tromethamine, and zinc salts. Hemisalts of acids and bases may also be formed, e.g., hemisulphate and hemicalcium salts.

The term “N(alpha)Methylation”, as used herein, describes the methylation of the alpha amine of an amino acid, also generally termed as an N-methylation.

As used herein, a “therapeutically effective amount” of the peptide inhibitor of the invention is meant to describe a sufficient amount of the peptide inhibitor to treat an IL-23/IL-23R-related disease, including but not limited to any of the diseases and disorders described herein (for example, to reduce inflammation associated with IBD). In particular embodiments, the therapeutically effective amount will achieve a desired benefit/risk ratio applicable to any medical treatment.

An “analog” of an amino acid, e.g., a “Phe analog” or a “Tyr analog” means an analog of the referenced amino acid. A variety of amino acid analogs are known and available in the art, including Phe and Tyr analogs. In certain embodiments, an amino acid analog, e.g., a Phe analog or a Tyr analog comprises one, two, three, four or five substitutions as compared to Phe or Tyr, respectively. In certain embodiments, the substitutions are present in the side chains of the amino acids. In certain embodiments, a Phe analog has the structure Phe(R²), wherein R² is a Hy, OH, CH₃, CO₂H, CONH₂, CONH₂OCH₂CH₂NH₂, t-Bu, OCH₂CH₂NH₂, phenoxy, OCH₃, OAllyl, Br, Cl, F, NH₂, N3, or guanadino. In certain embodiments, R² is CONH₂OCH₂CH₂NH₂, OCH₃, CONH₂, OCH₃ or CO₂H. Examples of Phe analogs include, but are not limited to: hPhe, Phe(4-OMe), α-Me-Phe, hPhe(3,4-dimethoxy), Phe(4-CONH₂), Phe(4-phenoxy), Phe(4-guanadino), Phe(4-tBu), Phe(4-CN), Phe(4-Br), Phe(4-OBzl), Phe(4-NH₂), BhPhe(4-F), Phe(4-F), Phe(3,5 DiF), Phe(CH₂CO₂H), Phe(penta-F), Phe(3,4-Cl₂), Phe (3,4-F₂), Phe(4-CF₃), ββ-diPheAla, Phe(4-N₃), Phe[4-(2-aminoethoxy)], 4-Phenylbenzylalanine, Phe(4-CONH₂), Phe(3,4-Dimethoxy), Phe(4-CF₃), Phe(2,3-Cl₂), and Phe(2,3-F₂). Examples of Tyr analogs include, but are not limited to: hTyr, N-Me-Tyr, Tyr(3-tBu), Tyr(4-N₃) and βhTyr.

Details of the disclosure are set forth herein. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

The practice of the present invention will employ, unless indicated specifically to the contrary, conventional methods of virology, immunology, microbiology, molecular biology, and recombinant DNA techniques within the skill of the art, many of which are described below for the purpose of illustration. Such techniques are explained fully in the literature. See, e.g., Current Protocols in Molecular Biology or Current Protocols in Immunology, John Wiley & Sons, New York, N.Y. (2009); Ausubel et al., Short Protocols in Molecular Biology, 3^{rd} ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) and other like references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

### Bioassays and Methods of Treatment

The present invention provides methods and reagents for determining the level of one or more biomarkers of inflammation, e.g., inflammation associated with an inflammatory disease or disorder, in a subject, e.g., a subject diagnosed with or considered at risk of an inflammatory disease or disorder. In certain embodiments, the level of the biomarker protein expression is determined, and in certain embodiments, the level of the biomarker mRNA expression is determined. In certain embodiments, the biomarker is an miRNA that may be detected. In particular embodiments, the methods are practiced by determining the level of the one or more biomarkers in a biological sample obtained from the subject. In particular embodiments, the inflammatory disease or disorder is associated with inflammation in gastrointestinal system, e.g., the stomach, small intestine, large intestine, or bowel, e.g., colon, such as an inflammatory bowel disease, and in certain embodiments, the biological sample comprises feces. In some embodiments, the biological sample comprises serum. In some embodiments, the biological sample is a cell or tissue sample obtained from the subject, e.g., a biopsy sample, such as a sample of distal colon tissue. Methods of the present invention may be used to detect, determine or monitor the amount of inflammation in the subject, e.g., in the subject's gastrointestinal system. In certain embodiments of any of the methods described herein, the marker is MPO, LCN2, or MMP9, and the biological sample is serum or feces.

In certain embodiments of any of the assays disclosed herein, the one or more biomarkers are selected from: myeloperoxidase (MPO), interleukin-1β (IL-1β), interleukin-6 (IL-6), interleukin-22 (IL-22), interleukin-17A (IL-17A), interleukin-17F (IL-17F), lipocalin 2 (LCN2), matrix metallopeptidase 9 (MMP9), S100 calcium-binding protein A8 (S100A8), claudin 8 (CLDN8), microRNA-223-3p (miR223-3p), or phosphorylated signal transducer and activator of transcription 3 (pSTAT3). S100 calcium-binding protein A8 (S100A8), also known as calgranulin A, heterodimerizes with S100A9 to form calprotectin. Accordingly, in certain embodiments of any of the methods disclosed herein, calprotectin or S100A9 may be used as a biomarker in addition to or instead of S100A8, e.g., in assays of feces or serum. In particular embodiments, the biomarkers are human proteins or nucleic acids, the sequence which are known in the art.

In certain embodiments, any of the assays measure levels or expression of a panel of these biomarkers, including any subset thereof. In particular embodiments, higher levels of the markers are associated with inflammation as compared to levels in normal tissue, except for CLDN8, which has lower levels associated with inflammation as compared to normal tissue.

In particular embodiments, the present invention provides assays and reagents for determining an amount of one or more biomarkers before, during, or after providing to a subject a peptide inhibitor of an interleukin-23 receptor (IL-23R). In certain embodiments, the method comprises determining an amount of one or more biomarkers both before and after providing the peptide inhibitor to the subject. In certain aspects, these methods may be advantageously used for any of the following: (i) to determine whether a subject is a candidate for treatment with a peptide inhibitor of an interleukin-23 receptor (IL-23R); (ii) to determine the effectiveness of treatment of a subject with a peptide inhibitor of an interleukin-23 receptor (IL-23R); (iii) to monitor the effect of treatment of a subject with a peptide inhibitor of an interleukin-23 receptor (IL-23R) at different times during treatment; or (iv) to determine whether a subject treated with a peptide inhibitor of an interleukin-23 receptor (IL-23R) should receive additional treatments with a peptide inhibitor of an interleukin-23 receptor (IL-23R). In certain embodiments, the marker is MPO or LCN2 and the biological sample is serum or feces, and the peptide inhibitor is any of those described herein, e.g., any selected from those shown in any of Tables 2, 3, 4, 5A, 5B or 6, or Peptides 993, 980, or 1185.

Certain embodiments of the present disclosure are directed to a method of determining a level of a biomarker in a biological sample comprising (i) contacting the sample with an agent that specifically binds the biomarker, and (ii) detecting the presence or absence of, or an amount of, the reagent bound to the biomarker by an assay, e.g., an immunoassay, and thereby determining the level of the biomarker. In one embodiment of this aspect of the disclosure, the sample is a liquid matrix and is selected from the group comprising serum, plasma, blood, urine, or sputum. In a further embodiment, the sample is feces. In particular embodiments, the feces is processed to extract proteins before being contacted with one or more biomarker. Methods of processing feces to obtain proteins are known in the art and described in the accompanying examples. In a further embodiment of this aspect of the disclosure, the sample was obtained from a subject to whom a peptide inhibitor of an interleukin-23-receptor (IL23-R) has been administered. In a further embodiment of this aspect of the disclosure, the immunoassay is selected from the group comprising cloned enzyme donor immunoassay (CEDIA), turbidity assay, ELISA, and competitive ELISA. In certain embodiments, the reagent is bound to a detectable marker. In certain embodiments, the one or more biomarkers are selected from: myeloperoxidase (MPO), interleukin-1β (IL-1b), interleukin-6 (IL-6), interleukin-22 (IL-22), interleukin-17A (IL-17A), interleukin-17F (IL-17F), lipocalin 2 (LCN2), matrix metallopeptidase 9 (MMP9), S100 calcium-binding protein A8 (S100A8), claudin 8 (CLDN8), microRNA-223-3p (miR223-3p), or phosphorylated signal transducer and activator of transcription 3 (pSTAT3), or any combination or subset thereof. In particular embodiments, the biomarker is selected from one or more of MPO, interleukin-6 (IL-6), interleukin-1β (IL-1β), interleukin-22 (IL-22), interleukin-17A (IL-17A), interleukin-17F (IL-17F), pSTAT3, CLDN8, LCN2, or MMP9. In one embodiment, the level of the biomarker in a biological sample obtained from the subject is determined prior to treatment for, and, in some embodiments, after being diagnosed with inflammation or an immune disorder. In certain embodiments, the biological sample is serum or feces. In certain embodiments, the peptide inhibitor is any described herein, e.g., any of those shown in Tables 2, 3, 4, 5A, 5B or 6, or Peptides 993, 980, or 1185. In certain embodiments, the marker is MPO, LCN2, or MMP9, and the biological sample is serum or feces, and the peptide inhibitor is any of those described herein, e.g., selected from any of those shown in Tables 2, 3, 4, 5A, 5B or 6, or Peptides 993, 980, or 1185.

In certain embodiments of any of the methods described herein, the marker is pSTAT3, a transcription factor regulated by IL-23. In particular embodiments, levels or amounts of pSTAT3 are measured using an antibody that specifically binds to signal transducer and activator of transcription 3 (STAT3) phosphorylated at Tyrosine 705 (pSTAT3). In some embodiments, the levels of pSTAT3 are determined by immunoperoxidase staining of colon tissue obtained from an animal, e.g., as described in the accompanying Examples. In certain embodiments, immunoperoxidase staining is performed by fixing the biological sample, e.g., in 10% neutral buffered formalin; processing the sample by routine paraffin embedding; sectioning the fixed and embedded sample into 3-5 micron sections onto positively charged slides; and performing Automated Leica Biond Rx™ staining protocol by incubating the sample with a primary antibody that specifically recognizes STAT3 phosphorylated at Tyrosine 705 (Abcam Cat. No. ab76315 PUR), a secondary antibody conjugated to horse radish peroxidase (HRP), and DAB (3,3′-diaminobenzidine tetrahydrochloride; MW=214.1), which reacts with HRP in the presence of peroxide to yield an insoluble brown-colored product at locations where peroxidase-conjugated antibodies are bound to samples. The percentage of pSTAT3 can be normalized to the area of the distal colon quantified by immuno-histochemistry (IHC).

In certain embodiments, the present disclosure includes a method for determining the efficacy of treatment of a subject having an inflammatory disease or disorder with a pharmaceutical agent, e.g., an inhibitor of the interleukin-23 signalling pathway, such as, e.g., a peptide inhibitor of interleukin-23 receptor (IL-23R), including but not limited to any of those described herein, comprising: (i) contacting a biological sample obtained from the subject after treatment with the pharmaceutical agent (e.g., IL-23R inhibitor) with one or more reagent that binds one or more biomarker; and (ii) detecting the presence or absence of, or an amount of, the reagent bound to the biomarker, and thereby determining the level of the biomarker in the biological sample, wherein if the level is reduced as compared to a pre-determined cut-off value (e.g., lower than the pre-determined cut-off value) or as compared to the level of the biomarker before treatment with the pharmaceutical agent (e.g., IL-23R inhibitor), it indicates that the treatment is efficacious (except for CLDN8, where it indicated the treatment is not efficacious), and wherein if the level is the same or increased as compared to a pre-determined cut-off value (e.g., equal to or higher than the pre-determined cut-off value) or as compared to the level of the biomarker before the treatment, it indicates that the treatment is not efficacious (except for CLDN8, where it indicates the treatment is efficacious). In one embodiment, the level of the biomarker before treatment is determined in a biological sample obtained from the subject prior to the treatment, and, in some embodiments, after being diagnosed with inflammation or an immune disorder. In certain embodiments, the one or more biomarkers are selected from: myeloperoxidase (MPO), interleukin-1β (IL-1b), interleukin-6 (IL-6), interleukin-22 (IL-22), interleukin-17A (IL-17A), interleukin-17F (IL-17F), lipocalin 2 (LCN2), matrix metallopeptidase 9 (MMP9), S100 calcium-binding protein A8 (S100A8), claudin 8 (CLDN8), microRNA-223-3p (miR223-3p), or phosphorylated signal transducer and activator of transcription 3 (pSTAT3), or any combination or subset thereof. In particular embodiments, the marker is selected from one or more of MPO, interleukin-6 (IL-6), interleukin 1β (IL-1β), interleukin-22 (IL-22), interleukin-17A (IL-17A), interleukin-17F (IL-17F), pStat3, CLDN8, or LCN2. In certain embodiments, the biological sample is serum or feces. In certain embodiments, the peptide inhibitor is any described herein, e.g., any shown in Tables 2, 3, 4, 5A, 5B or 6, or Peptides 993, 980, or 1185. In certain embodiments, the marker is MPO, LCN2, or MMP9, and the biological sample is serum or feces, and the peptide inhibitor is any of those described herein, e.g., selected from any of those shown in Tables 2, 3, 4, 5A, 5B or 6, or Peptides 993, 980, or 1185. In certain embodiment, the marker is pSTAT3, a transcription factor regulated by IL-23. In particular embodiments, levels or amounts of pSTAT3 are measured using an antibody that specifically binds to STAT3 phosphorylated at Tyrosine 705 (pSTAT3), e.g., as described herein. In certain embodiments, the method further comprises the step of determining the level of the biomarker before treatment, comprising: (i) contacting a biological sample obtained from the subject before treatment with the pharmaceutical agent (e.g., IL-23R inhibitor) with a reagent that binds the biomarker; and (ii) detecting the presence or absence of, or an amount of, the reagent bound to the biomarker, and thereby determining the level of the biomarker in the biological sample.

Particular embodiments of the present invention are directed to a method of determining whether treatment of a subject suffering from an inflammatory disease or disorder, e.g., an inflammatory bowel disease (IBD) such as Crohn's disease or ulcerative colitis, with a pharmaceutical agent, e.g., an inhibitor of the interleukin-23 signalling pathway, such as, e.g., a peptide inhibitor of interleukin-23 receptor (IL-23R), including but not limited to any of those described herein, is efficacious, the method comprising (i) contacting a biological sample obtained from the subject following treatment with a peptide inhibitor described herein, wherein the biological sample is optionally serum or feces, with an agent, optionally an antibody or antigen-binding fragment thereof, that specifically binds a biomarker, e.g., MPO or LCN2, and (ii) detecting the presence or absence of, or an amount of, the reagent bound to the biomarker by an assay, e.g., an immunoassay, and thereby determining the amount or level of the biomarker in the biological sample, wherein the treatment is considered efficacious if the amount or level is below a cut-off value or below the level of the biomarker before treatment with the pharmaceutical agent (except for CLDN8, where treatment is considered efficacious if the amount or level is above a cut-off value or above the level of the biomarker before treatment with the pharmaceutical agent). In one embodiment, the level of the biomarker before treatment is determined in a biological sample obtained from the subject prior to treatment, and, in some embodiments, after being diagnosed with inflammation or an immune disorder. In one embodiment of this aspect of the disclosure, the sample is a liquid matrix and is selected from the group comprising serum, plasma, blood, urine, or sputum. In a further embodiment, the sample is feces. In a further embodiment of this aspect of the disclosure, the assay is performed using an immunoassay selected from the group comprising cloned enzyme donor immunoassay (CEDIA), turbidity assay, and competitive ELISA. In certain embodiments, the one or more biomarkers are selected from: myeloperoxidase (MPO), interleukin-1β (IL-1b), interleukin-6 (IL-6), interleukin-22 (IL-22), interleukin-17A (IL-17A), interleukin-17F (IL-17F), lipocalin 2 (LCN2), matrix metallopeptidase 9 (MMP9), S100 calcium-binding protein A8 (S100A8), claudin 8 (CLDN8), microRNA-223-3p (miR223-3p), or phosphorylated signal transducer and activator of transcription 3 (pSTAT3), or any combination or subset thereof. In particular embodiments, the marker(s) is selected from one or more of MPO, interleukin-6 (IL-6), interleukin 1β (IL-1β), interleukin-22 (IL-22), interleukin-17A (IL-17A), interleukin-17F (IL-17F), pStat3, CLDN8, or LCN2. In certain embodiments, the biological sample is serum or feces. In certain embodiments, the peptide inhibitor is any of those described herein, e.g., any of those described here, e.g., any of those shown in Tables 2, 3, 4, 5A, 5B or 6, or Peptides 993, 980, or 1185. In certain embodiments, the marker is MPO, LCN2, or MMP9, and the biological sample is serum or feces, and the peptide inhibitor is any of those described herein, e.g., selected from any of those shown in Tables 2, 3, 4, 5A, 5B or 6, or Peptides 993, 980, or 1185. In certain embodiment, the marker is pSTAT3, a transcription factor regulated by IL-23. In particular embodiments, levels or amounts of pSTAT3 are measured using an antibody that specifically binds to STAT3 phosphorylated at Tyrosine 705 (pSTAT3), e.g., as described herein. In certain embodiments, the method further comprises the step of determining the level of the biomarker before treatment, comprising: (i) contacting a biological sample obtained from the subject before treatment with the pharmaceutical agent (e.g., IL-23R inhibitor) with a reagent that binds the biomarker; and (ii) detecting the presence or absence of, or an amount of, the reagent bound to the biomarker, and thereby determining the level of the biomarker in the biological sample. These and other methods disclosed herein may be used to monitor disease progression or monitor the efficacy (or not) of treatment with an inhibitor of IL-23R, including any of the peptide inhibitors disclosed herein. In certain embodiments, the levels of the one or more biomarkers are determined before treatment and at one or more time points following treatment, e.g., daily, every other day, weekly bi-weekly or monthly. In one embodiment, the levels of the one or more biomarkers are determined throughout the course of treatment, e.g., weekly over the course of a 12 week treatment regimen. In particular embodiments, the treatment may last for one week, two weeks, three weeks, four weeks, five weeks, six weeks, eight weeks, ten weeks, 12 weeks, 16 weeks or more.

Embodiments disclosed herein include a method of determining the presence of an inflammatory disease or disorder, optionally intestinal inflammation, in a subject, comprising: (i) contacting a serum sample obtained from the subject with a reagent that binds lipocalin 2 (LCN2); and (ii) detecting the presence or absence of, or determining an amount of, the reagent bound to the LCN2, and thereby determining the level of LCN2 in the serum sample, wherein if the level of LCN2 in the serum sample is greater than 200 ng/mL, greater than 250 ng/mL, or greater than 300 ng/mL, it is determined that the subject has an inflammatory disease or disorder. In particular embodiments, if the level of LCN2 in the serum sample is greater than 294 ng/mL, intestinal inflammation is determined to be present in the subject with a sensitivity of 89% and a specificity of 95%. In certain embodiments, if the level of LCN2 in the serum sample is less than 200 ng/mL, less than 150 ng/nL, less than 100 ng/mL, less than 50 ng/mL, or less than 20 ng/mL, intestinal inflammation is determined to not be present in the subject.

Certain embodiments include methods for determining or monitoring the efficacy of treatment of a subject having an inflammatory disease or disorder, optionally gastrointestinal inflammation, with a peptide inhibitor of interleukin-23 receptor (IL-23R), comprising: (i) contacting a biological sample obtained from the subject during or after treatment with the IL-23R inhibitor with one or more reagent that binds one or more biomarker of inflammation, wherein one or more of the biomarkers are selected from the group consisting of: myeloperoxidase (MPO), interleukin-1β (IL-1β), interleukin-6 (IL-6), interleukin-22 (IL-22), interleukin-17A (IL-17A), interleukin-17F (IL-17F), lipocalin 2 (LCN2), matrix metallopeptidase 9 (MMP9), S100 calcium-binding protein A8 (S100A8), microRNA-223-3p (miR223-3p), and phosphorylated signal transducer and activator of transcription 3 (pSTAT3) proteins, polynucleotides encoding any of the proteins, and polynucleotides comprising a region complementary to microRNA-223-3p or any of the polynucleotides that encode any of the proteins; and (ii) detecting the presence or absence of, or determining an amount of, the reagent bound to the biomarker, and thereby determining the level of the one or more biomarker in the biological sample, wherein if the level is reduced as compared to a pre-determined cut-off value or the level of the biomarker before treatment with the IL-23R inhibitor, it indicates that the treatment is efficacious, and wherein if the level is the same or increased as compared to a pre-determined cut-off value or the level of the biomarker before treatment with the IL-23R inhibitor, it indicates that the treatment is not efficacious.

Related embodiments include methods for determining or monitoring the efficacy of treatment of a subject having an inflammatory disease or disorder, optionally gastrointestinal inflammation, with a peptide inhibitor of interleukin-23 receptor (IL-23R), comprising: (i) contacting a biological sample obtained from the subject during or after treatment with the IL-23R inhibitor with one or more reagent that binds one or more biomarker of inflammation, wherein one or more of the biomarkers is a Claudin 8 (CLDN8) biomarker, optionally a protein, a polynucleotide that encodes the CLDN8 protein, or a polynucleotide comprising a region complementary to the polynucleotide that encodes the CLDN8 protein; and (ii) detecting the presence or absence of, or determining an amount of, the reagent bound to the CLDN8 biomarker, and thereby determining the level of the CLDN8 biomarker in the biological sample, wherein if the level is increased as compared to a pre-determined cut-off value or the level of the CLDN8 biomarker before treatment with the IL-23R inhibitor, it indicates that the treatment is efficacious, and wherein if the level is the same or reduced as compared to a pre-determined cut-off value or the level of the CLDN8 biomarker before treatment with the IL-23R inhibitor, it indicates that the treatment is not efficacious.

In particular embodiments of any of the methods, the biological sample is a gastrointestinal tissue sample, optionally a colon tissue sample, and one or more of the biomarker is selected from the group consisting of: LCN2, MPO, IL-1β, IL-6, IL-17A, IL-17F, IL-22, pSTAT3, S100A8, CLDN8, and miR-223-3p. In certain embodiments, the biological sample is feces, and one or more biomarker is selected from the group consisting of: LCN2, MPO, and MMP9. In certain embodiments, the biological sample is a liquid matrix, wherein the liquid matrix is optionally serum, plasma, blood, or urine. In particular embodiments, the liquid matrix is serum, and one of the biological markers is LCN2, MPO, and MMP9, optionally LCN2.

Some embodiments of the present disclosure are directed to a method of treating a disease or disorder in a subject in need thereof, comprising (i) providing to the subject an effective amount of a peptide inhibitor of an interleukin-23-receptor (IL23-R), (ii) waiting for a first period of time, and (iii) determining the amount of or level of a biomarker in a biological sample obtained from the subject according to the methods described herein. Certain embodiments of this aspect of the disclosure further comprise: (iv) providing additional peptide inhibitor of an interleukin-23-receptor (IL23-R) to the subject, if the determined level of the biomarker is at or above a cut-off value or if the determined level of the biomarker is equal to or higher to an amount determined prior to the subject being provided the peptide inhibitor; or (v) not providing additional peptide inhibitor of an interleukin-23-receptor (IL23-R) to the subject, if the determined level of biomarker is below a cut-off value or if the determined level of the biomarker is lower than the amount determined prior to the subject being provided the peptide inhibitor. With respect to CLDN8, the additional peptide inhibitor would be provided if the level of CLDN8 was below a cut-off value or if the determined level of CLDN8 is lower than an amount determined prior to the subject being provided the peptide inhibitor, and the additional peptide inhibitor would not be provided if the level of CLDN8 was at or above a cut-off value or if the determined level of CLDN8 was high than an amount determined prior to the subject being provided the peptide inhibitor. In particular, certain embodiments comprise providing additional peptide inhibitor to the subject if the determined level of the biomarker is at or above a defined cut-off value. In further embodiments, additional peptide inhibitor is not provided to the subject, or future dosage levels are decreased, if the determined level of biomarker is below a defined cut-off value. In one embodiment, the level of the biomarker before treatment is determined in a biological sample obtained from the subject prior to treatment, and, in some embodiments, after being diagnosed with inflammation or an immune disorder. In certain embodiments, the one or more biomarkers are selected from: myeloperoxidase (MPO), interleukin-1β (IL-1b), interleukin-6 (IL-6), interleukin-22 (IL-22), interleukin-17A (IL-17A), interleukin-17F (IL-17F), lipocalin 2 (LCN2), matrix metallopeptidase 9 (MMP9), S100 calcium-binding protein A8 (S100A8), claudin 8 (CLDN8), microRNA-223-3p (miR223-3p), or phosphorylated signal transducer and activator of transcription 3 (pSTAT3), or any combination or subset thereof. In particular embodiments, the marker is selected from one or more of MPO, interleukin-6 (IL-6), interleukin 1β (IL-1β), interleukin-22 (IL-22), interleukin-17A (IL-17A), interleukin-17F (IL-17F), pStat3, CLDN8, or LCN2. In certain embodiments, the biological sample is serum or feces. In certain embodiments, the peptide inhibitor is any of those described herein, e.g., any shown in Tables 2, 3, 4, 5A, 5B or 6, or Peptides 993, 980, or 1185. In certain embodiments, the marker is MPO, LCN2, or MMP9, and the biological sample is serum or feces, and the peptide inhibitor is any of those described herein, e.g., selected from any of those shown in Tables 2, 3, 4, 5A, 5B or 6, or Peptides 993, 980, or 1185. In certain embodiment, the marker is pSTAT3, a transcription factor regulated by IL-23. In particular embodiments, levels or amounts of pSTAT3 are measured using an antibody that specifically binds to STAT3 phosphorylated at Tyrosine 705 (pSTAT3), e.g., as described herein. In certain embodiments, the method further comprises the step of determining the level of the biomarker before treatment, comprising: (i) contacting a biological sample obtained from the subject before treatment with the pharmaceutical agent (e.g., IL-23R inhibitor) with a reagent that binds the biomarker; and (ii) detecting the presence or absence of, or an amount of, the reagent bound to the biomarker, and thereby determining the level of the biomarker in the biological sample.

Some embodiments of the present invention are directed to a method of determining the epithelial integrity in the colon in a subject, comprising: (i) determining the amount of or level of a biomarker in a biological sample obtained from the subject according to any of the methods described herein, and (ii) determining a colonic score for the biological sample of the subject. In certain embodiments, the marker is a member of the claudin family of proteins, e.g., CLDN8 (claudin-8 gene). In a specific embodiment, the colonic score correlates with the level of CLDN8. In certain embodiments, the subject is treated with a pharmaceutical agent (e.g., a peptide inhibitor of IL-23R) prior to determining the amount or level of the biomarker and/or determine the colonic score. In certain embodiments, the peptide inhibitor is any of those described herein, e.g., any shown in Tables 2, 3, 4, 5A, 5B or 6, or Peptides 993, 980, or 1185. In some embodiments, the biological sample is serum or feces. In other embodiments, the biological sample is a whole colon, or a section of a colon. In one embodiment, the level of the biomarker before treatment is determined in a biological sample obtained from the subject prior to treatment, and, in some embodiments, after being diagnosed with inflammation or an immune disorder. In related methods, the level of the biomarker and the colon score is determined for a biological sample obtained from the subject before treatment with a pharmaceutical agent, and in some embodiments, the level of the biomarker and the colon score is determined for a biological sample obtained from the subject after treatment with a pharmaceutical agent. In related methods, the level of the biomarker and the colon score is determined for a biological sample obtained from the subject before treatment with a pharmaceutical agent, and the level of the biomarker and the colon score is determined for a biological sample obtained from the subject after treatment with a pharmaceutical agent. Certain embodiments, include comparing the level of CLDN-8 and/or the colonic score before treatment to the level of CLDN-8 and/or the colonic score after treatment, wherein a higher level of CLDN-8 after treatment indicates efficacy and a lower colonic score after treatment indicates efficacy of the treatment. Methods of determining colonic score are known in the art and described herein.

In certain embodiments, any of the methods disclosed herein comprise determining the presence of or an amount of one or more biomarkers at two or more different time points, e.g., before and during treatment, before and after treatment, two or more times during treatment, or during and after treatment. In particular embodiments, a treatment regimen may include treatment for at least one day, at least one week, at least two weeks, at least four weeks, at least six weeks, at least 8 weeks, at least 12 weeks, at least 16 weeks or more. So, for example, in particular embodiments, the presence of or an amount of the one or more biomarkers may be determined before and after treatment, or daily, weekly, bi-weekly, or monthly throughout the course of treatment, e.g., to monitor the presence of inflammation following treatment and/or determine whether the treatment is efficacious.

In particular embodiments of any of the methods described herein, the presence of or an amount of a biomarker (e.g., a protein biomarker) is determined using an immunoassay. As used herein, “immunoassay” refers to a biochemical test that measures the presence of or concentration of an analyte, e.g., the biomarker in the biological sample, through the use of an antibody, or antigen-binding fragment thereof. In some embodiments, the biomarker is the antigen. Certain immunoassays employ the use of antigen-specific antibodies labeled with a detectable label or “primary antibodies”, to directly detect the presence of or concentration of a biomarker or antigen. Certain immunoassays utilize antigen-specific antibodies that are not labeled, and require the application of one or more additional antibodies, or “secondary antibodies”, to detect the presence of the primary antibody and thus indirectly detect the presence or concentration of the biomarker or antigen. When performing an immunoassay, controls typically include a reaction well or tube that contains an antibody or antigen-binding fragment thereof alone (i.e., in the absence of antigen), wherein an amount of reactivity (e.g., non-specific binding to the well) by the antibody or antigen-binding fragment thereof in the absence of the antigen is considered to be background. This allows for the accurate assessment of antigen specific-binding. Non-limiting examples of immunoassays include direct enzyme-linked immunosorbent assays (ELISAs), competitive ELISAs, cloned enzyme donor immunoassay (CEIDA), real-time immunoquantitative polymerase chain reaction (iqPCR), polymerase chain reaction (PCR), quantitative reverse transcription-PCR (RT-PCR), RNA sequencing, lateral flow tests, magnetic immunoassays, radioimmunoassays, surround optical fiber immunoassays (SOFIA), and enzyme multiplied immunoassay technique (EMIT), and turbidity assays.

In some embodiments, an immunoassay is a competitive immunoassay that includes use of a biomarker bound to a solid support, e.g. an ELISA plate. In such an embodiment, an antibody that specifically binds the biomarker can be contacted in solution with a biological sample. In this instance, the amount of antibody left unbound after contact with the biological sample comprising the biomarker is inversely proportional to the concentration of biomarker in the sample. Upon application of this sample/anti-biomarker antibody solution to the biomarker-bound solid support, the unbound anti-biomarker antibodies are free to bind to the biomarker affixed to the support. In further embodiments, the anti-biomarker antibodies are labeled with a detectable label described herein. Measurements of the detectable label inform the amount of anti-biomarker antibody bound to the biomarker affixed to the solid support. In the above-described “competitive” immunoassay, the level of detected anti-biomarker is indirectly correlated with the amount of biomarker present in the sample. In additional embodiments, the anti-biomarker antibodies are not labeled with a detectable label. In such embodiments, a secondary antibody labeled with a detectable label can be used to measure the amount of anti-biomarker antibodies bound to the solid support.

In additional embodiments, any of the anti-biomarker antibodies are bound to a solid support, e.g., an ELISA plate. Application of samples comprising biomarker to the solid support results in biomarker binding to the affixed antibodies. The amount of biomarker bound to the anti-biomarker antibodies is then measured by application of an additional anti-biomarker antibody. The additional anti-biomarker antibody can be labeled with a detectable label described herein. Measurement of the detectable label informs the amount of biomarker bound to the anti-biomarker antibodies. In the above described “direct” immunoassay, the level of detectable label measured is positively correlated with the amount of biomarker bound to the additional anti-biomarker antibody. In additional embodiments, the anti-biomarker antibodies are not labeled with a detectable label. In such embodiments, a secondary antibody labeled with a detectable label can be used to measure the amount of anti-biomarker antibodies bound to the solid support.

Certain embodiments comprise determining a level of biomarker in a biological sample, wherein the sample was taken from a subject to whom a peptide inhibitor described herein was administered. In certain embodiments, levels of biomarkers (e.g., mRNA biomarkers) is determined by a method that comprises contacting a biological sample, e.g., after being processed to extract mRNA, with a polynucleotide that binds to or is complementary to the biomarker mRNA or its cognate cDNA, and then determining an amount of bound mRNA. In certain embodiments, the method employs quantitative PCR or RT-PCR, e.g. quantitative RT-PCR. In particular embodiments, RNA sequencing (e.g., RNA-seq) methodologies are employed. In particular embodiments, RNA expression levels are determined for tissue samples or serum samples.

Some embodiments are directed to methods of treating an inflammatory disease or disorder in a subject in need thereof comprising (i) providing to the subject an effective amount of a peptide inhibitor described herein, (ii) waiting for a first period of time, and (iii) determining the amount or level of one or more biomarkers described herein in a biological sample collected from the subject through the use of an immunoassay. Further embodiments comprise (iv) providing additional peptide inhibitor described herein to the subject if the determined level or amount of the one or more biomarker is above a defined cut-off value, or not providing additional peptide inhibitor, or providing a reduced amount of peptide inhibitor to the subject, if the level or amount of the one or more biomarker is below a defined cut-off value (or the opposite for CLDN8). The method may be repeated once or more, e.g., over the course of treatment of a subject with a peptide inhibitor described herein. Methods of the present invention may be used to monitor biomarker levels in a subject over the course of treatment with a peptide inhibitor described herein, allowing the amount of peptide inhibitor provided to the subject to be adjusted to maintain a desired level of peptide inhibitor in the subject. In certain embodiments, the marker is MPO or LCN2 and the biological sample is serum or feces, and the peptide inhibitor is selected from those described herein, e.g., any of those shown in Table 6, e.g., Peptide A-D or S-V.

A “period of time” refers to the amount of time allowed to elapse after an initial or previous treatment with a peptide inhibitor described herein (e.g., “a first period of time”). The first period of time may be at least 24 hours, at least 48 hours, at least 72 hours, at least 4 days, at least 5 days, at least 6 days, at least 7 days, at least 14 days, at least 21 days, at least one month, at least two months, at least three months or at least four months.

As used herein, a “cut-off value” refers to a value used to determine providing of additional peptide inhibitor or not providing additional peptide inhibitor. The cut-off value may be binary in nature, e.g. “one” and “zero”, wherein “one” indicates the presence of a biomarker in a biological sample and wherein “zero” indicates the absence (or an amount below the limit of detection) of a biomarker in a biological sample. The cut-off value may be a concentration, e.g., μg/L. In some embodiments, additional peptide inhibitor is administered to the subject for a second period of time if the determined level of biomarker is above or equal to a cut-off value. In some embodiments, additional peptide inhibitor is not administered to the subject for a second period of time if the determined level of peptide inhibitor is below a cut-off value.

In certain embodiments, the cut-off value of a biomarker is defined as 80%, 90%, 100%, 110%, 120%, 150%, 200%, 300%, 400%, 500%, 1000%, 1500%, 2000%, 2500%, or 3000% of the determined amount or concentration of the biomarker in a corresponding biological sample obtained from a control subject not diagnosed with the disease or disorder. As used herein, a corresponding biological sample indicates a biological sample of the same type, such as, e.g., serum or feces obtained from the same type of subject, e.g., human. In certain embodiments, the cut-off value of CLDN8 is defined as 80%, 70%, 60%, 50%, 40%, 30%, 20%, 10%, or 5% of the determined amount or concentration of CLDN8 in a corresponding biological sample obtained from a control subject not diagnosed with the disease or disorder.

In certain embodiments, the cut-off value for fecal NGAL levels is 0.3 mg/kg, 0.4 mg/kg, 0.5 mg/kg, 1.0 mg/kg, 1.2 mg/kg, 1.3 mg/kg, 1.4 mg/kg, 1.5 mg/kg, 2.0 mg/kg, 4.0 mg/kg, 5.0 mg/kg, 6.0 mg/kg, or 7 mg/kg of feces. In certain embodiments, for fecal NGAL, the range of concentration of fecal NGAL indicative of effective treatment is 0.5-1.7 mg/kg for Crohns disease and 0.4-2.6 mg/kg for ulcerative colitis.

In certain embodiments, the cut-off value for plasma NGAL levels is 90 ng/ml, 100 ng/ml, 105 ng/ml, 108 ng/ml, 110 ng/ml, 120 ng/ml, 130 ng/ml, 140 ng/ml or 150 ng/ml. In certain embodiments, for serum NGAL, the cut-off value to distinguish normal and patients with inflammation is about 108 ng/ml.

In certain embodiments, the cut-off value for fecal MPO levels is 0.3 mg/kg, 0.4 mg/kg, 0.5 mg/kg, 1.0 mg/kg, 1.2 mg/kg, 1.3 mg/kg, 1.4 mg/kg, 1.5 mg/kg, 2.0 mg/kg, 4.0 mg/kg, 5.0 mg/kg, 6.0 mg/kg, 7 mg/kg of feces, 10 mg/kg, 20 mg/kg, 50 mg/kg or 100 mg/kg of feces. In certain embodiments, for fecal MPO, the targeted value in patients responding to treatment is ≤2 mg/kg of feces.

“Providing additional peptide inhibitor” may comprise providing the same or another peptide inhibitor described herein, at the same or a different amount, e.g., an additional amount of the same peptide inhibitor equal to an initial dose of the peptide inhibitor. As used herein, “dose” refers to the amount of administered peptide inhibitor. In further embodiments, an additional dose of peptide inhibitor that is equal to an initial dose of peptide inhibitor is administered at least once after initial peptide inhibitor administration. In further embodiments, an additional dose of peptide inhibitor equal to an initial dose of peptide inhibitor is administered at least 2, 3, 4, 5, 6, 7, 8, 9, 10, 15 times or more after initial peptide inhibitor administration. In some embodiments, “providing additional peptide inhibitor” may comprise providing an additional dose of peptide inhibitor greater than an initial dose of peptide inhibitor. In further embodiments, an additional dose of peptide inhibitor that is greater than an initial dose of peptide inhibitor is administered at least once after initial peptide inhibitor administration. In further embodiments, an additional dose of peptide inhibitor greater than an initial dose of peptide inhibitor is administered at least 2, 3, 4, 5, 6, 7, 8, 9, 10, 15, or more time after initial peptide inhibitor administration. In any of the above described embodiments, the presence or absence of, or an amount of peptide inhibitor can be detected before or after each, some, or all administrations of additional peptide inhibitor.

As used herein, “not providing additional peptide inhibitor” may comprise cessation of peptide inhibitor administration. In some embodiments, “not providing additional peptide inhibitor” may comprise providing a dose of peptide inhibitor less than an initial or previous dose of peptide inhibitor. In further embodiments, a dose of peptide inhibitor less than an initial dose of peptide inhibitor is administered at least once. In further embodiments, a dose of peptide inhibitor less than an initial dose of peptide inhibitor is administered at least 2, 3, 4, 5, 6, 7, 8, 9, 10, 15 or more times after initial peptide inhibitor administration. In any of the above described embodiments, the presence or absence of, or an amount of peptide inhibitor can be detected before or after each, some, or all administrations of peptide inhibitor.

According to any method described herein in certain embodiments, the disease or disorder is an inflammatory disease or disorder. In particular embodiments, the disease or disorder is an inflammatory disease or disorder of the gastrointestinal tract. In certain embodiments, the disease or disorder is an inflammatory bowel disease. In certain embodiments, the disease or disorder is autoimmune inflammation or related diseases and disorders, such as multiple sclerosis, asthma, rheumatoid arthritis, inflammatory bowel diseases (IBDs), juvenile IBD, adolescent IBD, Crohn's disease, sarcoidosis, Systemic Lupus Erythematosus, ankylosing spondylitis (axial spondyloarthritis), psoriatic arthritis, or psoriasis. In particular embodiments, the disease or disorder is psoriasis (e.g., plaque psoriasis, guttate psoriasis, inverse psoriasis, pustular psoriasis, Palmo-Plantar Pustulosis, psoriasis vulgaris, or erythrodermic psoriasis), atopic dermatitis, acne ectopica, ulcerative colitis, Crohn's disease, Celiac disease (nontropical Sprue), enteropathy associated with seronegative arthropathies, microscopic colitis, collagenous colitis, eosinophilic gastroenteritis/esophagitis, colitis associated with radio- or chemo-therapy, colitis associated with disorders of innate immunity as in leukocyte adhesion deficiency-1, chronic granulomatous disease, glycogen storage disease type 1b, Hermansky-Pudlak syndrome, Chediak-Higashi syndrome, Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, primary biliary cirrhosis, viral-associated enteropathy, pericholangitis, chronic bronchitis, chronic sinusitis, asthma, uveitis, or graft versus host disease.

In certain embodiments, the one or more biomarker is a protein having upregulated expression in cell, tissues or organs having inflammation. In particular embodiments, the one or more biomarker is selected from LCN2/lipocalin, myeloperoxidase (MPO), interleukin-1beta (IL-1B), interleukin-6 (IL-6), interleukin-17A (IL-17A), IL-17F, pSTAT3, and interleukin-22 (IL-22). In particular embodiments, the one or more biomarker is selected from MMP-9, S100A8, or miR223-3p. Lipocalins are a protein family, for which the sequences of most members of the family, the core or kernel lipocalins, are characterised by three short conserved stretches of residues, while others, the outlier lipocalin group, share only one or two of these. Proteins known to belong to this family include alpha-1-microglobulin (protein HC); major urinary proteins; alpha-1-acid glycoprotein (orosomucoid); aphrodisin; apolipoprotein D; beta-lacoglobulin; complement component C8 gamma chain; crustacyanin; epididymal-retinoic acid binding protein (E-RABP); insectacyanin; odorant binding protein (OBP); human pregnancy-associated endometrial alpha-2 globulin (PAEP); probasin (PB), a prostatic protein; prostaglandin D synthase; purpurin; Von Ebner's gland protein (VEGP); and lizard epididymal secretory protein IV (LESP IV). In certain embodiments, the one or more biomarker is lipocalin 2 (LCN2). In particular embodiments, the biomarker is a human proteins that contain lipocalin domain selected from: Alpha-1-Microglobulin/Bikunin Precursor (AMBP), apolipoprotein D (APOD), complement C8 gamma chain (C8G), Cellular Retinoic Acid Binding Protein (CRABP) 1 (CRABP1), CRABP2, fatty acid binding protein (FABP) 1 (FABP1), FABP2, FABP3, FABP4, FABP5, FABP6, FABP7, lipocalin (LCN) 1 (LCN1), LCN2, LCN8, LCN9, LCN10, LCN12, odorant binding protein (OBP) 2A (OBP2A), OBP2B, Alpha-1-acid glycoprotein (ORM) 1 (1ORM1), ORM2, Progestagen Associated Endometrial Protein (PAEP), PERF15, peripheral myelin protein 2 (PMP2), Prostaglandin D2 Synthase (PTGDS), retinol binding protein (RBP) 1 (RBP1), RBP2, RBP4, RRB5, RBP7 and UNQ2541. In certain embodiments, the biomarker is a human protein that is involved in the regulation of colonic tight junctions and epithelial integrity, such as claudin-8 (CLDN8).

In certain embodiments of any of the methods described herein, the peptide inhibitor is provided to the subject in a pharmaceutical composition comprising the peptide inhibitor and a pharmaceutically acceptable carrier, excipient, or diluent. In certain embodiments, the pharmaceutical composition is provided to the subject by an oral, parenteral, intravenous, peritoneal, intradermal, subcutaneous, intramuscular, intrathecal, inhalation, vaporization, nebulization, sublingual, buccal, parenteral, rectal, intraocular, inhalation, topically, vaginal, or topical route of administration. In particular embodiments, the pharmaceutical composition is formulated for oral, intravenous or topical administration. In certain embodiments, it comprises an enteric coating, e.g., wherein the enteric coating protects and releases the pharmaceutical composition within a subject's lower gastrointestinal system. In particular embodiments, the method is used for treating Inflammatory Bowel Disease (IBD), ulcerative colitis, or Crohn's disease, and the pharmaceutical composition is provided to the subject orally. In certain embodiments, the method is used for treating psoriasis, and the pharmaceutical composition is provided to the subject orally, topically, parenterally, intravenously, subcutaneously, peritoneally, or intravenously. In certain embodiments, the peptide inhibitor or the peptide dimer inhibitor inhibits binding of an interleukin-23 (IL-23) to the interleukin-23 receptor (IL-23R). In particular embodiments, the total daily dosage of a peptide inhibitor that is provided to a human or other mammal host in single or divided doses may be in amounts, for example, from 0.0001 to 300 mg/kg body weight daily or 1 to 300 mg/kg body weight daily.

In certain embodiments, a reagent that binds to the biomarker is an antibody or an antigen-binding fragment thereof, which may collectively be referred to as “antibody” herein. The antibody or antigen-binding fragment thereof is said to specifically bind a biomarker when it preferentially recognizes the biomarker in a complex mixture of proteins and/or macromolecules. An antibody may also be said to specifically bind a biomarker when the equilibrium dissociation constant is ≤10⁻⁷ or 10⁻⁸ M. In some embodiments, the equilibrium dissociation constant may be ≤10⁻⁹ M or ≤10⁻¹⁰ M. In further embodiments, the equilibrium dissociation constant may be ≤10⁻¹¹ M or less.

In certain embodiments, antibodies and antibody-binding fragments thereof may be provided in the form of a UniBody®. A UniBody® is an IgG4 antibody with the hinge region removed (see GenMab Utrecht, The Netherlands; see also, e.g., US20090226421). In certain embodiments, the antibodies of the present disclosure may take the form of a Nanobody. Nanobodies are encoded by single genes and are efficiently produced in almost all prokaryotic and eukaryotic hosts, e.g. E. coli (see e.g. U.S. Pat. No. 6,765,087), molds (for example Aspergillus or Trichoderma) and yeast (for example Saccharomyces, Kluyvermyces, Hansenula or Pichia (see e.g. U.S. Pat. No. 6,838,254). In certain embodiments, the antibodies of the present disclosure may be chimeric antibodies. In this regard, a chimeric antibody is comprised of an antigen-binding fragment of an anti-biomarker antibody operably linked or otherwise fused to a heterologous Fc portion of a different antibody. In certain embodiments, the heterologous Fc domain is of human origin. In further embodiments, the heterologous Fc domain may be from a different Ig class from the parent antibody, including IgA (including subclasses IgA1 and IgA2), IgD, IgE, IgG (including subclasses IgG1, IgG2, IgG3, and IgG4), and IgM. In further embodiments, the heterologous Fc domain may be comprised of CH2 and CH3 domains from one or more of the different Ig classes. In certain embodiments, the antibodies of the present disclosure may be “non-naturally occurring” antibodies. Non-naturally occurring antibodies can refer to antibodies that comprise one or more amino acid modifications, such that the resultant antibody is substantially non-naturally occurring (e.g., does not exists in nature). These amino acid modifications can include point mutations, wherein a naturally occurring amino acid is substituted for another naturally occurring amino acid. In some embodiments, the amino acid modifications can include point mutations wherein a non-naturally occurring amino acid is substituted for a naturally occurring amino acid. Non-naturally occurring antibodies can also refer to antibodies that are conjugated to a heterologous protein or compound, such as a detectable marker. In certain embodiments, an antibody is a monoclonal antibody (mAb). The term “monoclonal antibody” encompasses not only intact monoclonal antibodies and full-length monoclonal antibodies, but also fragments thereof.

The present invention also includes variants and fragments of the antibodies, and antigen-binding fragments thereof, described herein, wherein the variants and fragments bind to a biomarker. In particular embodiments, the variants comprise one or more amino acid modification as compared to any of the antibodies, or antigen-binding fragments thereof, described herein, e.g., an amino acid substitution, deletion, or addition

Antibodies that specifically bind to the biomarkers disclosed herein and that can be used to practice the methods described herein are commercially available or can be readily produced using routine methods in the art. For example commercially available human LCN2 ELISA kit may be obtained from BioVendor R&D (Cat. RD191102200R), and commercially available human MPO ELISA kit may be obtained from Immundiagnostik (Cat. K 6631B).

In certain embodiments, a reagent that binds to a biomarker is conjugated to a detectable label. A “detectable label” as use herein refers to a molecule or material that can produce a detectable (visually, electronically, or otherwise) signal that indicates the presence and/or concentration of the label in a sample. In some embodiments, an antibody, or antigen-binding fragment thereof, that binds the biomarker is linked to a detectable label. Non-limiting examples of detectable labels include fluorescent labels, polymer particles, metal particles, haptens, enzyme labels, luminescent labels, electrochemiluminescent labels, bioluminescent labels, radioisotopes, oligonucleotides, and nanoparticles.

Examples of fluorescent labels include 5-(and 6)-carboxyfluorescein, 5- or 6-carboxyfluorescein, 6-(fluorescein)-5-(and 6)-carboxamido hexanoic acid, fluorescein isothiocyanate (FITC), rhodamine, tetramethylrhodamine, and dyes such as Cy2, Cy3, and Cy5, optionally substituted coumarin including AMCA, PerCP, phycobiliproteins including R-phycoerythrin (RPE) and allophycoerythrin (APC), Texas Red, Princeton Red, green fluorescent protein (GFP) and analogs thereof, and conjugates of R-phycoerythrin or allophycoerythrin, inorganic fluorescent labels such as particles based on semiconductor material like coated CdSe nanocrystallites.

Examples of polymer particle labels include micro particles or latex particles of polystyrene, PMMA or silica, which can be embedded with fluorescent dyes, or polymer micelles or capsules which contain dyes, enzymes or substrates.

Examples of metal particle labels include gold particles and coated gold particles, which can be converted by silver stains. Examples of haptens include dinitrophenyl (DNP), fluorescein isothiocyanate (FITC), biotin, and digoxigenin. Examples of enzymatic labels include horseradish peroxidase (HRP), alkaline phosphatase (ALP or AP), β-galactosidase (GAL), glucose-6-phosphate dehydrogenase, β-N-acetylglucosamimidase, β-glucuronidase, invertase, Xanthine Oxidase, firefly luciferase and glucose oxidase (GO). Examples of commonly used substrates for horseradish peroxidase include 3,3′-diaminobenzidine (DAB), diaminobenzidine with nickel enhancement, 3-amino-9-ethylcarbazole (AEC), Benzidine dihydrochloride (BDHC), Hanker-Yates reagent (HYR), Indophane blue (IB), tetramethylbenzidine (TMB), 4-chloro-1-naphtol (CN), .alpha.-naphtol pyronin (.alpha.-NP), o-dianisidine (OD), 5-bromo-4-chloro-3-indolylphosphate (BCIP), Nitro blue tetrazolium (NBT), 2-(p-iodophenyl)-3-p-nitropheny-1-5-phenyl tetrazolium chloride (INT), tetranitro blue tetrazolium (TNBT), 5-bromo-4-chloro-3-indoxyl-beta-D-galactoside/ferro-ferricyanide (BCIG/FF).

Examples of commonly used substrates for Alkaline Phosphatase include Naphthol-AS-B 1-phosphate/fast red TR (NABP/FR), Naphthol-AS-MX-phosphate/fast red TR (NAMP/FR), Naphthol-AS-B1-phosphate/-fast red TR (NABP/FR), Naphthol-AS-MX-phosphate/fast red TR (NAMP/FR), Naphthol-AS-B1-phosphate/new fuschin (NABP/NF), bromochloroindolyl phosphate/nitroblue tetrazolium (BCIP/NBT), 5-Bromo-4-chloro-3-indolyl-b-d-galactopyranoside (BCIG).

Examples of luminescent labels include luminol, isoluminol, acridinium esters, 1,2-dioxetanes and pyridopyridazines. Examples of electrochemiluminescent labels include ruthenium derivatives. Examples of radioactive labels include radioactive isotopes of iodide, cobalt, selenium, tritium, carbon, sulfur and phosphorous.

Nanoparticles particles range from 1-1000 nm in size and include diverse chemical structures such as gold and silver particles and quantum dots. When irradiated with angled incident white light, silver or gold nanoparticles ranging from 40-120 nm will scatter monochromatic light with high intensity. The wavelength of the scattered light is dependent on the size of the particle. Four to five different particles in close proximity will each scatter monochromatic light, which when superimposed will give a specific, unique color. The particles are being manufactured by companies such as Genicon Sciences (Carlsbad, Calif.). Derivatized silver or gold particles can be attached to a broad array of molecules including, proteins, antibodies, small molecules, receptor ligands, and nucleic acids.

Further examples of nanoparticles include quantum dots. Quantum dots are fluorescing crystals 1-5 nm in diameter that are excitable by light over a large range of wavelengths. Upon excitation by light having an appropriate wavelength, these crystals emit light, such as monochromatic light, with a wavelength dependent on their chemical composition and size. Quantum dots such as CdSe, ZnSe, InP, or InAs possess unique optical properties; these and similar quantum dots are available from a number of commercial sources (e.g., NN-Labs, Fayetteville, Ark.; Ocean Nanotech, Fayetteville, Ark.; Nanoco Technologies, Manchester, UK; Sigma-Aldrich, St. Louis, Mo.).

Many dozens of classes of particles can be created according to the number of size classes of the quantum dot crystals. The size classes of the crystals are created either 1) by tight control of crystal formation parameters to create each desired size class of particle, or 2) by creation of batches of crystals under loosely controlled crystal formation parameters, followed by sorting according to desired size and/or emission wavelengths. Two examples of references in which quantum dots are embedded within intrinsic silicon epitaxial layers of semiconductor light emitting/detecting devices are U.S. Pat. Nos. 5,293,050 and 5,354,707 to Chapple Sokol, et al.

Detectable labels may be linked to antibodies or to any other molecule that specifically binds to a biomarker of interest, e.g., an antibody, a nucleic acid probe, or a polymer. Furthermore, one of ordinary skill in the art would appreciate that detectable labels can also be conjugated to second, and/or third, and/or fourth, and/or fifth binding agents or antibodies, etc. Moreover, the skilled artisan would appreciate that each additional binding agent or antibody used to characterize a biomarker of interest may serve as a signal amplification step. In certain embodiments, the biomarker may be detected and/or quantitated visually using, e.g., light microscopy, fluorescent microscopy, electron microscopy where the detectable substance is for example a dye, a colloidal gold particle, a luminescent reagent. Visually detectable substances bound to a biomarker may also be detected using a spectrophotometer. Where the detectable substance is a radioactive isotope, detection and/or quantification can be performed visually by autoradiography, or non-visually using a scintillation counter. See, e.g., Larsson, 1988, Immunocytochemistry: Theory and Practice, (CRC Press, Boca Raton, Fla.); Methods in Molecular Biology, vol. 80 1998, John D. Pound (ed.) (Humana Press, Totowa, N.J.). In certain embodiments, reagent that binds a biomarker is bound to a detectable label that emits an altered or different signal (e.g., increased or reduced) when bound to the biomarker.

In certain embodiments, an agent that binds to a marker is a polynucleotide, e.g., an oligonucleotide that binds to mRNA or cDNA, such as an oligonucleotide comprising a sequence complementary to a biomarker mRNA or cDNA sequence.

The present disclosure include kits comprising one or more reagent capable of detecting one or more biomarkers are selected from: myeloperoxidase (MPO), interleukin-1β (IL-1β), interleukin-6 (IL-6), interleukin-22 (IL-22), interleukin-17A (IL-17A), interleukin-17F (IL-17F), lipocalin 2 (LCN2), matrix metallopeptidase 9 (MMP9), S100 calcium-binding protein A8 (S100A8), claudin 8 (CLDN8), microRNA-223-3p (miR223-3p), or phosphorylated signal transducer and activator of transcription 3 (pSTAT3). In particular embodiments, the one or more reagents bind to one or more of MMP9, MPO and LCN2. In particular embodiments, the one or more reagent binds to the one or more biomarker. In particular embodiments, the one or more reagents are antibodies that bind to one or more of the biomarkers, wherein the biomarker is a protein. The antibodies may be bound to a solid support, e.g., an array. In particular embodiments, the one or more reagents are polynucleotides that bind to one or more biomarkers, wherein the one or more biomarkers are mRNA, cDNA, or miRNA. In particular embodiments, the kit further provides reagent for performing RNA sequencing or RT-PCR. Various reagents may be present in different or the same containers.

In certain embodiments, any of the kits further comprise reagents for extracting protein from feces or serum.

In certain embodiments, the kit comprises one or more peptide inhibitor disclosed herein.

### Peptide Inhibitors of Interleukin-23 Receptor

Peptide inhibitors of interleukin-23 receptor (IL-23R) that may be used according to the present invention include, but are not limited to, any of those described in International Patent Application PCT/US2015/040658 filed on Jul. 15, 2015, or International Patent Application PCT/US2016/042680 filed on Jul. 15, 2016, each of which is incorporated by reference herein in its entirety. Peptide inhibitors also include pharmaceutically acceptable salts of any of the peptide inhibitor described herein.

In certain embodiments, the peptide inhibitor comprises an amino acid sequence of Formula (Xa):

X1-X2-X3-X4-X5-X6-X7-X8-X9-X10-X11-X12-X13-X14-X15-X16-X17-X18-X19-X20 (Xa),

wherein:
X1, X2 and X3 are any amino acid or absent
X4 is any amino acid or chemical moiety capable of forming a bond with X9;
X5, X6, X7 and X8 are any amino acid;
X9 is any amino acid or chemical moiety capable of forming a bond with X4;
X10, X11, X12, X13, X14 and X15 are any amino acid; and
X16, X17, X18, X19 and X20 are any amino acid or absent;
wherein the peptide inhibitor is cyclized via a bond between X4 and X9, and
wherein the peptide inhibitor inhibits the binding of an interleukin-23 (IL-23) to an IL-23 receptor.

In certain embodiments of peptide inhibitors of Xa: X1 is absent; X2 is absent; X3 is absent; X4 is Cys, Abu or Pen; X5 is Ala, α-MeOm, α-MeSer, Cit, Dap, Dab, Dap(Ac), Gly, Lys, Asn, N-MeGln, N-MeArg, Om, Gln, Arg, Ser or Thr; X6 is Asp or Thr; X7 is Trp or 6-Chloro-Trp; X8 is Glu, Gln or Val; X9 is Cys, Abu or Pen; X10 is 2-Nal, a Phe analog, Tyr, or a Tyr analog; X11 is 1-Nal, 2-Nal, Phe(3,4-dimethoxy), 5-HydroxyTrp, Phe(3,4-Cl2), Trp or Tyr(3-tBu); X12 is 3-Pal, Acpc, Acbc, Acvc, Achc, Agp, Aib, α-DiethylGly, α-MeLys, α-MeLys(Ac), α-MeLeu, α-MeOm, α-MeSer, α-MeVal, Cav, Cha, Cit, Cpa, D-Asn, Glu, His, hLeu, hArg, Lys, Leu, Octgly, Orn, 4-amino-4-carboxy-piperidine, Arg, Ser, Thr or THP; X13 is Cit, Asp, Dab, Dap, Phe, His, Dap(Peg2-Ac), Dap(pyroglutaric acid), Glu, HomoArg, Lys, Lys(Ac), Lys(Benzoic acid), Lys(glutaric acid), Lys(IVA), Lys(Peg4-isoGlu-Palm), Lys(pyroglutaric acid), Lys(succinic acid), Asn, Om, Gln, Arg, Thr or Val; X14 is Asp, Dab(Ac), Dap(Ac), Phe, His, Lys(Ac), Met, Asn(isobutyl), Gln, Arg, Tyr or Asp(1,4-diaminobutane); and X15 is Ala, βAla, Glu, Gly, Asn, Gln, Arg or Ser.

In certain embodiments of Xa: X1 is absent; X2 is absent; X3 is absent; X4 is Cys, Abu or Pen; X5 is Ala, α-MeOm, α-MeSer, Cit, Dap, Dab, Dap(Ac), Gly, Lys, Asn, Om, Gln, Arg, Ser or Thr; X6 is Asp or Thr; X7 is Trp or 6-Chloro-Trp; X8 is Gln or Val; X9 is Cys, Abu or Pen; X10 is 2-Nal, a Phe analog, Tyr, or a Tyr analog; X11 is 1-Nal, 2-Nal, Phe(3,4-dimethoxy), 5-HydroxyTrp, Phe(3,4-Cl₂), Trp or Tyr(3-tBu); X12 is 3-Pal, Acpc, Acbc, Acvc, Achc, Agp, Aib, α-DiethylGly, α-MeLys, α-MeLys(Ac), α-MeLeu, α-MeOm, α-MeSer, α-MeVal, Cav, Cha, Cit, Cpa, D-Asn, His, hLeu, hArg, Lys, Leu, Octgly, Orn, 4-amino-4-carboxy-piperidine, or THP; X13 is Cit, Asp, Dab, Dap, Phe, His, Dap(Peg2-Ac), Dap(pyroglutaric acid), Glu, hArg, Lys, Lys(Ac), Lys(Benzoic acid), Lys(glutaric acid), Lys(IVA), Lys(Peg4-isoGlu-Palm), Lys(pyroglutaric acid), Lys-(succinic acid), Asn, Orn, Gln, Arg, Thr or Val; X14 is Dab(Ac), Dap(Ac), Phe, His, Lys(Ac), Met, Asn, Gln, Arg, or Tyr; and X15 is Ala, betaAla, Gly, Asn, Gln, or Ser.

In certain embodiments of Xa: X1 is absent; X2 is absent; X3 is absent; X4 is Cys, Abu or Pen; X5 is Dap, Dap(Ac), Gly, Lys, Gln, Arg, Ser, Thr or Asn; X6 is Thr; X7 is Trp or 6-Chloro-Trp; X8 is Gln; X9 is Cys, Abu or Pen; X10 is 2-Nal, a Phe analog, Tyr, or a Tyr analog; X11 is 1-Nal, 2-Nal, Phe(3,4-dimethoxy), Phe(3,4-Cl₂), or Trp; X12 is Acpc, Acbc, Acvc, Achc, Aib, α-DiethylGly, α-MeLys, α-MeLys(Ac), α-MeLeu, α-MeOm, α-MeSer, α-MeVal, Cha, Cit, hLeu, Lys, Leu, Arg or THP; X13 is Cit, Asp, Dap, Dap(Peg2-Ac), Dap(pyroglutaric acid), Glu, hArg, Lys, Lys(Ac), Lys(Benzoic acid), Lys(glutaric acid), Lys(IVA), Lys(Peg4-isoGlu-Palm), Lys(pyroglutaric acid), Lys-(succinic acid), Asn, Orn, Gln, Arg, or Val; X14 is Dab(Ac), Dap(Ac), His, Lys(Ac), Asn, Gln, or Tyr; and X15 is Ala, betaAla, Gly, Asn, Gln, or Ser.

In certain embodiments of Xa: X1 is absent; X2 is absent; X3 is absent; X4 is Cys, Abu or Pen; X5 is Dap, Dap(Ac), Gln, Ser, Thr or Asn; X6 is Thr; X7 is Trp; X8 is Gln; X9 is Cys, Abu or Pen; X10 is a Phe analog, Tyr, or a Tyr analog; X11 is 2-Nal or Trp; X12 is Acpc, Acbc, Acvc, Achc, Aib, α-DiethylGly, α-MeLys, α-MeLys(Ac), α-MeLeu, α-MeOm, α-MeSer, α-MeVal, hLeu, Leu, or THP; X13 is Cit, Asp, Glu, Lys, Lys(Ac), Asn, or Gln; X14 is Dab(Ac), Asn, or His; and X15 is Ala, betaAla, Gly, Asn, or Gln.

In certain embodiments of Xa: X4 is Cys, Pen, hCys, D-Pen, D-Cys, D-hCys, Met, Glu, Asp, Lys, Orn, Dap, Dab, D-Dap, D-Dab, D-Asp, D-Glu, D-Lys, Sec, 2-chloromethylbenzoic acid, mercapto-propanoic acid, mercapto-butyric acid, 2-chloro-acetic acid, 3-choropropanoic acid, 4-chlorobutyric acid, 3-chloroisobutyric acid, Abu, β-azido-Ala-OH, propargylglycine, 2-(3′-butenyl)glycine, 2-allylglycine, 2-(3′-butenyl)glycine, 2-(4′-pentenyl)glycine, 2-(5′-hexenyl)glycine, or Abu; X7 is Trp, Glu, Gly, Ile, Asn, Pro, Arg, Thr or OctGly, or a corresponding α-methyl amino acid form of any of the foregoing; X9 is Cys, Pen, hCys, D-Pen, D-Cys, D-hCys, Glu, Lys, Orn, Dap, Dab, D-Dap, D-Dab, D-Asp, D-Glu, D-Lys, Asp, Leu, Val, Phe, or Ser, Sec, Abu, β-azido-Ala-OH, propargylglycine, 2-2-allylglycine, 2-(3′-butenyl)glycine, 2-(4′-pentenyl)glycine, Ala, hCys, Abu, Met, MeCys, (D)Tyr or 2-(5′-hexenyl)glycine; X10 is Tyr, Phe(4-OMe), 1-Nal, 2-Nal, Aic, α-MePhe, Bip, (D)Cys, Cha, DMT, (D)Tyr, Glu, His, hPhe(3,4-dimethoxy), hTyr, N-Me-Tyr, Trp, Phe(4-CONH₂), Phe(4-phenoxy), Thr, Tic, Tyr(3-tBu), Phe(4-tBu), Phe(4-CN), Phe(4-Br), Phe(4-NH₂), Phe(4-F), Phe(3,5-F₂), Phe(4-CH₂CO₂H), Phe(penta-F), Phe(3,4-Cl₂), Phe(4-CF₃), Phe(4-OCH₃), Bip, Cha, 4-PyridylAlanine, βhTyr, OctGly, Phe(4-N₃), Phe(4-Br), Phe[4-(2-aminoethoxy)] or Phe, a Phe analog, a Tyr analog, or a corresponding α-methyl amino acid form of any of the foregoing; X11 is 2-Nal, 1-Nal, 2,4-dimethylPhe, Bip, Phe(3,4-Cl₂), Phe (3,4-F₂), Phe(4-CO₂H), βhPhe(4-F), α-Me-Trp, 4-phenylcyclohexyl, Phe(4-CF₃), α-MePhe, βhNal, βhPhe, βhTyr, βhTrp, Nva(5-phenyl), Phe, His, hPhe, Tic, Tqa, Trp, Tyr, Phe(4-OMe), Phe(4-Me), Trp(2,5,7-tri-tert-Butyl), Phe(4-Oallyl), Tyr(3-tBu), Phe(4-tBu), Phe(4-guanidino, Phe(4-OBzl), Octgly, Glu(Bzl), 4-Phenylbenzylalanine, Phe[4-(2-aminoethoxy)], 5-Hydroxy-Trp, 6-Chloro-Trp, N-MeTrp, 1,2,3,4-tetrahydro-norharman, Phe(4-CONH₂), Phe(3,4-Dimethoxy), Phe(2,3-Cl₂), Phe(2,3-F₂), Phe(4-F), 4-phenylcyclohexylalanine, Bip, or a corresponding α-methyl amino acid form of any of the foregoing; X12 is His, Phe, Arg, N-Me-His, Val, Cav, Cpa, Leu, Cit, hLeu, 3-Pal, t-butyl-Ala, 4-amino-4-carboxy-tetrahydropyran, Achc Acpc, Acvc, Acbc, Agp, Aib, α-DiethylGly, α-MeLys, α-MeLys(Ac), α-Me-Leu, α-MeOm, α-MeSer, α-MeVal, Aib, D-Ala, (D)Asn, (D)Asp, (D)Leu, (D)Phe, (D)Tyr, Aib, α-MeLeu, α-MeOm, β-Aib, β-Ala, βhAla, βhArg, βhLeu, βhVal, β-spiro-pip, Glu, hArg, Ile, Lys, N-MeLeu, N-MeArg, Ogl, Orn, Pro, Gln, Ser, Thr, Tle, t-butyl-Gly, or a corresponding α-methyl amino acid form of any of the foregoing; X13 is Thr, Sarc, Glu, Phe, Arg, Leu, Lys, Arg, Orn, Val, βhAla, Lys(Ac), (D)Asn, (D)Leu, (D)Phe, (D)Thr, Ala, α-MeLeu, Aib, β-Ala, β-Glu, βhLeu, βhVal, β-spiro-pip, Cha, Chg, Asp, Dab, Dap, α-DiethylGly, hLeu, Asn, Ogl, Pro, Gln, Ser, β-spiro-pip, Thr, Tba, Tle or Aib, Cit, hArg, Lys, Asn, Om, Gln or a corresponding α-methyl amino acid form of any of the foregoing; X14 is Phe, Tyr, Glu, Gly, His, Lys, Leu, Met, Asn, Pro, Gln, Arg, Ser, Thr, TicpβhPhe, Arg, Lys(Ac), His; Dap(Ac), Dab(Ac), Asp or a corresponding α-methyl amino acid form of any of the foregoing; X15 is Gly, Ser, Thr, Gln, Ala, (D)Ala, (D)Asn, (D)Asp, (D)Leu, (D)Phe, (D)Thr, Aea, Asp, Asn, Glu, Phe, Gly, Lys, Leu, Pro, Arg, β-Ala, Sarc, or a corresponding α-methyl amino acid form of any of the foregoing; X16 is Asp, Glu, Ala, AEA, AEP, βhAla, Gaba, Gly, Ser, Pro, Asn, Thr or absent, or a corresponding α-methyl amino acid form of any of the foregoing; and X17 is Leu, Lys, Arg, Glu, Ser, Gly, Gln or absent, or a corresponding α-methyl amino acid form of any of the foregoing.

In certain embodiments of peptide inhibitors of Xa, the bond is a disulfide bond, a thioether bond, a lactam bond, a triazole ring, a selenoether bond, a diselenide bond, or an olefin bond.

In particular embodiments of peptide inhibitors of Xa, X4 is Cys and X9 is Cys, and the bond is a disulfide bond. In particular embodiments, X4 is Pen and X9 is Pen, and the bond is a disulfide bond. In certain embodiments: X7 is Trp; X10 is Phe, Tyr, a Phe analog, or a Tyr analog; X11 is Trp, 1-Nal or 2-Nal; and X12 is Aib, α-Me-Lys, α-Me-Leu, Achc, Acvc, Acpc, Acbc or THP. In certain embodiments: X7 is Trp; X10 is Phe, Tyr, a Phe analog, or a Tyr analog; X11 is Trp, 1-Nal or 2-Nal; and X12 is Aib, α-Me-Lys or α-Me-Leu. In particular embodiments, the peptide inhibitor comprises any of the following the amino acid sequences: Pen-Q-T-W-Q-Pen-[Phe(4-OMe)]-[2-Nal]-[α-Me-Lys]-E-N-G; Pen-N-T-W-Q-[Pen]-[Phe[4-(2-aminoethoxy)]-[2-Nal]-[Aib]-[Lys(Ac)]-N-N; Pen-Q-T-W-Q-[Pen]-[Phe[4-(2-aminoethoxy)]-[2-Nal]-[α-MeLeu]-[Lys(Ac)]-N-N; or Pen-Q-T-W-Q-[Pen]-[Phe(4-CONH₂)]-[2-Nal]-[α-MeLys]-[Lys(Ac)]-N-N, wherein the peptide inhibitor comprises a disulfide bond between the two Pen amino acids.

In particular embodiments of peptide inhibitors of Xa, X4 is an amino acid, aliphatic acid, alicyclic acid or modified 2-methyl aromatic acid having a carbon side chain capable of forming a thioether bind with X9; X9 is a sulfur-containing amino acid capable of forming a thioether bond with X4, and the bond between X4 and X9 is a thioether bond. In certain embodiments, X4 is Abu, 2-chloromethylbenzoic acid, mercapto-propanoic acid, mercapto-butyric acid, 2-chloro-acetic acid, 3-chloro-propanoic acid, 4-chloro-butyric acid, 3-chloro-isobutyric acid; and X9 is Abu, Cys, Pen, hCys, D-Pen, D-Cys, or D-hCys. In certain embodiments, X4 is Abu; and X9 is Cys. In certain embodiments, X7 is Trp; X10 is Phe, Tyr, a Phe analog, or a Tyr analog; X11 is Trp, 1-Nal or 2-Nal; and X12 is α-Me-Lys, α-Me-Leu, α-Me-Ser, α-Me-Val, Achc, Acvc, Acpc, Acbc, or [4-amino-4-carboxy-tetrahydropyran]. In certain embodiments, X7 is Trp; X10 is Phe, Tyr, a Phe analog, or a Tyr analog; X11 is Trp, 1-Nal or 2-Nal; and X12 is α-Me-Lys or [4-amino-4-carboxy-tetrahydropyran]. In particular embodiments, the peptide inhibitor comprises any of the following amino acid sequences: [Abu]-Q-T-W-Q-C-[Phe(4-OMe)]-[2-Nal]-[α-MeLys]-E-N-G; [Abu]-Q-T-W-Q-C-[Phe(4-(2-aminoethoxy))]-W-[α-MeLys]-E-N-G; or [Abu]-Q-T-W-Q-C-[Phe[4-(2-aminoethoxy)]]-[2-Nal]-[4-amino-4-carboxy-tetrahydropyran]-E-N-N, wherein the peptide inhibitor comprises a thioether bond between the Abu and the C.

In certain embodiments of peptide inhibitors of Xa: X4 is Pen, Cys or hCys; X5 is any amino acid; X6 is any amino acid; X7 is Trp, Bip, Gln, His, Glu(Bzl), 4-Phenylbenzylalanine, Tic, Phe[4-(2-aminoethoxy)], Phe(3,4-Cl₂), Phe(4-OMe), 5-Hydroxy-Trp, 6-Chloro-Trp, N-MeTrp, α-Me-Trp, 1,2,3,4-tetrahydro-norharman, Phe(4-CO₂H), Phe(4-CONH₂), Phe(3,4-Dimethoxy), Phe(4-CF₃), Phe(4-tBu), P1-diPheAla, Glu, Gly, Ile, Asn, Pro, Arg, Thr or Octgly, or a corresponding α-methyl amino acid form of any of the foregoing; X8 is any amino acid; X9 is Pen, Cys or hCys; X10 is 1-Nal, 2-Nal, Aic, Bip, (D)Cys, Cha, DMT, (D)Tyr, Glu, Phe, His, Trp, Thr, Tic, Tyr, 4-pyridylAla, Octgly, a Phe analog or a Tyr analog (optionally, Phe(3,4-F₂), Phe(3,4-Cl₂), F(3-Me), Phe[4-(2-aminoethoxy)], Phe[4-(2-(acetyl-aminoethoxy)], Phe(4-Br), Phe(4-CONH₂), Phe(4-Cl), Phe(4-CN), Phe(4-guanidino), Phe(4-Me), Phe(4-NH₂), Phe(4-N₃), Phe(4-OMe), or Phe(4-OBzl)), or a corresponding α-methyl amino acid form of any of the foregoing; X11 is 2-Nal, 1-Nal, 2,4-dimethylPhe, Bip, Phe(3,4-Cl₂), Phe (3,4-F₂), Phe(4-CO₂H), βhPhe(4-F), α-Me-Trp, 4-phenylcyclohexyl, Phe(4-CF₃), α-MePhe, βhNal, βhPhe, βhTyr, βhTrp, Nva(5-phenyl), Phe, His, hPhe, Tic, Tqa, Trp, Tyr, Phe(4-OMe), Phe(4-Me), Trp(2,5,7-tri-tert-Butyl), Phe(4-Oallyl), Tyr(3-tBu), Phe(4-tBu), Phe(4-guanidino, Phe(4-OBzl), Octgly, Glu(Bzl), 4-Phenylbenzylalanine, Phe[4-(2-aminoethoxy)], 5-Hydroxy-Trp, 6-Chloro-Trp, N-MeTrp, 1,2,3,4-tetrahydro-norharman, Phe(4-CONH₂), Phe(3,4-OMe₂) Phe(2,3-Cl₂), Phe(2,3-F₂), Phe(4-F), 4-phenylcyclohexylalanine or Bip, or a corresponding α-methyl amino acid form of any of the foregoing; X12 is α-MeLys, α-MeOm, α-MeLeu, α-MeVal, 4-amino-4-carboxy-tetrahydropyran, Achc, Acpc, Acbc, Acvc, MeLeu, Aib, (D)Ala, (D)Asn, (D)Leu, (D)Asp, (D)Phe, (D)Thr, 3-Pal, Aib, β-Ala, βhGlu, βhAla, βhLeu, βhVal, β-spiro-pip, Cha, Chg, Asp, Dab, Dap, α-DiethylGly, Glu, Phe, hLeu, hArg, hLeu, Ile, Lys, Leu, Asn, N-MeLeu, N-MeArg, Ogl, Om, Pro, Gln, Arg, Ser, Thr or Tle, or a corresponding α-methyl amino acid form of any of the foregoing; X13 is Lys(Ac), (D)Asn, (D)Leu, (D)Thr, (D)Phe, Ala, Aib, α-MeLeu, R-Ala, βhGlu, βhAla, βhLeu, βhVal, β-spiro-pip, Cha, Chg, Asp, Lys, Arg, Om, Dab, Dap, α-DiethylGly, Glu, Phe, hLeu, Lys, Leu, Asn, Ogl, Pro, Gln, Asp, Arg, Ser, spiro-pip, Thr, Tba, Tlc, Val or Tyr, or a corresponding α-methyl amino acid form of any of the foregoing; X14 is Asn, Glu, Phe, Gly, His, Lys, Leu, Met, Asn, Pro, Gln, Arg, Ser, Thr, Tic or Tyr, Lys(Ac), Om or a corresponding α-methyl amino acid form of any of the foregoing; X15 is Gly, (D)Ala, (D)Asn, (D)Asp, Asn, (D)Leu, (D)Phe, (D)Thr, Ala, AEA, Asp, Glu, Phe, Gly, Lys, Leu, Pro, Gln, Arg or Ser, R-Ala, Arg or a corresponding α-methyl amino acid form of any of the foregoing; X16 is absent, Gly, Ala, Asp, Ser, Pro, Asn or Thr, or a corresponding α-methyl amino acid form of any of the foregoing; X17 is absent, Glu, Ser, Gly or Gln, or a corresponding α-methyl amino acid form of any of the foregoing; X18 is absent or any amino acid; X19 is absent or any amino acid; and X20 is absent or any amino acid. In particular embodiments, the bond between X4 and X9 is a disulfide bond. In certain embodiments, X1, X2, and X3 are absent. In certain embodiments, X17, X19 and X20 are absent. In certain embodiments, one or both of X4 or X9 is Pen. In certain embodiments, both X4 and X9 are Pen. In particular embodiments, X18 is (D)-Lys. In certain embodiments, the peptide inhibitors comprise one or more, two or more, three or more, or four of the following: X5 is Arg, Asn, Gln, Dap, Om; X6 is Thr or Ser; X7 is Trp, 2-Nal, 1-Nal, Phe(4-OAllyl), Tyr(3-tBu), Phe(4-tBu), Phe(4-guanidino), Phe(Bzl) or Phe(4-Me), 5-Hydroxy-Trp, 6-Chloro-Trp, N-MeTrp, α-MeTrp or 1,2,3,4-tetrahydro-norharman; and X8 is Gln, Val, Phe, Glu, Lys. In certain embodiments, the peptide inhibitors comprise one or more, two or more, three or more, four or more, five or more, six or more, or seven of the following: X10 is Tyr, Phe(4-OBzl), Phe(4-OMe), Phe(4-CONH₂), Phe(3,4-Cl₂), Phe(4-tBu), Phe(4-NH₂), Phe(4-Br), Phe(4-CN), Phe(4-CO₂H), Phe(4-(2aminoethoxy)) or Phe(4-guanadino); X11 is Trp, 2-Nal, 1-Nal, Phe(4-OAllyl), Tyr(3-tBu), Phe(4-tBu), Phe(4-guanidino), Phe(Bzl) or Phe(4-Me), 5-Hydroxy-Trp, 6-Chloro-Trp, N-MeTrp, α-MeTrp or 1,2,3,4-tetrahydro-norharman; X12 is Arg, α-MeLys α-MeLeu, Aib or α-MeOm; X13 is Lys, Glu or Lys(Ac); X14 is Phe or Asn; X15 is Gly, Sr or Ala; and X16 is absent or AEA. In certain embodiments, X4 and X9 are Pen; X5 is Gln; X6 is Thr; X7 is Trp; X8 is Gln; X10 is Tyr, Phe(4-OMe) or 2-Nal; X11 is Trp, 2-Nal or 1-Nal; X12 is Arg, αMeLys or α-MeOm; X13 is Lys, Glu or Lys(Ac); X14 is Phe or Asn; X15 is Gly; and X16 is absent. In certain embodiments, one or more of X1, X2 and X3 are absent; and one or more, two or more, three or more, or four of X17, X18, X19 and X20 are absent.

In certain embodiments of peptide inhibitors of Xa: X4 is Abu, Pen, or Cys; X7 is Trp, Bip, Gln, His, Glu(Bzl), 4-Phenylbenzylalanine, Tic, Phe[4-(2-aminoethoxy)], Phe(3,4-Cl2), Phe(4-OMe), 5-Hydroxy-Trp, 6-Chloro-Trp, N-MeTrp, α-MeTrp, 1,2,3,4-tetrahydro-norharman, Phe(4-CO₂H), Phe(4-CONH₂), Phe(3,4-Dimethoxy), Phe(4-CF₃), ββ-diPheAla, Phe(4-tBu), Glu, Gly, Ile, Asn, Pro, Arg, Thr or Octgly, or a corresponding α-methyl amino acid form of any of the foregoing; X9 is Abu, Pen, or Cys; X10 is 1-Nal, 2-Nal, Aic, Bip, (D)Cys, Cha, DMT, (D)Tyr, Glu, Phe, His, Trp, Thr, Tic, Tyr, 4-pyridylAla, Octgly a Phe analog or a Tyr analog, or a corresponding α-methyl amino acid form of any of the foregoing; X11 is 2-Nal, 1-Nal, 2,4-dimethylPhe, Bip, 4-phenylcyclohexyl, Glu(Bzl), 4-Phenylbenzylalanine, Tic, Phe[4-(2-aminoethoxy)], Phe(3,4-Cl₂), Phe(3,4-F₂), phPhe(4-F), Phe(4-OMe), 5-Hydroxy-Trp, 6-Chloro-Trp, N-MeTrp, α-MeTrp, 1,2,3,4-tetrahydro-norharman, Phe(4-CO₂H), Phe(4-CONH₂), Phe(3,4-Dimethoxy), Phe(4-CF₃), Phe(2,3-Cl2), Phe(2,3-F₂), Phe(4-F), 4-phenylcyclohexylalanine, α-MePhe, βhNal, βhPhe, βhTyr, βhTrp, Bip, Nva(5-phenyl), Phe, His, hPhe, Tqa, Trp, Tyr, Phe(4-Me), Trp(2,5,7-tri-tertButyl), Phe(4-OAllyl), Tyr(3-tBu), Phe(4-tBu), Phe(4-guanidino), Phe(4-OBzl), or Octgly, or a corresponding α-methyl amino acid form of any of the foregoing; X12 is α-MeLys, α-MeOrn, α-MeLeu, MeLeu, Aib, (D)Ala, (D)Asn, (D)Leu, (D)Asp, (D)Phe, (D)Thr, 3-Pal, Aib, β-Ala, βhGlu, βhAla, βhLeu, βhVal, β-spiro-pip, Cha, Chg, Asp, Dab, Dap, α-DiethylGly, Glu, Phe, hLeu, hArg, hLeu, Ile, Lys, Leu, Asn, N-MeLeu, N-MeArg, Ogl, Orn, Pro, Gln, Arg, Ser, Thr or Tle, or a corresponding α-methyl amino acid form of any of the foregoing; X13 is Lys(Ac), (D)Asn, (D)Leu, (D)Thr, (D)Phe, Ala, Aib, α-MeLeu, βAla, βhGlu, βhAla, βhLeu, βhVal, β-spiro-pip, Cha, Chg, Asp, Arg, Om, Dab, Dap, α-DiethylGly, Glu, Phe, hLeu, Lys, Leu, Asn, Ogl, Pro, Gln, Asp, Arg, Ser, spiro-pip, Thr, Tba, Tic, Val or Tyr, or a corresponding α-methyl amino acid form of any of the foregoing; X14 is Asn, Glu, Phe, Gly, His, Lys, Leu, Met, Asn, Pro, Gln, Arg, Ser, Thr, Tic or Tyr, or a corresponding α-methyl amino acid form of any of the foregoing; X15 is Gly, (D)Ala, (D)Asn, (D)Asp, Asn, (D)Leu, (D)Phe, (D)Thr, Ala, AEA, Asp, Glu, Phe, Gly, Lys, Leu, Pro, Gln, Arg or Ser, or a corresponding α-methyl amino acid form of any of the foregoing, or X15 is Gly, (D)Ala, (D)Asn, (D)Asp, Asn, (D)Leu, (D)Phe, (D)Thr, Ala, Asn, Ser, AEA, Asp, Glu, Phe, Gly, Lys, Leu, Pro, Gln, Arg or Ser, or a corresponding α-methyl amino acid form of any of the foregoing; X16 is absent, Gly, Ala, Asp, Ser, Pro, Asn or Thr, or a corresponding α-methyl amino acid form of any of the foregoing; and X17 is absent, Glu, Ser, Gly or Gln, or a corresponding α-methyl amino acid form of any of the foregoing. In particular embodiments, the peptide inhibitor is cyclized via an intramolecular bond between X4 and X9. In certain embodiments, one or more of X1, X2, and X3 are absent. In certain embodiments, one or more of X17, X19 and X20 are absent. In certain embodiments, one of X4 or X9 is Abu, and the other of X4 or X9 is not Abu. In certain embodiments, the peptide inhibitors comprise one or more, two or more, three or more, or four of the following: X5 is Arg, Gln, Dap or Om; X6 is Thr or Ser; X7 is Trp, 2-Nal, 1-Nal, Phe(4-OAllyl), Tyr(3-tBu), Phe(4-tBu), Phe(4-guanidino), Phe(4-OBzl), Phe(4-Me), 5-Hydroxy-Trp, 6-Chloro-Trp, N-MeTrp, or α-MeTrp, 1,2,3,4-tetrahydro-norharman; and X8 is Gln, Val, Phe, Glu or Lys. In certain embodiments, the peptide inhibitors comprise one or more, two or more, three or more, four or more, five or more, six or more, or seven of the following: X10 is Tyr, Phe(4-OBzl), Phe(4-OMe), Phe(4-CONH₂), Phe(3,4-Cl₂), Phe(4-tBu), Phe(4-NH₂), Phe(4-Br), Phe(4-CN), Phe(4-CO₂H), Phe(4-(2aminoethoxy)) or Phe(4-guanadino); X11 is Trp, 2-Nal, 1-Nal, Phe(4-OAllyl), Tyr(3-tBu), Phe(4-tBu), Phe(4-guanidino), Phe(Bzl) or Phe(4-Me), 5-Hydroxy-Trp, 6-Chloro-Trp, N-MeTrp, α-MeTrp or 1,2,3,4-tetrahydro-norharman; X12 is Arg, hLeu, (D)Asn, Aib, α-MeLys, α-MeLeu or α-MeOrn; X13 is Lys, Glu or Lys(Ac); X14 is Phe or Asn; X15 is Gly, Ser or Ala, or X15 is Asn, Gly, Ser, βAla or Ala; and X16 is absent or AEA.

In certain embodiments, the peptide inhibitor has the structure of Formula I:

R¹-X-R² (I)

or a pharmaceutically acceptable salt or solvate thereof,

wherein R¹ is a bond, hydrogen, an C1-C6 alkyl, a C6-C12 aryl, a C6-C12 aryl C1-C6 alkyl, a C1-C20 alkanoyl, and including PEGylated versions alone or as spacers of any of the foregoing;

R² is a bond, OH or NH₂; and

X is an amino acid sequence, e.g., an amino acid comprising 7 to 35 amino acid residues. In certain embodiments, R² is OH or NH₂.

In certain embodiments, X comprises a sequence of Formula Xa.

In particular embodiments of formula (I), X comprises the sequence of Formula Ia:

X1-X2-X3-X4-X5-X6-W-X8-X9-X10-X11-X12-X13-X14-X15-X16-X17-X18-X19-X20 (Ia)

wherein
X1 is any amino acid or absent;
X2 is any amino acid or absent;
X3 is any amino acid or absent;
X4 is Cys, Pen, hCys, D-Pen, D-Cys, D-hCys, Met, Glu, Asp, Lys, Orn, Dap, Dab, D-Dap, D-Dab, D-Asp, D-Glu, D-Lys, Sec, 2-chloromethylbenzoic acid, mercapto-propanoic acid, mercapto-butyric acid, 2-chloro-acetic acid, 3-chloro-propanoic acid, 4-chloro-butyric acid, 3-chloro-isobutyric acid, Abu, β-azido-Ala-OH, propargylglycine, 2-(3′-butenyl)glycine, 2-allylglycine, 2-(3′-butenyl)glycine, 2-(4′-pentenyl)glycine, 2-(5′-hexenyl)glycine or absent;

### X5 is Ala, Arg, Glu, Phe, Leu, Thr, Ser, Aib, Sarc, D-Ala, D-Arg, D-Glu, D-Phe, D-Leu, D-Thr, D-Ser, α-MeOm, α-MeSer, CitDap, Dab, Dap (Ac), Gly, Lys, Asn, N-Me-Gln, N-Me-Arg, Om or Gln,

### X6 is Asp, Thr, Asn, Phe, D-Asp, D-Thr, D-Asn, or D-Phe;

### X8 is Val, Gln, Glu, or Lys;

X9 is Cys, Pen, hCys, D-Pen, D-Cys, D-hCys, Glu, Lys, Om, Dap, Dab, D-Dap, D-Dab, D-Asp, D-Glu, D-Lys, Asp, Leu, Val, Phe, Ser, Sec, Abu, β-azido-Ala-OH, propargylglycine, 2-2-allylglycine, 2-(3′-butenyl)glycine, 2-(4′-pentenyl)glycine, or 2-(5′-hexenyl)glycine;
X10 is Tyr, Phe, Phe(3,4-F₂), Phe(3,4-Cl₂), F(3-Me), Phe[4-(2-aminoethoxy)], Phe[4-(2-(acetyl-aminoethoxy)], Phe(4-Br), Phe(4-CONH₂), Phe(4-Cl), Phe(4-CN), Phe(4-guanidino), Phe(4-Me), Phe(4-NH₂), Phe(4-N₃), Phe(4-OMe), Phe(4-OBzl) or Tyr;
X11 is Trp, 1-Nal, 2-Nal, Phe(3,4-OMe₂) 5-Hydroxy-Trp, Phe(3,4-Cl₂) or Tyr(3-t-Bu)
X12 is His, Phe, Arg, N-Me-His, or Val, Cav, Cpa, Leu, Cit, hLeu, 3-Pal, t-butyl-Ala, t-butyl-Gly 4-amino-4-carboxy-tetrahydropyran, Achc Acpc, Acbc, Acvc, Agp, Aib, α-DiethylGly, α-MeLys, α-MeLys(Ac), α-Me-Leu, α-MeOm, α-MeSer, α-MeVal, Cha, Cit, Cpa, (D)Asn, Glu, hArg, or Lys;
X13 is Thr, Sarc, Glu, Phe, Arg, Leu, Lys, Val, βhAla, Aib, Lys(Ac), Cit, Asp, Dab, Dap, Glu, hArg, Lys, Asn, Om, or Gln;
X14 is Phe, Tyr βhPhe, Asn, Arg, Qln, Lys(Ac), His; Dap(Ac), Dab(Ac), or Asp;

### X15 is Gly, Ser, Thr, Gln, Ala, Sarc, 1-Ala, Glu, Arg or Asn;

X16 is any amino acid or absent;
X17 is any amino acid or absent;
X18 is any amino acid or absent;
X19 is any amino acid or absent; and
X20 is any amino acid or absent.

In particular embodiments of Ia: X5 is Ala, Arg, Glu, Phe, Leu, Thr, Ser, Aib, Sarc, D-Ala, D-Arg, D-Glu, D-Phe, D-Leu, D-Thr, D-Ser, D-Aib or D-Sarc; X10 is Tyr or Phe; X11 is Trp, 1-Nal or 2-Nal; X12 is His, Phe, Arg, N-Me-His, or Val, Cav, Cpa, Leu, Cit, hLeu, 3-Pal, t-butyl-Ala or t-butyl-Gly; X13 is Thr, Sarc, Glu, Phe, Arg, Leu, Lys, Val, βhAla, or Aib; X14 is Phe, Tyr or βhPhe; X15 is Gly, Ser, Thr, Gln, Ala or Sarc; X16 is Asp, Glu, Ala, AEA, AEP, βhAla, Gaba, or absent; and X17 is Leu, Lys, Arg, or absent.

In particular embodiments, X4 is present.

In certain embodiments, the peptide inhibitor is cyclized.

In certain embodiments, the peptide inhibitor is linear or not cyclized.

In certain embodiments, the peptide inhibitor is cyclized, or contains an intramolecular bond, between X4 and X9.

In particular embodiments of any of the peptide inhibitors, X4 and X9 are Pen. In particular embodiments, X4 and X9 form a disulfide bond.

In particular embodiments, X4 is Abu and X9 is Cys. In particular embodiments, X4 and X9 form a thioether bond.

In particular embodiments, the peptide inhibitor is cyclized via a bond between X4 and X9, and the peptide inhibitor inhibits the binding of an interleukin-23 (IL-23) to an IL-23 receptor.

### Illustrative Peptide Inhibitors Comprising Pen-Pen Disulfide Bonds

In certain embodiments, the present invention includes a peptide inhibitor of an interleukin-23 receptor, wherein the peptide inhibitor has the structure of Formula II:

R¹-X-R² (II)

or a pharmaceutically acceptable salt or solvate thereof,

wherein R¹ is a bond, hydrogen, a C1-C6 alkyl, a C6-C12 aryl, a C6-C12 aryl, a C1-C6 alkyl, a C1-C20 alkanoyl, an alkylsulphonate, an acid, γ-Glu or pGlu, appended to the N-terminus, and including PEGylated versions (e.g., 200 Da to 60,000 Da), alone or as a spacer of any of the foregoing;

R² is a bond, OH or NH₂; and

X is an amino acid sequence of 8 to 20 amino acids or 8 to 35 amino acids.

In particular embodiments of peptide inhibitor of Formula II, X comprises or consists of the sequence of Formula IIa:

X1-X2-X3-X4-X5-X6-X7-X8-X9-X10-X11-X12-X13-X14-X15-X16-X17-X18-X19-X20 (IIa)

wherein
X1 is absent or any amino acid;
X2 is absent or any amino acid;
X3 is absent or any amino acid;
X4 is Pen, Cys or homo-Cys;
X5 is any amino acid;
X6 is any amino acid;
X7 is Trp, Bip, Gln, His, Glu(Bzl), 4-Phenylbenzylalanine, Tic, Phe[4-(2-aminoethoxy)], Phe(3,4-Cl₂), Phe(4-OMe), 5-Hydroxy-Trp, 6-Chloro-Trp, N-MeTrp, α-Me-Trp, 1,2,3,4-tetrahydro-norharman, Phe(4-CO₂H), Phe(4-CONH₂), Phe(3,4-Dimethoxy), Phe(4-CF₃), Phe(4-tBu), ββ-diPheAla, Glu, Gly, Ile, Asn, Pro, Arg, Thr or Octgly, or a corresponding α-methyl amino acid form of any of the foregoing;
X8 is any amino acid;
X9 is Pen, Cys or hCys;
X10 is 1-Nal, 2-Nal, Aic, Bip, (D)Cys, Cha, DMT, (D)Tyr, Glu, Phe, His, Trp, Thr, Tic, Tyr, 4-pyridylAla, Octgly, a Phe analog or a Tyr analog (optionally, Phe(3,4-F₂), Phe(3,4-Cl₂), F(3-Me), Phe[4-(2-aminoethoxy)], Phe[4-(2-(acetyl-aminoethoxy)], Phe(4-Br), Phe(4-CONH₂), Phe(4-Cl), Phe(4-CN), Phe(4-guanidino), Phe(4-Me), Phe(4-NH₂), Phe(4-N₃), Phe(4-OMe), or Phe(4-OBzl)), or a corresponding α-methyl amino acid form of any of the foregoing;
X11 is 2-Nal, 1-Nal, 2,4-dimethylPhe, Bip, Phe(3,4-Cl₂), Phe (3,4-F₂), Phe(4-CO₂H), βhPhe(4-F), α-Me-Trp, 4-phenylcyclohexyl, Phe(4-CF₃), α-MePhe, βhNal, βhPhe, βhTyr, βhTrp, Nva(5-phenyl), Phe, His, hPhe, Tic, Tqa, Trp, Tyr, Phe(4-OMe), Phe(4-Me), Trp(2,5,7-tri-tert-Butyl), Phe(4-Oallyl), Tyr(3-tBu), Phe(4-tBu), Phe(4-guanidino, Phe(4-OBzl), Octgly, Glu(Bzl), 4-Phenylbenzylalanine, Phe[4-(2-aminoethoxy)], 5-Hydroxy-Trp, 6-Chloro-Trp, N-MeTrp, 1,2,3,4-tetrahydro-norharman, Phe(4-CONH₂), Phe(3,4-OMe₂) Phe(2,3-Cl₂), Phe(2,3-F₂), Phe(4-F), 4-phenylcyclohexylalanine or Bip, or a corresponding α-methyl amino acid form of any of the foregoing;
X12 is α-MeLys, α-MeOm, α-MeLeu, α-MeVal, 4-amino-4-carboxy-tetrahydropyran, Achc Acpc, Acbc, Acvc, MeLeu, Aib, (D)Ala, (D)Asn, (D)Leu, (D)Asp, (D)Phe, (D)Thr, 3-Pal, Aib, β-Ala, βhGlu, βhAla, βhLeu, βhVal, β-spiro-pip, Cha, Chg, Asp, Dab, Dap, α-DiethylGly, Glu, Phe, hLeu, hArg, hLeu, Ile, Lys, Leu, Asn, N-MeLeu, N-MeArg, Ogl, Orn, Pro, Gln, Arg, Ser, Thr or Tle, or a corresponding α-methyl amino acid form of any of the foregoing;
X13 is Lys(Ac), (D)Asn, (D)Leu, (D)Thr, (D)Phe, Ala, Aib, α-MeLeu, β-Ala, βhGlu, βhAla, βhLeu, βhVal, β-spiro-pip, Cha, Chg, Asp, Lys, Arg, Om, Dab, Dap, α-DiethylGly, Glu, Phe, hLeu, Lys, Leu, Asn, Ogl, Pro, Gln, Asp, Arg, Ser, spiro-pip, Thr, Tba, Tic, Val or Tyr, or a corresponding α-methyl amino acid form of any of the foregoing;
X14 is Asn, Glu, Phe, Gly, His, Lys, Leu, Met, Asn, Pro, Gln, Arg, Ser, Thr, Tic or Tyr, Lys(Ac), Om or a corresponding α-methyl amino acid form of any of the foregoing;
X15 is Gly, (D)Ala, (D)Asn, (D)Asp, Asn, (D)Leu, (D)Phe, (D)Thr, Ala, AEA, Asp, Glu, Phe, Gly, Lys, Leu, Pro, Gln, Arg or Ser, β-Ala, Arg or a corresponding α-methyl amino acid form of any of the foregoing;
X16 is absent, Gly, Ala, Asp, Ser, Pro, Asn or Thr, or a corresponding α-methyl amino acid form of any of the foregoing;
X17 is absent, Glu, Ser, Gly or Gln, or a corresponding α-methyl amino acid form of any of the foregoing;
X18 is absent or any amino acid;
X19 is absent or any amino acid; and
X20 is absent or any amino acid.

In certain embodiments of IIa: X10 is 1-Nal, 2-Nal, Aic, Bip, (D)Cys, Cha, DMT, (D)Tyr, Glu, Phe, His, Trp, Thr, Tic, Tyr, 4-pyridylAla, Octgly, a Phe analog or a Tyr analog, or a corresponding α-methyl amino acid form of any of the foregoing; X11 is 2-Nal, 1-Nal, 2,4-dimethylPhe, Bip, Phe(3,4-Cl₂), Phe (3,4-F₂), Phe(4-CO₂H), βhPhe(4-F), α-Me-Trp, 4-phenylcyclohexyl, Phe(4-CF₃), α-MePhe, βhNal, βhPhe, βhTyr, βhTrp, Nva(5-phenyl), Phe, His, hPhe, Tic, Tqa, Trp, Tyr, Phe(4-OMe), Phe(4-Me), Trp(2,5,7-tri-tert-Butyl), Phe(4-Oallyl), Tyr(3-tBu), Phe(4-tBu), Phe(4-guanidino, Phe(4-OBzl), Octgly, Glu(Bzl), 4-Phenylbenzylalanine, Phe[4-(2-aminoethoxy)], 5-Hydroxy-Trp, 6-Chloro-Trp, N-MeTrp, 1,2,3,4-tetrahydro-norharman, Phe(4-CONH₂), Phe(3,4-Dimethoxy), Phe(2,3-Cl₂), Phe(2,3-F₂), Phe(4-F), 4-phenylcyclohexylalanine or Bip, or a corresponding α-methyl amino acid form of any of the foregoing; X12 is α-MeLys, α-MeOm, α-MeLeu, MeLeu, Aib, (D)Ala, (D)Asn, (D)Leu, (D)Asp, (D)Phe, (D)Thr, 3-Pal, Aib, β-Ala, βhGlu, βhAla, βhLeu, βhVal, β-spiro-pip, Cha, Chg, Asp, Dab, Dap, α-DiethylGly, Glu, Phe, hLeu, hArg, hLeu, Ile, Lys, Leu, Asn, N-MeLeu, N-MeArg, Ogl, Om, Pro, Gln, Arg, Ser, Thr or Tle, or a corresponding α-methyl amino acid form of any of the foregoing; X13 is Lys(Ac), (D)Asn, (D)Leu, (D)Thr, (D)Phe, Ala, Aib, α-MeLeu, α-Ala, βhGlu, βhAla, βhLeu, βhVal, β-spiro-pip, Cha, Chg, Asp, Lys, Arg, Om, Dab, Dap, α-DiethylGly, Glu, Phe, hLeu, Lys, Leu, Asn, Ogl, Pro, Gln, Asp, Arg, Ser, spiro-pip, Thr, Tba, Tlc, Val or Tyr, or a corresponding α-methyl amino acid form of any of the foregoing; X14 is Asn, Glu, Phe, Gly, His, Lys, Leu, Met, Asn, Pro, Gln, Arg, Ser, Thr, Tic or Tyr, or a corresponding α-methyl amino acid form of any of the foregoing; and X15 is Gly, (D)Ala, (D)Asn, (D)Asp, Asn, (D)Leu, (D)Phe, (D)Thr, Ala, AEA, Asp, Glu, Phe, Gly, Lys, Leu, Pro, Gln, Arg or Ser, or a corresponding α-methyl amino acid form of any of the foregoing.

In certain embodiments, X3 is present. In particular embodiments, X3 is Glu, (D)Glu, Arg, (D)Arg, Phe, (D)Phe, 2-Nal, Thr, Leu, (D)Gln. In certain embodiments, X3 is (D)Arg or (D)Phe.

In particular embodiments, X1 and X2 are absent and X3 is present.

In certain embodiments, X5 is Gln, Ala, Cit, Asp, Dab, Dap, Cit Glu, Phe, Gly, His, hCys, Lys, Leu, Met, Asn, N-Me-Ala, N-Me-Asn, N-Me-Lys, α-Me-Lys, α-Me-Om, N-Me-Gln, N-Me-Arg, α-MeSer, Om, Pro, Arg, Ser, Thr, or Val. In certain embodiments, X5 is Gln, Ala, Cit, Asp, Dab, Dap, Glu, Phe, Gly, His, hCys, Lys, Leu, Met, Asn, N-Me-Ala, N-Me-Asn, N-Me-Lys, αMe-Lys, αMe-Om, N-Me-Gln, N-Me-Arg, Om, Pro, Arg, Ser, Thr, or Val. In certain embodiments, X5 is Gln or Asn.

In certain embodiments, X6 is Thr, Asp, Glu, Phe, Asn, Pro, Arg, or Ser.

In certain embodiments, X7 is Trp.

In certain embodiments, X8 is Gln, Glu, Phe, Lys, Asn, Pro, Arg, Val, Thr, or Trp.

In certain embodiments, X10 is a Tyr analog or a Phe analog. In particular embodiments, X10 is a Phe analog.

In certain embodiments wherein X10 is a Phe analog, X10 is selected from hPhe, Phe(4-OMe), α-Me-Phe, hPhe(3,4-dimethoxy), Phe(4-CONH₂), Phe(4-phenoxy), Phe(4-guanadino), Phe(4-tBu), Phe(4-CN), Phe(4-Br), Phe(4-OBzl), Phe(4-NH₂), Phe(4-F), Phe(3,5 DiF), Phe(CH₂CO₂H), Phe(penta-F), Phe(3,4-Cl₂), Phe(4-CF₃), ββ-diPheAla, Phe(4-N₃) and Phe[4-(2-aminoethoxy)]. In particular embodiments, X10 is Phe(4-OMe) or Phe[4-(2-aminoethoxy)]. In particular embodiments, X10 is Phe(4-OMe), Phe(4-CONH₂) or Phe[4-(2-aminoethoxy)]. In certain embodiments where X10 wherein X10 is a Phe analog, X10 is selected from hPhe, Phe(4-OMe), α-Me-Phe, hPhe(3,4-dimethoxy), Phe(4-CONH₂), Phe(4-phenoxy), Phe(4-guanadino), Phe(4-tBu), Phe(4-CN), Phe(4-Br), Phe(4-OBzl), Phe(4-NH₂), Phe(4-F), Phe(3,5 DiF), Phe(CH₂CO₂H), Phe(penta-F), Phe(3,4-Cl₂), Phe(4-CF₃), ββ-diPheAla, Phe(4-N₃) and Phe[4-(2-aminoethoxy)]. In particular embodiments, X10 is Phe(4-OMe). In certain embodiments where X10 is a Tyr analog, X10 is selected from hTyr, α-MeTyr, N-Me-Tyr, Tyr(3-tBu), Phe(4-CONH₂), Phe[4-(2-aminoethoxy)], and bhTyr. In certain embodiments where X10 is a Tyr analog, X10 is selected from hTyr, α-MeTyr, N-Me-Tyr, Tyr(3-tBu), and bhTyr.

In certain embodiments, X10 is Tyr, Phe(4-OMe), Phe[4-(2-aminoethoxy)], Phe(4-CONH₂), or 2-Nal. In certain embodiments, X10 is Phe(4-OMe) or Phe[4-(2-aminoethoxy)]. In certain embodiments, X10 is not Tyr.

In certain embodiments, X11 is a Trp analog. In particular embodiments, X11 is 2-Nal or 1-Nal. In certain embodiments, X11 is 2-Nal.

In certain embodiments, X12 is Aib, α-MeLys or α-MeLeu.

In particular embodiments of a peptide inhibitor of Formula II, one or both of X4 or X9 is Pen. In particular embodiments, both X4 and X9 are Pen.

In certain embodiments, the peptide inhibitor is cyclized. In particular embodiments, the peptide inhibitor is cyclized via an intramolecular bond between X4 and X9. In particular embodiments, the intramolecular bond is a disulfide bond. In particular embodiments, X4 and X9 are both Pen.

In certain embodiments, the peptide inhibitor of Formula II is linear or not cyclized. In particular embodiments of the linear peptide inhibitor of Formula I, X4 and/or X9 are any amino acid.

In particular embodiments of a peptide inhibitor of Formula II, one or more, two or more, or all three of X1, X2, and X3 are absent. In certain embodiments, X1 is absent. In certain embodiments, X1 and X2 are absent. In certain embodiments, X1, X2 and X3 are absent.

In particular embodiments of a peptide inhibitor of Formula II, one or more, two or more, three or more, four or more, or all of X16, X17, X18, X19 and X20 are absent. In particular embodiments of a peptide inhibitor of Formula I, one or more, two or more, three or more, or all of X17, X18, X19 and X20 are absent. In certain embodiments, one or more, two or more, or all three of X17, X19 and X20 are absent. In certain embodiments, one or more of X1, X2 and X3 are absent; and one or more, two or more, three or more, or four of X17, X18, X19 and X20 are absent.

In particular embodiments of a peptide inhibitor of Formula II, X18 is (D)-Lys. In certain embodiments, X18 is (D)-Lys and X17 is absent.

In particular embodiments of a peptide inhibitor of Formula II, the peptide inhibitor comprises one or more, two or more, three or more, or four of the following features: X5 is Asn, Arg or Gln; X6 is Thr; X7 is Trp; and X8 is Gln. In particular embodiments of a peptide inhibitor of Formula I, X4 is Pen; X5 is Gln, Asn or Arg; X6 is Thr; X7 is Trp, 5-hydroxy-Trp, 6-chloro-Trp, N-MeTrp, alpha-Me-Trp, or 1,2,3,4-tetrahydro-norharman; X8 is Gln; and X9 is Pen. In particular embodiments, X5 is Gln. In certain embodiments, X1, X2 and X3 are absent. In particular embodiments, both X4 and X9 are Pen.

In particular embodiments of a peptide inhibitor of Formula II, the peptide inhibitor comprises one or more, two or more, three or more, four or more, five or more, six or more, or seven of the following features: X10 is Tyr, a Phe analog, a Tyr analog or 2-Nal; X11 is Trp, 5-hydroxy-Trp, 6-chloro-Trp, N-MeTrp, alpha-Me-Trp, 1,2,3,4-tetrahydro-norharman, 2-Nal or 1-Nal; X12 is Aib, α-MeLys, α-MeOm and α-MeLeu; X13 is Lys, Glu or Lys(Ac); X14 is Phe or Asn; X15 is Gly, Ser or Ala; and X16 is absent or AEA. In certain embodiments, X10 is Tyr, Phe(4-OMe), Phe[4-(2-aminoethoxy)], Phe(CONH₂), or 2-Nal. In certain embodiments, X11 is 2-Nal or 1-Nal. In certain embodiments, X10 is not Tyr. In certain embodiments, X1, X2 and X3 are absent. In particular embodiments, both X4 and X9 are Pen

In particular embodiments of a peptide inhibitor of Formula II, the peptide inhibitor comprises one or more, two or more, three or more, four or more, five or more, six or more, seven or more, eight or more, nine or more, ten or more, or eleven of the following features: X5 is Arg or Gln; X6 is Thr; X7 is Trp; X8 is Gln; X10 is a Phe analog; X11 is Trp, 2-Nal or 1-Nal; X12 is Aib, α-MeLys or α-MeOm; X13 is Lys, Glu or Lys(Ac); X14 is Asn; X15 is Gly, Ser or Ala; and X16 is absent or AEA. In certain embodiments, X10 is Phe(4-OMe) or Phe[4-(2-aminoethoxy)]. In certain embodiments, X11 is 2-Nal or 1-Nal. In certain embodiments, X1, X2 and X3 are absent. In particular embodiments, both X4 and X9 are Pen.

In particular embodiments of a peptide inhibitor of Formula II, the peptide is cyclized via X4 and X9; X4 and X9 are Pen; X5 is Gln; X6 is Thr; X7 is Trp; X8 is Gln; X10 is Tyr, a Phe analog or 2-Nal; X11 is Trp, 2-Nal or 1-Nal; X12 is Arg, α-MeLys, α-MeOm, or α-MeLeu; X13 is Lys, Glu or Lys(Ac); X14 is Phe or Asn; X15 is Gly, Ser or Ala; and X16 is absent. In certain embodiments, X10 is Tyr, Phe(4-OMe), Phe[4-(2-aminoethoxy)], Phe(4-OMe) or 2-Nal. In certain embodiments, X10 is Phe(4-OMe). In certain embodiments, X10 is not Tyr. In certain embodiments, X11 is 2-Nal or 1-Nal. In certain embodiments, X1, X2 and X3 are absent.

In particular embodiments of a peptide inhibitor of Formula II, the peptide is cyclized via X4 and X9; X4 and X9 are Pen; X5 is Gln; X6 is Thr; X7 is Trp; X8 is Gln; X10 is Tyr, Phe(4-OMe) or 2-Nal; X11 is Trp, 2-Nal or 1-Nal; X12 is Arg, α-MeLys or α-MeOrn; X13 is Lys, Glu or Lys(Ac); X14 is Phe or Asn; X15 is Gly; and X16 is absent. In certain embodiments, X10 is Phe(4-OMe). In certain embodiments, X11 is 2-Nal or 1-Nal. In certain embodiments, X1, X2 and X3 are absent.

In particular embodiments of a peptide inhibitor of Formula II, the peptide is cyclized via X4 and X9; X4 and X9 are Pen; X5 is Gln; X6 is Thr; X7 is Trp; X8 is Gln; X10 is Phe(4-OMe) or Phe[4-(2-aminoethoxy)]; X11 is Trp, 2-Nal or 1-Nal; X12 is α-MeLys, α-MeOrn, or α-MeLeu; X13 is Lys, Glu or Lys(Ac); X14 is Asn; X15 is Gly, Ser or Ala; and X16 is absent. In certain embodiments, X10 is Phe(4-OMe). In certain embodiments, X11 is 2-Nal or 1-Nal. In certain embodiments, X1, X2 and X3 are absent.

In particular embodiments of a peptide inhibitor of Formula II, X10 is not Tyr. In certain embodiments, the present invention includes a peptide, optionally 8 to 35, 8 to 20, 8 to 16 or 8 to 12 amino acids in length, optionally cyclized, comprising or consisting of having a core sequence of Formula IIb:

Pen-Xaa5-Xaa6-Trp-Xaa8-Pen-Xaa10-[(2-Nal)] (IIb)

wherein Xaa5, Xaa6 and Xaa8 are any amino acid residue; and Xaa10 is a Phe analogue, wherein the peptide inhibits binding of IL-23 to IL-23R. In particular embodiments, X10 is a Phe analog selected from α-Me-Phe, Phe(4-OMe), Phe(4-OBzl), Phe(4-OMe), Phe(4-CONH₂), Phe(3,4-Cl₂), Phe(4-tBu), Phe(4-NH₂), Phe(4-Br), Phe(4-CN), Phe(4-CO₂H), Phe[4-(2-aminoethoxy)] or Phe(4-guanadino). In particular embodiments, Xaa10 is Phe(4-OMe) or Phe[4-(2-aminoethoxy)]. In one embodiment, Xaa10 is Phe(4-OMe). In certain embodiments, the peptide is cyclized via an intramolecular bond between Pen at Xaa4 and Pen at Xaa9. In particular embodiments, the peptide is a peptide inhibitor of Formula II, and wherein in certain embodiments, X1, X2 and X3 are absent. In particular embodiments, the peptide inhibits the binding of IL-23 to IL-23R. In certain embodiments, a peptide of Formula IIb further comprises an amino acid bound to the N-terminal Pen residue. In particular embodiments, the bound amino acid is Glu, (D)Glu, Arg, (D)Arg, Phe, (D)Phe, 2-Nal, Thr, Leu, or (D)Gln. In certain embodiments, it is (D)Arg or (D)Phe.

In certain embodiments, the present invention includes a peptide, optionally 8 to 35, 8 to 20, 8 to 16, or 8 to 12 amino acids in length, optionally cyclized, comprising or consisting of a core sequence of Formula IIc:

Pen-Xaa5-Xaa6-Trp-Xaa8-Pen-Xaa10-[(2-Nal)] (IIc)

wherein Xaa5, Xaa6 and Xaa8 are any amino acid residue; and Xaa10 is Tyr, a Phe analog, α-Me-Tyr, α-Me-Trp or 2-Nal, wherein the peptide inhibits binding of IL-23 to IL-23R. In certain embodiments, X10 is Tyr, Phe(4-OMe), Phe[4-(2-aminoethoxy)], α-Me-Tyr, α-Me-Phe, α-Me-Trp or 2-Nal. In certain embodiments, Xaa10 is Tyr, Phe(4-OMe), Phe(CONH₂), Phe[4-(2-aminoethoxy)] or 2-Nal. In certain embodiments, Xaa10 is Tyr, Phe(4-OMe), Phe[4-(2-aminoethoxy)] or 2-Nal. In particular embodiments, Xaa10 is Phe(4-OMe) or Phe[4-(2-aminoethoxy)]. In one embodiment, Xaa10 is Phe[4-(2-aminoethoxy)] or Phe(CONH₂). In particular embodiments, Xaa10 is Phe(4-OMe) or Phe[4-(2-aminoethoxy)]. In one embodiment, Xaa10 is Phe[4-(2-aminoethoxy)]. In certain embodiments, Xaa10 is not Tyr. In certain embodiments, the peptide is cyclized via an intramolecular bond between Pen at Xaa4 and Pen at Xaa9. In particular embodiments, the peptide is a peptide inhibitor of Formula II, and wherein in certain embodiments, X1, X2 and X3 are absent. In particular embodiments, the peptide inhibits the binding of IL-23 to IL-23R. In certain embodiments, a peptide of Formula IIc further comprises an amino acid bound to the N-terminal Pen residue. In particular embodiments, the bound amino acid is Glu, (D)Glu, Arg, (D)Arg, Phe, (D)Phe, 2-Nal, Thr, Leu, or (D)Gln. In certain embodiments, it is (D)Arg or (D)Phe.

In certain embodiments, the present invention includes a peptide, optionally 8 to 35, 8 to 20, 8 to 16 or 8 to 12 amino acids in length, optionally cyclized, comprising or consisting of a core sequence of Formula IId:

Pen-Xaa5-Xaa6-Trp-Xaa8-Pen-Phe[4-(2-aminoethoxy)]-[2-Nal] (IId)

wherein Xaa5, Xaa6 and Xaa8 are any amino acid residue. In certain embodiments, the peptide comprises a disulfide bond between Xaa4 and Xaa9. In certain embodiments, the peptide is a peptide inhibitor of Formula I, and wherein in certain embodiments, X1, X2 and X3 are absent. In particular embodiments, the peptide inhibits the binding of IL-23 to IL-23R. In certain embodiments, a peptide of Formula IId further comprises an amino acid bound to the N-terminal Pen residue. In particular embodiments, the bound amino acid is Glu, (D)Glu, Arg, (D)Arg, Phe, (D)Phe, 2-Nal, Thr, Leu, or (D)Gln. In certain embodiments, it is (D)Arg or (D)Phe.

In particular embodiments of a peptide inhibitor of Formula II, the peptide inhibitor has a sequence or structure shown in any of Tables 2 or 3 or comprises an amino acid sequence set forth in any of Tables 2 or 3, wherein the peptide comprises a disulfide bond between the two Pen residues.

TABLE 2
Illustrative Di-Pen Inhibitors

[Palm]-[isoGlu]-[PEG4]-[Pen]-NTWQ-[Pen]-[Phe[4-(2-aminoethoxy)]-[2-Nal]-[Aib]-[Lys(Ac)]-NN-NH₂
Ac-[Pen]-NTWQ-[Pen]-[Phe[4-(2-aminoethoxy)]-[2-Nal]-[Aib]-[Lys(PEG4-isoGlu-Palm)]-NN-NH₂
Ac-[Pen]-QTWQ-[Pen]-Phe(4-CONH₂)-[2-Nal]-[α-MeLys(Ac)]-[Lys(Ac)]-NN-NH₂
[Octanyl]-[IsoGlu]-[PEG4]-[Pen]-NTWQ-[Pen]-[Phe[4-(2-aminoethoxy)]-[2-Nal]-[Aib]-[Lys(Ac)]-NN-NH₂
[Octanyl]-[PEG4]-[Pen]-NTWQ-[Pen]-[Phe[4-(2-aminoethoxy)]-[2-Nal]-[Aib]-[Lys(Ac)]-NN-NH₂
[Palm]-[PEG4]-[Pen]-NTWQ-[Pen]-[Phe[4-(2-aminoethoxy)]-[2-Nal]-[Aib]-[Lys(Ac)]-NN-NH₂
Ac-[Pen]-NTWQ-[Pen]-[Phe[4-(2-aminoethoxy)]-[2-Nal]-[Aib]-[Lys(PEG4-Octanyl)]-NN-NH₂
Ac-[Pen]-NTWQ-[Pen]-[Phe[4-(2-aminoethoxy)]-[2-Nal]-[Aib]-[Lys(PEG4-Palm)]-NN-NH₂
Ac-[Pen]-NTWQ-[Pen]-[Phe[4-(2-aminoethoxy)-(PEG4-Palm)]-[2-Nal]-[Aib]-[Lys(Ac)]NN-NH₂
Ac-[Pen]-NTWQ-[Pen]-[Phe[4-(2-aminoethoxy)-(PEG4-Lauryl)]-[2-Nal]-[Aib]-[Lys(Ac)]-NN-NH₂
Ac-[Pen]-QTWQ-[Pen]-Phe(4-CONH₂)-[2-Nal]-[α-MeLys(PEG4-Palm)-[Lys(Ac)]-NN-NH₂
Ac-[Pen]-QTWQ-[Pen]-Phe(4-CONH₂)-[2-Nal]-[α-MeLys(PEG4-Lauryl)]-[Lys(Ac)]-NN-NH₂
Ac-[Pen]-NTWQ-[Pen]-[Phe[4-(2-aminoethoxy)-(PEG4-IsoGlu-Palm)]-[2-Nal]-[Aib]-[Lys(Ac)]-NN-NH₂
Ac-[Pen]-NTWQ-[Pen]-[Phe[4-(2-aminoethoxy)-(PEG4-IsoGLu-Lauryl)]-[2-Nal]-[Aib]-[Lys(Ac)]-NN-NH₂
Ac-[Pen]-QTWQ-[Pen]-Phe(4-CONH₂)-[2-Nal]-[α-MeLys(PEG4-IsoGlu-Palm)]-[Lys(Ac)]-NN-NH₂
Ac-[Pen]-QTWQ-[Pen]-Phe(4-CONH₂)-[2-Nal]-a-Me-K(PEG4-IsoGlu-Lauryl)]-[Lys(Ac)]-NN-NH₂
Ac-[Pen]-QTWQ-[Pen]-Phe(4-CONH₂)-[2-Nal]-[α-MeLys(IVA)]-[Lys(Ac)]-NN-NH₂
Ac-[Pen]-QTWQ-[Pen]-Phe(4-CONH₂)-[2-Nal]-[α-MeLys(Biotin)]-[Lys(Ac)]-NN-NH₂
Ac-[Pen]-QTWQ-[Pen]-Phe(4-CONH₂)-[2-Nal]-[α-MeLys(Octanyl)]-[Lys(Ac)]-NN-NH₂
Ac-[Pen]-[Lys(IVA)]-TWQ-[Pen]-[Phe[4-(2-aminoethoxy)]-[2-Nal]-[Aib]-[Lys(Ac)]-NN-NH₂
Ac-[Pen]-NTWQ-[Pen]-[Phe[4-(2-aminoethoxy)]-[2-Nal]-[Aib]-[Lys(Ac)]-[Lys(IVA)]-N-NH₂
Ac-[Pen]-[Lys(Biotin)]-TWQ-[Pen]-[Phe[4-(2-aminoethoxy)]-[2-Nal]-[Aib]-[Lys(Ac)]-NN-NH₂
Ac-[Pen]-NTWQ-[Pen]-[Phe[4-(2-aminoethoxy)]-[2-Nal]-[Aib]-[Lys(Ac)]-[Lys(Biotin)]-N-NH₂
Ac-[Pen]-[Lys(Octanyl)]-TWQ-[Pen]-[Phe[4-(2-aminoethoxy)]-[2-Nal]-[Aib]-[Lys(Ac)]-NN-NH₂
Ac-[Pen]-NTWQ-[Pen]-[Phe[4-(2-aminoethoxy)]-[2-Nal]-[Aib]-[Lys(Ac)]-[Lys(octanyl)]-N-NH₂
Ac-[Pen]-[Lys(Palm)]-TWQ-[Pen]-[Phe[4-(2-aminoethoxy)]-[2-Nal]--[Aib]-[Lys(Ac)]-NN-NH₂
Ac-[Pen]-NTWQ-[Pen]-[Phe[4-(2-aminoethoxy)]-[2-Nal]-[Aib]-[Lys(Ac)]-Lys(Palm)]-N-NH₂
Ac-[Pen]-[Lys(PEG8)]-TWQ-[Pen]-[Phe[4-(2-aminoethoxy)]-[2-Nal]--[Aib]-[Lys(Ac)]-NN-NH₂
Ac-[Pen]-NTWQ-[Pen]-[Phe[4-(2-aminoethoxy)]-[2-Nal]--[Aib]-[Lys(Ac)]-[Lys(PEG8)]-N-NH₂
Ac-[Pen]-K(Peg11-Palm)TWQ-[Pen]-[Phe[4-(2-aminoethoxy)]-[2-Nal]-[Aib]-[Lys(Ac)]-NN-NH₂
Ac-[Pen]-NTWQ-[Pen]-[Phe[4-(2-aminoethoxy)]-[2-Nal]--[Aib]-[Lys(Ac)]-[Lys(Peg11-palm)]-N-NH₂
Ac-[Pen]-[Cit]-TW-[Cit]-[Pen]-[Phe[4-(2-aminoethoxy)]-[2-Nal]--[Aib]-[Lys(Ac)]-NN-NH₂
Ac-[Pen]-[Lys(Ac)]-TW-[Cit]-[Pen]-[Phe[4-(2-aminoethoxy)]-[2-Nal]-[Aib]-[Lys(Ac)]-NN-NH₂
Ac-[Pen]-NT-[Phe(3,4-OCH₃)₂]-Q-[Pen]-[Phe[4-(2-aminoethoxy)]-[2-Nal]-[Aib]-[Lys(Ac)]-NN-NH₂
Ac-[Pen]-NT-[Phe(2,4-CH₃)₂]-Q-[Pen]-[Phe[4-(2-aminoethoxy)]-[2-Nal]-[Aib]-[Lys(Ac)]-NN-NH₂
Ac-[Pen]-NT-[Phe(3-CH₃)]-Q-[Pen]-[Phe[4-(2-aminoethoxy)]-[2-Nal]--[Aib]-[Lys(Ac)]-NN-NH₂
Ac-[Pen]-NT-[Phe(4-CH₃)]-Q-[Pen]-[Phe[4-(2-aminoethoxy)]-[2-Nal]-[Aib]-[Lys(Ac)]-NN-NH₂
Ac[(D)Arg]-[Pen]-NTWQ-[Pen]-[Phe[4-(2-aminoethoxy)]-[2-Nal]--[Aib]-[Lys(Ac)]-N-[βAla]-NH₂
Ac-[(D)Tyr]-[Pen]-NTWQ-[Pen]-[Phe[4-(2-aminoethoxy)]-[2-Nal]-[Aib]-[Lys(Ac)]-N-[βAla]-NH₂
Ac-[Pen]-NTWQ-[Pen]-[Phe[4-(2-aminoethoxy)]-[2-Nal]-[Aib]-[Lys(Ac)]-QN-NH₂
Ac-[Pen]-NTWQ-[Pen]-[Phe[4-(2-aminoethoxy)]-[2-Nal]-[Aib]-[Lys(Ac)]-[Lys(Ac)]-N-NH₂
Ac-[Pen]-NTWQ-[Pen]-[Phe[4-(2-aminoethoxy)]-[2-Nal]-[Aib]-[Lys(Ac)]-N-[Lys(Ac)]-NH₂
Ac-[Pen]-NTWQ-[Pen]-[Phe[4-(2-aminoethoxy)]-[2-Nal]-[Aib]-[Lys(Ac)]-QQ-NH₂
Ac-[Pen]-NTWQ-[Pen]-[Phe[4-(2-aminoethoxy)]-[2-Nal]-[Aib]-[Lys(Ac)]-Q-[βAla]-NH₂
Ac-[Pen]-NTWQ-[Pen]-[Phe[4-(2-aminoethoxy)]-[2-Nal]-[Aib]-[Lys(Ac)]-N-[Cit]-NH₂
Ac-[Pen]-NTWQ-[Pen]-[Phe[4-(2-aminoethoxy)]-[2-Nal]-[Aib]-[Lys(Ac)]-[Cit]-NNH₂
Ac-[Pen]-NTWQ-[Pen]-[Phe[4-(2-aminoethoxy)]-[2-Nal]-[Aib]-[Lys(Ac)]-[Cit]-Q-NH₂
Ac-[Pen]-NTWQ-[Pen]-[Phe[4-(2-aminoethoxy)]-[2-Nal]-[Aib]-[Lys(Ac)]-[Cit]-[Lys(Ac)]-NH₂
Ac-[Pen]-NTWQ-[Pen]-[Phe[4-(2-aminoethoxy)]-[2-Nal]-[Aib]-[Lys(Ac)]-[Lys(Ac)]-[Cit]-NH₂
Ac-[Pen]-NTWQ-[Pen]-[Phe[4-(2-aminoethoxy)]-[2-Nal]-[Aib]-QN-[βAla]-NH₂
AC-[Pen]-NTWQ-[Pen]-[Phe[4-(2-aminoethoxy)]-[2-Nal]-[Aib]-E-[Cit]-Q-NH₂
Ac-[Pen]-NTWQ-[Pen]-[Phe[4-(2-aminoethoxy)]-[2-Nal]-[Aib]-CitNCitNH₂
Ac-[Pen]-NTWQ-[Pen]-[Phe[4-(2-aminoethoxy)]-[2-Nal]-[Aib]-[Cit]-Q-[Cit]-NH₂
Ac-[Pen]-[Cit]-TWQ-[Pen]-[Phe[4-(2-aminoethoxy)]-[2-Nal]-[Aib]-[Lys(Ac)]-NN-NH₂
Ac-[Pen]-NTWQ-[Pen]-[Phe[4-(2-aminoethoxy)]-[2-Nal]-[Aib]-[Lys(Ac)]-NN-NH₂
Ac-[Pen]-NTWQ-[Pen]-[Phe[4-(2-aminoethoxy)]-[2-Nal]-[Aib]-QNN-NH₂
Ac-[Pen]-NTWQ-[Pen]-[Phe[4-(2-aminoethoxy)]-[2-Nal]-[Aib]-ENQ-NH₂
Ac-[Pen]-GPWQ-[Pen]-[Phe[4-(2-aminoethoxy)]-[2-Nal]-[Aib]-[Lys(Ac)]-NN-NH₂
Ac-[Pen]-PGWQ-[Pen]-[Phe[4-(2-aminoethoxy)]-[2-Nal]-[Aib]-[Lys(Ac)]-NN-NH₂
Ac-[Pen]-NTWN-[Pen]-[Phe[4-(2-aminoethoxy)]-[2-Nal]-[Aib]-[Lys(Ac)]-NN-NH₂
Ac-[Pen]-NSWQ-[Pen]-[Phe[4-(2-aminoethoxy)]-[2-Nal]-[Aib]-[Lys(Ac)]-NN-NH₂
Ac-[Pen]-N-[Aib]-WQ-[Pen]-[Phe[4-(2-aminoethoxy)]-[2-Nal]-[Aib]-[Lys(Ac)]-NN-NH₂
Ac-[Pen]-NTW-[Aib]-[Pen]-[Phe[4-(2-aminoethoxy)]-[2-Nal]-[Aib]-[Lys(Ac)]-N-[Aib]-NH₂
Ac-[Pen]-QTW-[Lys(Ac)]-[Pen]-[Phe[4-(2-aminoethoxy)]-[2-Nal]-[Aib]-[Lys(Ac)]-NN-NH₂
Ac-[Pen]-[Lys(Ac)]-TWQ-[Pen]-[Phe[4-(2-aminoethoxy)]-[2-Nal]--[Aib]-[Lys(Ac)]NNNH₂
Ac-[Pen]-QVWQ-[Pen]-[Phe[4-(2-aminoethoxy)]-[2-Nal]-[Aib]-[Lys(Ac)]-NN-NH₂
Ac-[Pen]-NT-[2-Nal]-Q-[Pen]-[Phe[4-(2-aminoethoxy)]-[2-Nal]-[Aib]-[Lys(Ac)]-NN-NH₂
Ac-[Pen]-NT-[1-Nal]-Q-[Pen]-[Phe[4-(2-aminoethoxy)]-[2-Nal]-[Aib]-[Lys(Ac)]-NN-NH₂
Ac-[Pen]-NTWQ-[Pen]-[Phe[4-(2-aminoethoxy)]-[2-Nal]-[α-MeLeu]-[Lys(Ac)]-NN-NH₂
Ac-[Pen]-NTWQ-[Pen]-[Phe[4-(2-aminoethoxy)]-[2-Nal]-[α-MeLys]-[Lys(Ac)]-NN-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-NN-
NH₂
Ac-[Pen]-NTWQ-[Pen]-[Phe[4-(2-aminoethoxy)]-[2-Nal]-[α-MeLeu]-[Lys(Ac)]-N-[βAla]-NH₂
Ac-[Pen]-NTWQ-[Pen]-[Phe[4-(2-aminoethoxy)]-[2-Nal]-[α-MeLys]-[Lys(Ac)]-N-[βAla]-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-N-
[βAla]-NH₂
Ac-[Pen]-NTWQ-[Pen]-[Phe[4-(2-aminoethoxy)]-[2-Nal]-[Aib]-[Lys(Ac))]-LN-NH₂
Ac-[Pen]-NTWQ-[Pen]-[Phe[4-(2-aminoethoxy)]-[2-Nal]-[Aib]-[Lys(Ac)]-GN-NH₂
Ac-[Pen]-NTWQ-[Pen]-[Phe[4-(2-aminoethoxy)]-[2-Nal]-[Aib]-[Lys(Ac)]-SN-NH₂
Ac-[Pen]-NTWQ-[Pen]-[Phe[4-(2-aminoethoxy)]-[2-Nal]-[Aib]-[Lys(Ac)]-[Aib]-N-NH₂
Ac-[Pen]-NTWQ-[Pen]-[Phe[4-(2-aminoethoxy)]-[2-Nal]-[Aib]-[Lys(Ac)]-FN-NH₂
Ac-[Pen]-NTW-[Cit]-[Pen]-[Phe[4-(2-aminoethoxy)]-[2-Nal]-[Aib]-[Lys(Ac)]-NN-NH₂
Ac-[Pen]-NTWQ-[Pen]-[Phe[4-(2-aminoethoxy)]-[2-Nal]-[Aib]-[Lys(Ac)]-[Tic]-[βAla]-NH₂
Ac-[Pen]-NTWQ-[Pen]-[Phe[4-(2-aminoethoxy)]-[2-Nal]-[Aib]-[Lys(Ac)]-[nLeu]-[βAla]-NH₂
Ac-[Pen]-NTWQ-[Pen]-[Phe[4-(2-aminoethoxy)]-[2-Nal]-[Aib]-[Lys(Ac)]-G-[βAla]-NH₂
Ac-[Pen]-NTWQ-[Pen]-[Phe[4-(2-aminoethoxy)]-[2-Nal]-[Aib]-[Lys(Ac)]-R-[βAla]-NH₂
Ac-[Pen]-NTWQ-[Pen]-[Phe[4-(2-aminoethoxy)]-[2-Nal]--[Aib]-[Lys(Ac)]-W-[βAla]-NH₂
Ac-[Pen]-NTWQ-[Pen]-[Phe[4-(2-aminoethoxy)]-[2-Nal]--[Aib]-[Lys(Ac)]-S-[βAla]-NH₂
Ac-[Pen]-NTWQ-[Pen]-[Phe[4-(2-aminoethoxy)]-[2-Nal]--[Aib]-[Lys(Ac)]-L-[βAla]-NH₂
Ac-[Pen]-NTWQ-[Pen]-[Phe[4-(2-aminoethoxy)]-[2-Nal]--[Aib]-[Lys(Ac)]-[AIB]-[βAla]-NH₂
Ac-[Pen]-NTWQ-[Pen]-[Phe[4-(2-aminoethoxy)]-[2-Nal]--[Aib]-[Lys(Ac)]-N-MeAla]-[βAla]-NH₂
Ac-[Pen]-NTWQ-[Pen]-[Phe[4-(2-aminoethoxy)]-[2-Nal]-[Aib]-[Lys(Ac)]-[2-Nap]-[βAla]-NH₂
Ac-[Pen]-NTWQ-[Pen]-[Phe[4-(2-aminoethoxy)]-[2-Nal]-[Aib]-[Lys(Ac)]-F-[βAla]-NH₂
Ac-[(D)Arg[Pen]-NTWQ-[Pen]-[Phe[4-(2-aminoethoxy)[2-Nal]-[4-amino-4-carboxy-tetrahydropyran]-
[Lys(Ac)]-NN-NH₂

TABLE 3
Illustrative Peptides Containing the Ac-[Pen]-XXWX-[Pen]-XXXX Motif and
Analogues Thereof

Ac-[Pen]-NTWQ-[Pen]-[Phe[4-(2-aminoethoxy)]-[2-Nal]-[4-amino-4-carboxy-tetrahydropyran]-[Lys(Ac)]-
NN-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNE-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNF-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNK-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNN-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNW-NH
Ac-[Pen]-NTWQ-[Pen]-[Phe[4-(2-aminoethoxy)]-[2-Nal]-[4-amino-4-carboxy-tetrahydropyran]-[Lys(Ac)]-
NNG-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNT-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNPK-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNPG-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNEP-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNGK-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNPT-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNKGF-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNGW-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNGQ-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNGGG-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNKKK-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNEEE-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNFFF-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNTTT-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNGGGR-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNGGGF-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNGGGE-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNGGGQ-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNGGGT-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNGGGGR-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNGGGGF-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNGGGGE-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNGGGGQ-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNGGGGT-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNRRRRR-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNFFFFF-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNEEEEE-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNQQQQQ-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NNTTTTT-NH₂
Ac-GGG-[Pen]-NTWQ-[Pen]-[Phe[4-(2-aminoethoxy)]-[2-Nal]-[4-amino-4-carboxy-tetrahydropyran]-
[Lys(Ac)]-NN-NH₂
Ac-RRR-[Pen]-NTWQ-[Pen]-[Phe[4-(2-aminoethoxy)]-[2-Nal]-[4-amino-4-carboxy-tetrahydropyran]-
[Lys(Ac)]-NN-NH₂
Ac-FFF-[Pen]-NTWQ-[Pen]-[Phe[4-(2-aminoethoxy)]-[2-Nal]-[4-amino-4-carboxy-tetrahydropyran]-
[Lys(Ac)]-NN-NH₂
Ac-EEE-[Pen]-NTWQ-[Pen]-[Phe[4-(2-aminoethoxy)]-[2-Nal]-[4-amino-4-carboxy-tetrahydropyran]-
[Lys(Ac)]-NN-NH₂
Ac-QQQ-[Pen]-NTWQ-[Pen]-[Phe[4-(2-aminoethoxy)]-[2-Nal]-[4-amino-4-carboxy-tetrahydropyran]-
[Lys(Ac)]-NN-NH₂
Ac-TTT-[Pen]-NTWQ-[Pen]-[Phe[4-(2-aminoethoxy)]-[2-Nal]-[4-amino-4-carboxy-tetrahydropyran]-
[Lys(Ac)]-NN-NH₂
Ac-RG-[Pen]-NTWQ-[Pen]-[Phe[4-(2-aminoethoxy)]-[2-Nal]-[4-amino-4-carboxy-tetrahydropyran]-[Lys(Ac)]-
NN-NH₂
Ac-FG-[Pen]-NTWQ-[Pen]-[Phe[4-(2-aminoethoxy)]-[2-Nal]-[4-amino-4-carboxy-tetrahydropyran]-[Lys(Ac)]-
NN-NH₂
Ac-EG-[Pen]-NTWQ-[Pen]-[Phe[4-(2-aminoethoxy)]-[2-Nal]-[4-amino-4-carboxy-tetrahydropyran]-[Lys(Ac)]-
NN-NH₂
Ac-QG-[Pen]-NTWQ-[Pen]-[Phe[4-(2-aminoethoxy)]-[2-Nal]-[4-amino-4-carboxy-tetrahydropyran]-
[Lys(Ac)]-NN-NH₂
Ac-TG-[Pen]-NTWQ-[Pen]-[Phe[4-(2-aminoethoxy)]-[2-Nal]-[4-amino-4-carboxy-tetrahydropyran]-[Lys(Ac)]-
NN-NH₂
Ac-[Pen]-NTWQ-[Pen]-[Phe[4-(2-aminoethoxy)]-[2-Nal]-[4-amino-4-carboxy-tetrahydropyran]-[Lys(Palm)]-
NN-NH₂
Ac-[Pen]-NTWQ-[Pen]-[Phe[4-(2-aminoethoxy)]-[2-Nal]-[4-amino-4-carboxy-tetrahydropyran]-[Lys(isoGlu-
Palm)]-NN-NH₂
Ac-[Pen]-NTWQ-[Pen]-[Phe[4-(2-aminoethoxy)]-[2-Nal]-[4-amino-4-carboxy-tetrahydropyran]-[Lys(PEG11-
Palm)]-NN-NH₂
Ac-[Pen]-NTWQ-[Pen]-[Phe[4-(2-aminoethoxy)]-[2-Nal]-[4-amino-4-carboxy-tetrahydropyran]-[Lys(Ahx-
Palm)]-NN-NH₂
Ac-[Pen]-NTWQ-[Pen]-[Phe[4-(2-aminoethoxy)]-[2-Nal]-[4-amino-4-carboxy-tetrahydropyran]-[Lys(isoGlu-
Ahx-Palm)]-NN-NH₂
[Palm]-[Pen]-NTWQ-[Pen]-[Phe[4-(2-aminoethoxy)]-[2-Nal]-[4-amino-4-carboxy-tetrahydropyran]-
[Lys(Ac)]-NN-NH₂
[Palm-isoGlu]-[Pen]-NTWQ-[Pen]-[Phe[4-(2-aminoethoxy)]-[2-Nal]-[4-amino-4-carboxy-tetrahydropyran]-
[Lys(Ac)]-NN-NH₂
[Palm-PEG11]-[Pen]-NTWQ-[Pen]-[Phe[4-(2-aminoethoxy)]-[2-Nal]-[4-amino-4-carboxy-tetrahydropyran]-
[Lys(Ac)]-NN-NH₂
[Palm-Ahx]-[Pen]-NTWQ-[Pen]-[Phe[4-(2-aminoethoxy)]-[2-Nal]-[4-amino-4-carboxy-tetrahydropyran]-
[Lys(Ac)]-NN-NH₂
[Palm-Ahx-isoGlu]-[Pen]-NTWQ-[Pen]-[Phe[4-(2-aminoethoxy)]-[2-Nal]-[4-amino-4-carboxy-
tetrahydropyran]-[Lys(Ac)]-NN-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NN-Lys[Palm]-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NN-Lys[isoGlu-Palm]-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NN-Lys[PEG11-Palm]-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NN-Lys[Ahx-Palm]-NH₂
Ac-[Pen]-NTWQ-[Pen]-[Phe[4-(2-aminoethoxy)]-[2-Nal]-[4-amino-4-carboxy-tetrahydropyran]-[Lys(Ac)]-
NN-Lys[isoGlu-Ahx-Palm]-NH₂

### Illustrative Peptide Inhibitors Comprising Thioether Bonds

In certain embodiments, the present invention includes a peptide inhibitor of an interleukin-23 receptor, wherein the peptide inhibitor has the structure of Formula III:

R¹-X-R² (III)

or a pharmaceutically acceptable salt or solvate thereof,

wherein R¹ is a bond, hydrogen, a C1-C6 alkyl, a C6-C12 aryl, a C6-C12 aryl, a C1-C6 alkyl, a C1-C20 alkanoyl, an alkylsulphonate, an acid, γ-Glu or pGlu, appended to the N-terminus, and including PEGylated versions (e.g., 200 Da to 60,000 Da), alone or as a spacer of any of the foregoing;

R² is a bond, OH or NH₂; and

X is an amino acid sequence of 8 to 20 amino acids or 8 to 35 amino acids,

In particular embodiments of peptide inhibitors of Formula III, X comprises or consists of the sequence of Formula IIIa:

X1-X2-X3-X4-X5-X6-X7-X8-X9-X10-X11-X12-X13-X14-X15-X16-X17-X18-X19-X20 (IIIa)

wherein
X1 is absent or any amino acid;
X2 is absent or any amino acid;
X3 is absent or any amino acid;

### X4 is Abu, Pen, or Cys;

X5 is any amino acid;
X6 is any amino acid;
X7 is Trp, Bip, Gln, His, Glu(Bzl), 4-Phenylbenzylalanine, Tic, Phe[4-(2-aminoethoxy)], Phe(3,4-Cl₂), Phe(4-OMe), 5-Hydroxy-Trp, 6-Chloro-Trp, N-MeTrp, α-MeTrp, 1,2,3,4-tetrahydro-norharman, Phe(4-CO₂H), Phe(4-CONH₂), Phe(3,4-(OCH₃)2), Phe(4-CF₃), ββ-diPheAla, Phe(4-tBu), Glu, Gly, Ile, Asn, Pro, Arg, Thr or Octgly, or a corresponding α-methyl amino acid form of any of the foregoing;
X8 is any amino acid;

### X9 is Abu, Pen, or Cys;

X10 is 1-Nal, 2-Nal, Aic, Bip, (D)Cys, Cha, DMT, (D)Tyr, Glu, Phe, His, Trp, Thr, Tic, Tyr, 4-pyridylAla, Octgly a Phe analog or a Tyr analog (optionally, Phe(3,4-F₂), Phe(3,4-Cl₂), F(3-Me), Phe[4-(2-aminoethoxy)], Phe[4-(2-(acetyl-aminoethoxy)], Phe(4-Br), Phe(4-CONH₂), Phe(4-Cl), Phe(4-CN), Phe(4-guanidino), Phe(4-Me), Phe(4-NH₂), Phe(4-N₃), Phe(4-OMe), Phe(4-OBzl)), or a corresponding α-methyl amino acid form of any of the foregoing;
X11 is 2-Nal, 1-Nal, 2,4-dimethylPhe, Bip, 4-phenylcyclohexyl, Glu(Bzl), 4-Phenylbenzylalanine, Tic, Phe[4-(2-aminoethoxy)], Phe(3,4-Cl₂), Phe(3,4-F₂), βhPhe(4-F), Phe(4-OMe), 5-Hydroxy-Trp, 6-Chloro-Trp, N-MeTrp, α-MeTrp, 1,2,3,4-tetrahydro-norharman, Phe(4-CO₂H), Phe(4-CONH₂), Phe(3,4-Dimethoxy), Phe(4-CF₃), Phe(2,3-Cl2), Phe(3,4-Cl₂), Phe(2,3-F₂), Phe(4-F), 4-phenylcyclohexylalanine, α-MePhe, βhNal, βhPhe, βhTyr, βhTrp, Bip, Nva(5-phenyl), Phe, His, hPhe, Tqa, Trp, Tyr, Phe(4-Me), Trp(2,5,7-tri-tertButyl), Phe(4-OAllyl), Tyr(3-tBu), Phe(4-tBu), Phe(4-guanidino), Phe(4-OBzl), or Octgly, or a corresponding α-methyl amino acid form of any of the foregoing;
X12 is α-MeLys, α-MeOm, α-MeLeu, MeLeu, Aib, (D)Ala, (D)Asn, (D)Leu, (D)Asp, (D)Phe, (D)Thr, 3-Pal, Aib, β-Ala, βhGlu, βhAla, βhLeu, βhVal, β-spiro-pip, Cha, Chg, Asp, Dab, Dap, α-DiethylGly, Glu, Phe, hLeu, hArg, hLeu, Ile, Lys, Leu, Asn, N-MeLeu, N-MeArg, Ogl, Om, Pro, Gln, Arg, Ser, Thr or Tle, amino-4-carboxy-tetrahydropyran (THP), Achc Acpc, Acbc, Acvc, Aib, or a corresponding α-methyl amino acid form of any of the foregoing;
X13 is Lys Lys(Ac), (D)Asn, (D)Leu, (D)Thr, (D)Phe, Ala, Aib, α-MeLeu, βAla, βhGlu, βhAla, βhLeu, βhVal, β-spiro-pip, Cha, Chg, Asp, Arg, Orn, Dab, Dap, α-DiethylGly, Glu, Phe, hLeu, Lys, Leu, Asn, Ogl, Pro, Gln, Asp, Arg, Ser, spiro-pip, Thr, Tba, Tlc, Val or Tyr, or a corresponding α-methyl amino acid form of any of the foregoing;
X14 is Asn, Glu, Phe, Gly, His, Lys, Lys (Ac), Leu, Met, Asn, Pro, Gln, Arg, Ser, Thr, Tic, Asp or Tyr, or a corresponding α-methyl amino acid form of any of the foregoing;
X15 is Gly, (D)Ala, (D)Asn, (D)Asp, Asn, (D)Leu, (D)Phe, (D)Thr, Ala, AEA, Asp, Glu, Phe, Gly, Lys, Leu, Pro, Gln, Arg, β-Ala, or Ser, or a corresponding α-methyl amino acid form of any of the foregoing;
X16 is absent, Gly, Ala, Asp, Ser, Pro, Asn or Thr, or a corresponding α-methyl amino acid form of any of the foregoing;
X17 is absent, Glu, Ser, Gly or Gln, or a corresponding α-methyl amino acid form of any of the foregoing;
X18 is absent or any amino acid;
X19 is absent or any amino acid; and
X20 is absent or any amino acid.

In certain embodiments, X14 is Asn, Glu, Phe, Gly, His, Lys, Lys (Ac), Leu, Met, Asn, Pro, Gln, Arg, Ser, Thr, Tic, or Tyr, or a corresponding α-methyl amino acid form of any of the foregoing.

In certain embodiments of IIIa: X7 is Trp, Bip, Gln, His, Glu(Bzl), 4-Phenylbenzylalanine, Tic, Phe[4-(2-aminoethoxy)], Phe(3,4-Cl₂), Phe(4-OMe), 5-Hydroxy-Trp, 6-Chloro-Trp, N-MeTrp, α-MeTrp, 1,2,3,4-tetrahydro-norharman, Phe(4-CO₂H), Phe(4-CONH₂), Phe(3,4-Dimethoxy), Phe(4-CF₃), ββ-diPheAla, Phe(4-tBu), Glu, Gly, Ile, Asn, Pro, Arg, Thr or Octgly, or a corresponding α-methyl amino acid form of any of the foregoing; X10 is 1-Nal, 2-Nal, Aic, Bip, (D)Cys, Cha, DMT, (D)Tyr, Glu, Phe, His, Trp, Thr, Tic, Tyr, 4-pyridylAla, Octgly a Phe analog or a Tyr analog, or a corresponding α-methyl amino acid form of any of the foregoing; X11 is 2-Nal, 1-Nal, 2,4-dimethylPhe, Bip, 4-phenylcyclohexyl, Glu(Bzl), 4-Phenylbenzylalanine, Tic, Phe[4-(2-aminoethoxy)], Phe(3,4-Cl₂), Phe(3,4-F₂), βhPhe(4-F), Phe(4-OMe), 5-Hydroxy-Trp, 6-Chloro-Trp, N-MeTrp, α-MeTrp, 1,2,3,4-tetrahydro-norharman, Phe(4-CO₂H), Phe(4-CONH₂), Phe(3,4-Dimethoxy), Phe(4-CF₃), Phe(2,3-Cl₂), Phe(2,3-F₂), Phe(4-F), 4-phenylcyclohexylalanine, α-MePhe, βhNal, βhPhe, βhTyr, βhTrp, Bip, Nva(5-phenyl), Phe, His, hPhe, Tqa, Trp, Tyr, Phe(4-Me), Trp(2,5,7-tri-tertButyl), Phe(4-OAllyl), Tyr(3-tBu), Phe(4-tBu), Phe(4-guanidino), Phe(4-OBzl), or Octgly, or a corresponding α-methyl amino acid form of any of the foregoing; X12 is α-MeLys, α-MeOm, α-MeLeu, MeLeu, Aib, (D)Ala, (D)Asn, (D)Leu, (D)Asp, (D)Phe, (D)Thr, 3-Pal, Aib, β-Ala, βhGlu, βhAla, βhLeu, βhVal, β-spiro-pip, Cha, Chg, Asp, Dab, Dap, α-DiethylGly, Glu, Phe, hLeu, hArg, hLeu, Ile, Lys, Leu, Asn, N-MeLeu, N-MeArg, Ogl, Om, Pro, Gln, Arg, Ser, Thr or Tle, or a corresponding α-methyl amino acid form of any of the foregoing; X13 is Lys(Ac), (D)Asn, (D)Leu, (D)Thr, (D)Phe, Ala, Aib, α-MeLeu, βAla, βhGlu, βhAla, βhLeu, βhVal, β-spiro-pip, Cha, Chg, Asp, Arg, Om, Dab, Dap, α-DiethylGly, Glu, Phe, hLeu, Lys, Leu, Asn, Ogl, Pro, Gln, Asp, Arg, Ser, spiro-pip, Thr, Tba, Tic, Val or Tyr, or a corresponding α-methyl amino acid form of any of the foregoing; X14 is Asn, Glu, Phe, Gly, His, Lys, Leu, Met, Asn, Pro, Gln, Arg, Ser, Thr, Tic or Tyr, or a corresponding α-methyl amino acid form of any of the foregoing; and X15 is Gly, (D)Ala, (D)Asn, (D)Asp, Asn, (D)Leu, (D)Phe, (D)Thr, Ala, AEA, Asp, Glu, Phe, Gly, Lys, Leu, Pro, Gln, Arg or Ser, or a corresponding α-methyl amino acid form of any of the foregoing.

In certain embodiments, X3 is present. In particular embodiments, X3 is Glu, (D)Glu, Arg, (D)Arg, Phe, (D)Phe, 2-Nal, Thr, Leu, or (D)Gln. In certain embodiments, it is (D)Arg or (D)Phe.

In particular embodiments, X5 is Gln, Ala, Cys, Cit, Asp, Dab, Dap, Glu, Phe, Gly, His, hCys, Lys, Leu, Met, Asn, N-Me-Ala, N-M-Asn, N-Me-Lys, N-Me-Gln, N-Me-Arg, Om, Pro, Pen, Gln, Arg, Ser, Thr, or Val.

In particular embodiments, X6 is Thr, Asp, Glu, Phe, Asn, Pro, Arg, Ser, or Thr. In particular embodiments, X8 is Gln, Glu, Phe, Lys, Asn, Pro, Arg, Val, Thr, or Trp.

In certain embodiments, X10 is a Tyr analog or a Phe analog. In particular embodiments, X10 is Phe(4-OMe), Phe(CONH₂) or Phe[4-(2-aminoethoxy)]. In certain embodiments, X10 is a Tyr analog or a Phe analog. In particular embodiments, X10 is Phe(4-OMe) or Phe[4-(2-aminoethoxy)].

In certain embodiments where X10 is a the Phe analog, X10 is selected from hPhe, Phe(4-OMe), α-MePhe, hPhe(3,4-dimethoxy), Phe(4-CONH₂), Phe(4-O-Bzl)), Phe(4-guanadino), Phe(4-tBu), Phe(4-CN), Phe(4-Br), Phe(4-NH₂), Phe(4-F), Phe(3,5 DiF), Phe(CH₂CO₂H), Phe(penta-F), Phe(3,4-Cl₂), Phe(4-CF₃), ββ-diPheAla, Phe(4-N₃) and Phe[4-(2-aminoethoxy)]. In particular embodiments, X10 is Phe[4-(2-aminoethoxy)] or Phe(CONH₂). In particular embodiments, X10 is Phe[4-(2-aminoethoxy)].

In certain embodiments where X10 is a Tyr analog, X10 is selected from hTyr, N-Me-Tyr, Tyr(3-tBu), Phe(4-OMe) and bhTyr. In particular embodiments, X10 is Phe(4-OMe). In particular embodiments, X10 is Tyr, Phe(4-OMe), Phe(4-OBzl), Phe(4-OMe), Phe(4-CONH₂), Phe(3,4-Cl₂), Phe(4-tBu), Phe(4-NH₂), Phe(4-Br), Phe(4-CN), Phe(4-carboxy), Phe[4-(2aminoethoxy)] or Phe(4-guanadino). In particular embodiments, X10 is not Tyr.

In certain embodiments, X11 is Trp or a Trp analog. In particular embodiments, X11 is 2-Nal or 1-Nal.

In particular embodiments, the peptide inhibitor of Formula III is cyclized. In certain embodiments, the peptide inhibitor is cyclized via an intramolecular bond between X4 and X9. In certain embodiments, the intramolecular bond is a thioether bond.

In certain embodiments, the peptide inhibitor of Formula III is linear or not cyclized. In particular embodiments of the linear peptide inhibitor of Formula III, X4 and/or X9 are any amino acid.

In particular embodiments of a peptide inhibitor of Formula III, one or more, two or more, or all three of X1, X2, and X3 are absent. In certain embodiments, X1 is absent. In certain embodiments, X1 and X2 are absent. In certain embodiments, X1, X2 and X3 are absent.

In particular embodiments of a peptide inhibitor of Formula III, one or more, two or more, three or more, four or more, or all of X16, X17, X18, X19 and X20 are absent. In particular embodiments of a peptide inhibitor of Formula III, one or more, two or more, three or more, or all of X17, X18, X19 and X20 are absent. In certain embodiments, one or more, two or more, or all three of X17, X19 and X20 are absent. In certain embodiments, one or more of X1, X2 and X3 are absent; and one or more, two or more, three or more, or four of X17, X18, X19 and X20 are absent.

In particular embodiments of a peptide inhibitor of Formula III, one of X4 or X9 is Abu, and the other of X4 or X9 is not Abu. In certain embodiments, X4 is Abu and X9 is Cys.

In particular embodiments, a peptide inhibitor of Formula III comprises one or more, two or more, three or more, or four of the following features: X5 is Arg or Gln; X6 is Thr; X7 is Trp; and X8 is Gln. In particular embodiments, X5 is Gln, X6 is Thr, X7 is Trp, and X8 is Gln. In certain embodiments, X5 is Gln. In certain embodiments, X1, X2 and X3 are absent. In certain embodiments, X4 is Abu and X9 is Cys.

In particular embodiments, a peptide inhibitor of Formula III comprises one or more, two or more, three or more, four or more, five or more, six or more, or seven of the following features: X10 is Tyr or a Phe analog; X11 is Trp, 2-Nal, 1-Nal, Phe(4-O-Allyl), Tyr(3-tBu), Phe(4-tBu), Phe(4-guanidino), Phe(4-OBzl) or Phe(4-Me); X12 is Arg, hLeu, (D)Asn, or any alpha methyl amino acids including, Aib, α-MeLys, α-MeLeu or α-MeOrn; X13 is Lys, Glu or Lys(Ac); X14 is Phe or Asn; X15 is β-Ala, Gln, Gly, Ser, Ala; and X16 is absent or AEA. In particular embodiments, a peptide inhibitor of Formula III comprises one or more, two or more, three or more, four or more, five or more, six or more, or seven of the following features: X10 is Tyr or a Phe analog; X11 is Trp, 2-Nal, 1-Nal, Phe(4-O-Allyl), Tyr(3-tBu), Phe(4-tBu), Phe(4-guanidino), Phe(4-OBzl) or Phe(4-Me); X12 is Arg, hLeu, (D)Asn, or any alpha methyl amino acids including, Aib, α-MeLys, α-MeLeu or α-MeOm; X13 is Lys, Glu or Lys(Ac); X14 is Phe or Asn; X15 is Gly, Ser, Ala; and X16 is absent or AEA. In certain embodiments, the Phe analog is Phe(4-OBzl), Phe(4-OMe), Phe(4-CONH₂), Phe(3,4-Cl₂), Phe(4-tBu), Phe(4-NH₂), Phe(4-Br), Phe(4-CN), Phe(4-carboxy), Phe[4-(2aminoethoxy)] or Phe(4-guanadino). In certain embodiments, X11 is 2-Nal or 1-Nal. In certain embodiments, X1, X2 and X3 are absent. In certain embodiments, X4 is Abu and X9 is Cys.

In particular embodiments, a peptide inhibitor of Formula III comprises one or more, two or more, three or more, four or more, five or more, six or more, or seven of the following features: X10 is Tyr or a Phe analog; X11 is Trp, 2-Nal, 1-Nal, Phe(4-O-Allyl), Tyr(3-tBu), Phe(4-tBu), Phe(4-guanidino), Phe(4-OBzl) or Phe(4-Me); X12 is Arg, hLeu, (D)Asn, 4-amino-4-carboxy-tetrahydropyran, Achc Acpc, Acbc, Acvc, Agp, Aib, α-DiethylGly, α-MeLys, α-MeLys(Ac), α-Me-Leu, α-MeOm, α-MeSer, α-MeVal; X13 is Lys, Glu or Lys(Ac); X14 is Phe or Asn; X15 is Gly; and X16 is absent or AEA. In certain embodiments, the Phe analog is Phe(4-OBzl), Phe(4-OMe), Phe(4-CONH₂), Phe(3,4-Cl2), Phe(4-tBu), Phe(4-NH₂), Phe(4-Br), Phe(4-CN), Phe(4-carboxy), Phe(4-(2aminoethoxy)) or Phe(4-guanadino). In certain embodiments, X11 is 2-Nal or 1-Nal. In certain embodiments, X1, X2 and X3 are absent. In certain embodiments, X4 is Abu and X9 is Cys.

In particular embodiments, a peptide inhibitor of Formula III comprises one or more, two or more, three or more, four or more, five or more, six or more, seven or more, eight or more, nine or more, ten or more, or eleven of the following features: X5 is Arg or Gln; X6 is Thr; X7 is Trp; X8 is Gln; X10 is a Phe analog; X11 is Trp, 2-Nal, 1-Nal, Phe(4-O-Allyl), Tyr(3-tBu), Phe(4-tBu), Phe(4-guanidino), Phe(Bzl) or Phe(4-Me); X12 is Aib, α-MeLys, α-MeLeu, 4-amino-4-carboxy-tetrahydropyran, Achc Acpc, Acbc, Acvc, Agp, Aib, α-DiethylGly, α-MeLys, α-MeLys(Ac), α-Me-Leu, α-MeSer, α-MeVal, α-MeOm; X13 is Lys, Glu or Lys(Ac); X14 is Phe or Asn; X15 is β-ala, Gly, Ser, Ala; and X16 is absent or AEA. In particular embodiments, a peptide inhibitor of Formula III comprises one or more, two or more, three or more, four or more, five or more, six or more, seven or more, eight or more, nine or more, ten or more, or eleven of the following features: X5 is Arg or Gln; X6 is Thr; X7 is Trp; X8 is Gln; X10 is a Phe analog; X11 is Trp, 2-Nal, 1-Nal, Phe(4-O-Allyl), Tyr(3-tBu), Phe(4-tBu), Phe(4-guanidino), Phe(Bzl) or Phe(4-Me); X12 is Aib, α-MeLys, α-MeLeu or α-MeOm; X13 is Lys, Glu or Lys(Ac); X14 is Phe or Asn; X15 is Gly, Ser, Ala; and X16 is absent or AEA. In certain embodiments, the Phe analog is Phe(4-OBzl), Phe(4-OMe), Phe[4-(2aminoethoxy)], Phe(4-CONH₂), Phe(3,4-Cl₂), Phe(4-tBu), Phe(4-NH₂), Phe(4-Br), Phe(4-CN), Phe(4-CO₂H), or Phe(4-guanadino). In certain embodiments, X11 is 2-Nal or 1-Nal. In certain embodiments, X1, X2 and X3 are absent. In certain embodiments, X4 is Abu and X9 is Cys.

In particular embodiments, a peptide inhibitor of Formula III comprises one or more, two or more, three or more, four or more, five or more, six or more, seven or more, eight or more, nine or more, ten or more, or eleven of the following features: X5 is Arg or Gln; X6 is Thr; X7 is Trp; X8 is Gln; X10 is Tyr or a Phe analog; X11 is Trp, 2-Nal, 1-Nal, Phe(4-O-Allyl), Tyr(3-tBu), Phe(4-tBu), Phe(4-guanidino), Phe(Bzl) or Phe(4-Me); X12 is Arg, hLeu, (D)Asn, 4-amino-4-carboxy-tetrahydropyran, Achc Acpc, Acbc, Acvc, Aib, α-DiethylGly, α-MeLys, α-MeLys(Ac), α-Me-Leu, α-MeSer, α-MeVal; X13 is Lys, Glu or Lys(Ac); X14 is Phe or Asn; X15 is β-Ala, Asn or Gly; and X16 is absent or AEA. In particular embodiments, a peptide inhibitor of Formula III comprises one or more, two or more, three or more, four or more, five or more, six or more, seven or more, eight or more, nine or more, ten or more, or eleven of the following features: X5 is Arg or Gln; X6 is Thr; X7 is Trp; X8 is Gln; X10 is Tyr or a Phe analog; X11 is Trp, 2-Nal, 1-Nal, Phe(4-O-Allyl), Tyr(3-tBu), Phe(4-tBu), Phe(4-guanidino), Phe(Bzl) or Phe(4-Me); X12 is Arg, hLeu, (D)Asn, α-MeLys, α-MeLeu or α-MeOm, Aib; X13 is Lys, Glu or Lys(Ac); X14 is Phe or Asn; X15 is Gly; and X16 is absent or AEA. In certain embodiments, the Phe analog is Phe(4-OBzl), Phe(40Me), Phe(4-CONH₂), Phe(3,4-Cl₂), Phe(4-tBu), Phe(4-NH₂), Phe(4-Br), Phe(4-CN), Phe(4-CO₂H), Phe(4-(2-aminoethoxy)) or Phe(4-guanidino). In certain embodiments, X11 is 2-Nal or 1-Nal. In certain embodiments, X1, X2 and X3 are absent. n certain embodiments, X4 is Abu and X9 is Cys.

In certain embodiments, the present invention includes a peptide of 8 to 20, 8 to 16 or 8 to 12 amino acids, optionally cyclized, comprising or consisting of a core sequence of Formula IIIb:

Xaa4-Xaa5-Xaa6-Trp-Xaa8-Xaa9-Xaa10-Xaa11 (IIIb)

wherein Xaa4 and Xaa9 are each independently selected from Abu and Cys, wherein Xaa4 and Xaa9 are not both the same; Xaa5, Xaa6 and Xaa8 are any amino acid residue; Xaa10 is Tyr, a Phe analog or 2-Nal, and Xaa11 is 2-Nal or Trp, wherein the peptide inhibits binding of IL-23 to IL-23R. In particular embodiments, Xaa10 is Phe(4-OMe), 2-Nal, or Phe[4-(2-aminoethoxy)]. In one embodiment, Xaa10 is Phe(4-OMe). In one embodiment, Xaa7 is Phe[4-(2-aminoethoxy)].

In one embodiment, Xaa11 is 2-Nal. In certain embodiments, the peptide is cyclized via Xaa4 and Xaa9. In particular embodiments, the Phe analog is Phe[4-(2aminoethoxy)] or Phe(4-OMe). In certain embodiments, Xaa4 is Abu and Xaa9 is Cys, and the peptide is cyclized via Xaa4 and Xaa9. In particular embodiments, the peptide is a peptide inhibitor of Formula III, and wherein in certain embodiments, X1, X2 and X3 are absent. In particular embodiments, the peptide inhibits the binding of IL-23 to IL-23R. In certain embodiments, a peptide of Formula IIIb comprises a Glu, (D)Glu, Arg, (D)Arg, Phe, (D)Phe, 2-Nal, Thr, Leu, or (D)Gln bound to Xaa4. In certain embodiments, it is (D)Arg or (D)Phe.

In certain embodiments, the present invention includes a peptide of 8 to 20, 8 to 16 or 8 to 12 amino acids, optionally cyclized, comprising or consisting of a core sequence of Formula IIIc:

Abu-Xaa5-Xaa6-Trp-Xaa8-Cys-[Phe(4-OMe)]-(2-Nal) (IIIc)

wherein Xaa5, Xaa6 and Xaa8 are any amino acid residue; and wherein the peptide inhibits binding of IL-23 to IL-23R. In certain embodiments, the peptide is cyclized via Abu at Xaa4 and Cys at Xaa9. In certain embodiments, the peptide is a peptide inhibitor of Formula III, and wherein in certain embodiments, X1, X2 and X3 are absent. In particular embodiments, the peptide inhibits the binding of IL-23 to IL-23R. In certain embodiments, a peptide of Formula IIIc comprises a Glu, (D)Glu, Arg, (D)Arg, Phe, (D)Phe, 2-Nal, Thr, Leu, or (D)Gln bound to Abu. In certain embodiments, it is (D)Arg or (D)Phe.

In certain embodiments, the present invention includes a peptide of 8 to 20, 8 to 16 or 8 to 12 amino acids, optionally cyclized, comprising or consisting of a core sequence of Formula IIId:

Abu-Xaa5-Xaa6-Trp-Xaa8-Cys-Xaa10-Trp (IIId)

wherein Xaa5, Xaa6 and Xaa8 are any amino acid residue; Xaa10 is a modified Phe; and wherein the peptide inhibits binding of IL-23 to IL-23R. In particular embodiments, the modified Phe is Phe(4-tBu), Phe(4-guanidino), Phe[4-(2-aminoethoxy)], Phe(4-CO₂H), Phe(4-CN), Phe(4-Br), Phe(4-NH₂), PHe(CONH₂) or Phe(4-Me). In particular embodiments, the modified Phe is Phe(4-tBu), Phe(4-guanidino), Phe[4-(2-aminoethoxy)], Phe(4-CO₂H), Phe(4-CN), Phe(4-Br), Phe(4-NH₂), or Phe(4-Me). In one embodiment, Xaa10 is Phe[4-(2-aminoethoxy)] or Phe(4-OMe). In one embodiment, Xaa10 is Phe[4-(2-aminoethoxy)]. In certain embodiments, the peptide is cyclized via Abu at Xaa4 and Cys at Xaa9. In certain embodiments, the peptide is a peptide inhibitor of Formula III, and wherein in certain embodiments, X1, X2 and X3 are absent. In particular embodiments, the peptide inhibits the binding of IL-23 to IL-23R. In certain embodiments, a peptide of Formula IIId comprises a Glu, (D)Glu, Arg, (D)Arg, Phe, (D)Phe, 2-Nal, Thr, Leu, or (D)Gln bound to Abu. In certain embodiments, it is (D)Arg or (D)Phe.

In certain embodiments, the present invention includes a peptide, optionally 8 to 20, 8 to 16 or 8 to 12 amino acids, optionally cyclized, comprising or consisting of a core sequence of Formula IIIe:

Abu-Xaa5-Xaa6-Trp-Xaa8-Cys-Phe[4-(2-aminoethoxy)]-[2-Nal] (IIIe)

wherein Xaa5, Xaa6 and Xaa8 are any amino acid residue. In certain embodiments, the peptide is cyclized via Abu at Xaa4 and Cys at Xaa9. In certain embodiments, the peptide is a peptide inhibitor of Formula III, and wherein in certain embodiments, X1, X2 and X3 are absent. In particular embodiments, the peptide inhibits the binding of IL-23 to IL-23R. In certain embodiments, a peptide of Formula IIIb comprises a Glu, (D)Glu, Arg, (D)Arg, Phe, (D)Phe, 2-Nal, Thr, Leu, or (D)Gln bound to Abu. In certain embodiments, it is (D)Arg or (D)Phe.

In one embodiment, Xaa5 and Xaa8 is Gln. In one embodiment, Xaa6 is Thr. In certain embodiments, the peptide is cyclized via Abu at Xaa4 and Cys at Xaa9.

In particular embodiments of a peptide inhibitor of Formula III, the peptide inhibitor has a structure shown in any of Tables 4, 5A, 5B or 6, or comprises an amino acid sequence set forth in Tables 4, 5A, 5B or 6.

In certain aspects, the present invention provides a peptide inhibitor of an interleukin-23 receptor, or a pharmaceutically acceptable salt or solvate thereof, wherein the peptide inhibitor comprises an amino acid sequence of Formula (Vf):

X1-X2-X3-Abu-X5-X6-X7-X8-Cys-X10-X11-X12-X13-X14-X15-X16-X17-X18-X19-X20 (Vf),

wherein:
X1 is absent;
X2 is absent or X2 is D-Asp, E, R, D-Arg, F, D-Phe, 2-Nal, T, L, D-Gln, or D-Asn;

### X3 is D-Arg;

X5 is N, Q, Cit, Lys, or a Lys conjugate (e.g., Lys(IVA), Lys(biotin), Lys(octanyl), Lys(Palm), Lys(PEG), Lys(PEG8), Lys(PEG11-Palm), Lys(Ac));

### X6 is T, S or V;

### X7 is W, 1-Nal, or 2-Nal;

### X8 is Q, Cit, N, Aib or Lys(Ac);

X10 is Phe[4-(2-aminoethoxy)], Phe[4-(2-acetylaminoethoxy)] or Phe(4-CONH₂);

### X11 is 2-Nal;

X12 is 4-amino-4-carboxy-tetrahydropyran, Aib, αMeLeu, αMeLys, or an αMeLys conjugate (e.g., αMeLys(Ac), αMeLys(PEG4-Palm), αMeLys(PEG4-Lauryl), αMeLys(PEG4IsoGluPalm), αMeLys(PEG4IsoGluLauryl), αMeLys(IVA), αMeLys (biotin), or αMeLys(octanyl));
X13 is Q, E, Cit or a Lys conjugate (e.g., Lys(Ac), Lys(PEG4-isoGlu-Palm), Lys(PEG4-octanyl), Lys(PEG4-Palm), Lys(biotin), Lys(octanyl), Lys(Palm), Pys(PEG8), or Lys(PEG11-Palm));
X14 is N, Cit, Q, L, G, S, Aib, F, 2-Nap, N-Me-Ala, R, W, nLeu, Tic or a Lys conjugate (e.g., Lys(Ac));

### X15 is N, Cit, Q, βAla, Lys(Ac) or Aib; and

X16, X17, X18, X19 and X20 are absent.

In particular embodiments, X2 is D-Asp, E, R, D-Arg, F, D-Phe, 2-Nal, T, L, D-Gln, or D-Asn.

In certain aspects, the present invention provides a peptide inhibitor of an interleukin-23 receptor, or a pharmaceutically acceptable salt or solvate thereof, wherein the peptide inhibitor comprises an amino acid sequence of Formula (Vh):

X1-X2-X3-Abu-X5-X6-X7-X8-Cys-X10-X11-X12-X13-X14-X15-X16-X17-X18-X19-X20 (Vh),

wherein:
X1 is any amino acid or absent;
X2 is any amino acid or absent;
X3 is any D-amino acid or absent;
X4 is Cys, hCys, Pen, hPen, Abu, Ser, hSer or chemical moiety capable of forming a bond with X9;

### X5 is Ala, α-MeOrn, α-MeSer, Cit, Dap, Dab, Dap(Ac), Gly, Lys, Asn, N-MeGln, N-MeArg, Om, Gln, Arg, Ser, Glu or Thr;

X6 is Thr, Ser, Asp, Ile or any amino acid;

### X7 is Trp, 6-Chloro-Trp, 1-Nap or 2-Nap;

X8 is Glu, Gln, Asn, Lys(Ac), Cit, Cav, Lys(N-ε-(N-α-Palmitoyl-L-γ-glutamyl)), or Lys(N-ε-Palmitoyl;
X9 is Cys, hCys, Pen, hPen Abu, or any amino acid or chemical moiety capable of forming a bond with X4;
X10 is 2-Nal, a Phe analog, Tyr, or a Tyr analog;
X11 is 1-Nal, 2-Nal, Phe(3,4-dimethoxy), 5-HydroxyTrp, Phe(3,4-Cl2), Trp or Tyr(3-tBu);
X12 is Aib, 4-amino-4-carboxy-tetrahydropyran, any alpha-methylamino acid, alpha-ethyl-amino acid, Achc, Acvc, Acbc Acpc, 4-amino-4-carboxy-piperidine, 3-Pal, Agp, D-DiethylGly, α-MeLys, α-MeLys(Ac), α-MeLeu, α-MeOrn, α-MeSer, α-MeVal, Cav, Cha, Cit, Cpa, D-Asn, Glu, His, hLeu, hArg, Lys, Leu, Octgly, Om, piperidine, Arg, Ser, Thr or THP;
X13 is Lys(Ac), Gln, Cit, Glu, or any amino acid;
X14 is Asn, Gln, Lys(Ac), Cit, Cav, Lys(N-ε-(N-α-Palmitoyl-L-γ-glutamyl)), Lys(N-ε-Palmitoyl), Asp, or any amino acid;

### X15 is β-Ala, Asn, Gly, Gln, Ala, Ser, Aib, Asp or Cit;

X16 is any amino acid or absent;
X17 is any amino acid or absent;
X18 is any amino acid or absent;
X19 is any amino acid or absent; and
X20 is any amino acid or absent.

In certain embodiments of any of the peptide inhibitors described herein, including but not limited to those of Formula (If) and (Ih), the peptide inhibitor is cyclized via a bond, e.g., a thioether bond, between X4 and X9. In certain embodiments, the peptide inhibitor inhibits the binding of an interleukin-23 (IL-23) to an IL-23 receptor.

In certain embodiments, X1, X2 and X3 are absent. In certain embodiments, X1 and X2 are absent. In certain embodiments, X1 is a D-amino acid or absent. In certain embodiments, X2 is a D-amino acid or absent.

In certain embodiments, X5 is Ala, α-MeOrn, α-MeSer, Cit, Dap, Dab, Dap(Ac), Gly, Lys, Asn, N-MeGln, N-MeArg, Om, Gln, Arg, Ser, or Thr.

In certain embodiments, X5 is N, X6 is T, X7 is W, or X8 is Q. In certain embodiments, X5 is N, X6 is T, X7 is W, and X8 is Q.

In certain embodiments, X5 is Q, X6 is T, X7 is W, or X8 is Q. In certain embodiments, X5 is Q, X6 is T, X7 is W, and X8 is Q.

In certain embodiments, X5 is N, X6 is T, X7 is W, and X8 is Cit.

In certain embodiments, X10 is Phe[4-(2-aminoethoxy)].

In certain embodiments, X12 is 4-amino-4-carboxy-tetrahydropyran, Aib, αMeLeu, or αMeLys. In certain embodiments, X12 is 4-amino-4-carboxy-tetrahydropyran. In certain embodiments, X13 is E or Lys(Ac). In certain embodiments, X13 is Lys(Ac).

In certain embodiments, X14 is Asn, Gln, Lys(Ac), Cit, Cav, Lys(N-ε-(N-α-Palmitoyl-L-γ-glutamyl)), Lys(N-ε-Palmitoyl), or any amino acid.

In certain embodiments, X15 is R-Ala, Asn, Gly, Gln, Ala, Ser, Aib, or Cit.

In certain embodiments, X14 is N.

In certain embodiments, X15 is N.

In certain embodiments, X16 is a D-amino acid or absent. In certain embodiments, X17 is a D-amino acid or absent. In certain embodiments, X18 is a D-amino acid or absent. In certain embodiments, X19 is a D-amino acid or absent. In certain embodiments, X20 is a D-amino acid or absent.

In certain embodiments, X2 is absent; X3 is absent; X5 is Q, X6 is T, X7 is W, and X8 is Q; X10 is Phe[4-(2-aminoethoxy)]; X12 is 4-amino-4-carboxy-tetrahydropyran, Aib, αMeLeu, or αMeLys; X13 is E or Lys(Ac); X14 is N; and X15 is N. In certain embodiments, X12 is 4-amino-4-carboxy-tetrahydropyran and X13 is Lys(Ac).

In certain embodiments, any of the amino acids of the peptide inhibitor are connected by a linker moiety, e.g., a PEG.

In certain embodiments, the N-terminus of the peptide inhibitor comprises an Ac group.

In certain embodiments, the C-terminus of the peptide inhibitor comprises an NH₂ group.

In certain embodiments, the present invention includes a peptide comprising or consisting of an amino acid sequence shown in any of Tables 4, 5A, 5B or 6, or a peptide inhibitor comprising or consisting of a structure shown in any of the Table 4, 5A, 5B or 6 (or a pharmaceutically acceptable salt thereof). In particular embodiments, the peptide does not include the conjugated moieties but does include the Abu residue. In particular embodiments, the peptide or inhibitor comprises a thioether bond between the two Abu and Cys residues, or between the two outermost amino acids within the brackets following the term “cyclo”, which indicated the presence of a cyclic structure. In particular embodiments, the inhibitor is an acetate salt. The peptide sequence of illustrative inhibitors is shown in Tables 4, 5A, 5B and 6 from N-term to C-term, with conjugated moieties, and N-terminal Ac and/or C-terminal NH₂ groups indicated. The peptides depicted in Tables 4, 5A, 5B, and 6 are cyclic. The cyclic structure is indicated by “Cyclo” as illustrated in Table 5A, indicating the presence of a thioether bond between the bracketed Abu at X4 and Cys at X9.

TABLE 4
Illustrative Thioether Peptide Inhibitors

Biotin-[PEG4]-cyclo[[Abu]-QTWQC]-[Phe[4-(2-aminoethoxy)]-[2-Nal]-[4-amino-4-carboxy-tetrahydropyran]-
ENN-NH₂
Ac-cyclo[[Abu]-QTWQC]-[Phe[4-(2-aminoethoxy)]-[2-Nal]-[4-amino-4-carboxy-tetrahydropyran]-[Lys(Ac)]-NN-
NH₂
Ac-[(D)Arg]-cyclo[[Abu]-QTWQC]-[Phe[4-(2-aminoethoxy)]-[2-Nal]-[4-amino-4-carboxy-tetrahydropyran]-
[Lys(Ac)]-NN-NH₂
Ac-[(D)Arg]-cyclo[[Abu]-QTWQC]-[Phe[4-(2-aminoethoxy)]-[2-Nal]-[4-amino-4-carboxy-tetrahydropyran]-
[Lys(Ac)]-NN-NH₂
Ac-E-[(D)Arg]-cyclo[[Abu]-QTWQC]-[Phe[4-(2-aminoethoxy)]-[2-Nal]-[4-amino-4-carboxy-tetrahydropyran]-
ENN-NH₂
Ac-[(D)Asp]-[(D)Arg]-cyclo[[Abu]-QTWQC]-[Phe[4-(2-aminoethoxy)]-[2-Nal]-[4-amino-4-carboxy-
tetrahydropyran]-ENN-NH₂
Ac-R-[(D)Arg]-cyclo[[Abu]-QTWQC]-[Phe[4-(2-aminoethoxy)]-[2-Nal]-[4-amino-4-carboxy-tetrahydropyran]-
ENN-NH₂
Ac-[(D)Arg]-[(D)Arg]-cyclo[[Abu]-QTWQC]-[Phe[4-(2-aminoethoxy)]-[2-Nal]-[4-amino-4-carboxy-
tetrahydropyran]-ENN-NH₂
Ac-F-[(D)Arg]-cyclo[[Abu]-QTWQC]-[Phe[4-(2-aminoethoxy)]-[2-Nal]-[4-amino-4-carboxy-tetrahydropyran]-
ENN-NH₂
Ac-[(D)Phe]-[(D)Arg]-cyclo[[Abu]-QTWQC]-[Phe[4-(2-aminoethoxy)]-[2-Nal]-[4-amino-4-carboxy-
tetrahydropyran]-ENN-NH₂
Ac-[2-Nal]-[(D)Arg]-cyclo[[Abu]-QTWQC]-[Phe[4-(2-aminoethoxy)]-[2-Nal]-[4-amino-4-carboxy-
tetrahydropyran]-ENN-NH₂
Ac-T-[(D)Arg]-cyclo[[Abu]-QTWQC]-[Phe[4-(2-aminoethoxy)]-[2-Nal]-[4-amino-4-carboxy-tetrahydropyran]-
ENN-NH₂
Ac-L-[(D)Arg]-cyclo[[Abu]-QTWQC]-[Phe[4-(2-aminoethoxy)]-[2-Nal]-[4-amino-4-carboxy-tetrahydropyran]-
ENN-NH₂
Ac-[(D)Gln]-[(D)Arg]-cyclo[[Abu]-QTWQC]-[Phe[4-(2-aminoethoxy)]-[2-Nal]-[4-amino-4-carboxy-
tetrahydropyran]-ENN-NH₂
Ac-[(D)Asn]-[(D)Arg]-cyclo[[Abu]-QTWQC]-[Phe[4-(2-aminoethoxy)]-[2-Nal]-[4-amino-4-carboxy-
tetrahydropyran]-ENN-NH₂
Ac-cyclo[[Abu]-QTWQC]-[Phe[4-(2-aminoethoxy)-(PEG4-Alexa488)]-[2-Nal]-[4-amino-4-carboxy-
tetrahydropyran]-ENN-NH₂
[Alexa488]-[PEG4]-cyclo[[Abu]-QTWQC]-[Phe[4-(2-aminoethoxy)]--[2-Nal]-[4-amino-4-carboxy-
tetrahydropyran]-ENN-NH₂
[Alexa647]-[PEG4]-cyclo[[Abu]-QTWQC]-[Phe[4-(2-aminoethoxy)]-[2-Nal]-[4-amino-4-carboxy-
tetrahydropyran]-ENN-NH₂
[Alexa-647]-[PEG4]-[(D)Arg]-cyclo[[Abu]-QTWQC]-[Phe[4-(2-aminoethoxy)]-[2-Nal]-[4-amino-4-carboxy-
tetrahydropyran]-[Lys(Ac)]-NN-NH₂
[Alexa647]-[PEG12]-[(D)Arg]-cyclo[[Abu]-QTWQC]-[Phe[4-(2-aminoethoxy)]-[2-Nal]-[4-amino-4-carboxy-
tetrahydropyran]-[Lys(Ac)]-NN-NH₂
[Alexa488]-[PEG4]-[(D)Arg]-cyclo[[Abu]-QTWQC]-[Phe[4-(2-aminoethoxy)]-[2-Nal]-[4-amino-4-carboxy-
tetrahydropyran]-[Lys(Ac)]-NN-NH₂

TABLE 5A
Illustrative Thioether Peptide Inhibitors

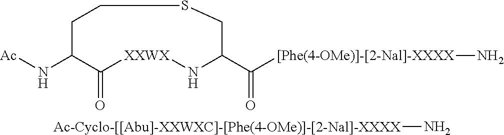

Sequence
Ac-cyclo[[Abu]-QTWQC]-[Phe[4-(2-aminoethoxy)]-[2-Nal]-[4-amino-4-carboxy-tetrahydropyran]-
ENN—NH₂
Ac-cyclo[[Abu]-QTWQC]-[Phe[4-(2-aminoethoxy)]-[2-Nal]-[4-amino-4-carboxy-tetrahydropyran]-
ENNE—NH₂
Ac-cyclo[[Abu]-QTWQC]-[Phe[4-(2-aminoethoxy)]-[2-Nal]-[4-amino-4-carboxy-tetrahydropyran]-
ENNF—NH₂
Ac-cyclo[[Abu]-QTWQC]-[Phe[4-(2-aminoethoxy)]-[2-Nal]-[4-amino-4-carboxy-tetrahydropyran]-
ENNK—NH₂
Ac-cyclo[[Abu]-QTWQC]-[Phe[4-(2-aminoethoxy)]-[2-Nal]-[4-amino-4-carboxy-tetrahydropyran]-
ENNN—NH₂
Ac-cyclo[[Abu]-QTWQC]-[Phe[4-(2-aminoethoxy)]-[2-Nal]-[4-amino-4-carboxy-tetrahydropyran]-
ENNW—NH₂
Ac-cyclo[[Abu]-QTWQC]-[Phe[4-(2-aminoethoxy)]-[2-Nal]-[4-amino-4-carboxy-tetrahydropyran]-
ENNT—NH₂
Ac-cyclo[[Abu]-QTWQC]-[Phe[4-(2-aminoethoxy)]-[2-Nal]-[4-amino-4-carboxy-tetrahydropyran]-
ENNG—NH₂
Ac-cyclo[[Abu]-QTWQC]-[Phe[4-(2-aminoethoxy)]-[2-Nal]-[4-amino-4-carboxy-tetrahydropyran]-
ENNPK—NH₂
Ac-cyclo[[Abu]-QTWQC]-[Phe[4-(2-aminoethoxy)]-[2-Nal]-[4-amino-4-carboxy-tetrahydropyran]-
ENNPG—NH₂
Ac-cyclo[[Abu]-QTWQC]-[Phe[4-(2-aminoethoxy)]-[2-Nal]-[4-amino-4-carboxy-tetrahydropyran]-
ENNEP—NH₂
Ac-cyclo[[Abu]-QTWQC]-[Phe[4-(2-aminoethoxy)]-[2-Nal]-[4-amino-4-carboxy-tetrahydropyran]-
ENNGK—NH₂
Ac-cyclo[[Abu]-QTWQC]-[Phe[4-(2-aminoethoxy)]-[2-Nal]-[4-amino-4-carboxy-tetrahydropyran]-
ENNPT—NH₂
Ac-cyclo[[Abu]-QTWQC]-[Phe[4-(2-aminoethoxy)]-[2-Nal]-[4-amino-4-carboxy-tetrahydropyran]-
ENNGF—NH₂
Ac-cyclo[[Abu]-QTWQC]-[Phe[4-(2-aminoethoxy)]-[2-Nal]-[4-amino-4-carboxy-tetrahydropyran]-
ENNGW—NH₂
Ac-cyclo[[Abu]-QTWQC]-[Phe[4-(2-aminoethoxy)]-[2-Nal]-[4-amino-4-carboxy-tetrahydropyran]-
ENNGQ—NH₂
Ac-cyclo[[Abu]-QTWQC]-[Phe[4-(2-aminoethoxy)]-[2-Nal]-[4-amino-4-carboxy-tetrahydropyran]-
ENNGGG—NH₂
Ac-cyclo[[Abu]-QTWQC]-[Phe[4-(2-aminoethoxy)]-[2-Nal]-[4-amino-4-carboxy-tetrahydropyran]-
ENNKKK—NH₂
Ac-cyclo[[Abu]-QTWQC]-[Phe[4-(2-aminoethoxy)]-[2-Nal]-[4-amino-4-carboxy-tetrahydropyran]-
ENNEEE—NH₂
Ac-cyclo[[Abu]-QTWQC]-[Phe[4-(2-aminoethoxy)]-[2-Nal]-[4-amino-4-carboxy-tetrahydropyran]-
ENNFFF—NH₂
Ac-cyclo[[Abu]-QTWQC]-[Phe[4-(2-aminoethoxy)]-[2-Nal]-[4-amino-4-carboxy-tetrahydropyran]-
ENNTTT—NH₂
Ac-cyclo[[Abu]-QTWQC]-[Phe[4-(2-aminoethoxy)]-[2-Nal]-[4-amino-4-carboxy-tetrahydropyran]-
ENNGGGR—NH₂
Ac-cyclo[[Abu]-QTWQC]-[Phe[4-(2-aminoethoxy)]-[2-Nal]-[4-amino-4-carboxy-tetrahydropyran]-
ENNGGGF—NH₂
Ac-cyclo[[Abu]-QTWQC]-[Phe[4-(2-aminoethoxy)]-[2-Nal]-[4-amino-4-carboxy-tetrahydropyran]-
ENNGGGE—NH₂
Ac-cyclo[[Abu]-QTWQC]-[Phe[4-(2-aminoethoxy)]-[2-Nal]-[4-amino-4-carboxy-tetrahydropyran]-
ENNGGGQ—NH₂
Ac-cyclo[[Abu]-QTWQC]-[Phe[4-(2-aminoethoxy)]-[2-Nal]-[4-amino-4-carboxy-tetrahydropyran]-
ENNGGGT—NH₂
Ac-cyclo[[Abu]-QTWQC]-[Phe[4-(2-aminoethoxy)]-[2-Nal]-[4-amino-4-carboxy-tetrahydropyran]-
ENNGGGGR—NH₂
Ac-cyclo[[Abu]-QTWQC]-[Phe[4-(2-aminoethoxy)]-[2-Nal]-[4-amino-4-carboxy-tetrahydropyran]-
ENNGGGGF—NH₂
Ac-cyclo[[Abu]-QTWQC]-[Phe[4-(2-aminoethoxy)]-[2-Nal]-[4-amino-4-carboxy-tetrahydropyran]-
ENNGGGGE—NH₂
Ac-cyclo[[Abu]-QTWQC]-[Phe[4-(2-aminoethoxy)]-[2-Nal]-[4-amino-4-carboxy-tetrahydropyran]-
ENNGGGGQ—NH₂
Ac-cyclo[[Abu]-QTWQC]-[Phe[4-(2-aminoethoxy)]-[2-Nal]-[4-amino-4-carboxy-tetrahydropyran]-
ENNGGGGT—NH₂
Ac-cyclo[[Abu]-QTWQC]-[Phe[4-(2-aminoethoxy)]-[2-Nal]-[4-amino-4-carboxy-tetrahydropyran]-
ENNRRRRR—NH₂
Ac-cyclo[[Abu]-QTWQC]-[Phe[4-(2-aminoethoxy)]-[2-Nal]-[4-amino-4-carboxy-tetrahydropyran]-
ENNGFFFFF—NH₂
Ac-cyclo[[Abu]-QTWQC]-[Phe[4-(2-aminoethoxy)]-[2-Nal]-[4-amino-4-carboxy-tetrahydropyran]-
ENNEEEEE—NH₂
Ac-cyclo[[Abu]-QTWQC]-[Phe[4-(2-aminoethoxy)]-[2-Nal]-[4-amino-4-carboxy-tetrahydropyran]-
ENNQQQQQ—NH₂
Ac-cyclo[[Abu]-QTWQC]-[Phe[4-(2-aminoethoxy)]-[2-Nal]-[4-amino-4-carboxy-tetrahydropyran]-
ENNTTTTT—NH₂
Ac-cyclo[[Abu]-QTWQC]-[Phe[4-(2-aminoethoxy)]-[2-Nal]-[4-amino-4-carboxy-tetrahydropyran]-
ENN—NH₂
Ac-GGG-cyclo[[Abu]-QTWQC]-[Phe[4-(2-aminoethoxy)]-[2-Nal]-[4-amino-4-carboxy-tetrahydropyran]-
ENN—NH₂
Ac-RRR-cyclo[[Abu]-QTWQC]-[Phe[4-(2-aminoethoxy)]-[2-Nal]-[4-amino-4-carboxy-tetrahydropyran]-
ENN—NH₂
Ac-FFF-cyclo[[Abu]-QTWQC]-[Phe[4-(2-aminoethoxy)]-[2-Nal]-[4-amino-4-carboxy-tetrahydropyran]-
ENN—NH₂
Ac-EEE—cyclo[[Abu]-QTWQC]-[Phe[4-(2-aminoethoxy)]-[2-Nal]-[4-amino-4-carboxy-tetrahydropyran]-
ENN—NH₂
Ac-QQQ-cyclo[[Abu]-QTWQC]-[Phe[4-(2-aminoethoxy)]-[2-Nal]-[4-amino-4-carboxy-tetrahydropyran]-
ENN—NH₂
Ac-TTT-cyclo[[Abu]-QTWQC]-[Phe[4-(2-aminoethoxy)]-[2-Nal]-[4-amino-4-carboxy-tetrahydropyran]-
ENN—NH₂
Ac-RG-cyclo[[Abu]-QTWQC]-[Phe[4-(2-aminoethoxy)]-[2-Nal]-[4-amino-4-carboxy-tetrahydropyran]-
ENN—NH₂
Ac-FG-cyclo[[Abu]-QTWQC]-[Phe[4-(2-aminoethoxy)]-[2-Nal]-[4-amino-4-carboxy-tetrahydropyran]-
ENN—NH₂
Ac-EG-cyclo[[Abu]-QTWQC]-[Phe[4-(2-aminoethoxy)]-[2-Nal]-[4-amino-4-carboxy-tetrahydropyran]-
ENN—NH₂
Ac-QG-cyclo[[Abu]-QTWQC]-[Phe[4-(2-aminoethoxy)]-[2-Nal]-[4-amino-4-carboxy-tetrahydropyran]-
ENN—NH₂
Ac-TG-cyclo[[Abu]-QTWQC]-[Phe[4-(2-aminoethoxy)]-[2-Nal]-[4-amino-4-carboxy-tetrahydropyran]-
ENN—NH₂
Ac-cyclo[[Abu]-QTWQC]-[Phe[4-(2-aminoethoxy)]-[2-Nal]-[4-amino-4-carboxy-tetrahydropyran]-
[Lys(Palm)]-NN—NH₂
Ac-cyclo[[Abu]-QTWQC]-[Phe[4-(2-aminoethoxy)]-[2-Nal]-[4-amino-4-carboxy-tetrahydropyran]-
[Lys(PEG11-Palm)]-NN—NH₂
Ac-cyclo[[Abu]-QTWQC]-[Phe[4-(2-aminoethoxy)]-[2-Nal]-[4-amino-4-carboxy-tetrahydropyran]-
[Lys(isoGlu-Palm)]-NN—NH₂
Ac-cyclo[[Abu]-QTWQC]-[Phe[4-(2-aminoethoxy)]-[2-Nal]-[4-amino-4-carboxy-tetrahydropyran]-
[Lys(Ahx-Palm)]-NN—NH₂
Ac-cyclo[[Abu]-QTWQC]-[Phe[4-(2-aminoethoxy)]-[2-Nal]-[4-amino-4-carboxy-tetrahydropyran]-
[Lys(isoGlu-Ahx-Palm)]-NN—NH₂
Ac-cyclo[[Abu]-QTWQC]-[Phe[4-(2-aminoethoxy)]-[2-Nal]-[4-amino-4-carboxy-tetrahydropyran]-
[Lys(isoGlu-Ahx-Palm)]-NN—NH₂
[Palm]-cyclo[[Abu]-QTWQC]-[Phe[4-(2-aminoethoxy)]-[2-Nal]-[4-amino-4-carboxy-tetrahydropyran]-
ENN—NH₂
[Palm-isoGlu]-cyclo[[Abu]-QTWQC]-[Phe[4-(2-aminoethoxy)]-[2-Nal]-[4-amino-4-carboxy-
tetrahydropyran]-ENN—NH₂
[Palm-PEG11]-cyclo[[Abu]-QTWQC]-[Phe[4-(2-aminoethoxy)]-[2-Nal]-[4-amino-4-carboxy-
tetrahydropyran]-ENN—NH₂
[Palm-Ahx]-cyclo[[Abu]-QTWQC]-[Phe[4-(2-aminoethoxy)]-[2-Nal]-[4-amino-4-carboxy-
tetrahydropyran]-ENN—NH₂
[Palm-Ahx-isoGlu]-cyclo[[Abu]-QTWQC]-[Phe[4-(2-aminoethoxy)]-[2-Nal]-[4-amino-4-carboxy-
tetrahydropyran]-ENN—NH₂
Ac-cyclo[[Abu]-QTWQC]-[Phe[4-(2-aminoethoxy)]-[2-Nal]-[4-amino-4-carboxy-tetrahydropyran]-ENN-
Lys[Palm]—NH₂
Ac-cyclo[[Abu]-QTWQC]-[Phe[4-(2-aminoethoxy)]-[2-Nal]-[4-amino-4-carboxy-tetrahydropyran]-ENN-
Lys[Peg11-Palm]—NH₂
Ac-cyclo[[Abu]-QTWQC]-[Phe[4-(2-aminoethoxy)]-[2-Nal]-[4-amino-4-carboxy-tetrahydropyran]-ENN-
Lys[isoGlu-Palm]—NH₂
Ac-cyclo[[Abu]-QTWQC]-[Phe[4-(2-aminoethoxy)]-[2-Nal]-[4-amino-4-carboxy-tetrahydropyran]-ENN-
Lys[Ahx-Palm]—NH₂
Ac-cyclo[[Abu]-QTWQC]-[Phe[4-(2-aminoethoxy)]-[2-Nal]-[4-amino-4-carboxy-tetrahydropyran]-ENN-
Lys[isoGlu-Ahx-Palm]—NH₂

TABLE 5B
Illustrative Thioether Peptides

Ac-[D-Arg]-Cyclo-[Abu-QTWQC]-[Phe(4-2ae)]-[2-Nal]-[THP]-ENN-NH₂
Ac-[D-Arg]-Cyclo-[Abu-QTWQC]-[Phe(4-2ae)]-[2-Nal]-[THP]-END-NH₂
Ac-[D-Arg]-Cyclo-[Abu-QTWQC]-[Phe(4-2ae)]-[2-Nal]-[THP]-EDN-NH₂
Ac-[D-Arg]-Cyclo-[Abu-QTWEC]-[Phe(4-2ae)]-[2-Nal]-[THP]-ENN-NH₂
Ac-[D-Arg]-Cyclo-[Abu-ETWQC]-[Phe(4-2ae)]-[2-Nal]-[THP]-ENN-NH₂
Ac-[D-Arg]-Cyclo-[Abu-QTWQC]-[Phe(4-2ae)]-[2-Nal]-[THP]-EDD-NH₂
Ac-[D-Arg]-Cyclo-[Abu-QTWEC]-[Phe(4-2ae)]-[2-Nal]-[THP]-END-NH₂
Ac-[D-Arg]-Cyclo-[Abu-ETWQC]-[Phe(4-2ae)]-[2-Nal]-[Tetrahydropyran-A]-END-NH₂
Ac-[D-Arg]-Cyclo-[Abu-QTWEC]-[Phe(4-2ae)]-[2-Nal]-[THP]-EDN-NH₂
Ac-[D-Arg]-Cyclo-[Abu-ETWQC]-[Phe(4-2ae)]-[2-Nal]-[Tetrahydropyran-A]-EDN-NH₂
Ac-[D-Arg]-Cyclo-[Abu-ETWEC]-[Phe(4-2ae)]-[2-Nal]-[THP]-ENN-NH₂
Ac-[D-Arg]-Cyclo-[Abu-QTWQC]-[Phe(4-2ae)]-[2-Nal]-[THP]-ENN-NH₂
Ac-[D-Arg]-Cyclo-[Abu-QTWQC]-[Phe(4-2ae)]-[2-Nal]-[THP]-END-NH₂
Ac-[D-Arg]-Cyclo-[Abu-QTWQC]-[Phe(4-2ae)]-[2-Nal]-[Tetrahydropyran-A]-EDN-NH₂
Ac-[D-Arg]-Cyclo-[Abu-ETWEC]-[Phe(4-2ae)]-[2-Nal]-[THP]-ENN-NH₂
Ac-[D-Arg]-Cyclo-[Abu-ETWQC]-[Phe(4-2ae)]-[2-Nal]-[Tetrahydropan-A]-ENN-OH

In certain embodiments, the one or more peptide inhibitor comprises or consists of any of the following sequences or structures:

[Palm]-[isoGlu]-[PEG4]-[Pen]-NTWQ-[Pen]-[Phe[4-(2-aminoethoxy)]-[2-Nal]-[Aib]-[Lys(Ac)]-NNNH₂;
Ac-[Pen]-NTWQ-[Pen]-[Phe[4-(2-aminoethoxy)]-[2-Nal]-[Aib]-[Lys(PEG4-isoGlu-Palm)]-NN-NH₂;
Ac-[Pen]-QTWQ-[Pen]-Phe(4-CONH₂)-[2-Nal]-[o-MeLys(Ac)]-[Lys(Ac)]-NN-NH₂;
[Octanyl]-[IsoGlu]-[PEG4]-[Pen]-NTWQ-[Pen]-[Phe[4-(2-aminoethoxy)]-[2-Nal]-[Aib]-[Lys(Ac)]-NN-NH₂;
[Octanyl]-[PEG4]-[Pen]-NTWQ-[Pen]-[Phe[4-(2-aminoethoxy)]-[2-Nal]-[Aib]-[Lys(Ac)]-NN-NH₂;
[Palm]-[PEG4]-[Pen]-NTWQ-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-[Lys(PEG4-Octanyl)]-NN-NH₂;
Ac-[Pen]-NTWQ-[Pen]-[Phe[4-(2-aminoethoxy)]-[2-Nal]-[Aib]-[Lys(PEG4-Palm)]-NN-NH₂;
Ac-[Pen]-NTWQ-[Pen]-[Phe[4-(2-aminoethoxy)-(PEG4-Palm)]-[2-Nal]-[Aib]-[Lys(Ac)]NN-NH₂;
Ac-[Pen]-NTWQ-[Pen]-[Phe[4-(2-aminoethoxy)-(PEG4-Lauryl)]-[2-Nal]-[Aib]-[Lys(Ac)]-NN-NH₂;
Ac-[Pen]-QTWQ-[Pen]-Phe(4-CONH₂)-[2-Nal]-[α-MeLys(PEG4-Palm)-[Lys(Ac)]-NN-NH₂;
Ac-[Pen]-QTWQ-[Pen]-Phe(4-CONH₂)-[2-Nal]-[α-MeLys(PEG4-Lauryl)]-[Lys(Ac)]-NN-NH₂;
Ac-[Pen]-NTWQ-[Pen]-[Phe[4-(2-aminoethoxy)-(PEG4-IsoGlu-Palm)]-[2-Nal]-[Aib]-[Lys(Ac)]-NN-NH₂;
Ac-[Pen]-NTWQ-[Pen]-[Phe[4-(2-aminoethoxy)-(PEG4-IsoGLu-Lauryl)]-[2-Nal]-[Aib]-[Lys(Ac)]-NN-NH₂;
Ac-[Pen]-QTWQ-[Pen]-Phe(4-CONH₂)-[2-Nal]-[α-MeLys(PEG4-IsoGlu-Palm)]-[Lys(Ac)]-NN-NH₂;
Ac-[Pen]-QTWQ-[Pen]-Phe(4-CONH₂)-[2-Nal]-[α-MeLys(PEG4-IsoGlu-Lauryl)]-[Lys(Ac)]-NN-NH₂;
Ac-[Pen]-QTWQ-[Pen]-Phe(4-CONH₂)-[2-Nal]-[α-MeLys(IVA)]-[Lys(Ac)]-NN-NH₂;
Ac-[Pen]-QTWQ-[Pen]-Phe(4-CONH₂)-[2-Nal]-[α-MeLys(Biotin)]-[Lys(Ac)]-NN-NH₂;
Ac-[Pen]-QTWQ-[Pen]-Phe(4-CONH₂)-[2-Nal]-[α-MeLys(Octanyl)]-[Lys(Ac)]-NN-NH₂;
Ac-[Pen]-[Lys(IVA)]-TWQ-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-[Lys(Ac)]-[Lys(IVA)]-N-NH₂;
Ac-[Pen]-[Lys(Biotin)]-TWQ-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-[Lys(Ac)]-[Lys(Biotin)]-N-NH₂;
Ac-[Pen]-[Lys(Octanyl)]-TWQ-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-[Lys(Ac)]-[Lys(octanyl)]-N-NH₂;
Ac-[Pen]-[Lys(Palm)]-TWQ-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-[Lys(Ac)]-Lys (Palm)]-N-NH₂;
Ac-[Pen]-[Lys(PEG8)]-TWQ-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-[Lys(Ac)]-[Lys(PEG8)]-N-NH₂;
Ac-[Pen]-K(Peg11-Palm)TWQ-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-[Lys(Ac)]-[Lys(Peg11-palm)]-N-NH₂;
Ac-[Pen]-[Cit]-TW-[Cit]-[Pen]-[Phe[4-(2-aminoethoxy)]-[2-Nal]-[Aib]-[Lys(Ac)]-NN-NH₂;
Ac-[Pen]-[Lys(Ac)]-TW-[Cit]-[Pen]-[Phe[4-(2-aminoethoxy)]-[2-Nal]-[Aib]-[Lys(Ac)]-NN-NH₂;
Ac-[Pen]-NT-[Phe(3,4-OCH3)2]-Q-[Pen]-[Phe[4-(2-aminoethoxy)]-[2-Nal]-[Aib]-[Lys(Ac)]-NN-NH₂;
Ac-[Pen]-NT-[Phe(2,4-CH3)2]-Q-[Pen]-[Phe[4-(2-aminoethoxy)]-[2-Nal]-[Aib]-[Lys(Ac)]-NN-NH₂;
Ac-[Pen]-NT-[Phe(3-CH3)]-Q-[Pen]-[Phe[4-(2-aminoethoxy)]-[2-Nal]-[Aib]-[Lys(Ac)]-NN-NH₂;
Ac-[Pen]-NT-[Phe(4-CH3)]-Q-[Pen]-[Phe[4-(2-aminoethoxy)]-[2-Nal]-[Aib]-[Lys(Ac)]-NN-NH₂;
Ac[(D)Arg]-[Pen]-NTWQ-[Pen]-[Phe[4-(2-aminoethoxy)]-[2-Nal]-[Aib]-[Lys(Ac)]-N-[βAla]-NH₂;
Ac-[(D)Tyr]-[Pen]-NTWQ-[Pen]-[Phe[4-(2-aminoethoxy)]-[2-Nal]-[Aib]-[Lys(Ac)]-N-[βAla]-NH₂;
Ac-[Pen]-NTWQ-[Pen]-[Phe[4-(2-aminoethoxy)]-[2-Nal]-[Aib]-[Lys(Ac)]-QN-NH₂;
Ac-[Pen]-NTWQ-[Pen]-[Phe[4-(2-aminoethoxy)]-[2-Nal]-[Aib]-[Lys(Ac)]-[Lys(Ac)]-N-NH₂;
Ac-[Pen]-NTWQ-[Pen]-[Phe[4-(2-aminoethoxy)]-[2-Nal]-[Aib]-[Lys(Ac)]-N-[Lys(Ac)]-NH₂;
Ac-[Pen]-NTWQ-[Pen]-[Phe[4-(2-aminoethoxy)]-[2-Nal]-[Aib]-[Lys(Ac)]-QQ-NH₂;
Ac-[Pen]-NTWQ-[Pen]-[Phe[4-(2-aminoethoxy)]-[2-Nal]-[Aib]-[Lys(Ac)]-Q-[βAla]-NH₂;
Ac-[Pen]-NTWQ-[Pen]-[Phe[4-(2-aminoethoxy)]-[2-Nal]-[Aib]-[Lys(Ac)]-N-[Cit]-NH₂;
Ac-[Pen]-NTWQ-[Pen]-[Phe[4-(2-aminoethoxy)]-[2-Nal]-[Aib]-[Lys(Ac)]-[Cit]-NH₂;
Ac-[Pen]-NTWQ-[Pen]-[Phe[4-(2-aminoethoxy)]-[2-Nal]-[Aib]-[Lys(Ac)]-[Cit]-Q-NH₂;
Ac-[Pen]-NTWQ-[Pen]-[Phe[4-(2-aminoethoxy)]-[2-Nal]-[Aib]-[Lys(Ac)]-[Cit]-[Lys(Ac)]-NH₂;
Ac-[Pen]-NTWQ-[Pen]-[Phe[4-(2-aminoethoxy)]-[2-Nal]-[Aib]-[Lys(Ac)]-[Lys(Ac)]-[Cit]-NH₂;
Ac-[Pen]-NTWQ-[Pen]-[Phe[4-(2-aminoethoxy)]-[2-Nal]-[Aib]-QN-[βAla]-NH₂;
Ac-[Pen]-NTWQ-[Pen]-[Phe[4-(2-aminoethoxy)]-[2-Nal]-[Aib]-E-[Cit]-Q-NH₂;
Ac-[Pen]-NTWQ-[Pen]-[Phe[4-(2-aminoethoxy)]-[2-Nal]-[Aib]-[Cit]-N-[Cit]-NH₂;
Ac-[Pen]-NTWQ-[Pen]-[Phe[4-(2-aminoethoxy)]-[2-Nal]-[Aib]-[Cit]-Q-[Cit]-NH₂;
Ac-[Pen]-[Cit]-TWQ-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-QNN-NH₂;
Ac-[Pen]-NTWQ-[Pen]-[Phe[4-(2-aminoethoxy)]-[2-Nal]-[Aib]-ENQ-NH₂;
Ac-[Pen]-GPWQ-[Pen]-[Phe[4-(2-aminoethoxy)]-[2-Nal]-[Aib]-[Lys(Ac)]-NN-NH₂;
Ac-[Pen]-PGWQ-[Pen]-[Phe[4-(2-aminoethoxy)]-[2-Nal]-[Aib]-[Lys(Ac)]-NN-NH₂;
Ac-[Pen]-NTWN-[Pen]-[Phe[4-(2-aminoethoxy)]-[2-Nal]-[Aib]-[Lys(Ac)]-NN-NH₂;
Ac-[Pen]-NSWQ-[Pen]-[Phe[4-(2-aminoethoxy)]-[2-Nal]-[Aib]-[Lys(Ac)]-NN-NH₂;
Ac-[Pen]-N-[Aib]-WQ-[Pen]-[Phe[4-(2-aminoethoxy)]-[2-Nal]-[Aib]-[Lys(Ac)]-NN-NH₂;
Ac-[Pen]-NTW-[Aib]-[Pen]-[Phe[4-(2-aminoethoxy)]-[2-Nal]-[Aib]-[Lys(Ac)]N-[Aib]-NH₂;
Ac-[Pen]-QTW-[Lys(Ac)]-[Pen]-[Phe[4-(2-aminoethoxy)]-[2-Nal]-[Aib]-[Lys(Ac)]-NN-NH₂;
Ac-[Pen]-[Lys(Ac)]-TWQ-[Pen]-[Phe[4-(2-aminoethoxy)]-[2-Nal]-[Aib]-[Lys(Ac)]NNNH₂;
Ac-[Pen]-QVWQ-[Pen]-[Phe[4-(2-aminoethoxy)]-[2-Nal]-[Aib]-[Lys(Ac)]-NN-NH₂;
Ac-[Pen]-NT-[2-Nal]-Q-[Pen]-[Phe[4-(2-aminoethoxy)]-[2-Nal]-[Aib]-[Lys(Ac)]-NN-NH₂;
Ac-[Pen]-NT-[1-Nal]-Q-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[α-MeLeu]-[Lys(Ac)]-NN-NH₂;
Ac-[Pen]-NTWQ-[Pen]-[Phe[4-(2-aminoethoxy)]-[2-Nal]-[α-MeLys]-[Lys(Ac)]-NN-NH₂;
Ac-[Pen]-NTWQ-[Pen]-[Phe[4-(2-aminoethoxy)]-[2-Nal]-[4-amino-4-carboxy-tetrahydropyran]-[Lys(Ac)]-NN-NH₂;
Ac-[Pen]-NTWQ-[Pen]-[Phe[4-(2-aminoethoxy)]-[2-Nal]-[α-MeLeu]-[Lys(Ac)]-N-[Ala]-NH₂;
Ac-[Pen]-NTWQ-[Pen]-[Phe[4-(2-aminoethoxy)]-[2-Nal]-[α-MeLys]-[Lys(Ac)]-N-[Ala]-NH₂;
Ac-[Pen]-NTWQ-[Pen]-[Phe[4-(2-aminoethoxy)]-[2-Nal]-[4-amino-4-carboxy-tetrahydropyran]-[Lys(Ac)]-N-[βAla]-NH₂;
Ac-[Pen]-NTWQ-[Pen]-[Phe[4-(2-aminoethoxy)]-[2-Nal]-[Aib]-[Lys(Ac)]-LN-NH₂;
Ac-[Pen]-NTWQ-[Pen]-[Phe[4-(2-aminoethoxy)]-[2-Nal]-[Aib]-[Lys(Ac)]-GN-NH₂;
Ac-[Pen]-NTWQ-[Pen]-[Phe[4-(2-aminoethoxy)]-[2-Nal]-[Aib]-[Lys(Ac)]-SN-NH₂;
Ac-[Pen]-NTWQ-[Pen]-[Phe[4-(2-aminoethoxy)]-[2-Nal]-[Aib]-[Lys(Ac)]-[Aib]-N-NH₂;
Ac-[Pen]-NTWQ-[Pen]-[Phe[4-(2-aminoethoxy)]-[2-Nal]-[Aib]-[Lys(Ac)]-FN-NH₂;
Ac-[Pen]-NTW-[Cit]-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-[Lys(Ac)]-[Tic]-[βAla]-NH₂;
Ac-[Pen]-NTWQ-[Pen]-[Phe[4-(2-aminoethoxy)]-[2-Nal]-[Aib]-[Lys(Ac)]-[nLeu]-[βAla]-NH₂;
Ac-[Pen]-NTWQ-[Pen]-[Phe[4-(2-aminoethoxy)]-[2-Nal]-[Aib]-[Lys(Ac)]-G-[Ala]-NH₂;
Ac-[Pen]-NTWQ-[Pen]-[Phe[4-(2-aminoethoxy)]-[2-Nal]-[Aib]-[Lys(Ac)]-R-[Ala]-NH₂;
Ac-[Pen]-NTWQ-[Pen]-[Phe[4-(2-aminoethoxy)]-[2-Nal]-[Aib]-[Lys(Ac)]-W-[Ala]-NH₂;
Ac-[Pen]-NTWQ-[Pen]-[Phe[4-(2-aminoethoxy)]-[2-Nal]-[Aib]-[Lys(Ac)]-S-[Ala]-NH₂;
Ac-[Pen]-NTWQ-[Pen]-[Phe[4-(2-aminoethoxy)]-[2-Nal]-[Aib]-[Lys(Ac)]-L-[Ala]-NH₂;
Ac-[Pen]-NTWQ-[Pen]-[Phe[4-(2-aminoethoxy)]-[2-Nal]-[Aib]-[Lys(Ac)]-[AIB]-[βAla]-NH₂;
Ac-[Pen]-NTWQ-[Pen]-[Phe[4-(2-aminoethoxy)]-[2-Nal]-[Aib]-[Lys(Ac)]-[N-MeAla]-[βAla]-NH₂;
Ac-[Pen]-NTWQ-[Pen]-[Phe[4-(2-aminoethoxy)]-[2-Nal]-[Aib]-[Lys(Ac)]-[2-Nap]-[βAla]-NH₂;
Ac-[Pen]-NTWQ-[Pen]-[Phe[4-(2-aminoethoxy)]-[2-Nal]-[Aib]-[Lys(Ac)]-F-[βAla]-NH₂;
Ac-[(D)Arg]-[Pen]-NTWQ-[Pen]-[Phe[4-(2-aminoethoxy)]-[2-Nal]-[4-amino-4-carboxy-tetrahydropyran]-[Lys(Ac)]NNNH₂;
Biotin-[PEG4]-cyclo[[Abu]-QTWQC]-[Phe[4-(2-aminoethoxy)]-[2-Nal]-[4-amino-4-carboxy-tetrahydropyran]-ENN-NH₂;
Ac-cyclo[[Abu]-QTWQC]-[Phe[4-(2-aminoethoxy)]-[2-Nal]-[4-amino-4-carboxy-tetrahydropyran]-[Lys(Ac)]-NN-NH₂;
Ac-[(D)Arg]-cycl[[[Abu]-QTWQC]-[Phe[4-(2-aminoethoxy)]-[2-Nal]-[4-amino-4-carboxy-tetrahydropyran]-[Lys(Ac)]-NN-NH₂;
Ac-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-E-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-[(D)Asp]-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-R-[(D)Arg]-cyclo[[Abu]-QTWQC]-[Phe[4-(2-aminoethoxy)]-[2-Nal]-[4-amino-4-carboxy-tetrahydropyran]-ENN-NH₂;
inoethoxy)]-[2-Nal]-[4-amino-4-carboxy-tetrahydropyran]-ENN-NH₂;
Ac-F-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-[(D)Phe]-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-[2-Nal]-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-T-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-L-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-[(D)Gln]-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-[(D)Asn]-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-cyclo[[Abu]-QTWQC]-[Phe[4-(2-aminoethoxy)-(PEG4-Alexa488)]-[2-Nal]-[4-amino-4-carboxy-tetrahydropyran]-ENN-NH₂;
[Alexa488]-[PEG4]-cyclo[[Abu]-QTWQC]-[Phe[4-(2-aminoethoxy)]-[2-Nal]-[4-amino-4-carboxy-tetrahydropyran]-ENN-NH₂;
[Alexa647]-[PEG4]-cyclo[[Abu]-QTWQC]-[Phe[4-(2-aminoethoxy)]-[2-Nal]-[4-amino-4-carboxy-tetrahydropyran]-ENN-NH₂;
[Alexa-647]-[PEG4]-[(D)Arg]-cyclo[[Abu]-QTWQC]-[Phe[4-(2-aminoethoxy)]-[2-Nal]-[4-amino-4-carboxy-tetrahydropyran]-[Lys(Ac)]-NN-NH₂;
[Alexa647]-[PEG12]-[(D)Arg]-cyclo[[Abu]-QTWQC]-[Phe[4-(2-aminoethoxy)]-[2-Nal]-[4-amino-4-carboxy-tetrahydropyran]-[Lys(Ac)]-NN-NH₂; and
[Alexa488]-[PEG4]-[(D)Arg]-cyclo[[Abu]-QTWQC]-[Phe[4-(2-aminoethoxy)]-[2-Nal]-[4-amino-4-carboxy-tetrahydropyran]-[Lys(Ac)]-NN-NH₂. In particular embodiments, the peptide inhibitor is cyclized via a disulfide bond between two Pen residues or by a thioether bond between Abu and a Cys residue, and wherein the peptide inhibitor inhibits the binding of an interleukin-23 (IL-23) to an IL-23 receptor.

In certain embodiments, the peptide inhibitor comprises a sequence shown in Table 6, wherein Peptides A-R comprise a thioether bond between the amino acids or moieties at positions 2 and 7, and wherein Peptides S-CC comprise a disulfide bond between the amino acid residues or moieties at positions 2 and 7. Dimers comprising two peptides having the sequence show are indicated by brackets followed by a “2”, e.g., Peptides L, Z and AA.

TABLE 6
Illustrative Peptide Inhibitors

| | | | | | | | | | | | | | | | |
|---|---|---|---|---|---|---|---|---|---|---|---|---|---|---|---|
| Peptide | 0 | 1 | 2 | 3 | 4 | 5 | 6 | 7 | 8 | 9 | 10 | 11 | 12 | 13 | 14 |

Thioethers

| | | | | | | | | | | | | | | | |
|---|---|---|---|---|---|---|---|---|---|---|---|---|---|---|---|
| A | Ac- | dR | Abu | Q | T | W | Q | C | F(4- | 2- | THP | E | N | N | —NH2 |
| | | | | | | | | | 2ae) | Nal | | | | | |
| B | | Ac- | Abu | Q | T | W | Q | C | F(4- | 2- | THP | E | N | N | —NH2 |
| | | | | | | | | | 2ae) | Nal | | | | | |
| C | | Ac- | Abu | Q | T | W | Q | C | F(4- | 2- | a- | E | N | N | —NH2 |
| | | | | | | | | | 2ae) | Nal | Me-K | | | | |
| D | | Ac- | Abu | Q | T | W | Q | C | F(4- | 2- | a- | E | N | Q | —NH2 |
| | | | | | | | | | 2ae) | Nal | Me-K | | | | |
| E | Ac- | dF | Abu | Q | T | W | Q | C | F(4- | 2- | a- | E | N | N | —NH2 |
| | | | | | | | | | 2ae) | Nal | Me-K | | | | |
| F | | Ac- | Abu | Q | T | W | Q | C | F(4- | 2- | a- | K(Ac) | N | G | —NH2 |
| | | | | | | | | | 2ae) | Nal | Me-V | | | | |
| G | | Ac- | Abu | Q | T | W | Q | C | F(4- | 2- | a- | E | N | G | —NH2 |
| | | | | | | | | | 2ae) | Nal | Me-K | | | | |
| H | Ac- | T | Abu | Q | T | W | Q | C | F(4- | 2- | a- | E | N | N | —NH2 |
| | | | | | | | | | 2ae) | Nal | Me-K | | | | |
| I | Ac- | dR | Abu | Q | T | W | Q | C | F(4- | 2- | THP | K(Ac) | N | N | —NH2 |
| | | | | | | | | | 2ae) | Nal | | | | | |
| J | | Ac- | Abu | Q | T | W | Q | C | F(4- | 2- | a- | Cit | N | N | —NH2 |
| | | | | | | | | | 2ae) | Nal | Me-L | | | | |
| K | | Ac- | Abu | Q | T | W | Q | C | F(4- | 2- | Achc | E | N | N | —NH2 |
| | | | | | | | | | 2ae) | Nal | | | | | |
| L | DIG | [Ac- | Abu | Q | T | W | Q | C | Y(Me) | 2- | a- | E | N | G | —NH2]2 |
| | | | | | | | | | | Nal | Me-K | | | | |
| M | Ac- | dF | Abu | Q | T | W | Q | C | F(4- | 2- | THP | E | N | N | —NH2 |
| | | | | | | | | | 2ae) | Nal | | | | | |
| N | | Biotin- | Abu | Q | T | W | Q | C | F(4- | 2- | THP | E | N | N | —NH2 |
| | | PEG4 | | | | | | | 2ae) | Nal | | | | | |
| O | | Ac- | Abu | Q | T | W | Q | C | F(4- | 2- | Aib | K(Ac) | N | A | —NH2 |
| | | | | | | | | | 2ae) | Nal | | | | | |
| P | | Ac- | Abu | Q | T | W | Q | C | F(4- | 2- | a- | K(IVA) | N | G | —NH2 |
| | | | | | | | | | 2ae) | Nal | Me-K | | | | |
| Q | | Ac- | Abu | Q | T | W | Q | C | F(4- | 2- | a- | Q | N | bA | —NH2 |
| | | | | | | | | | 2ae) | Nal | Me-L | | | | |
| R | | Ac- | Abu | Q | T | W | Q | C | F(4- | 2- | Aib | Q | N | G | —NH2 |
| | | | | | | | | | 2ae) | Nal | | | | | |

DiPen sequences

| | | | | | | | | | | | | | | |
|---|---|---|---|---|---|---|---|---|---|---|---|---|---|---|
| S | Ac- | Pen | N | T | W | Q | Pen | F(4- | 2- | THP | K(Ac) | N | N | —NH2 |
| | | | | | | | | 2ae) | Nal | | | | | |
| T | Ac- | Pen | Q | T | W | Q | Pen | F(4- | 2- | a- | Q | N | N | —NH2 |
| | | | | | | | | 2ae) | Nal | Me-L | | | | |
| U | Ac- | Pen | N | T | W | Q | Pen | F(4- | 2- | a- | K(Ac) | N | N | —NH2 |
| | | | | | | | | 2ae) | Nal | Me-L | | | | |
| V | Ac- | Pen | N | T | W | Q | Pen | F(4- | 2- | Aib | K(Ac) | N | N | —NH2 |
| | | | | | | | | 2ae) | Nal | | | | | |
| W | Ac- | Pen | N | T | W | Cit | Pen | F(4- | 2- | Aib | K(Ac) | N | N | —NH2 |
| | | | | | | | | 2ae) | Nal | | | | | |
| X | Ac- | Pen | Q | T | W | Q | Pen | F(4- | 2- | a- | K(Ac) | N | N | —NH2 |
| | | | | | | | | 2ae) | Nal | Me-L | | | | |
| Y | Ac- | Pen | N | T | W | Q | Pen | F(4- | 2- | Aib | K(Ac) | N | N | —NH2 |
| | | | | | | | | 2ae) | Nal | | | | | |
| Z | [Ac- | Pen | Q | T | W | Q | Pen | F(4- | 2- | a- | K(Ac) | N | N | —NH2]2 |
| | | | | | | | | Cmd) | Nal | Me-K | | | | |
| AA | [Ac- | Pen | Q | T | W | Q | Pen | Y(Me) | 2- | a- | E | N | G | —NH2]2 |
| | | | | | | | | | Nal | Me-K | | | | |
| BB | Ac- | Pen | N | T | W | Q | Pen | F(4- | 2- | Achc | K(Ac) | N | N | —NH2 |
| | | | | | | | | Cmd) | Nal | | | | | |
| CC | Ac- | Pen | N | T | W | Q | Pen | F(4- | 2- | THP | K(Ac) | N | bA | —NH2 |
| | | | | | | | | 2ae) | Nal | | | | | |

The present invention also includes any of the peptide inhibitors described herein in either a free or a salt form. Thus, embodiments of any of the peptide inhibitors described herein (and related methods of use thereof) include a pharmaceutically acceptable salt of the peptide inhibitor

The present invention also includes variants of any of the peptide inhibitors described herein, wherein one or more L-amino acid residue is substituted with the D isomeric form of the amino acid residue, e.g., an L-Ala is substituted with a D-Ala.

In particular embodiments of the peptide inhibitors described herein, they comprise one or more unnatural or non-natural amino acid residues.

The present invention also include dimers of any of the peptides described herein, optionally comprising a linker connecting the two dimer subunit peptides

The present invention also includes any of the peptide monomer inhibitors described herein linked to a linker moiety, including any of the specific linker moieties described herein. In particular embodiments, a linker is attached to an N-terminal or C-terminal amino acid, while in other embodiments, a linker is attached to an internal amino acid. In particular embodiments, a linker is attached to two internal amino acids, e.g., an internal amino acid in each of two monomer subunits that form a dimer. In some embodiments of the present invention, a peptide inhibitor is attached to one or more linker moieties shown

The present invention also includes peptides and peptide dimers comprising a peptide having at least 90%, at least 95%, at least 98%, or at least 99% sequence identity to the peptide sequence of a peptide inhibitor described herein. In particular embodiments, peptide inhibitors of the present invention comprise a core peptide sequence and one or more N-terminal and/or C-terminal modification (e.g., Ac and NH₂) and/or one or more conjugated linker moiety and/or half-life extension moiety. As used herein, the core peptide sequence is the amino acid sequence of the peptide absent such modifications and conjugates. For example, for the peptide inhibitor: [Palm]-[isoGlu]-[PEG4]-[Pen]-NTWQ-[Pen]-[Phe[4-(2-aminoethoxy)]-[2-Nal]-[Aib]-[Lys(Ac)]-NN-NH₂, the core peptide sequence is: [Pen]-NTWQ-[Pen]-[Phe[4-(2-aminoethoxy)]-[2-Nal]-[Aib]-[Lys(Ac)]-NN.

Optional Characteristics of Peptide Inhibitors

Any of the peptide inhibitors of the present invention may be further defined, e.g., as described below. It is understood that each of the further defining features described herein may be applied to any peptide inhibitors where the amino acids designated at particular positions allow the presence of the further defining feature.

In certain embodiments of any of the peptide inhibitors described herein, the peptide inhibitor is cyclized.

In certain embodiments of any of the peptide inhibitors described herein, the peptide inhibitor or monomer subunit thereof is linear or not cyclized. In certain embodiments where the peptide is linear or not cyclized, X4 and X9 can be any amino acid.

In certain embodiments, the peptide inhibitor is cyclized, e.g., through X4 and X9.

In various embodiments, R¹ is a bond, hydrogen, a C1-C6 alkyl, a C6-C12 aryl, a C6-C12 aryl C1-C6 alkyl, or a C1-C20 alkanoyl, and including PEGylated versions alone or as spacers of any of the foregoing, e.g., acetyl. It is understood that the R¹ may replace or be present in addition to the typical amine group located at the amino terminus of a peptide. It is further understood that R¹ may be absent. In certain embodiments, the peptide inhibitor comprises an N-terminus selected from hydrogen, a C1-C6 alkyl, a C6-C12 aryl, a C6-C12 aryl C1-C6 alkyl, or a C1-C20 alkanoyl, and including PEGylated versions alone or as spacers of any of the foregoing, e.g., acetyl. In particular embodiments of any of the peptide inhibitors described herein, R¹ or the N-terminal moiety is hydrogen. In certain embodiments, R¹ is a bond, e.g., a covalent bond.

In certain embodiments of any of the peptide inhibitors having any of the various Formulas set forth herein, R¹ or the N-terminal moiety is selected from methyl, acetyl, formyl, benzoyl, trifluoroacetyl, isovaleryl, isobutyryl, octanyl, and the conjugated amides of lauric acid, hexadecanoic acid, and γ-Glu-hexadecanoic acid. In one embodiment, R¹ or the N-terminal moiety is pGlu. In certain embodiments, R¹ is hydrogen. In particular embodiments, R¹ is acetyl, whereby the peptide inhibitor is acylated at its N-terminus, e.g., to cap or protect an N-terminal amino acid residue, e.g., an N-terminal Pen or Abu residue.

In certain embodiments of any of the peptide inhibitors described herein, R¹ or the N-terminal moiety is an acid. In certain embodiments, R¹ or the N-terminal moiety is an acid selected from acetic acid, formic acid, benzoic acid, trifluoroacetic acid, isovaleric acid, isobutyric acid, octanoic acid, lauric acid, hexadecanoic acid, 4-Biphenylacetic acid, 4-fluorophenylacetic acid, gallic acid, pyroglutamic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl) benzoic acid, cinnamic acid, mandelic acid, 4-methylbicyclo(2.2.2)-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, an alkylsulfonic acid and an arylsulfonic acid.

In particular embodiments, R¹ or the N-terminal moiety is an alkylsulfonic acid selected from methanesulfonic acid, ethanesulfonic acid, 1,2-ethane-disulfonic acid, and 2-hydroxyethanesulfonic acid.

In particular embodiments, R¹ or the N-terminal moiety is an arylsulfonic acid selected from benzenesulfonic acid, 4-chlorobenzenesulfonic acid, 2-naphthalenesulfonic acid, 4-toluenesulfonic acid, and camphorsulfonic acid.

In some embodiments, wherein a peptide of the present invention comprises a conjugation to an acidic compound such as, e.g., isovaleric acid, isobutyric acid, valeric acid, and the like, the presence of such a conjugation is referenced in the acid form. So, for example, but not to be limited in any way, instead of indicating a conjugation of isovaleric acid to a peptide by referencing isovaleroyl (e.g., isovaleroyl-[Pen]-QTWQ[Pen]-[Phe(4-OMe)]-[2-Nal]-[α-MeLys]-[Lys(Ac)]-NG-NH₂, in some embodiments, the present application references such a conjugation as isovaleric acid-[Pen]-QTWQ[Pen]-[Phe(4-OMe)]-[2-Nal]-[α-MeLys]-[Lys(Ac)]-NG-NH₂. Reference to the conjugation in its acid form is intended to encompass the form present in the peptide inhibitor.

In certain embodiments, the peptide inhibitor comprises a C-terminus (e.g., R² or the C-terminal moiety) selected from a bond, OH or NH₂. In certain embodiments, R² is a bond. In various embodiments of any of the peptide inhibitors having any of the various Formulas set forth herein, R² or the C-terminal moiety is OH or NH₂. It is understood that the R² or the C-terminal moiety may replace or be present in addition to the carboxyl group typically located at the carboxy terminus of a peptide. It is further understood that R² may be absent.

In particular embodiments of any of the peptide inhibitors having any of the various Formulae set forth herein, X comprises or consists of 7 to 35 amino acid residues, 8 to 35 amino acid residues, 9 to 35 amino acid residues, 10 to 35 amino acid residues, 7 to 25 amino acid residues, 8 to 25 amino acid residues, 9 to 25 amino acid residues, 10 to 25 amino acid residues, 7 to 20 amino acid residues, 8 to 20 amino acid residues, 9 to 20 amino acid residues, 7 to 18 amino acid residues, 8 to 18 amino acid residues, 9 to 18 amino acid residues, or 10 to 18 amino acid residues.

In certain embodiments of any of the Formulae set forth herein, X either or both does not comprise or does not consist of an amino acid sequence set forth in US Patent Application Publication No. US2013/0029907. In certain embodiments of any of the Formulae set forth herein, X either or both does not comprise or does not consist of an amino acid sequence set forth in US Patent Application Publication No. US2013/0172272.

In certain embodiments of any of the peptide inhibitors described herein, the peptide inhibitor, or each monomer subunit thereof, comprises or consists of at least 3, at least 4 at least 5, at least 6, or at least 7 amino acid residues carboxy terminal of the X9 amino acid residue. In particular embodiments of any of the peptide inhibitors described herein, the peptide inhibitor comprises 3 to 11, 3 to 10, 3 to 9, 3 to 8, 3 to 7, 3 to 6, 3 to 5, 3 to 4, 3, 4, 5, 6, 7, 8, 9, 10, or 11 amino acid residues carboxy terminal of the X9 amino acid residue.

In certain embodiments of any of the peptide inhibitors described herein, the peptide inhibitor, or each monomer subunit thereof, comprises or consists of 4 amino acid residues between X4 and X9. In one embodiment, both X4 and X9 are cysteines.

In certain embodiments, a peptide inhibitor of any of the Formulae described herein comprises the amino acid residues or moieties indicated as X4-X15. In particular embodiments, the peptide inhibitor does not include X1-X3 or X16-X20. In certain embodiments, the peptide inhibitors include an N-terminal extension of one to three amino acid residues corresponding to any of X1-X3. In particular embodiments, any one or more of X1, X2 and X3, when present, are a D-amino acid. In certain embodiments, the peptide inhibitors include an C-terminal extension of one to five amino acid residues corresponding to any of X16-X20. In particular embodiments, any one or more of X16, X17, X18, X19 and X20, when present, are a D-amino acid. Illustrative amino acid residues that may be present in the N-terminal and/or C-terminal extensions are shown in Tables 3, 5A and 5B. These tables each show a first peptide inhibitor, with derivates thereof comprising N-terminal extensions, C-terminal extensions, and/or conjugated moieties. The present invention includes derivatives of any of the peptide inhibitors described herein comprising one or more such N-terminal extension, C-terminal extension, and/or conjugated moiety. In certain embodiments, any of the amino acid residues shown in the extended positions in Tables 3, 5A and 5B may be present in any combination in a peptide inhibitor of the present invention. In particular embodiments, the N-terminal and/or C-terminal extensions are associated with an increased half-life, e.g., upon administration to a subject.

In certain embodiments of any of the peptide inhibitors described herein, the peptide inhibitor, or each monomer subunit thereof, comprises the amino acid sequence motif, W-X-X-Y-W, e.g., at positions X7-X11. In certain embodiments, the peptide inhibitor, or each monomer subunit thereof, comprises the amino acid sequence motif, C-X-X-W-X-C-Y-W, e.g., at positions X4-X11. In certain embodiments, the peptide inhibitor, or each monomer subunit thereof, comprises the amino acid sequence motif, Pen-X-X-W-X-Pen-Y-W, e.g., at positions X4-X11. In certain embodiments of any of the peptide inhibitors described herein, the peptide inhibitor, or both monomer subunit thereof, does not comprise the amino acid sequence motif, W-X-X-Y-W, e.g., at positions X7-X11, where X is any amino acid.

In certain embodiments of any of the Formula or peptide inhibitors described herein, the peptide inhibitor comprises one or more amino acid residues N-terminal to X4. In particular embodiments, X3 is present. In certain embodiments, X3 is Glu, (D)Glu, Arg, (D)Arg, Phe, (D)Phe, 2-Nal, Thr, Leu, or (D)Gln. In certain embodiments, X3 is (D)Arg or (D)Phe.

In particular embodiments of any of the Formula or peptide inhibitors described herein, the peptide inhibitor comprises an amino acid at X2. In particular embodiments, X2 is Glu, (D)Asp, Arg, (D)Arg, Phe, (D)Phe, 2-Nal, Thr, Leu, (D)Gln, or (D)Asn. In certain embodiments, X2 and X3 are present. In particular embodiments, X2 is Glu, (D)Asp, Arg, (D)Arg, Phe, (D)Phe, 2-Nal, Thr, Leu, (D)Gln, or (D)As, and X3 is (D)Arg.

In certain embodiments, a peptide inhibitor of the present invention, or one or both monomer subunits thereof, comprises, optionally at its C-terminus, one of the following amino acid sequences:

ENG;

ENN;

[4-amino-4-carboxy-tetrahy dropyran]-ENN;

[Lys(Ac)]-NN;

[α-MeLys]-ENG;

[α-MeLys]-[Lys(Ac)]-NN;

[α-MeLeu]-[Lys(Ac)]-NN

[α-MeLeu]-ENG;

[α-MeOm]-[Lys(Ac)]-NG;

[α-MeLeu]-ENG;

Aib-[Lys(Ac)]-NG;

Aib-[Lys(Ac)]-NN;

NG-[AEA]-[(D)-Lys];

[Dapa]-NG-[AEA]-[(D)-Lys];

[Om]-NG-[AEA]-[(D)-Lys];

[α-MeLys]-ENN;

[4-amino-4-carboxy-tetrahydropyran]-[Lys(Ac)]-NN;

[Achc]-[Lys(Ac)]-NN; or

[Acpc]-[Lys(Ac)]-NN.

In particular embodiments, one of these amino acid sequences constitutes the terminal C-terminal amino acids of the peptide. In particular embodiment, these amino acid sequences correspond to X13-X15 or X12-X15 or X14-X16 or X13-X17.

In certain embodiments, a peptide inhibitor of the present invention, or one or both monomer subunits thereof, comprises, optionally at its C-terminus, one of the following amino acid sequences:

WQCY-[2-Nal]-[α-MeLys];

WQC-[Phe(4-OMe)]-[2-Nal]-[α-MeLys];

WQC-[Phe(4-OMe)]-[2-Nal]-[Aib];

WQ-[Pen]-[Phe(4-OMe)]-[2-Nal]-[α-MeLys];

W-Xaa8-C-Phe[4-(2-aminoethoxy)]-[2-Nal];

W-Xaa8-C-Phe[4-(2-aminoethoxy)]-[1-Nal];

W-Xaa8-C-Phe[4-(2-aminoethoxy)]; or

W-Xaa8-C-[Phe(4-OCH₃)]. In particular embodiments, one of these amino acid sequences constitutes the terminal C-terminal amino acids of the peptide. In particular embodiment, these amino acid sequences correspond to X7 to X12 or X7 to X11 or X7 to X10.

In certain embodiments of any of the peptide inhibitors described herein, including both peptide monomer inhibitors and monomer subunits of peptide dimer inhibitors, the peptide monomer inhibitor or monomer subunit is cyclized via a peptide bond between its N-terminal amino acid residue and its C-terminal amino acid residue. In particular embodiments, the peptide inhibitor (or monomer subunit thereof) comprises both an intramolecular bond between X4 and X9 and a peptide bond between its N-terminal amino acid residue and its C-terminal amino acid residue. In certain embodiments, the intramolecular bond is any of those described herein, e.g., a disulfide bond or a thioether bond.

In certain embodiments, the present invention includes a peptide inhibitor that comprises a core consensus sequence selected from one of the following (shown in N-terminal to C-terminal direction):

X1-X2-X3-Pen-X5-X6-W-X8-Pen-X10-X11-X12-X13-X14-X15;

Pen-X5-X6-W-Q-Pen;

Pen-X5-X6-W-X8-Pen;

Pen-X5-X6-W-X8-Pen-[Phe(4-CONH₂)]; and

Pen-X5-X6-W-X8-Pen-[Phe[4-(2-aminoethoxy)]],

wherein the Pen residues are joined by an intramolecular bond, e.g., disulphide bond. X1, X2, X3, X5, X6, X8, X10, X11, X12, X13, X14, and X15 may be any amino acid. In some embodiment X5 is Arg, Asn, Gln, Dap, Om; X6 is Thr or Ser; and X8 is Gln, Val, Phe, Glu, Lys. In particular embodiments, X1, X2, X3, X5, X6, X8, X10, X11, X12, X13, X14, and X15 are defined as described in any of the various Formulas and peptide inhibitors described herein.

In certain embodiments, the present invention includes a peptide inhibitor that comprises a core consensus sequence selected from one of the following (shown in N-terminal to C-terminal direction):

X1-X2-X3-Abu-X5-X6-W-X8-C-X9-X10-X11-X12-X13-X14-X15;

Abu-X5-X6-W-Q-C;

Abu-X5-X6-W-X8-C;

Abu-X5-X6-W-X8-C-[Phe(4-CONH₂)]; and

Abu-X5-X6-W-X8-C-[Phe[4-(2-aminoethoxy)]],

where Abu and C are linked through a intra moleculer thiother bond. X1, X2, X3, X5, X6, X8, X10, X11, X12, X13, X14, and X15 may be any amino acid. In some embodiments, X5 is Arg, Asn, Gln, Dap, Om; X6 is Thr or Ser; and X8 is Gln, Val, Phe, Glu, Lys. In particular embodiments, X1, X2, X3, X5, X6, X8, X10, X11, X12, X13, X14, and X15 are defined as described in any of the various Formulas and peptide inhibitors described herein.

In certain embodiments, any of the peptide inhibitors described herein may be further cyclized via a peptide bond between its N-terminal amino acid residue and its C-terminal amino acid residue. In particular embodiments, the peptide inhibitor comprises a peptide bond between X3 or X4 and any one of X9, X10, X11, X12, X13, X14, X15, X16, X17, X18, X19 or X20. In particular embodiments, peptide inhibitors of the present invention comprise a peptide bond between their N-terminal and C-terminal amino acid residues, and they also comprise an intramolecular bond between X4 and X9. In certain embodiments, the intramolecular bond is a disulfide bond, a thioether bond, a lactam bond or any of the other bonds described herein.

In certain embodiments, a peptide inhibitor monomer or dimer may comprise a linker moiety. The linker moieties may include any structure, length, and/or size that is compatible with the teachings herein. In at least one embodiment, a linker moiety is selected from the non-limiting group consisting of cysteine, lysine, DIG, PEG4, PEG4-biotin, PEG13, PEG25, PEG1K, PEG2K, PEG3.4K, PEG4K, PEG5K, IDA, ADA, Boc-IDA, Glutaric acid, Isophthalic acid, 1,3-phenylenediacetic acid, 1,4-phenylenediacetic acid, 1,2-phenylenediacetic acid, Triazine, Boc-Triazine, IDA-biotin, PEG4-Biotin, AADA, suitable aliphatics, aromatics, heteroaromatics, and polyethylene glycol based linkers having a molecular weight from approximately 400 Da to approximately 40,000 Da. Non-limiting examples of suitable linker moieties are provided in Table 7.

TABLE 7
Illustrative Linker Moieties

| Abbrivation | Discription | Structure |
|---|---|---|
| DIG | DIGlycolic acid, | 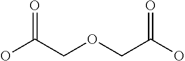 |
| PEG4 | Bifunctional PEG linker with 4 PolyEthylene Glycol units | 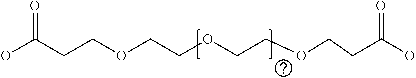 |
| PEG13 | Bifunctional PEG linker with 13 PolyEthylene Glycol units | 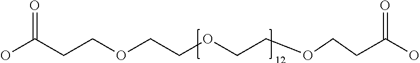 |
| PEG25 | Bifunctional PEG linker with 25 PolyEthylene Glycol units | 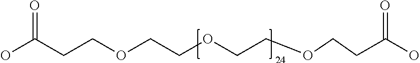 |
| PEG1K | Bifunctional PEG linker with PolyEthylene | |
| | Glycol Mol wt of 1000 Da | |
| PEG2K | Bifunctional PEG linker with PolyEthylene | |
| | Glycol Mol wt of 2000 Da | |
| PEG3.4K | Bifunctional PEG linker with PolyEthylene | |
| | Glycol Mol wt of 3400 Da | |
| PEG5K | Bifunctional PEG linker with PolyEthylene | |
| | Glycol Mol wt of 5000 Da | |
| DIG | DIGlycolic acid | 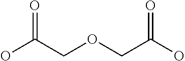 |
| β-Ala-IDA | β-Ala-Iminodiacetic acid | 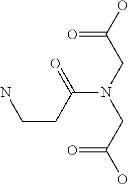 [SMILES: NCCC(=O)N(CC(=O)O)CC(=O)O] |
| Boc-β- Ala-IDA | Boc-β-Ala-Iminodiacetic acid | 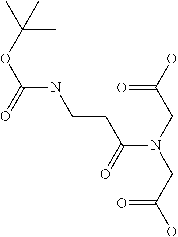 [SMILES: CC(C)(C)OC(=O)NCCC(=O)N(CC(=O)O)CC(=O)O] |
| Ac-β-Ala- IDA | Ac-β-Ala-Iminodiacetic acid | 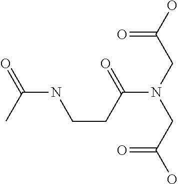 [SMILES: CC(=O)NCCC(=O)N(CC(=O)O)CC(=O)O] |
| IDA-β-Ala- Palm | Palmityl-β-Ala-Iminodiacetic acid | 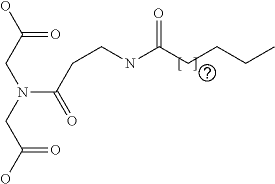 |
| GTA | Glutaric acid | 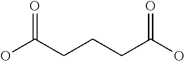 |
| PMA | Pemilic acid | 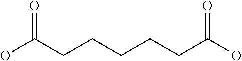 [SMILES: O=C(O)CCCCCC(=O)O] |
| AZA | Azelaic acid | 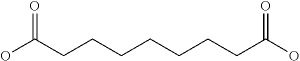 [SMILES: O=C(O)CCCCCCCC(=O)O] |
| DDA | Dodecanedioic acid | 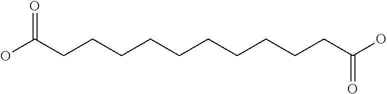 [SMILES: O=C(O)CCCCCCCCCCC(=O)O] |
| IPA | Isopthalic aicd | 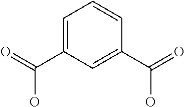 [SMILES: O=C(O)c1cccc(C(=O)O)c1] |
| 1,3-PDA | 1,3-Phenylenediacetic acid | 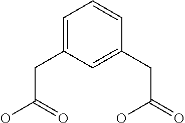 [SMILES: O=C(O)Cc1cccc(CC(=O)O)c1] |
| 1,4-PDA | 1,4-Phenylenediacetic acid | 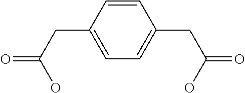 [SMILES: O=C(O)Cc1ccc(CC(=O)O)cc1] |
| 1,2-PDA | 1,2-Phenylenediacetic acid | 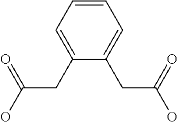 [SMILES: O=C(O)Cc1ccccc1CC(=O)O] |
| Triazine | Amino propyl Triazine di-acid | 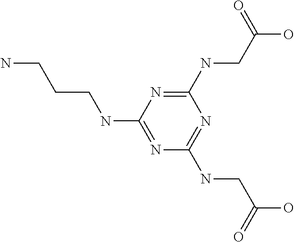 [SMILES: NCCCNc1nc(NCC(=O)O)nc(NCC(=O)O)n1] |
| Boc- Triazine | Boc-Triazine di-acid | 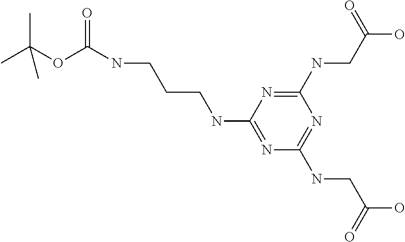 [SMILES: CC(C)(C)OC(=O)NCCCNc1nc(NCC(=O)O)nc(NCC(=O)O)n1] |
| ADA | Amino diacetic acid (which may also referred to as Iminodiacetic acid) | 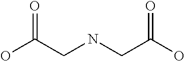 |
| AADA | n-Acetyl amino acetic acid (which may also referred to as N-acetyl Iminodiacetic acid) | 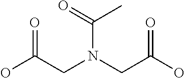 |
| PEG4- Biotin | PEG4-Biotin (Product number 10199, QuantaBioDesign) | 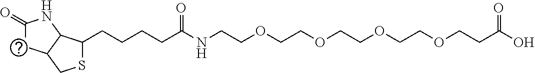 [SMILES: O=C(O)CCOCCOCCOCCOCCNC(=O)CCCCC1SCC2CC(=O)NC21] |
| IDA-Biotin | N-Biotin-β-Ala-Iminodiacetic acid | 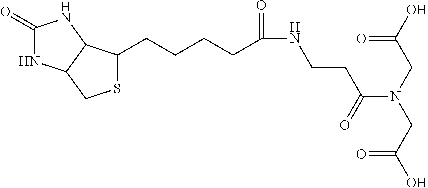 [SMILES: O=C(O)CN(CC(=O)O)C(=O)CCNC(=O)CCCCC1SCC2NC(=O)NC21] |
| Lys | Lysine | 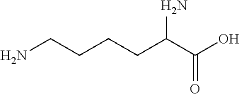 |

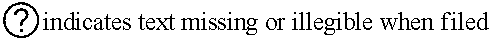

In certain embodiments, peptide inhibitors of the present invention, including both monomers and dimers, comprise one or more conjugated chemical substituents, such as lipophilic substituents and polymeric moieties, which may be referred to herein as half-life extension moieties. Without wishing to be bound by any particular theory, it is believed that the lipophilic substituent binds to albumin in the bloodstream, thereby shielding the peptide inhibitor from enzymatic degradation, and thus enhancing its half-life. In addition, it is believed that polymeric moieties enhance half-life and reduce clearance in the bloodstream.

In additional embodiments, any of the peptide inhibitors further comprise a linker moiety attached to an amino acid residue present in the inhibitor, e.g., a linker moiety may be bound to a side chain of any amino acid of the peptide inhibitor, to the N-terminal amino acid of the peptide inhibitor, or to the C-terminal amino acid of the peptide inhibitor.

In additional embodiments, any of the peptide inhibitors further comprise half-life extension moiety attached to an amino acid residue present in the inhibitor, e.g., a half-life extension moiety may be bound to a side chain of any amino acid of the peptide inhibitor, to the N-terminal amino acid of the peptide inhibitor, or to the C-terminal amino acid of the peptide inhibitor.

In additional embodiments, any of the peptide inhibitors e.g. peptides of Formulas (Va)-(Vh), further comprise half-life extension moiety attached to a linker moiety that is attached to an amino acid residue present in the inhibitor, e.g., a half-life extension moiety may be bound to a linker moiety that is bound to a side chain of any amino acid of the peptide inhibitor, to the N-terminal amino acid of the peptide inhibitor, or to the C-terminal amino acid of the peptide inhibitor.

In particular embodiments, a peptide inhibitor comprises a half-life extension moiety having the structure shown below, wherein n=0 to 24 or n=14 to 24:

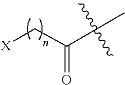

n=0 to 24
X=CH₃, CO₂H, NH₂, OH

In certain embodiments, a peptide inhibitor disclosed herein comprises a half-life extension moiety shown in Table 8.

TABLE 8
Illustrative Half-Life Extension Moieties

| # | Half-Life Extension Moietys |
|---|---|
| C1 | 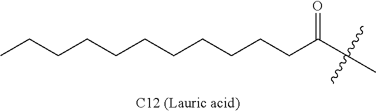 [SMILES: *C(=O)CCCCCCCCCCC] |
| C2 | 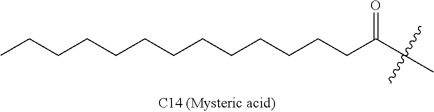 [SMILES: *C(=O)CCCCCCCCCCCCC] |
| C3 | 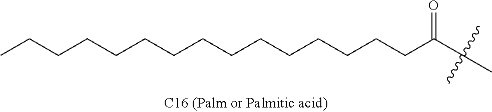 [SMILES: *C(=O)CCCCCCCCCCCCCCC] |
| C4 | 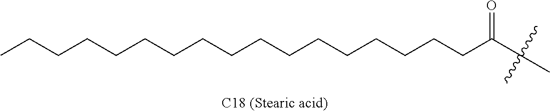 [SMILES: *C(=O)CCCCCCCCCCCCCCCCC] |
| C5 | 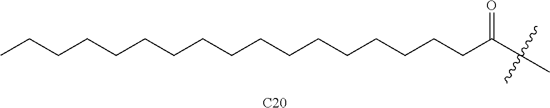 [SMILES: *C(=O)CCCCCCCCCCCCCCCCC ; "C20"] |
| C6 | 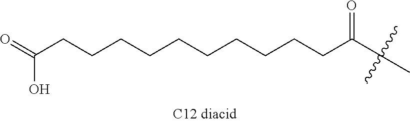 [SMILES: *C(=O)CCCCCCCCCCC(=O)O] |
| C7 | 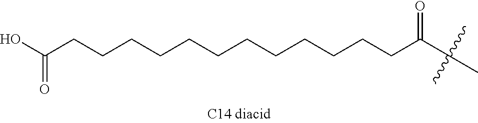 [SMILES: *C(=O)CCCCCCCCCCCCC(=O)O] |
| C8 | 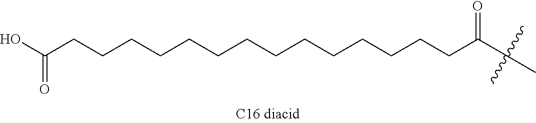 [SMILES: *C(=O)CCCCCCCCCCCCCCC(=O)O] |
| C9 | 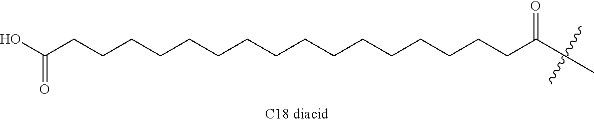 [SMILES: *C(=O)CCCCCCCCCCCCCCCCC(=O)O] |
| C10 | 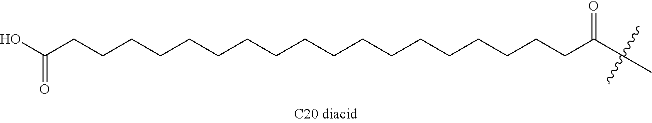 [SMILES: *C(=O)CCCCCCCCCCCCCCCCCCC(=O)O] |

In certain embodiments, a half-life extension moiety is bound directly to a peptide inhibitor, while in other embodiments, a half-life extension moiety is bound to the peptide inhibitor via a linker moiety, e.g., any of those depicted in Tables 7 or 9.

TABLE 9
Illustrative Linker Moieties

| # | Linker Moiety |
|---|---|
| L1 | 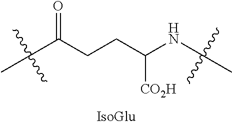 [SMILES: *NC(CCC(*)=O)C(=O)O] |
| L2 | 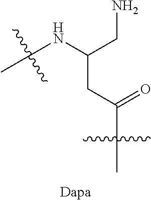 [SMILES: *NC(CN)CC(*)=O] |
| L3 | 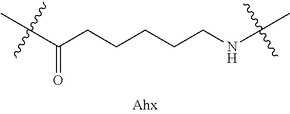 [SMILES: *NCCCCCC(*)=O] |
| L4 | 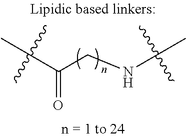 |
| L5 | 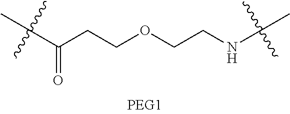 [SMILES: *NCCOCCC(*)=O] |
| L6 | 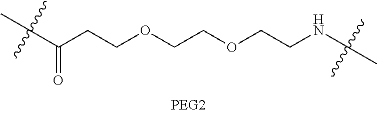 [SMILES: *NCCOCCOCCC(*)=O] |
| L7 | 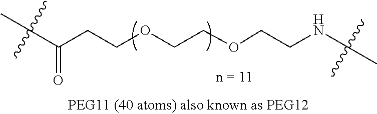 |
| L8 | 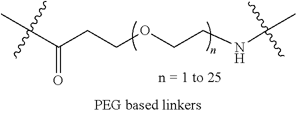 |
| L9 | 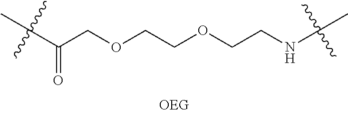 [SMILES: *NCCOCCOCC(*)=O] |
| L10 | 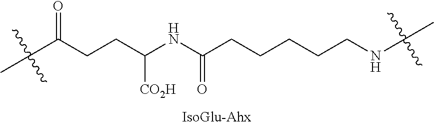 [SMILES: *NCCCCCC(=O)NC(CCC(*)=O)C(=O)O] |
| L11 | 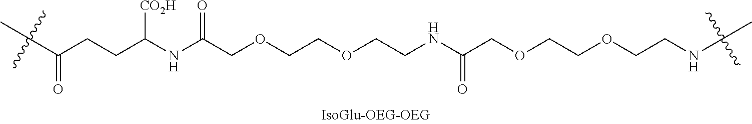 [SMILES: *NCCOCCOCC(=O)NCCOCCOCC(=O)NC(CCC(*)=O)C(=O)O] |
| L12 | 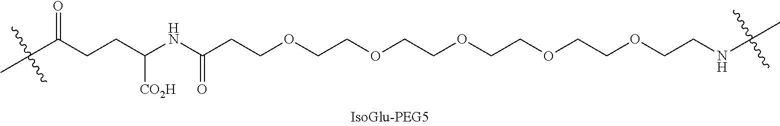 [SMILES: *NCCOCCOCCOCCOCCOCCC(=O)NC(CCC(*)=O)C(=O)O] |
| L13 | 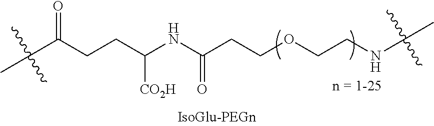 |
| L14 | 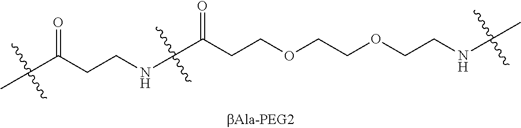 |
| L15 | 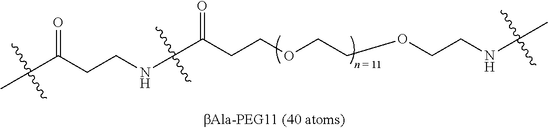 |

In particular embodiments, a peptide inhibitor of the present invention comprises any of the linker moieties shown in Tables 7 or 9 and any of the half-life extension moieties shown in Table 8, including any of the following combinations shown in Tables 10 or 11.

TABLE 10
Illustrative Combinations of Linkers and Half-Life
Extension Moieties in Peptide Inhibitors

| | Half-Life |
|---|---|
| | Extension |
| Linker | Moiety |
| L1 | C1 |
| L2 | C1 |
| L3 | C1 |
| L4 | C1 |
| L5 | C1 |
| L6 | C1 |
| L7 | C1 |
| L8 | C1 |
| L9 | C1 |
| L10 | C1 |
| L11 | C1 |
| L12 | C1 |
| L13 | C1 |
| L14 | C1 |
| L15 | C1 |
| L1 | C2 |
| L2 | C2 |
| L3 | C2 |
| L4 | C2 |
| L5 | C2 |
| L6 | C2 |
| L7 | C2 |
| L8 | C2 |
| L9 | C2 |
| L10 | C2 |
| L11 | C2 |
| L12 | C2 |
| L13 | C2 |
| L14 | C2 |
| L15 | C2 |
| L1 | C3 |
| L2 | C3 |
| L3 | C3 |
| L4 | C3 |
| L5 | C3 |
| L6 | C3 |
| L7 | C3 |
| L8 | C3 |
| L9 | C3 |
| L10 | C3 |
| L11 | C3 |
| L12 | C3 |
| L13 | C3 |
| L14 | C3 |
| L15 | C3 |
| L1 | C4 |
| L2 | C4 |
| L3 | C4 |
| L4 | C4 |
| L5 | C4 |
| L6 | C4 |
| L7 | C4 |
| L8 | C4 |
| L9 | C4 |
| L10 | C4 |
| L11 | C4 |
| L12 | C4 |
| L13 | C4 |
| L14 | C4 |
| L15 | C4 |
| L1 | C5 |
| L2 | C5 |
| L3 | C5 |
| L4 | C5 |
| L5 | C5 |
| L6 | C5 |
| L7 | C5 |
| L8 | C5 |
| L9 | C5 |
| L10 | C5 |
| L11 | C5 |
| L12 | C5 |
| L13 | C5 |
| L14 | C5 |
| L15 | C5 |
| L1 | C6 |
| L2 | C6 |
| L3 | C6 |
| L4 | C6 |
| L5 | C6 |
| L6 | C6 |
| L7 | C6 |
| L8 | C6 |
| L9 | C6 |
| L10 | C6 |
| L11 | C6 |
| L12 | C6 |
| L13 | C6 |
| L14 | C6 |
| L15 | C6 |
| L1 | C7 |
| L2 | C7 |
| L3 | C7 |
| L4 | C7 |
| L5 | C7 |
| L6 | C7 |
| L7 | C7 |
| L8 | C7 |
| L9 | C7 |
| L10 | C7 |
| L11 | C7 |
| L12 | C7 |
| L13 | C7 |
| L14 | C7 |
| L15 | C7 |
| L1 | C8 |
| L2 | C8 |
| L3 | C8 |
| L4 | C8 |
| L5 | C8 |
| L6 | C8 |
| L7 | C8 |
| L8 | C8 |
| L9 | C8 |
| L10 | C8 |
| L11 | C8 |
| L12 | C8 |
| L13 | C8 |
| L14 | C8 |
| L15 | C8 |
| L1 | C9 |
| L2 | C9 |
| L3 | C9 |
| L4 | C9 |
| L5 | C9 |
| L6 | C9 |
| L7 | C9 |
| L8 | C9 |
| L9 | C9 |
| L10 | C9 |
| L11 | C9 |
| L12 | C9 |
| L13 | C9 |
| L14 | C9 |
| L15 | C9 |
| L1 | C10 |
| L2 | C10 |
| L3 | C10 |
| L4 | C10 |
| L5 | C10 |
| L6 | C10 |
| L7 | C10 |
| L8 | C10 |
| L9 | C10 |
| L10 | C10 |
| L11 | C10 |
| L12 | C10 |
| L13 | C10 |
| L14 | C10 |
| L15 | C10 |

In some embodiments there may be multiple linkers present between the peptide the conjugated moiety, e.g., half-life extension moiety, e.g., as depicted in Table 11.

TABLE 11
Illustrative Combinations of Linkers and Half-Life
Extension Moieties in Peptide Inhibitors

| | Half-Life Extension |
|---|---|
| Linker | Moiety |
| L1-L2 | C10 |
| L2-L5-L3 | C10 |
| L3-L8 | C10 |
| L1-L2-L3 | C10 |
| L5-L3-L3-L3 | C10 |
| L1-L2 | C8 |
| L2-L5-L3 | C8 |
| L3-L8 | C8 |
| L1-L2-L3 | C8 |
| L5-L3-L3-L3 | C8 |

Illustrative examples of peptide inhibitors of the present invention, including those having a conjugates linker and/or half-life extension moiety are shown below. All amino acids are L amino acids unless otherwise stated. The present invention also includes salt forms of any of these peptide inhibitors, including, but not limited to, acetate salts thereof.

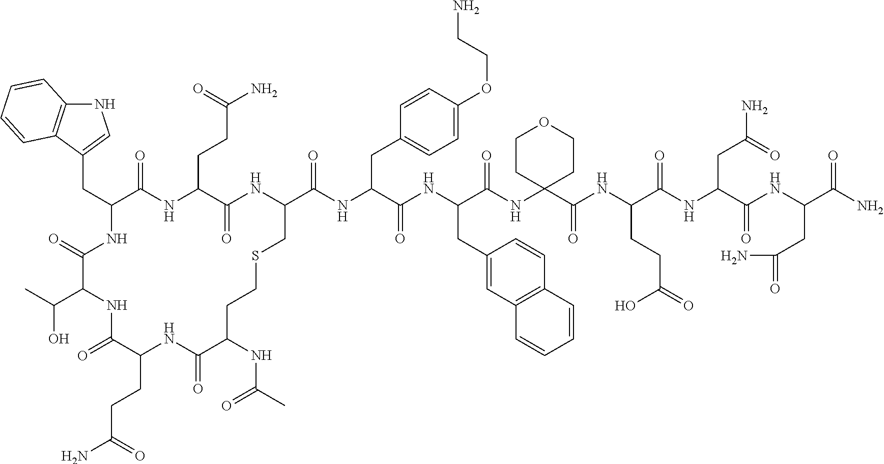 [SMILES: CC(=O)NC1CCSCC(C(=O)NC(Cc2ccc(OCCN)cc2)C(=O)NC(Cc2ccc3ccccc3c2)C(=O)NC2(C(=O)NC(CCC(=O)O)C(=O)NC(CC(N)=O)C(=O)NC(CC(N)=O)C(N)=O)CCOCC2)NC(=O)C(CCC(N)=O)NC(=O)C(Cc2c[nH]c3ccccc23)NC(=O)C(C(C)O)NC(=O)C(CCC(N)=O)NC1=O]

### Example 1: cyclo[[Abu]-QTWQC]-[Phe[4-(2-aminoethoxy)]-[2-Nal]-[4-amino-4-carboxy-tetrahydropyran]-ENN-NH₂

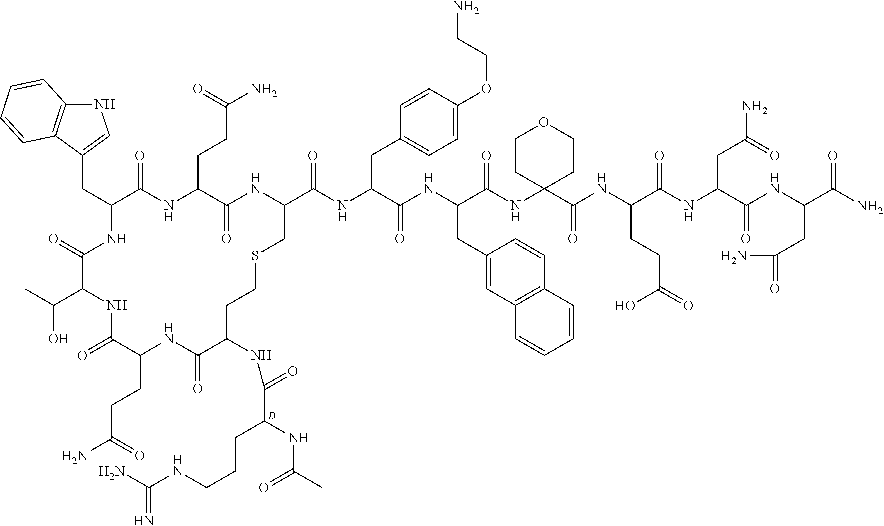

### Example 1a: Ac-[(D)-Arg]-cyclo[[Abu]-QTWQC]-[Phe[4-(2-aminoethoxy)]-[2-Nal]-[4-amino-4-carboxy-tetrahydropyran]-ENN-NH₂

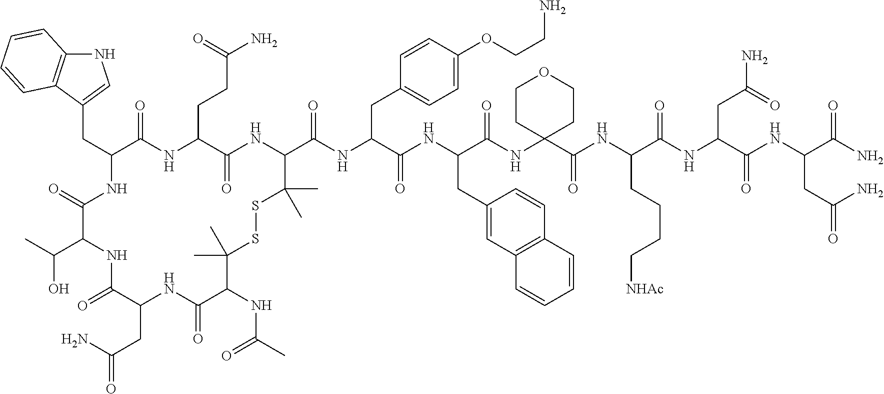 [SMILES: CC(=O)NCCCCC(NC(=O)C1(NC(=O)C(Cc2ccc3ccccc3c2)NC(=O)C(Cc2ccc(OCCN)cc2)NC(=O)C2NC(=O)C(CCC(N)=O)NC(=O)C(Cc3c[nH]c4ccccc34)NC(=O)C(C(C)O)NC(=O)C(CC(N)=O)NC(=O)C(NC(C)=O)C(C)(C)SSC2(C)C)CCOCC1)C(=O)NC(CC(N)=O)C(=O)NC(CC(N)=O)C(N)=O]

### Example 2: Ac-[Pen]-NTWQ-[Pen]-[Phe[4-(2-aminoethoxy)]-[2-Nal]-[4-amino-4-carboxy-tetrahydropyran]-[Lys(Ac)]-NN-NH₂

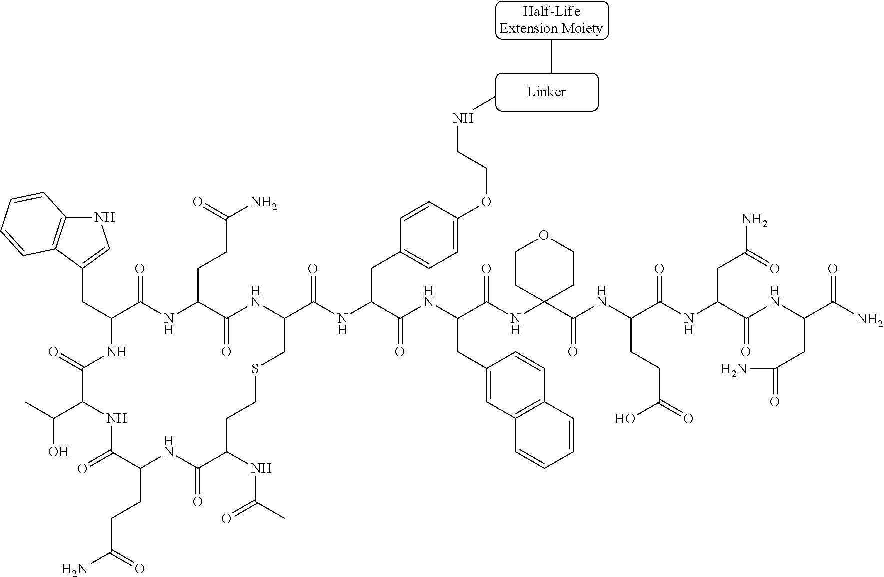

### Example 3: cyclo[[Abu]-QTWQC]-[Phe[4-(2-aminoethoxy)-(Linker-Half-Life Extension Moiety)]-[2-Nal]-[4-amino-4-carboxy-tetrahydropyran]-ENN-NH₂

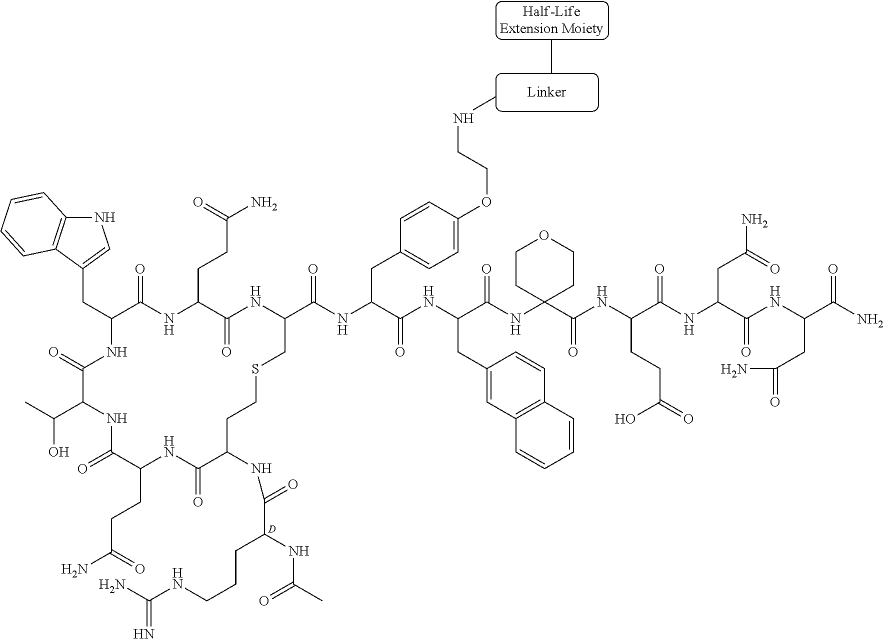

### Example 3a: Ac-[(D)-Arg]cyclo[[Abu]-QTWQC]-[Phe[4-(2-aminoethoxy)-(Linker-Half-Life Extension Moiety)]-[2-Nal]-[4-amino-4-carboxy-tetrahydropyran]-ENN-NH₂

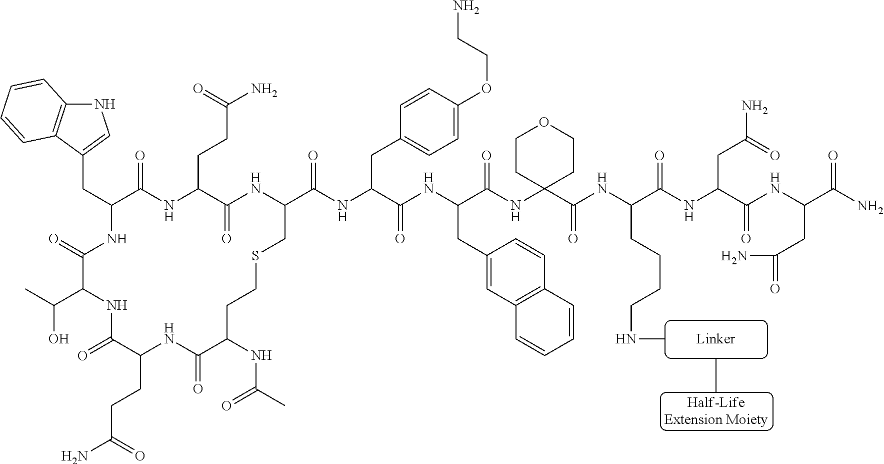

### Example 4: cyclo[[Abu]-QTWQC]-[Phe[4-(2-aminoethoxy)]-[2-Nal]-[4-amino-4-carboxy-tetrahydropyran]-[Lys(Linker-Half-Life Extension Moiety)]-NN-N-NN-NH₂

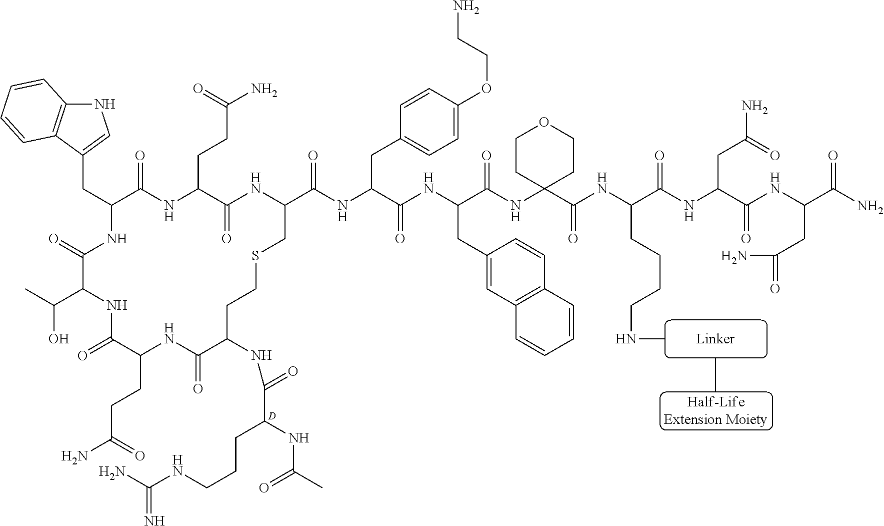

### Example 4a: Ac-[(D)-Arg]-cyclo[[Abu]-QTWQC]-[Phe[4-(2-aminoethoxy)]-[2-Nal]-[4-amino-4-carboxy-tetrahydropyran]-[Lys(Linker-Half-Life Extension Moiety)]-NN-NH₂

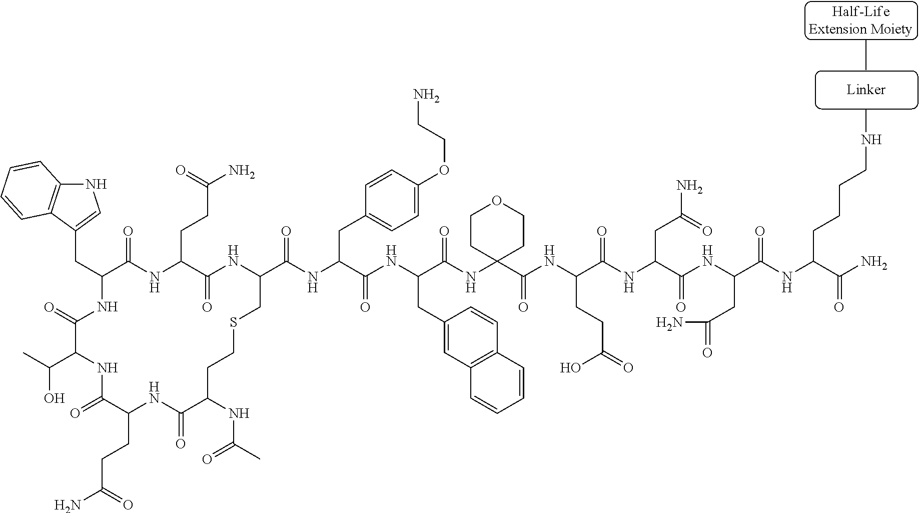

### Example 5: cyclo[[Abu]-QTWQC]-[Phe[4-(2-aminoethoxy)]-[2-Nal]-[4-amino-4-carboxy-tetrahydropyran]-ENN-[Lys(Linker-Half-Life Extension Moiety)]-NH₂

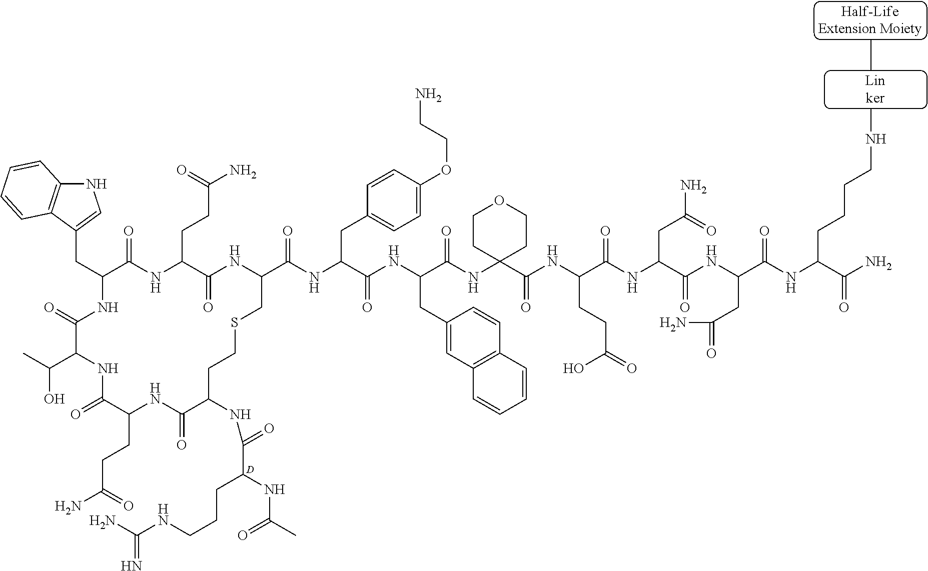

### Example 5a: Ac[(D)-Arg]-cyclo[[Abu]-QTWQC]-[Phe[4-(2-aminoethoxy)]-[2-Nal]-[4-amino-4-carboxy-tetrahydropyran]-ENN-[Lys(Linker-Half-Life Extension Moiety)]-NH₂

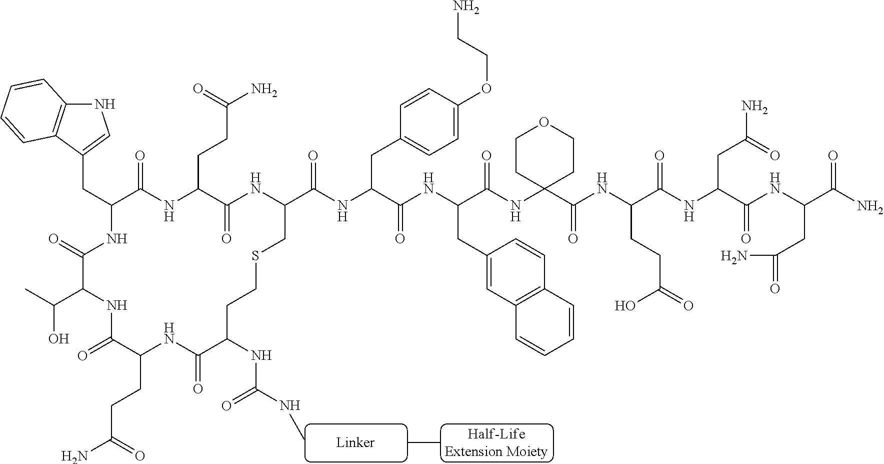

### Example 6: [Half-Life Extension Moiety-Linker]-[cyclo[[Abu]-QTWQC]-[Phe[4-(2-aminoethoxy)]-[2-Nal]-[4-amino-4-carboxy-tetrahydropyran]-ENN-NH₂

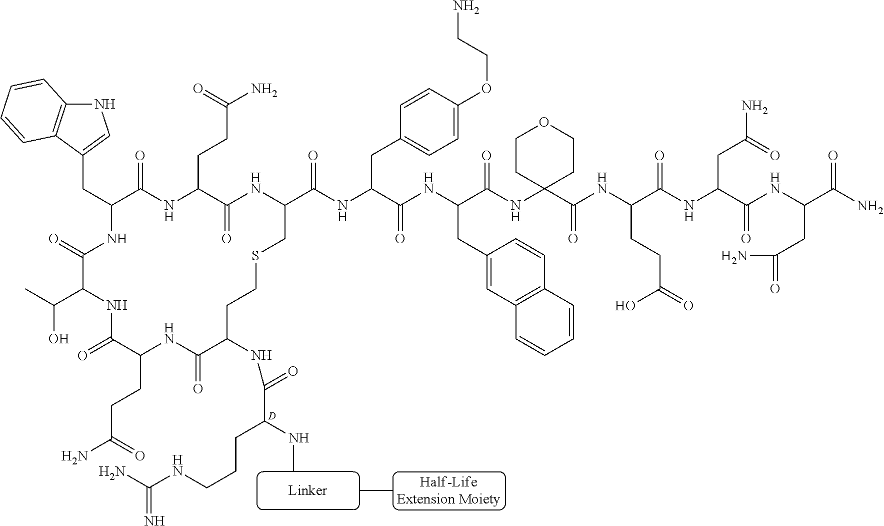

### Example 6a: [Half-Life Extension Moiety-Linker]-[(D)-Arg]-[cyclo[Abu]-QTWQC]-[Phe[4-(2-aminoethoxy)]-[2-Nal]-[4-amino-4-carboxy-tetrahydropyran]-ENN-NH₂

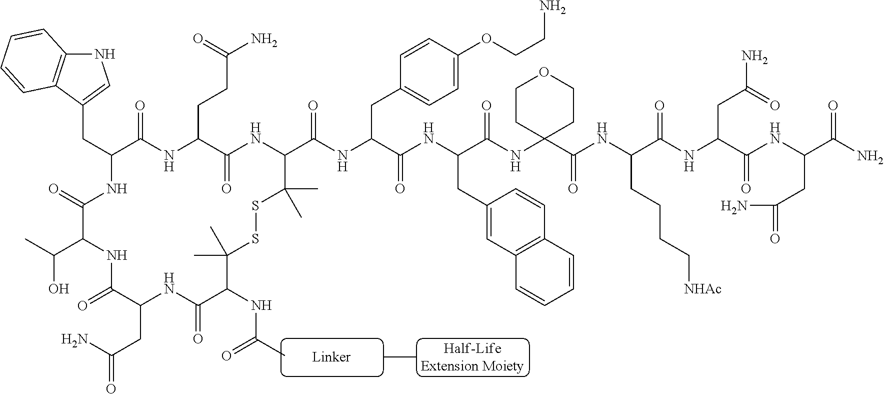

### Example 7: [Half-Life Extension Moiety-Linker]-[Pen]-NTWQ-[Pen]-[Phe[4-(aminoethoxy)]-[2-Nal]-[4-amino-4-carboxy-tetrahydropyran]-[Lys(Ac)]-NN-NH₂

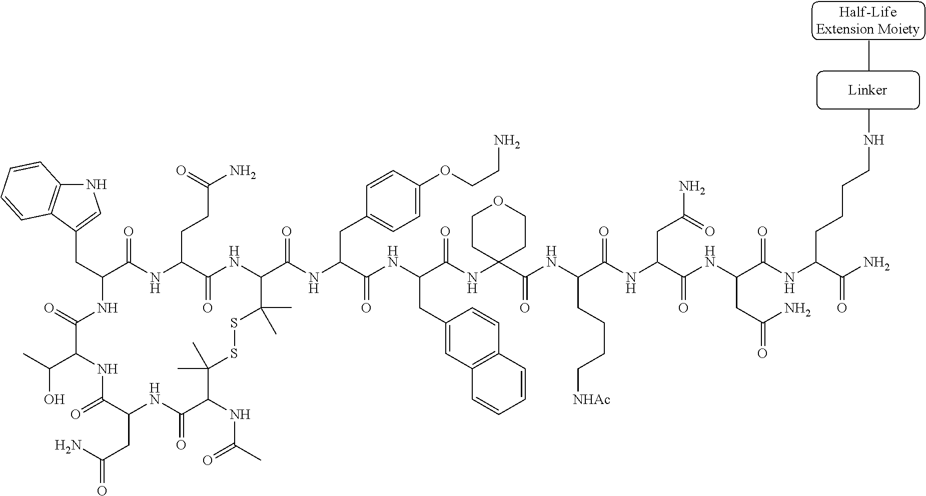

### Example 8: Ac-[Pen]-NTWQ-[Pen]-[Phe[4-(aminoethoxy)]-[2-Nal]-[4-amino-4-carboxy-tetrahydropyran]-[Lys(Ac)-NN-[Lys(Linker-Half-Life Extension Moiety)]-NH₂

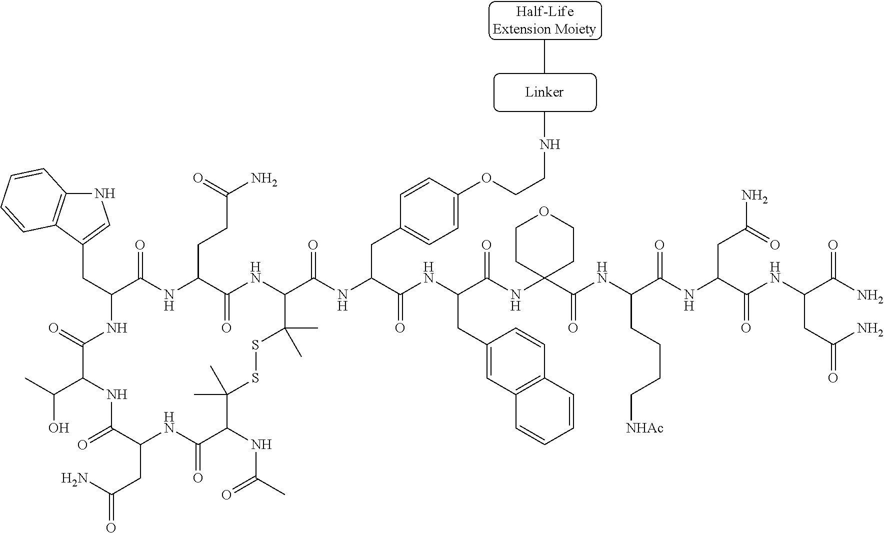

### Example 9: Ac-[Pen]-NTWQ-[Pen]-[Phe[4-(aminoethoxy)-(Linker-Half-Life Extension Moiety)]-[2-Nal]-[4-amino-4-carboxy-tetrahydropyran]-[Lys(Ac)]-NN-NH₂

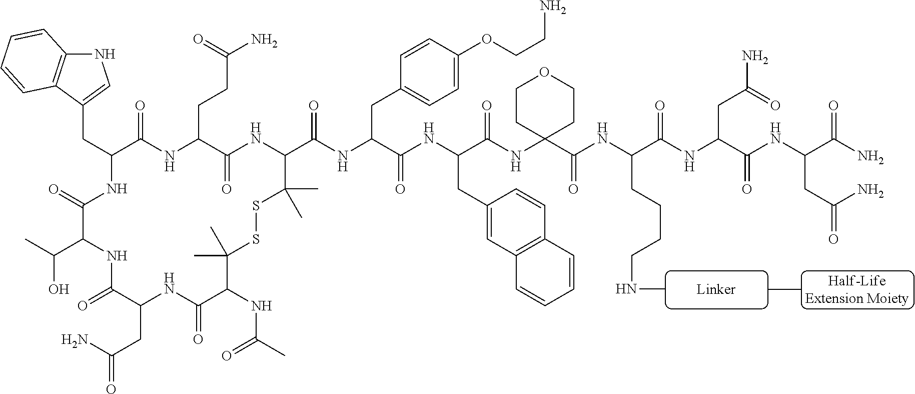

### Example 10: Ac-[Pen]-NTWQ-[Pen]-[Phe[4-(aminoethoxy)]-[2-Nal]-[4-amino-4-carboxy-tetrahydropyran]-[Lys(Linker-Half-Life Extension Moiety)]-NN-NH₂

In certain embodiments, the half-life of a peptide inhibitor of the invention that includes a conjugated chemical substituent, i.e., a half-life extension moiety, is at least 100%, at least 120%, at least 150%, at least 200%, at least 250%, at least 300%, at least 400%, or at least 500% of the half-life of the same peptide inhibitor but without the conjugated chemical substituent. In certain embodiments, the lipophilic substituents and/or polypermic moieties enhance the permeability of the peptide inhibitor through the epithelium and/or its retention in the lamina propria. In certain embodiments, the permeability through the epithelium and/or the retention in the lamina propria of a peptide inhibitor of the invention that includes a conjugated chemical substituent is at 100%, at least 120%, at least 150%, at least 200%, at least 250%, at least 300%, at least 400%, or at least 500% of the half-life of the same peptide inhibitor but without the conjugated chemical substituent.

In one embodiment, a side chain of one or more amino acid residues (e.g., Lys residues) in a peptide inhibitor of the invention is conjugated (e.g., covalently attached) to a lipophilic substituent. The lipophilic substituent may be covalently bonded to an atom in the amino acid side chain, or alternatively may be conjugated to the amino acid side chain via one or more spacers. The spacer, when present, may provide spacing between the peptide analogue and the lipophilic substituent. In particular embodiments, the peptide inhibitor comprises any of the conjugated moieties shown in Tables 2-5.

In certain embodiments, the lipophilic substituent may comprise a hydrocarbon chain having from 4 to 30 C atoms, for example at least 8 or 12 C atoms, and preferably 24 C atoms or fewer, or 20 C atoms or fewer. The hydrocarbon chain may be linear or branched and may be saturated or unsaturated. In certain embodiments, the hydrocarbon chain is substituted with a moiety which forms part of the attachment to the amino acid side chain or the spacer, for example an acyl group, a sulfonyl group, an N atom, an O atom or an S atom. In some embodiments, the hydrocarbon chain is substituted with an acyl group, and accordingly the hydrocarbon chain may form part of an alkanoyl group, for example palmitoyl, caproyl, lauroyl, myristoyl or stearoyl.

A lipophilic substituent may be conjugated to any amino acid side chain in a peptide inhibitor of the invention. In certain embodiment, the amino acid side chain includes a carboxy, hydroxyl, thiol, amide or amine group, for forming an ester, a sulphonyl ester, a thioester, an amide or a sulphonamide with the spacer or lipophilic substituent. For example, the lipophilic substituent may be conjugated to Asn, Asp, Glu, Gln, His, Lys, Arg, Ser, Thr, Tyr, Trp, Cys, Dap, Dab or Orn. In certain embodiments, the lipophilic substituent is conjugated to Lys. An amino acid shown as Lys in any of the formula provided herein may be replaced by, e.g., Dap, Dab or Orn where a lipophilic substituent is added.

In certain embodiments, the peptide inhibitors of the present invention may be modified, e.g., to enhance stability, increase permeability, or enhance drug like characteristics, through conjugation of a chemical moiety to one or more amino acid side chain within the peptide. For example, the N(epsilon) of lysine N(epsilon), the β-carboxyl of aspartic, or the γ-carboxyl of glutamic acid may be appropriately functionalized. Thus, to produce the modified peptide, an amino acid within the peptide may be appropriately modified. Further, in some instances, the side chain is acylated with an acylating organic compound selected from the group consisting of: Trifluoropentyl, Acetyl, Octonyl, Butyl, Pentyl, Hexyl, Palmityl, Trifluoromethyl butyric, cyclopentane carboxylic, cyclopropylacetic, 4-fluorobenzoic, 4-fluorophenyl acetic, 3-Phenylpropionic, tetrahedro-2H-pyran-4carboxylic, succinic acid glutaric acid or bile acids. One having skill is the art will appreciate that a series of conjugates can be linked, e.g., for example PEG4, isoglu and combinations thereof. One having skill is the art will appreciate that an amino acid with the peptide can be isosterically replaced, for example, Lys may be replaced for Dap, Dab, α-MeLys orOrn. Examples of modified residues within a peptide are shown in Table 12.

TABLE 12
Examples of modified Lysine, Asp and Asn within the peptide

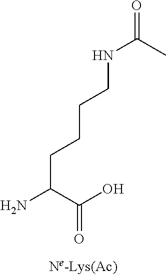

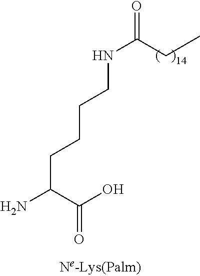

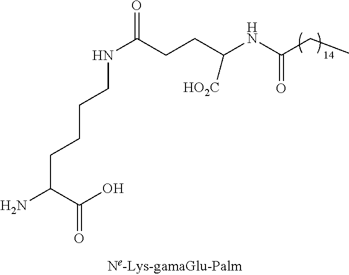

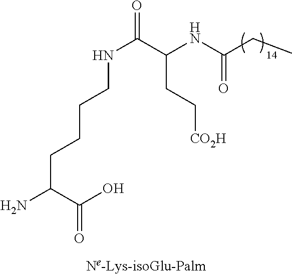

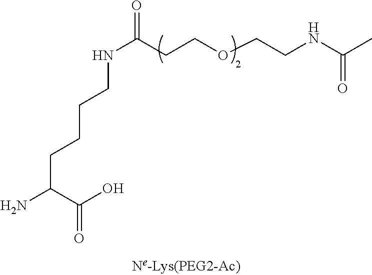

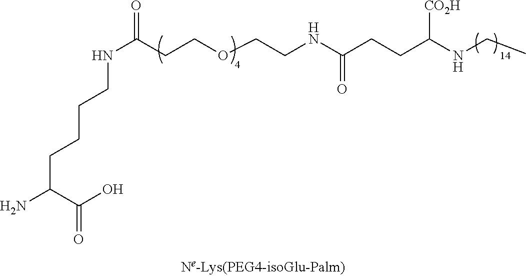

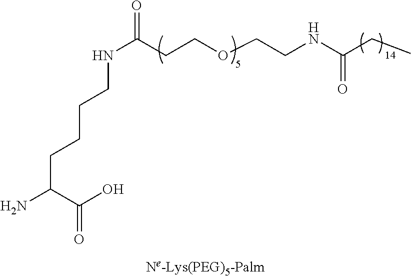

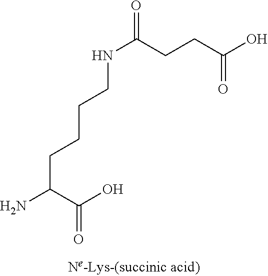 [SMILES: NC(CCCCNC(=O)CCC(=O)O)C(=O)O]

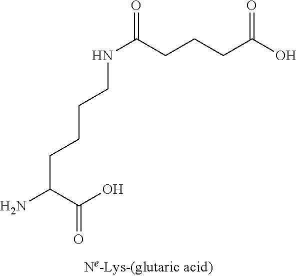 [SMILES: NC(CCCCNC(=O)CCCC(=O)O)C(=O)O]

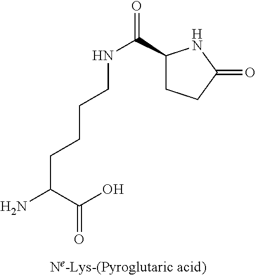 [SMILES: NC(CCCCNC(=O)[C@@H]1CCC(=O)N1)C(=O)O]

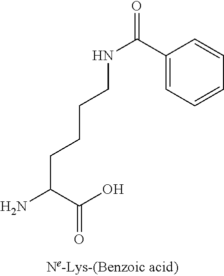 [SMILES: NC(CCCCNC(=O)c1ccccc1)C(=O)O]

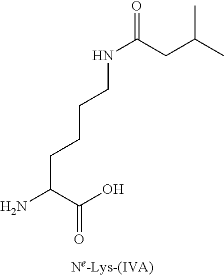 [SMILES: CC(C)CC(=O)NCCCCC(N)C(=O)O]

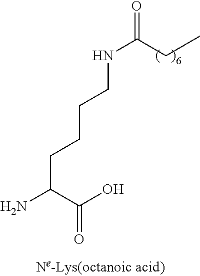

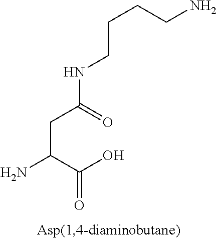 [SMILES: NCCCCNC(=O)CC(N)C(=O)O]

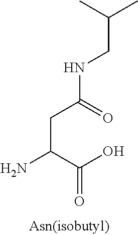

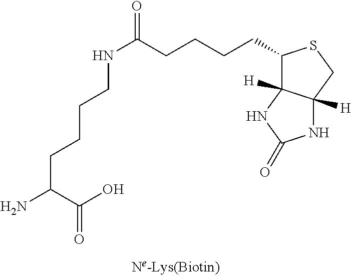 [SMILES: NC(CCCCNC(=O)CCCC[C@@H]1SC[C@@H]2NC(=O)N[C@@H]21)C(=O)O]

In further embodiments of the present invention, alternatively or additionally, a side-chain of one or more amino acid residues in a peptide inhibitor of the invention is conjugated to a polymeric moiety, for example, in order to increase solubility and/or half-life in vivo (e.g. in plasma) and/or bioavailability. Such modifications are also known to reduce clearance (e.g. renal clearance) of therapeutic proteins and peptides.

As used herein, “Polyethylene glycol” or “PEG” is a polyether compound of general formula H—(O—CH2-CH2)n-OH. PEGs are also known as polyethylene oxides (PEOs) or polyoxyethylenes (POEs), depending on their molecular weight PEO, PEE, or POG, as used herein, refers to an oligomer or polymer of ethylene oxide. The three names are chemically synonymous, but PEG has tended to refer to oligomers and polymers with a molecular mass below 20,000 Da, PEO to polymers with a molecular mass above 20,000 Da, and POE to a polymer of any molecular mass. PEG and PEO are liquids or low-melting solids, depending on their molecular weights. Throughout this disclosure, the 3 names are used indistinguishably. PEGs are prepared by polymerization of ethylene oxide and are commercially available over a wide range of molecular weights from 300 Da to 10,000,000 Da. While PEG and PEO with different molecular weights find use in different applications, and have different physical properties (e.g. viscosity) due to chain length effects, their chemical properties are nearly identical. The polymeric moiety is preferably water-soluble (amphiphilic or hydrophilic), non-toxic, and pharmaceutically inert. Suitable polymeric moieties include polyethylene glycols (PEG), homo- or co-polymers of PEG, a monomethyl-substituted polymer of PEG (mPEG), or polyoxyethylene glycerol (POG). See, for example, Int. J. Hematology 68:1 (1998); Bioconjugate Chem. 6:150 (1995); and Crit. Rev. Therap. Drug Carrier Sys. 9:249 (1992). Also encompassed are PEGs that are prepared for purpose of half life extension, for example, mono-activated, alkoxy-terminated polyalkylene oxides (POA's) such as mono-methoxy-terminated polyethylene glycols (mPEG's); bis activated polyethylene oxides (glycols) or other PEG derivatives are also contemplated. Suitable polymers will vary substantially by weights ranging from about 200 Da to about 40,000 Da or from about 200 Da to about 60,000 Da are usually selected for the purposes of the present invention. In certain embodiments, PEGs having molecular weights from 200 to 2,000 or from 200 to 500 are used. Different forms of PEG may also be used, depending on the initiator used for the polymerization process—a common initiator is a monofunctional methyl ether PEG, or methoxypoly(ethylene glycol), abbreviated mPEG.

Lower-molecular-weight PEGs are also available as pure oligomers, referred to as monodisperse, uniform, or discrete. These are used in certain embodiments of the present invention.

PEGs are also available with different geometries: branched PEGs have three to ten PEG chains emanating from a central core group; star PEGs have 10 to 100 PEG chains emanating from a central core group; and comb PEGs have multiple PEG chains normally grafted onto a polymer backbone. PEGs can also be linear. The numbers that are often included in the names of PEGs indicate their average molecular weights (e.g. a PEG with n=9 would have an average molecular weight of approximately 400 daltons, and would be labeled PEG 400.

As used herein, “PEGylation” is the act of covalently coupling a PEG structure to the peptide inhibitor of the invention, which is then referred to as a “PEGylated peptide inhibitor”. In certain embodiments, the PEG of the PEGylated side chain is a PEG with a molecular weight from about 200 to about 40,000. In some embodiments, a spacer of a peptide of formula I, formula I′, or formula I″ is PEGylated. In certain embodiments, the PEG of a PEGylated spacer is PEG3, PEG4, PEG5, PEG6, PEG7, PEG8, PEG9, PEG10, or PEG11. In certain embodiments, the PEG of a PEGylated spacer is PEG3 or PEG8.

Other suitable polymeric moieties include poly-amino acids such as poly-lysine, poly-aspartic acid and poly-glutamic acid (see for example Gombotz, et al. (1995), Bioconjugate Chem., vol. 6: 332-351; Hudecz, et al. (1992), Bioconjugate Chem., vol. 3, 49-57 and Tsukada, et al. (1984), J. Natl. Cancer Inst., vol. 73, :721-729. The polymeric moiety may be straight-chain or branched. In some embodiments, it has a molecular weight of 500-40,000 Da, for example 500-10,000 Da, 1000-5000 Da, 10,000-20,000 Da, or 20,000-40,000 Da.

In some embodiments, a peptide inhibitor of the invention may comprise two or more such polymeric moieties, in which case the total molecular weight of all such moieties will generally fall within the ranges provided above.

In some embodiments, the polymeric moiety is coupled (by covalent linkage) to an amino, carboxyl or thiol group of an amino acid side chain. Certain examples are the thiol group of Cys residues and the epsilon amino group of Lys residues, and the carboxyl groups of Asp and Glu residues may also be involved.

The skilled worker will be well aware of suitable techniques which can be used to perform the coupling reaction. For example, a PEG moiety bearing a methoxy group can be coupled to a Cys thiol group by a maleimido linkage using reagents commercially available from Nektar Therapeutics AL. See also WO 2008/101017, and the references cited above, for details of suitable chemistry. A maleimide-functionalised PEG may also be conjugated to the side-chain sulfhydryl group of a Cys residue.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Each embodiment in this specification is to be applied mutatis mutandis to every other embodiment unless expressly stated otherwise. Accordingly, the invention is not limited except as by the appended claims.

### Example E1

### Levels of Biomarkers Following Treatment with Peptide Inhibitors in a Rat Model of Acute Colitis

The effect of treatment with peptide inhibitors of IL23R on inflammatory markers associated with IL23 signaling was examined in a rat model of acute colitis. Acute colitis was induced in Sprague-Dawley rats by a single intra-rectal instillation of 2,4,6-Trinitrobenzenesulfonic acid (TNBS) followed by efficacy analysis at day seven. Pharmacodynamic (PD) biomarkers were examined using enzyme-linked immunosorbent assay (ELISA), quantitative reverse transcription polymerase chain reaction, or immunohistochemistry analysis of colon, feces, or serum samples obtained from colitic rats treated with Peptide 993, Peptide 1185, or Peptide 980.

The sequence of Peptide 993 is shown below: Ac-[(D)-Arg]-cyclo[[Abu]-QTWQC]-[Phe[4-(2-aminoethoxy)]]-[2-Nal]-[4-amino-4-carboxy-tetrahydropyran]-ENN-NH₂ (SEQ ID NO: 993), where “cyclo” indicates a thioether bond between Abu and C.

The sequence of Peptide 1185 is shown below: Ac-cyclo[[Pen]-NTWQ-[Pen]]-[Phe[4-(2-aminoethoxy)]]-[2-Nal]-[4-amino-4-carboxy-tetrahydropyran]-[Lys(Ac)]-NN-NH₂ (SEQ ID NO: 1185), where “cyclo” indicates a disulfide bond between the two Pen residues.

The sequence of Peptide 980 is shown below:

Ac-cyclo[[Abu]-QTWQC]-[Phe[4-(2-aminoethoxy)]]-[2-Nal]-[4-amino-4-carboxy-tetrahydropyran]-ENN-NH₂ (SEQ ID NO: 980), where “cyclo” indicates a thioether bond between Abu and C.

Acute colitis was induced by providing 7-week-old female Sprague-Dawley rats with various dosages of 64 mg/kg or 48 mg/kg of TNBS in 50% ethanol (TNBS/ethanol) administered intrarectally at Day 0. Peptide inhibitors were administered orally two or three times a day at various dosages and were provided in drinking water at various dosages, for 8 days starting approximately 24 hours (Day −1) prior TNBS inoculation.

Levels of a number of inflammatory biomarkers were examined in the colons. The distal colon tissue samples, designated for protein expression analysis, were flash frozen after collection. For protein extraction, the samples were thawed, weighed and homogenized in the extraction buffer (PBS pH 7.2 supplemented with Protease Inhibitors, 3× volume:weight). The homogenates were centrifuged at 13 krpm at 4° C. for 15 minutes, a total of two times to remove the debris. The supernatants were saved in multiple aliquots in −80° C. and subsequently used for protein expression analysis on ELISA. The total protein in each sample was quantified using BCA assay. MPO, IL-10, IL-6, IL-17A and IL-22 biomarker protein expression in the distal colon samples was analyzed using commercially available rat ELISA kits.

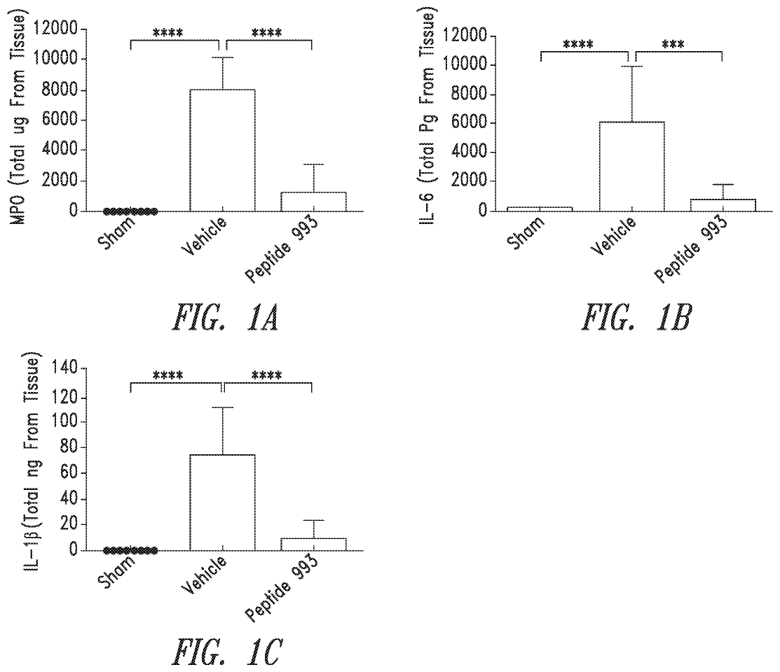

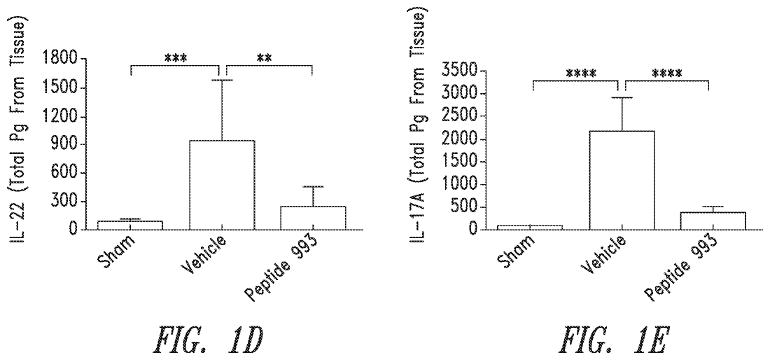

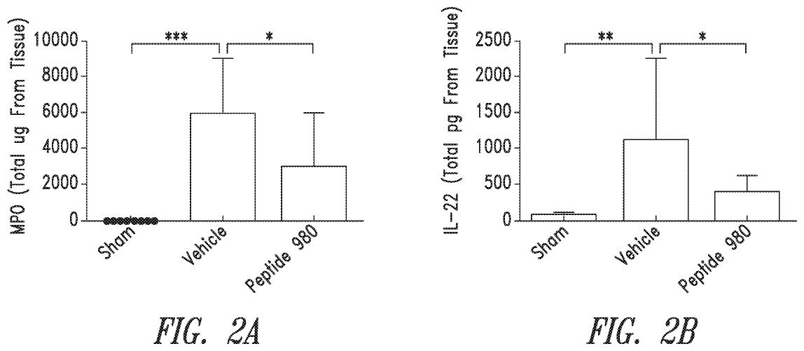

Treatment with Peptide 993 reduced levels of inflammatory markers present in the colon. Disease defining biomarkers (MPO, IL-6 and IL-1β) and IL-23 directed biomarkers (IL-22 and IL-17A) were reduced by treatment with Peptide 993 as compared to vehicle treated controls (FIGS. 1A-1E). A similar trend was observed at the gene expression level from distal colon for IL-6 but not IL-1β (data not shown). Treatment with Peptide 980 reduced levels MPO and IL-22 as compared to vehicle treated controls (see FIG. 2A-2B). Treatment with Peptide 1185 at the dose tested did not significantly reduce levels of MPO, IL-22, or IL-17A (data not shown). These data demonstrate that administration of Peptide 993 in amounts that can reduce pathology in vivo also decreased levels of biomarkers present in the colon that are associated with IL-23R activity.

### Example E2

### Levels of Biomarkers in Serum and Feces Following Treatment with Peptide Inhibitors in a Rat Model of Acute Colitis

The effect of treatment with Peptide 993 on levels of protein biomarkers present in the serum and feces of animals using the TNBS exposed rat colitis model described in Example E1 was demonstrated.

Sample Collection:

The animals were grouped into sham, vehicle, and Peptide 993 treatment at 31 mg/kg/day. Feces were collected from the distal colon of rats and were immediately frozen at the time of necropsy. The feces samples were weighed and homogenized in extraction buffer (PBS pH 7.4 containing Pierce Protease Inhibitors, 3× volume:weight). The samples were incubated on ice for 90 minutes, with intermittent vortexing for complete homogenization. After centrifugation at 3000 g at 4 C, the homogenates were transferred to fresh tubes and subjected to another centrifugation at 14000 g at 4 C. The cleared supernatants were saved in multiple aliquots and stored at −80 C until analysis.

The total protein in each sample was quantified using BCA assay. LCN2, MPO, and MMP-9 protein concentration in the feces samples were analysed using commercially available rat ELISA kits. For LCN2, the samples were diluted at different ratios with assay dilution buffer to capture the large variation in concentration amongst the different treatment groups. The dilution ratios used were 1:20 (sham), 1:200, 1:1000, 1:2000, and 1:4000 (vehicle and Peptide 993).

The serum samples were immediately frozen and thawed only once before use in the ELISA. For LCN2, the samples were diluted at different ratios with assay dilution buffer to capture the large variation in concentration amongst the different groups. The dilution ratios used were 1:10 (for sham), 1:1000, 1:2000 and 1:10000 (vehicle and Peptide 993). Fecal Lipocalin2(LCN2)/NGAL analysis:

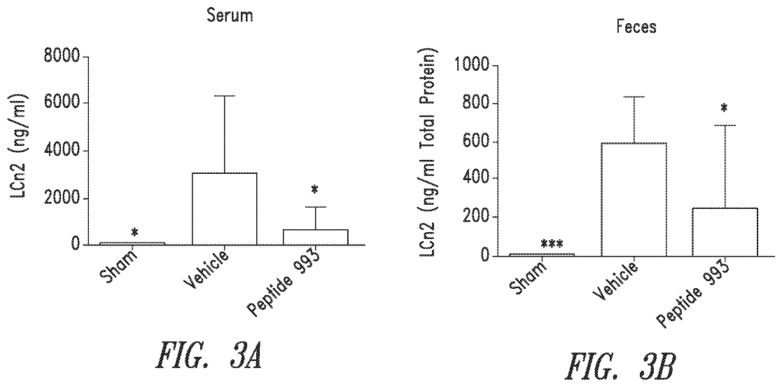

Fecal homogenates were analyzed for Lipocalin2 (LCN2) concentration using the Rat Lipocalin-2/NGAL DuoSet ELISA Development kit (Catalog # DY3508, R&D Systems. The samples were used at dilutions ranging from 1:20 to 1:12000, to ensure that the measurements were within the range and sensitivity of the kit. The ELISA protocol suggested for the kit DY3508 was followed for the samples and the standards provided with the kit. A four-parameter sigmoidal equation was used to fit the Standard Curve and the fitting parameters were used to calculate the concentration of LCN2 in the samples. The protein concentration of the homogenates was measured using the Pierce BCA protein assay kit (Catalog #23225, ThermoScientific). The concentration of LCN2 was normalized to the total protein measured for the sample. FIG. 3B shows LCN2 concentration normalized to total protein. Statistical analysis was performed using 1-Way-ANOVA with Dunnetts's multiple comparison. The group treated with Peptide 993 showed a statistically significant decrease in fecal LCN2 concentration.

### Serum Lipocalin2/NGAL Analysis:

Serum samples were analyzed for LCN2 concentration using the above ELISA protocol. Serum samples were used at dilutions ranging from 1:10 to 1:12000 to ensure that the measurements were within the range and sensitivity of the kit. FIG. 3A shows the LCN2 concentration per ml of serum. Statistical analysis was performed using 1-Way-ANOVA with Dunnetts's multiple comparison. The group treated with Peptide 993 showed a statistically significant decrease in serum LCN2 concentration.

### Fecal Myeloperoxidase (MPO) Analysis:

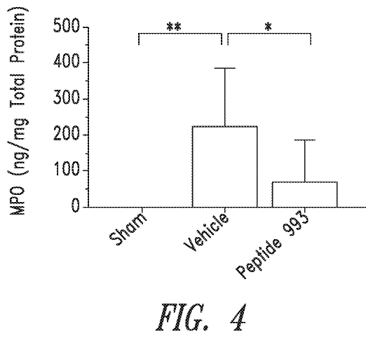

Fecal homogenates were analyzed for Myeloperoxidase (MPO) concentration using the Rat MPO ELISA kit (Catalog # HK105, Hycult biotech). The samples were used at dilutions ranging from 1:20 to 1:1000, to ensure that the measurements were within the range and sensitivity of the kit. The ELISA protocol suggested for the kit HK105 was followed for the samples and the standards provided with the kit. A linear equation was used to fit the Standard Curve and the fitting parameters were used to calculate the concentration of MPO. The concentration of MPO for each sample was normalized to the total protein measured for the sample as described above. FIG. 4 shows MPO concentration normalized to total protein. Statistical analyses were performed using 1-Way-ANOVA with Dunnetts's multiple comparison. The group treated with Peptide 993 showed a statistically significant decrease in fecal MPO concentration.

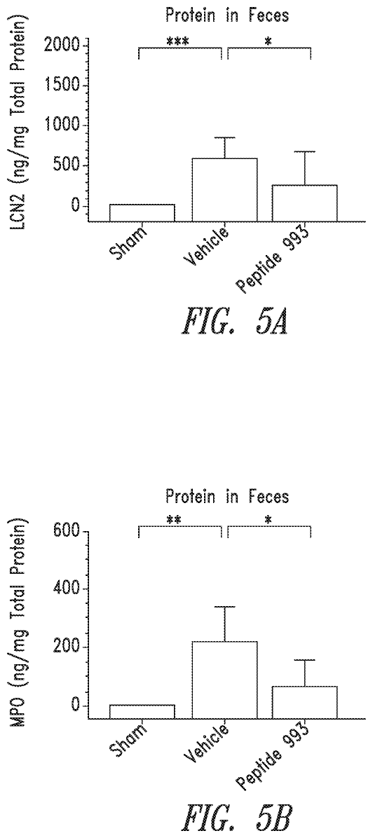

FIGS. 5A-5C summarizes results of experiments using feces, and shows the concentration of LCN2, MPO and MMP-9 in feces samples, normalized using total protein. These data show that the concentration of LCN2, MPO and MMP-9 were statistically significantly increased in vehicle-treated animals (TNBS exposed vehicle group) as compared to the sham group (control group not exposed to TNBS). The Peptide 993 treated group exhibited a stastically significant decrease in protein concentrations of LCN2, MPO, and MMP-9.

### Example E3

### Biomarker Gene Expression in Serum and Distal Colon Following Treatment with Peptide Inhibitors in a Rat Model of Acute Colitis

The effect of treatment with Peptide 993 on gene expression levels of biomarkers present in the serum and distal colon of animals using the TNBS exposed rat colitis model described in Example E1 was demonstrated.

Animals from the sham group (without TNBS exposure), the vehicle group (exposed to TNBS and treated with vehicle only) and the group treated with Peptide 993 (exposed to TNBS and treated with Peptide 993 at 31 mg/kg/day) were subjected for gene expression and miroRNA analysis

For extraction of total RNA from the distal colons, tissue samples stored at −20° C. in the buffer RNAlater, were thawed and homogenized in RLTplus Buffer (Qiagen) at 20× volume:wt, then subsequently purified using the RNeasy Plus Mini Kit (Qiagen). The concentration of the total RNA was measured and the quality of the RNAs was verified on a gel. The total RNA was used in QuantiTect Reverse Transcription Kit (Qiagen) to obtain cDNA templates for the quantative real time PCR. The expression of the genes LCN2 (Lipocalin 2), S100A8 (a subunit of calprotectin) and CLDN8 (Claudin 8 of the claudin family of tight junction proteins) were measured using Taqman and TaqMan Fast Advanced Master Mix (Applied Biosystems). The threshold cycle number (Ct) values, that are indicative of abundance of template, were normalized using the endogenous control gene HPRT1. The Relative expression of each gene was calculated using the normalized Ct values. Statistical analysis of relative expression was performed using One-way ANOVA method (with Dunnett's multiple comparisons) to compare the variations Vs Group 2.

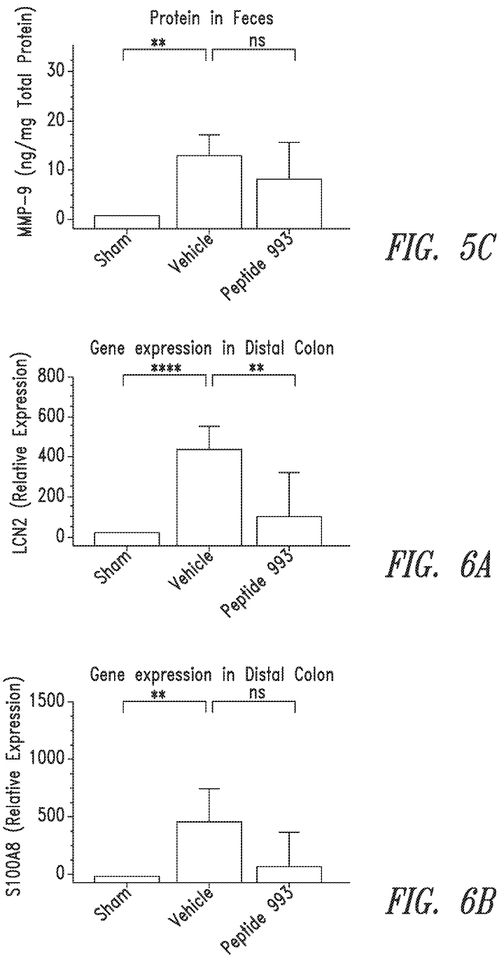

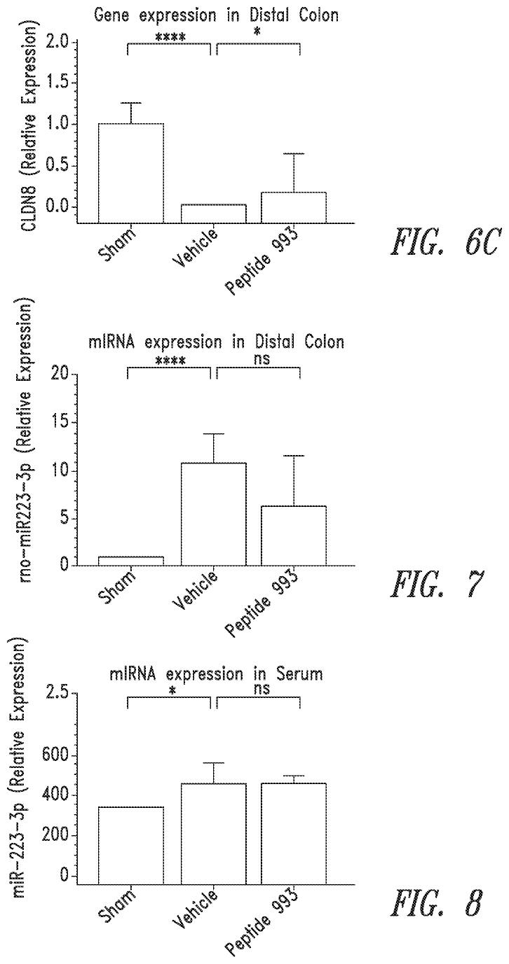

As shown in FIGS. 6A-6C, the relative expression of disease indicating genes LCN2, S100A8 and CLDN8 was statistically significantly altered in the vehicle-treated group. Treatment with Peptide 993 led to significant reduction or increase in the relative expression of LCN2 or CLDN8, respectively. The change in S100A8 expression in the peptide 993 treated was not statistically significant, however there was a marked reduction in the average expression of the group.

For extraction of microRNA from distal colon homogenate, the RNeasy Plus Mini Kit and the RNeasy MinElute Cleanup Kit (Qiagen) were used. For extraction of microRNA from serum, miRNeasy Serum/Plasma Kit (Qiagen) was used. To verify uniformity of microRNA extraction, a synthetic microRNA cel-miR-39-3p, at a fixed concentration, was spiked into both distal colon homogenates and serum samples. The microRNAs purified from distal colon and serum was used in the Advanced miRNA cDNA Synthesis Kit (Applied Biosystems) to obtain cDNA templates for the quantative real time PCR. The expression of miR-223-3p, a microRNA known to negatively regulate CLDN8 gene expression, was measured using TaqMan Advanced miRNA assay (Applied Biosystems). The endogenous control miR-16-5p was used for normalization in the distal colon, and the endogenous control miR-24-3p was used for normalization in the serum. Relative expression of miR-223-3p was calculated using the normalized Ct values. Statistical analysis of relative expression was performed using One-way ANOVA method (with Dunnett's multiple comparisons) to compare the variations Vs Group 2 animals.

As shown in FIG. 7, the relative expression of miR-223-3p in the distal colon showed statistically significant increase in the vehicle treated group. The change in miR-223-3p expression in the distal colon in the Peptide 993 treated group was not statistically significant, however there was a marked reduction in the average expression of the group. As shown in FIG. 8, the expression of miR-223-3p in the serum was statistically significantly increased in the vehicle treated group. There was no apparent change in miR-223-3p expression in the Peptide 993 treated group from serum samples.

### Example E4

### Correlation Between Efficacy and Levels of Disease Biomarkers Following Treatment with Peptide Inhibitors in Rat Models of Acute Colitis

Disease pathology and inflammatory markers were further examined in two rat models of acute colitis. The therapeutic potential of orally delivered Peptide 993 in a TNBS-induced rat model of IBD was demonstrated, and mechanism-specific and disease-related efficacy biomarkers in the TNBS-induced colitis model were profiled. In addition, target engagement biomarkers were examined in another rat model of colitis induced by dextran sulfate sodium salt (DSS).

In the first model, acute colitis was induced in Sprague-Dawley rats by a single intra-rectal instillation of TNBS followed by efficacy analysis at day seven. The animals were grouped into sham+vehicle (PO TID), TNBS+vehicle (PO TID), anti-IL23p19 antibody (4 mg/kg/day IP, Day −1 and Day 3), and Peptide 993 treatment at 9, 28, or 61 mg/kg/day (PO TID+in drinking water). Sham indicates animals not treated with TNBS.

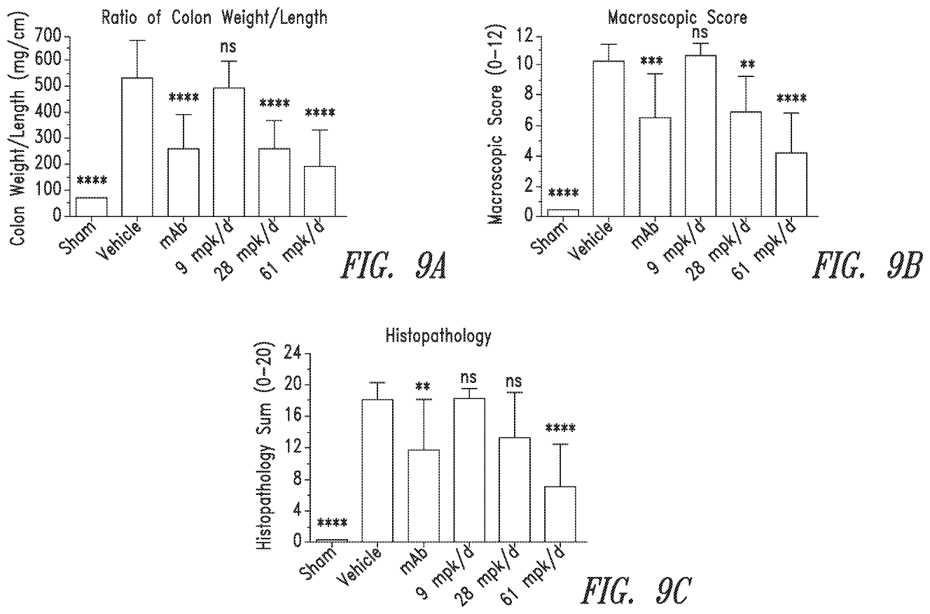

Following treatment, weight and length were determined from entire colon; colonic score was evaluated as sum of adhesion (0-2), stricture (0-3), ulcer (0-5), and colon wall thickness (0-2); histology was evaluated by a pathologist as sum of mucosal/submucosal inflammation (0-5), transmural inflammation (0-5), erosion (0-5), and gland loss (0-5). Values shown as mean±SD (FIGS. 9A-9C). Statistical significance was assessed by One-way ANOVA with post-hoc Dunnett's vs. Vehicle control: **p≤0.01; ***p≤0.001; ****p≤0.0001; ns, not significant. The data are provided in FIG. 9. These studies demonstrated that oral treatment with Peptide 993 resulted in significant and dose-dependent reduction in the colon weight-to-length ratio, and normalization in the macroscopic and histopathological changes in the colon.

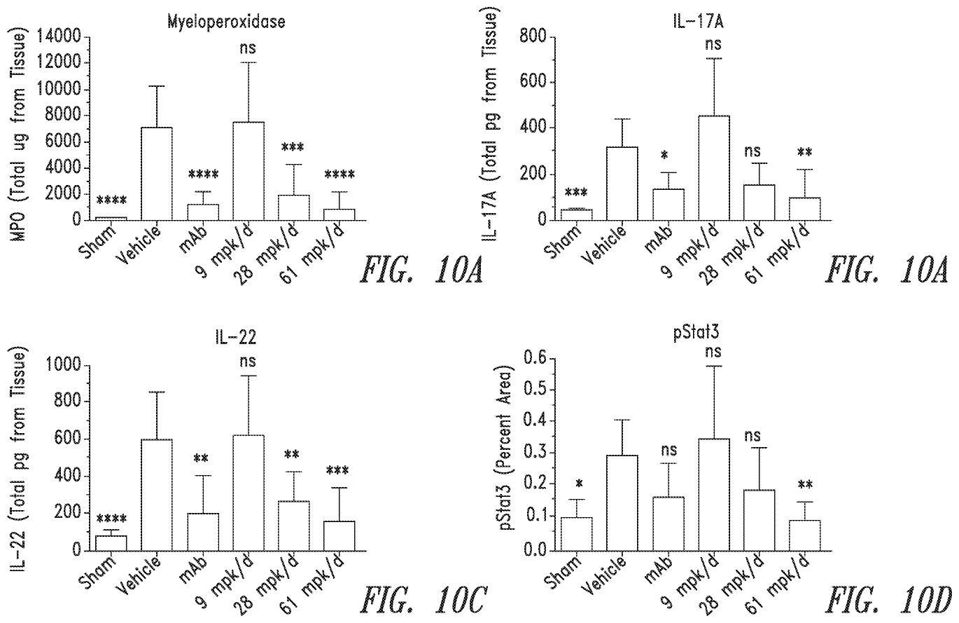

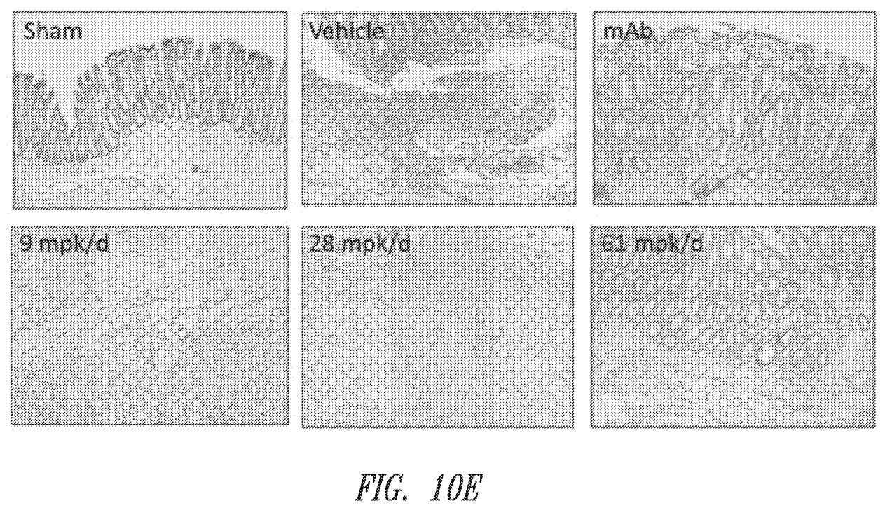

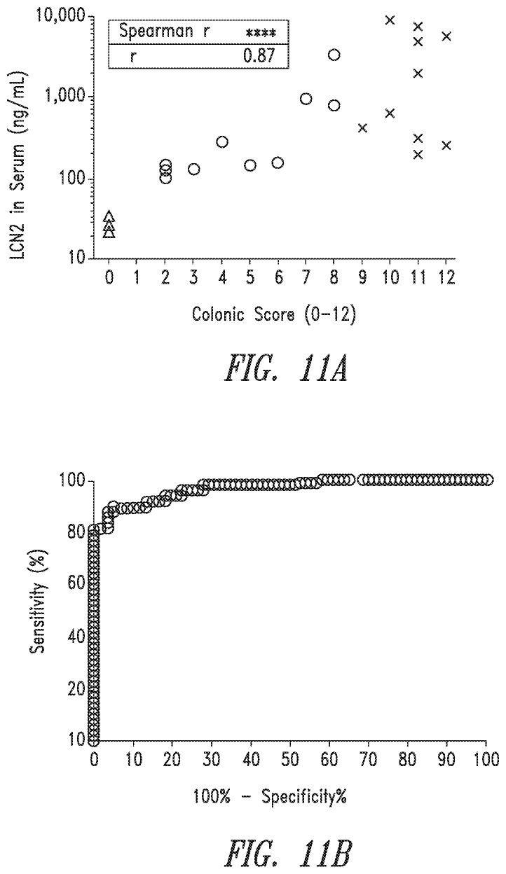

In addition, myeloperoxidase (MPO), IL-17A, and IL-22 detected from sampled tissue were quantified by enzyme-linked immunosorbent assay (ELISA), and the results are shown in FIGS. 10A-10C. The percentage of phosphorylated Signal Transducer and Activator of Transcription 3 (Stat3) was normalized to the area of the distal colon quantified by immuno-histochemistry (IHC) (FIG. 10D), and the micrographs shown in FIG. 10E are representative images from each indicated group for IHC analysis of pStat3 expression. Values shown as mean±SD. Statistical significance was assessed by One-way ANOVA with post-hoc Dunnett's vs. Vehicle control: *p≤0.05; **p≤0.01; ***p≤0.001; ****p≤0.0001; ns, not significant. These studies demonstrated that oral treatment with Peptide 993 resulted in significant and dose-dependent reduction of disease-related and IL-23-directed markers in the distal colons of TNBS-treated rats.

As shown in FIG. 11A, LCN2 serum levels were a good predictor of a reduction in local intestinal inflammation in response to treatment with Peptide 993, as there was a correlation between LCN2 expression levels in serum and colon score. This demonstrates that LCN2 serves as a good non-invasive efficacy marker of responsiveness to inhibitors of IL-23R. A ROC curve was generated based on multiple TNBS-induced colitis studies, based on a colonic score (0-12 scale for disease severity) cutoff of 5 (FIG. 11B). Animals with colonic scores of 1-5 were categorized as responders, and animals with colonic scores of 6-12 were categorized as non-responders. The area under the curve was calculated to be 0.97. Serum LCN2 at >294 ng/mL gave a sensitivity of 89% and a specificity of 95%, as shown in FIG. 11B.

In the second model, acute colitis was induced in Sprague-Dawley rats by ad libitum access to 5% DSS dissolved in drinking water every day until analysis at day five. The animals were grouped into sham+vehicle (PO TID), DSS+vehicle (PO TID), and DSS+Peptide 993 treatment at 61 mg/kg/day (PO TID+in drinking water).

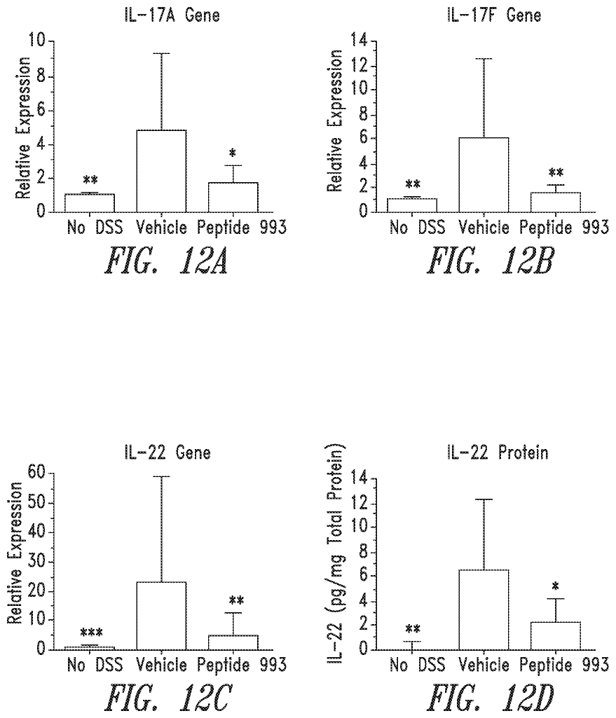

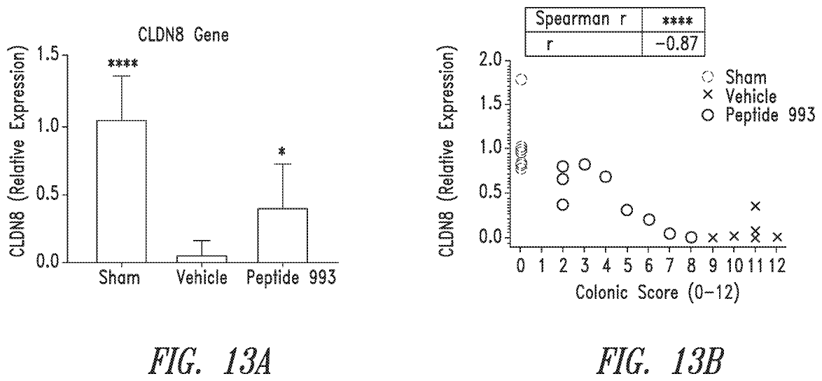

Expression of genes encoding IL-17A, IL-17F, and IL-22 was quantified by quantitative reverse transcription polymerase chain reaction (qRT-PCR), and production of IL-22 protein was quantified by ELISA. Relative expression was normalized to that of HPRT1; and values were determined as geometric mean±95% confidence interval. Protein concentration is shown as mean±SD. Statistical significance was assessed by One-way ANOVA with post-hoc Dunnett's vs. Vehicle control: *p≤0.05; **p≤0.01; ***p≤0.001; ns, not significant. The results of these assays are shown in FIGS. 12A-12D. These studies further show that oral treatment with Peptide 993 led to significant reduction in the mRNA and/or protein levels of IL-23-directed cytokines in the distal colons of DSS-treated rats. pSTAT3 analysis:

For the pSTAT3 assays, colon tissue samples were processed routinely, sectioned at approximately 3-5 microns, fixed in 10% neutral buffered formaldehyde, processed in paraffin, and immunolabeled with an anti-pSTAT3 primary antibody that binds STAT3 phosphorylated at Tyrosine 705 (Abcam, Cat. No. ab76315 PUR). pSTAT3 immunolabel as a percentage of total areas was measured using Visiopharm© image analysis software. Image analysis involved outlining the colon tissue as a region of interest (ROI), then an automated thresholding method was utilized for the classification of the pSTAT3 positive immunolabel. After classification, the total tissue area within the ROI was calculated, along with the count of pSTAT3 positive cells and the area (in microns) of the pSTAT3 positive cells. Lastly, the pSTAT3 immunolabel percentage of the total tissue area was calculated.

The details of the pSTAT3 Image Analysis Protocol were as follows:

1. ROI
a. Tissue was outlined, and non-colon tissues were excluded.

2. Pre-processing
a. Image was converted into grayscale-pixel values processed to be 0-255
b. Grayscale image was filtered into two channels:
   i. The DAB (or pSTAT3 positive) channel with pixels associated with the brown DAB staining; and
   ii. The hematoxylin channel with pixels associated with the tissue area not stained with DAB (includes all tissue area and background staining).

3. Analysis & Classification
a. Algorithm using a “threshold” method was used to classify the tissue area within the ROI.
   i. Threshold of the DAB channel was set to classify the positive pSTAT3 stained cells as any/all pixels (from the DAB channel of the pre-processed image) with values between 0-135. A green label was applied to these positively classified cells.
      1. Pixels with values outside the threshold of 0-135 were considered to be part of the tissue area (within the ROI).
b. Post-processing of the classified image (after the positive pSTAT3 cells were labeled) was performed to remove background staining.

4. Calculations
a. The total tissue area (within the ROI) was calculated-unit was in microns.
b. The total pSTAT3 positive area was calculated-unit is in microns.
c. The “count” of pSTAT3 positive cells was calculated.
d. The fraction of pSTAT3 positive cells was calculated by dividing the pSTAT3 positive area by the total tissue area-unit was in microns.
e. The nuclear density per square millimeter was calculated, simply to change the units from microns to square millimeter.

Additionally, the pSTAT3 area percentage was calculated, in the same manner as the pSTAT3 fraction, but displayed as a percentage of the total area.

### Conclusions

These studies further demonstrate the in vivo activity of the peptide inhibitors disclosed herein, including Peptide 993, and show (using two preclinical models of IBD) that the mechanism of action is mediated via the IL-23 pathway. In the acute TNBS-induced rat colitis model, blockade of IL-23R-mediated signaling by oral treatment with Peptide 993 led to significant and dose-dependent attenuation of disease parameters, with activity comparable to that of a neutralizing anti-IL-23p19 monoclonal antibody (mAb).

In the same TNBS-induced colitis model, oral treatment with Peptide 993 led to decreased colonic levels of MPO, an indicator of neutrophil infiltration and of the innate immune response. Importantly, the levels of IL-17A and IL-22, two cytokines in the IL-23 signaling pathway, were also significantly reduced. Furthermore, the levels of pStat3, a transcription factor known to be regulated by IL-23, are restored to control levels in response to Peptide 993 treatment. The dose-related responses in these markers track with Peptide 993 treatment effects.

In an acute DSS-induced rat colitis model, oral treatment with Peptide 993 led to significant decreases in the relative expression of genes encoding IL-23-directed cytokines (IL-17A, IL-17F, and IL-22), and in the abundance of IL-22 protein.

These results establish the value of these biomarkers in translating preclinical efficacy to early clinical proof-of-concept for anti-IL-23R therapy, including therapy with the peptide inhibitors described herein.

### Example E5

### Efficacy of CLDN8 as a Biomarker for Epithelial Integrity in the Colon and Correlation with Disease Severity

The claudin family of proteins are components of the tight junction complex. Through expression analysis and colonic scoring, claudins were analyzed as biomarkers for IBD disease severity. Specifically, CLDN8 (the claudin-8 gene) is highly expressed in normal tissues and down-regulated in the mucosa of IBD patients, and was chosen for further study in the TNBS-induced rat model of acute colitis. CLDN8 gene expression was analyzed in a TNBS-induced rat model of IBS following the protocols described in Example E1.

Treatment with the IL-23R antagonist Peptide 993 in subjects with diseased tissue restored levels of CLDN8 expression above vehicle control-treated subjects (FIG. 13A). Additionally, the treatment of diseased subjects with Peptide 993 reduced the colonic score (as determined following the protocol and scoring criteria for the TNBS-induced rat model of acute colitis described in Example E3) as compared to vehicle treated animals, indicating a restoration in the epithelial integrity in the colon of the treated subjects which correlated with heightened relative expression levels of CLDN8 (FIG. 13B).

What is claimed:

1. A method for determining or monitoring the efficacy of treatment of a subject having an inflammatory disease or disorder, optionally gastrointestinal inflammation, with a peptide inhibitor of interleukin-23 receptor (IL-23R), comprising:
(i) contacting a biological sample obtained from the subject during or after treatment with the IL-23R inhibitor with one or more reagent that binds one or more biomarker of inflammation, wherein one or more of the biomarkers are selected from the group consisting of: myeloperoxidase (MPO), interleukin-1β (IL-1f), interleukin-6 (IL-6), interleukin-22 (IL-22), interleukin-17A (IL-17A), interleukin-17F (IL-17F), lipocalin 2 (LCN2), matrix metallopeptidase 9 (MMP9), S100 calcium-binding protein A8 (S100A8), microRNA-223-3p (miR223-3p), and phosphorylated signal transducer and activator of transcription 3 (pSTAT3) proteins, polynucleotides encoding any of the proteins, and polynucleotides comprising a region complementary to microRNA-223-3p or any of the polynucleotides that encode any of the proteins; and
(ii) detecting the presence or absence of, or determining an amount of, the reagent bound to the biomarker, and thereby determining the level of the one or more biomarker in the biological sample, wherein if the level is reduced as compared to a pre-determined cut-off value or the level of the biomarker before treatment with the IL-23R inhibitor, it indicates that the treatment is efficacious, and wherein if the level is the same or increased as compared to a pre-determined cut-off value or the level of the biomarker before treatment with the IL-23R inhibitor, it indicates that the treatment is not efficacious.

2. A method for determining or monitoring the efficacy of treatment of a subject having an inflammatory disease or disorder, optionally gastrointestinal inflammation, with a peptide inhibitor of interleukin-23 receptor (IL-23R), comprising:
(i) contacting a biological sample obtained from the subject during or after treatment with the IL-23R inhibitor with one or more reagent that binds one or more biomarker of inflammation, wherein one or more of the biomarkers is a Claudin 8 (CLDN8) biomarker, optionally a protein, a polynucleotide that encodes the CLDN8 protein, or a polynucleotide comprising a region complementary to the polynucleotide that encodes the CLDN8 protein; and
(ii) detecting the presence or absence of, or determining an amount of, the reagent bound to the CLDN8 biomarker, and thereby determining the level of the CLDN8 biomarker in the biological sample, wherein if the level is increased as compared to a pre-determined cut-off value or the level of the CLDN8 biomarker before treatment with the IL-23R inhibitor, it indicates that the treatment is efficacious, and wherein if the level is the same or reduced as compared to a pre-determined cut-off value or the level of the CLDN8 biomarker before treatment with the IL-23R inhibitor, it indicates that the treatment is not efficacious.

3. The method of claim 1 or claim 2, wherein said biological sample is a gastrointestinal tissue sample, optionally a colon tissue sample, and one or more of the biomarker is selected from the group consisting of: LCN2, MPO, IL-1, IL-6, IL-17A, IL-17F, IL-22, pSTAT3, S100A8, CLDN8, and miR-223-3p.

4. The method of claim 1 or claim 2, wherein said biological sample is feces, and one or more biomarker is selected from the group consisting of: LCN2, MPO, and MMP9.

5. The method of claim 1 or claim 2, wherein said biological sample is a liquid matrix, wherein the liquid matrix is optionally serum, plasma, blood, or urine.

6. The method of claim 5, wherein the liquid matrix is serum, and one of the biological markers is LCN2.

7. The method of any of claims 1-6, wherein the biomarker is a polypeptide.

8. The method of claim 7, wherein said detecting is performed using an immunoassay, optionally selected from the group consisting of cloned enzyme donor immunoassay (CEDIA), turbidity assay, and competitive ELISA.

9. The method of any of claims 1-6, wherein the biomarker is a polynucleotide.

10. The method claim 9, wherein said detecting is performed by polymerase chain reaction (PCR), optionally quantitative reverse transcription-PCR (RT-PCR), or RNA sequencing.

11. The method of claim 10, wherein the time points occur within an about twelve week treatment regimen.

12. The method of any of claims 1-11, further comprising determining the levels of the one or more biomarkers before treatment.

13. A method of determining a level of one or more biomarker of intestinal inflammation in a serum sample, wherein one or more of the biomarkers is lipocalin 2 (LCN2), comprising:
(i) contacting the serum sample with a reagent that binds LCN2; and
(ii) detecting the presence or absence of, or determining an amount of, the reagent bound to the one or more biomarker, and thereby determining the level of the one or more biomarker in the biological sample.

14. A method of determining the presence of an inflammatory disease or disorder, optionally intestinal inflammation, in a subject, comprising:
(i) contacting a serum sample obtained from the subject with a reagent that binds lipocalin 2 (LCN2); and
(ii) detecting the presence or absence of, or determining an amount of, the reagent bound to the LCN2, and thereby determining the level of LCN2 in the serum sample, wherein if the level of LCN2 in the serum sample is greater than 294 ng/mL, intestinal inflammation is determined to be present in the subject with a sensitivity of 89% and a specificity of 95%.

15. The method of claim 14, wherein the subject was previously treated with a peptide inhibitor of an interleukin-23 receptor.

16. The method of claim 14 or claim 15, wherein said detecting is performed using an immunoassay, optionally selected from the group consisting of cloned enzyme donor immunoassay (CEDIA), turbidity assay, and competitive ELISA.

17. A method of determining a level of one or more biomarker of intestinal inflammation in a feces sample, wherein one or more of the biomarkers is lipocalin 2 (LCN2), myeloperoxidase (MPO), or matrix metallopeptidase 9 (MMP9), comprising:
(i) extracting proteins from the feces to produce extracted fecal proteins;
(ii) contacting the extracted fecal proteins with one or more reagent that binds the one or more biomarkers; and
(ii) detecting the presence or absence of, or determining an amount of, the reagent bound to the one or more biomarker, and thereby determining the level of the one or more biomarker in the feces sample.

18. A method of determining the presence of an inflammatory disease or disorder, optionally intestinal inflammation, in a subject, comprising:
(i) extracting proteins from a feces sample obtained from the subject to produce extracted fecal proteins;
(ii) contacting the extracted fecal proteins with one or more reagent that binds to one or more biomarkers of inflammation, wherein one of the biomarkers is lipocalin 2 (LCN2), myeloperoxidase (MPO), or matrix metallopeptidase 9 (MMP9); and
(ii) detecting the presence or absence of, or determining an amount of, the reagent bound to the one or more biomarkers, and thereby determining the level of the one or more biomarkers in the feces sample, wherein if the level of the one or more biomarkers is greater than a predetermined cut-off value or significantly greater than an average value obtained using feces obtained from healthy donors, intestinal inflammation is determined to be present in the subject.

19. The method of claim 18, wherein the subject was previously treated with a peptide inhibitor of an interleukin-23 receptor.

20. The method of claim 18 or claim 19, wherein said detecting is performed using an immunoassay, optionally selected from the group consisting of cloned enzyme donor immunoassay (CEDIA), turbidity assay, and competitive ELISA.

21. A method of treating an inflammatory disease or disorder in a subject in need thereof, comprising:
(i) providing to the subject an effective amount of a peptide inhibitor of an interleukin-23 receptor;
(ii) waiting for a first period of time; and
(iii) after the first period of time, contacting a biological sample obtained from the subject with one or more reagent that binds one or more biomarker of inflammation, wherein one or more of the biomarkers are selected from the group consisting of: myeloperoxidase (MPO), interleukin-1β (IL-1β), interleukin-6 (IL-6), interleukin-22 (IL-22), interleukin-17A (IL-17A), interleukin-17F (IL-17F), lipocalin 2 (LCN2), matrix metallopeptidase 9 (MMP9), S100 calcium-binding protein A8 (S100A8), microRNA-223-3p (miR223-3p), Claudin 8 (CLDN8), and phosphorylated signal transducer and activator of transcription 3 (pSTAT3) proteins, polynucleotides encoding any of the proteins, and polynucleotides comprising a region complementary to microRNA-223-3p or any of the polynucleotides that encode any of the proteins; and
(iv) detecting the presence or absence of, or determining an amount of, the reagent bound to the biomarker, and thereby determining the level of the one or more biomarker in the biological sample.

22. The method of claim 21, further comprising:
(iv) providing an additional amount of the peptide inhibitor, or an amount of the peptide inhibitor greater than the effective amount of step (i), to the subject after step (iv), if the determined level of the one or more biomarker is equal to or above a pre-determined cut-off value if the biomarker is MPO, IL-1(3, IL-6, IL-22, IL-17A, IL-17F, LCN2, MMP9, S100A8, miR223-3p, or pSTAT3, or equal to or below a pre-determined cut-off value if the biomarker is CLDN8; or
(v) not providing an additional amount of the peptide inhibitor, or providing an additional amount of the peptide inhibitor less than the effective amount of step (i) to the subject after step (iv), if the determined level of the biomarker is below a pre-determined cut-off value if the biomarker is MPO, IL-1(3, IL-6, IL-22, IL-17A, IL-17F, LCN2, MMP9, S100A8, miR223-3p, or pSTAT3, or above a pre-determined cut-off value if the biomarker is CLDN8.

23. The method of any of claims 1-12, 14, 16, 18, or 20-22, wherein the inflammatory disease or disorder is an Inflammatory Bowel Disease (IBD), ulcerative colitis, Crohn's disease, Celiac disease (nontropical Sprue), enteropathy associated with seronegative arthropathies, microscopic colitis, collagenous colitis, eosinophilic gastroenteritis, colitis associated with radio- or chemo-therapy, colitis associated with disorders of innate immunity as in leukocyte adhesion deficiency-1, chronic granulomatous disease, glycogen storage disease type 1b, Hermansky-Pudlak syndrome, Chediak-Higashi syndrome, and Wiskott-Aldrich Syndrome, pouchitis resulting after proctocolectomy and ileoanal anastomosis, gastrointestinal cancer, pancreatitis, insulin-dependent diabetes mellitus, mastitis, cholecystitis, cholangitis, pericholangitis, chronic bronchitis, chronic sinusitis, asthma, psoriasis, psoriatic arthritis, or graft versus host disease.

24. The method of any one of claims 1-12, 15, 16, or 19-22, wherein the peptide inhibitor has a structure or sequence of Formula (Xa), (I), (II), or (III), or a pharmaceutically acceptable salt thereof.

25. The method of claim 24, wherein the peptide inhibitor or pharmaceutically acceptable salt thereof comprises or consists of an amino acid sequence or structure as follows:
[Palm]-[isoGlu]-[PEG4]-[Pen]-NTWQ-[Pen]-[Phe[4-(2-aminoethoxy)]-[2-Nal]-[Aib]-[Lys(Ac)]-NNNH₂;
Ac-[Pen]-NTWQ-[Pen]-[Phe[4-(2-aminoethoxy)]-[2-Nal]-[Aib]-[Lys(PEG4-isoGlu-Palm)]-NN-NH₂;
Ac-[Pen]-QTWQ-[Pen]-Phe(4-CONH₂)-[2-Nal]-[α-MeLys(Ac)]-[Lys(Ac)]-NN-NH₂;
[Octanyl]-[IsoGlu]-[PEG4]-[Pen]-NTWQ-[Pen]-[Phe[4-(2-aminoethoxy)]-[2-Nal]-[Aib]-[Lys(Ac)]-NN-NH₂;
[Octanyl]-[PEG4]-[Pen]-NTWQ-[Pen]-[Phe[4-(2-aminoethoxy)]-[2-Nal]-[Aib]-[Lys(Ac)]-NN-NH₂;
[Palm]-[PEG4]-[Pen]-NTWQ-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-[Lys(PEG4-Octanyl)]-NN-NH₂;
Ac-[Pen]-NTWQ-[Pen]-[Phe[4-(2-aminoethoxy)]-[2-Nal]-[Aib]-[Lys(PEG4-Palm)]-NN-NH₂;
Ac-[Pen]-NTWQ-[Pen]-[Phe[4-(2-aminoethoxy)-(PEG4-Palm)]-[2-Nal]-[Aib]-[Lys(Ac)]NN-NH₂;
Ac-[Pen]-NTWQ-[Pen]-[Phe[4-(2-aminoethoxy)-(PEG4-Lauryl)]-[2-Nal]-[Aib]-[Lys(Ac)]-NN-NH₂;
Ac-[Pen]-QTWQ-[Pen]-Phe(4-CONH₂)-[2-Nal]-[α-MeLys(PEG4-Palm)-[Lys(Ac)]-NN-NH₂;
Ac-[Pen]-QTWQ-[Pen]-Phe(4-CONH₂)-[2-Nal]-[α-MeLys(PEG4-Lauryl)]-[Lys(Ac)]-NN-NH₂;
Ac-[Pen]-NTWQ-[Pen]-[Phe[4-(2-aminoethoxy)-(PEG4-IsoGlu-Palm)]-[2-Nal]-[Aib]-[Lys(Ac)]-NN-NH₂;
Ac-[Pen]-NTWQ-[Pen]-[Phe[4-(2-aminoethoxy)-(PEG4-IsoGLu-Lauryl)]-[2-Nal]-[Aib]-[Lys(Ac)]-NN-NH₂;
Ac-[Pen]-QTWQ-[Pen]-Phe(4-CONH₂)-[2-Nal]-[α-MeLys(PEG4-IsoGlu-Palm)]-[Lys(Ac)]-NN-NH₂;
Ac-[Pen]-QTWQ-[Pen]-Phe(4-CONH₂)-[2-Nal]-[α-MeLys(PEG4-IsoGlu-Lauryl)]-[Lys(Ac)]-NN-NH₂;
Ac-[Pen]-QTWQ-[Pen]-Phe(4-CONH₂)-[2-Nal]-[α-MeLys(IVA)]-[Lys(Ac)]-NN-NH₂;
Ac-[Pen]-QTWQ-[Pen]-Phe(4-CONH₂)-[2-Nal]-[α-MeLys(Biotin)]-[Lys(Ac)]-NN-NH₂;
Ac-[Pen]-QTWQ-[Pen]-Phe(4-CONH₂)-[2-Nal]-[α-MeLys(Octanyl)]-[Lys(Ac)]-NN-NH₂;
Ac-[Pen]-[Lys(IVA)]-TWQ-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-[Lys(Ac)]-[Lys(IVA)]-N-NH₂;
Ac-[Pen]-[Lys(Biotin)]-TWQ-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-[Lys(Ac)]-[Lys(Biotin)]-N-NH₂;
Ac-[Pen]-[Lys(Octanyl)]-TWQ-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-[Lys(Ac)]-[Lys(octanyl)]-N-NH₂;
Ac-[Pen]-[Lys(Palm)]-TWQ-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-[Lys(Ac)]-Lys(Palm)]-N-NH₂;
Ac-[Pen]-[Lys(PEG8)]-TWQ-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-[Lys(Ac)]-[Lys(PEG8)]-N-NH₂;
Ac-[Pen]-K(Peg11-Palm)TWQ-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-[Lys(Ac)]-[Lys(Peg11-palm)]-N-NH₂;
Ac-[Pen]-[Cit]-TW-[Cit]-[Pen]-[Phe[4-(2-aminoethoxy)]-[2-Nal]-[Aib]-[Lys(Ac)]-NN-NH₂;
Ac-[Pen]-[Lys(Ac)]-TW-[Cit]-[Pen]-[Phe[4-(2-aminoethoxy)]-[2-Nal]-[Aib]-[Lys(Ac)]-NN-NH₂;
Ac-[Pen]-NT-[Phe(3,4-OCH3)2]-Q-[Pen]-[Phe[4-(2-aminoethoxy)]-[2-Nal]-[Aib]-[Lys(Ac)]-NN-NH₂;
Ac-[Pen]-NT-[Phe(2,4-CH3)2]-Q-[Pen]-[Phe[4-(2-aminoethoxy)]-[2-Nal]-[Aib]-[Lys(Ac)]-NN-NH₂;
Ac-[Pen]-NT-[Phe(3-CH3)]-Q-[Pen]-[Phe[4-(2-aminoethoxy)]-[2-Nal]-[Aib]-[Lys(Ac)]-NN-NH₂;
Ac-[Pen]-NT-[Phe(4-CH3)]-Q-[Pen]-[Phe[4-(2-aminoethoxy)]-[2-Nal]-[Aib]-[Lys(Ac)]-NN-NH₂;
Ac-[(D)Arg]-[Pen]-NTWQ-[Pen]-[Phe[4-(2-aminoethoxy)]-[2-Nal]-[Aib]-[Lys(Ac)]-N-[βAla]-NH₂;
Ac-[(D)Tyr]-[Pen]-NTWQ-[Pen]-[Phe[4-(2-aminoethoxy)]-[2-Nal]-[Aib]-[Lys(Ac)]-N-[βAla]-NH₂;
Ac-[Pen]-NTWQ-[Pen]-[Phe[4-(2-aminoethoxy)]-[2-Nal]-[Aib]-[Lys(Ac)]-QN-NH₂;
Ac-[Pen]-NTWQ-[Pen]-[Phe[4-(2-aminoethoxy)]-[2-Nal]-[Aib]-[Lys(Ac)]-[Lys(Ac)]-N-NH₂;
Ac-[Pen]-NTWQ-[Pen]-[Phe[4-(2-aminoethoxy)]-[2-Nal]-[Aib]-[Lys(Ac)]-N-[Lys(Ac)]-NH₂;
Ac-[Pen]-NTWQ-[Pen]-[Phe[4-(2-aminoethoxy)]-[2-Nal]-[Aib]-[Lys(Ac)]-QQ-NH₂;
Ac-[Pen]-NTWQ-[Pen]-[Phe[4-(2-aminoethoxy)]-[2-Nal]-[Aib]-[Lys(Ac)]-Q-[βAla]-NH₂;
Ac-[Pen]-NTWQ-[Pen]-[Phe[4-(2-aminoethoxy)]-[2-Nal]-[Aib]-[Lys(Ac)]-N-[Cit]-NH₂;
Ac-[Pen]-NTWQ-[Pen]-[Phe[4-(2-aminoethoxy)]-[2-Nal]-[Aib]-[Lys(Ac)]-[Cit]-NH₂;
Ac-[Pen]-NTWQ-[Pen]-[Phe[4-(2-aminoethoxy)]-[2-Nal]-[Aib]-[Lys(Ac)]-[Cit]-Q-NH₂;
Ac-[Pen]-NTWQ-[Pen]-[Phe[4-(2-aminoethoxy)]-[2-Nal]-[Aib]-[Lys(Ac)]-[Cit]-[Lys(Ac)]-NH₂;
Ac-[Pen]-NTWQ-[Pen]-[Phe[4-(2-aminoethoxy)]-[2-Nal]-[Aib]-[Lys(Ac)]-[Lys(Ac)]-[Cit]-NH₂;
Ac-[Pen]-NTWQ-[Pen]-[Phe[4-(2-aminoethoxy)]-[2-Nal]-[Aib]-QN-[βAla]-NH₂;
Ac-[Pen]-NTWQ-[Pen]-[Phe[4-(2-aminoethoxy)]-[2-Nal]-[Aib]-E-[Cit]-Q-NH₂;
Ac-[Pen]-NTWQ-[Pen]-[Phe[4-(2-aminoethoxy)]-[2-Nal]-[Aib]-[Cit]-N-[Cit]-NH₂;
Ac-[Pen]-NTWQ-[Pen]-[Phe[4-(2-aminoethoxy)]-[2-Nal]-[Aib]-[Cit]-Q-[Cit]-NH₂;
Ac-[Pen]-[Cit]-TWQ-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-QNN-NH₂;
Ac-[Pen]-NTWQ-[Pen]-[Phe[4-(2-aminoethoxy)]-[2-Nal]-[Aib]-ENQ-NH₂;
Ac-[Pen]-GPWQ-[Pen]-[Phe[4-(2-aminoethoxy)]-[2-Nal]-[Aib]-[Lys(Ac)]-NN-NH₂;
Ac-[Pen]-PGWQ-[Pen]-[Phe[4-(2-aminoethoxy)]-[2-Nal]-[Aib]-[Lys(Ac)]-NN-NH₂;
Ac-[Pen]-NTWN-[Pen]-[Phe[4-(2-aminoethoxy)]-[2-Nal]-[Aib]-[Lys(Ac)]-NN-NH₂;
Ac-[Pen]-NSWQ-[Pen]-[Phe[4-(2-aminoethoxy)]-[2-Nal]-[Aib]-[Lys(Ac)]-NN-NH₂;
Ac-[Pen]-N-[Aib]-WQ-[Pen]-[Phe[4-(2-aminoethoxy)]-[2-Nal]-[Aib]-[Lys(Ac)]-NN-NH₂;
Ac-[Pen]-NTW-[Aib]-[Pen]-[Phe[4-(2-aminoethoxy)]-[2-Nal]-[Aib]-[Lys(Ac)]N-[Aib]-NH₂;
Ac-[Pen]-QTW-[Lys(Ac)]-[Pen]-[Phe[4-(2-aminoethoxy)]-[2-Nal]-[Aib]-[Lys(Ac)]-NN-NH₂;
Ac-[Pen]-[Lys(Ac)]-TWQ-[Pen]-[Phe[4-(2-aminoethoxy)]-[2-Nal]-[Aib]-[Lys(Ac)]NNNH₂;
Ac-[Pen]-QVWQ-[Pen]-[Phe[4-(2-aminoethoxy)]-[2-Nal]-[Aib]-[Lys(Ac)]-NN-NH₂;
Ac-[Pen]-NT-[2-Nal]-Q-[Pen]-[Phe[4-(2-aminoethoxy)]-[2-Nal]-[Aib]-[Lys(Ac)]-NN-NH₂;
Ac-[Pen]-NT-[1-Nal]-Q-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[α-MeLeu]-[Lys(Ac)]-NN-NH₂;
Ac-[Pen]-NTWQ-[Pen]-[Phe[4-(2-aminoethoxy)]-[2-Nal]-[α-MeLys]-[Lys(Ac)]-NN-NH₂;
Ac-[Pen]-NTWQ-[Pen]-[Phe[4-(2-aminoethoxy)]-[2-Nal]-[4-amino-4-carboxy-tetrahydropyran]-[Lys(Ac)]-NN-NH₂;
Ac-[Pen]-NTWQ-[Pen]-[Phe[4-(2-aminoethoxy)]-[2-Nal]-[α-MeLeu]-[Lys(Ac)]-N-[(Ala]-NH₂;
Ac-[Pen]-NTWQ-[Pen]-[Phe[4-(2-aminoethoxy)]-[2-Nal]-[α-MeLys]-[Lys(Ac)]-N-[βAla]-NH₂;
Ac-[Pen]-NTWQ-[Pen]-[Phe[4-(2-aminoethoxy)]-[2-Nal]-[4-amino-4-carboxy-tetrahydropyran]-[Lys(Ac)]-N-[βAla]-NH₂;
Ac-[Pen]-NTWQ-[Pen]-[Phe[4-(2-aminoethoxy)]-[2-Nal]-[Aib]-[Lys(Ac)]-LN-NH₂;
Ac-[Pen]-NTWQ-[Pen]-[Phe[4-(2-aminoethoxy)]-[2-Nal]-[Aib]-[Lys(Ac)]-GN-NH₂;
Ac-[Pen]-NTWQ-[Pen]-[Phe[4-(2-aminoethoxy)]-[2-Nal]-[Aib]-[Lys(Ac)]-SN-NH₂;
Ac-[Pen]-NTWQ-[Pen]-[Phe[4-(2-aminoethoxy)]-[2-Nal]-[Aib]-[Lys(Ac)]-[Aib]-N-NH₂;
Ac-[Pen]-NTWQ-[Pen]-[Phe[4-(2-aminoethoxy)]-[2-Nal]-[Aib]-[Lys(Ac)]-FN-NH₂;
Ac-[Pen]-NTW-[Cit]-[Pen]-[Phe[4-(2-aminoethoxy)]-[2-Nal]-[Aib]-[Lys(Ac)]-NN-NH₂;
Ac-[Pen]-NTWQ-[Pen]-[Phe[4-(2-aminoethoxy)]-[2-Nal]-[Aib]-[Lys(Ac)]-[Tic]-[βAla]-NH₂;
Ac-[Pen]-NTWQ-[Pen]-[Phe[4-(2-aminoethoxy)]-[2-Nal]-[Aib]-[Lys(Ac)]-[nLeu]-[βAla]-NH₂;
Ac-[Pen]-NTWQ-[Pen]-[Phe[4-(2-aminoethoxy)]-[2-Nal]-[Aib]-[Lys(Ac)]-G-[βAla]-NH₂;
Ac-[Pen]-NTWQ-[Pen]-[Phe[4-(2-aminoethoxy)]-[2-Nal]-[Aib]-[Lys(Ac)]-R-[βAla]-NH₂;
Ac-[Pen]-NTWQ-[Pen]-[Phe[4-(2-aminoethoxy)]-[2-Nal]-[Aib]-[Lys(Ac)]-W-[βAla]-NH₂;
Ac-[Pen]-NTWQ-[Pen]-[Phe[4-(2-aminoethoxy)]-[2-Nal]-[Aib]-[Lys(Ac)]-S-[Ala]-NH₂;
Ac-[Pen]-NTWQ-[Pen]-[Phe[4-(2-aminoethoxy)]-[2-Nal]-[Aib]-[Lys(Ac)]-L-[Ala]-NH₂;
Ac-[Pen]-NTWQ-[Pen]-[Phe[4-(2-aminoethoxy)]-[2-Nal]-[Aib]-[Lys(Ac)]-[AIB]-[(Ala]-NH₂;
Ac-[Pen]-NTWQ-[Pen]-[Phe[4-(2-aminoethoxy)]-[2-Nal]-[Aib]-[Lys(Ac)]-[N-MeAla]-[βAla]-NH₂;
Ac-[Pen]-NTWQ-[Pen]-[Phe[4-(2-aminoethoxy)]-[2-Nal]-[Aib]-[Lys(Ac)]-[2-Nap]-[(Ala]-NH₂;
Ac-[Pen]-NTWQ-[Pen]-[Phe[4-(2-aminoethoxy)]-[2-Nal]-[Aib]-[Lys(Ac)]-F-[βAla]-NH₂;
Ac-[(D)Arg]-[Pen]-NTWQ-[Pen]-[Phe[4-(2-aminoethoxy)]-[2-Nal]-[4-amino-4-carboxy-tetrahydropyran]-[Lys(Ac)]NNNH₂;
Biotin-[PEG4]-cyclo[[Abu]-QTWQC]-[Phe[4-(2-aminoethoxy)]-[2-Nal]-[4-amino-4-carboxy-tetrahydropyran]-ENN-NH₂;
Ac-cyclo[[Abu]-QTWQC]-[Phe[4-(2-aminoethoxy)]-[2-Nal]-[4-amino-4-carboxy-tetrahydropyran]-[Lys(Ac)]-NN-NH₂;
Ac-[(D)Arg]-cyclo[[Abu]-QTWQC]-[Phe[4-(2-aminoethoxy)]-[2-Nal]-[4-amino-4-carboxy-tetrahydropyran]-[Lys(Ac)]-NN-NH₂;
Ac-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-E-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-[(D)Asp]-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-R-[(D)Arg]-cyclo[[Abu]-QTWQC]-[Phe[4-(2-aminoethoxy)]-[2-Nal]-[4-amino-4-carboxy-tetrahydropyran]-ENN-NH₂;
inoethoxy)]-[2-Nal]-[4-amino-4-carboxy-tetrahydropyran]-ENN-NH₂;
Ac-F-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-[(D)Phe]-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-[2-Nal]-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-T-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-L-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-[(D)Gln]-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-[(D)Asn]-[(D)Arg]-cyclo[[Abu]-QTWQC]-[Phe[4-(2-aminoethoxy)]-[2-Nal]-[4-amino-4-carboxy-tetrahydropyran]-ENN-NH₂;
Ac-cyclo[[Abu]-QTWQC]-[Phe[4-(2-aminoethoxy)-(PEG4-Alexa488)]-[2-Nal]-[4-amino-4-carboxy-tetrahydropyran]-ENN-NH₂;
[Alexa488]-[PEG4]-cyclo[[Abu]-QTWQC]-[Phe[4-(2-aminoethoxy)]-[2-Nal]-[4-amino-4-carboxy-tetrahydropyran]-ENN-NH₂;
[Alexa647]-[PEG4]-cyclo[[Abu]-QTWQC]-[Phe[4-(2-aminoethoxy)]-[2-Nal]-[4-amino-4-carboxy-tetrahydropyran]-ENN-NH₂;
[Alexa-647]-[PEG4]-[(D)Arg]-cyclo[[Abu]-QTWQC]-[Phe[4-(2-aminoethoxy)]-[2-Nal]-[4-amino-4-carboxy-tetrahydropyran]-[Lys(Ac)]-NN-NH₂;
[Alexa647]-[PEG12]-[(D)Arg]-cyclo[[Abu]-QTWQC]-[Phe[4-(2-aminoethoxy)]-[2-Nal]-[4-amino-4-carboxy-tetrahydropyran]-[Lys(Ac)]-NN-NH₂; and
[Alexa488]-[PEG4]-[(D)Arg]-cyclo[[Abu]-QTWQC]-[Phe[4-(2-aminoethoxy)]-[2-Nal]-[4-amino-4-carboxy-tetrahydropyran]-[Lys(Ac)]-NN-NH₂,
wherein the peptide inhibitor is cyclized via a disulfide bond between the two Pen residues or by a thioether bond between the Abu and the Cys or Pen residue, and wherein the peptide inhibitor inhibits the binding of an interleukin-23 (IL-23) to an IL-23 receptor.
